(19) United States
(12) Patent Application Publication
(10) Pub. No.: US 2024/0336897 A1
(43) Pub. Date: Oct. 10, 2024

(54) COMPOSITIONS AND METHODS FOR CELL REPROGRAMMING

(71) Applicant: CORNELL UNIVERSITY, Ithaca, NY (US)

(72) Inventors: Vimal Selvaraj, Ithaca, NY (US); Viju Pillai, West Lafayette, IN (US)

(21) Appl. No.: 18/687,608

(22) PCT Filed: Aug. 26, 2022
(86) PCT No.: PCT/US2022/075503

§ 371 Date: Feb. 28, 2024

Related U.S. Application Data
(60) Provisional application No. 63/238,155, filed on Aug. 28, 2021.

(51) Int. Cl. C12N 5/074

(52) U.S. Cl. CPC C12N 5/0696; C12N 2501/15; C12N 2501/16; C12N 2501/2306; C12N 2501/235; C12N 2501/727; C12N 2510/00; C12N 2533/90

(57) ABSTRACT

Described in several exemplary embodiments herein are compositions and methods for reprogramming cells, such as somatic cells. Also described in several exemplary embodiments herein are induced pluripotent stem cells (iPSCs), particularly ruminant iPSCs.

### CROSS-REFERENCE TO RELATED APPLICATIONS

This application claims the benefit of and priority to U.S. Provisional Patent Application No. 63/238,155, filed on Aug. 28, 2021, entitled “Compositions and Methods for Cell Reprogramming,” the contents of which is incorporated by reference herein in its entirety.

### STATEMENT REGARDING FEDERALLY SPONSORED RESEARCH

This invention was made with government support under Grant No. USDA-NIFA 2013-00986 awarded by United States Department of Agriculture. The government has certain rights in the invention.

### SEQUENCE LISTING

This application contains a sequence listing filed in electronic form as an .XML file entitled CORNL-0710WP_ST26.xml created on Aug. 18, 2022 and having a size of 5,869 bytes. The content of the sequence listing is incorporated herein in its entirety.

### TECHNICAL FIELD

The subject matter disclosed herein is generally directed to compositions and methods for cell reprogramming and induced pluripotent stem cells.

### BACKGROUND

The discovery of induced pluripotent stem cells (iPSCs) that had characteristics identical to ESCs (Takahashi and Yamanaka, 2006; Takahashi et al., 2007), infused new enthusiasm for bovine pluripotency research. In the ensuing decade, numerous attempts to generate biPSCs have been made, incorporating the four core reprogramming genes POU5F1 (OCT4), SOX2, KLF4 and MYC (OSKM), and other factors (Bai et al., 2016; Canizo et al., 2018; Cao et al., 2012; Cravero et al., 2015; Han et al., 2011; Heo et al., 2015; Huang et al., 2011; Kawaguchi et al., 2015; Lin et al., 2014; Pillai et al., 2019b; Sumer et al., 2011; Talluri et al., 2015; Wang et al., 2013; Zhao et al., 2017), but without success. Thus, compositions, methods, and techniques are needed at least for the generation of bovine pluripotent stem cells and further compositions, methods and techniques for preparing and/or maintaining pluripotent stem cells such that they can be used in various applications.

Citation or identification of any document in this application is not an admission that such a document is available as prior art to the present invention.

### SUMMARY

Described in certain exemplary embodiments herein is a stem cell culture medium comprising an amount of a pluripotency composition comprising a glycogen synthase kinase 3 beta (GSK3beta) inhibitor; a mitogen-activated protein kinase kinase (MEK 1/2) inhibitor; and a transforming growth factor beta (TGF beta)/activin/nodal pathway inhibitor, wherein the pluripotency composition is effective to maintain pluripotency and/or inhibit and/or prevent differentiation of a cell.

In some exemplary embodiments, the cell comprises a reprogrammed and/or a pluripotent stem cell signature and/or program. In some exemplary embodiments, the reprogrammed and/or a pluripotent stem cell program comprises a TGFbeta signaling program. In some exemplary embodiments, the reprogrammed and/or a pluripotent stem signature comprises: TGFbeta receptor 1, TGFbeta receptor 2, or both.

In some exemplary embodiments, the cell comprises a 16-cell embryo signature and/or program.

In some exemplary embodiments, cell does not comprise a trophectoderm cell signature and/or program; a trophoblast stem cell signature and/or program; a trophoblast signature and/or program; an endoderm cell signature and/or program; a mesoderm cell signature and/or program; an ectoderm cell signature and/or program; a differentiated cell signature or program; or any combination thereof.

In some exemplary embodiments, the cell is not a trophectoderm cell, a trophoblast stem cell, a trophoblast, an endoderm cell, a mesoderm cell, or a differentiated cell.

In some exemplary embodiments, the cell is a pluripotent stem cell, optionally an induced pluripotent stem cell.

In some exemplary embodiments, the cell is a reprogrammed cell, optionally an induced pluripotent stem cell, that was reprogrammed by expression of Oct4, Sox2, Klf4, Myc, SV40 Large T antigen or any combination thereof, optionally Oct4, Sox2, Klf4, Myc, and SV40 Large T antigen.

In some exemplary embodiments, the cell is a non-human mammalian cell.

In some exemplary embodiments, the cell is a ruminant cell.

In some exemplary embodiments, the cell is a bovine cell, ovine cell, caprine cell, a cervine cell, a giraffe cell, or a camel cell.

In some exemplary embodiments, the cell is a murine cell, an equine cell, a feline cell, or a canine cell.

In some exemplary embodiments, the cell expresses or has expressed SV40 Large T antigen, Oct4, Sox2, Klf4, Myc, or any combination thereof. In some exemplary embodiments, the cell expresses or has expressed SV40 Large T antigen, Oct4, Sox2, Klf4, and Myc.

In some exemplary embodiments, the stem cell media further comprises an amount of leukemia inhibitory factor (LIF), an amount of interleukin (IL-6), or both.

In some exemplary embodiments, the cell culture medium comprises DMEM, MEM, Essential 6 medium, Neurobasal™, mTesR™, Leibovitz L-15, McCoy's 5A, a suitable whole or partial cell culture medium, or any combination thereof.

In some exemplary embodiments, the cell culture medium comprises Ham's F12 nutrient composition, Ham's F10 nutrient composition, serum, Knockout serum replacement, N2 supplement, B-27 supplement, nutrient supplement (e.g., an amino acid(s), a vitamin(s), mineral(s), and/or the like), one or more anti-infectives (e.g., one or more antibiotics, one or more antifungals and/or the like), a reducing agent, a pH indicator, a buffer, or any combination thereof.

In some exemplary embodiments, the GSK3beta inhibitor is selected from: a small molecule inhibitor, an antibody or fragment thereof, an aptamer, an RNAi molecule, a polypeptide ligand or antagonist, or any combination thereof.

In some exemplary embodiments, the GSK3beta inhibitor is selected from: CHIR99021, SB-216763, GSK-3 inhibitor IX, kenpaullone or an analog thereof, lithium chloride, GSK-3beta inhibitor XII, GSK-3beta inhibitor VII, GSK-3inhibitor XVI, 10Z-hymenialdsine, indirubin, CHIR-98014, GSK-3beta inhibitor VI, indirubin-3′-monoxime, GSK-3 inhibitor X, SB-415268, TWS 119 ditrifluoroacetate, 5-iodo-indirubin-3′-monoxime, GSK-3beta inhibitor I, indirubin-5-sulfonic acid sodium salt, hymenidin, 3F8, Bisindlylmaleimide X HCl, indirubin-3′-monoxime-5-sulphonic acid, GSK-3 inhibitor II, GSK-3beta inhibitor VIII, GSK-3beta inhibitor XI, TCS 2002, alsterpaullone 2-cyanoethyl, A 1070722, MeBIO, AR-AO 14418-d3, 6-N-[2-[[4-(2,4-dichlorophenyl)-5-(1H-imidazol-2-yl)pyrimidin-2-yl]amino]ethyl]-3-nitropyridine-2,6-diamine, or any combination thereof.

In some exemplary embodiments, the MEK 1/2 inhibitor is selected from: a small molecule inhibitor, an antibody or fragment thereof, an aptamer, an RNAi molecule, a polypeptide ligand or antagonist, or any combination thereof.

In some exemplary embodiments, the MEK 1/2 inhibitor is selected from: PD0325901, AS703988/MSC2015103B, PD184352, Selumetinib, MEK162, AZD8330, TAK-733, GDC-0623, Refametinib, RO4987655, WX-554, HL-085, Cobimetinib, Trametinib, binimetinib, Pimasertib, Mirdametinib, E6201, CH5126766, SHR7390, TQ-B3234, CS-3006, FCN-159, RO5126766, or any combination thereof.

In some exemplary embodiments, the TGF beta/activin/nodal pathway inhibitor is selected from: a small molecule inhibitor, an antibody or fragment thereof, an aptamer, an RNAi molecule, a polypeptide ligand or antagonist, or any combination thereof.

In some exemplary embodiments, the TGF beta/activin/nodal pathway inhibitor is selected from: A83-01, SB431542, SB505124, LY2157299 (Galunisertib), LY550410, fresolimumab, AP12009, AP1104/AP15012, Lucanix™, ISTH0036, GC1008, 2G7, 1D11, LY2382770, CAT-192, Ki 26894, SD208, LY2109761, IN-1130, LY2157299, TEW-7197, PF-03446962, Gemogenovatucel-T, Trx-xFoxH1b aptamer, an inhibitor of myosins (e.g., pentachloropseudilin (PClP) and pentabromopseudilin (PBrP)), sorafenib, 6.3G9 antibody, 264RAD, lerdelimumab, soluble TbetaRII/III, 5,6-dihydro-4H-pyrrolo[1,2-b]pyrazole and analogs thereof, PF-03446962, KRC203, KRC360, LDN193189, ACE-041/Dalantercept, SB-525334, LY-364947, GW-6604, SD-208, or any combination thereof.

In some exemplary embodiments, stem cell culture medium does not comprise a growth factor.

Described in certain exemplary embodiments herein are methods of maintaining pluripotency of and/or inhibiting or preventing differentiation of a cell or cell population, the method comprising culturing a cell or cell population in the stem cell culture medium as described above and elsewhere herein.

In some exemplary embodiments, the cell comprises a reprogrammed cell and/or a pluripotent stem cell signature and/or program. In some exemplary embodiments, the reprogrammed cell and/or pluripotent stem cell program comprises a TGFbeta signaling program. In some exemplary embodiments, the reprogrammed cell and/or pluripotent stem cell signature comprises TGFbeta receptor 1, TGFbeta receptor 2, or both.

In some exemplary embodiments, the cell comprises a 16-cell embryo signature and/or program.

In some exemplary embodiments, the cell does not comprise a trophectoderm cell signature and/or program; a trophoblast stem cell signature and/or program; a trophoblast signature and/or program; an endoderm cell signature and/or program; a mesoderm cell signature and/or program; an ectoderm cell signature and/or program; a differentiated cell signature and/or program; or any combination thereof.

In some exemplary embodiments, the cell expresses or has expressed SV40 Large T antigen; Oct4; Sox2; Klf4; Myc; or any combination thereof. In some exemplary embodiments, cell expresses or has expressed SV40 Large T antigen, Oct4, Sox2, Klf4, and Myc.

In some exemplary embodiments, the cell is a non-human mammalian cell.

In some exemplary embodiments, the cell is a ruminant cell.

In some exemplary embodiments, the cell is a bovine cell, an ovine cell, a caprine cell, a cervine cell, a giraffe cell, or a camel cell.

In some exemplary embodiments, the cell is a murine cell, an equine cell, a feline cell, or a cell.

In some exemplary embodiments, the cell is a pluripotent stem cell, optionally an induced pluripotent stem cell.

In some exemplary embodiments, the cell is a reprogrammed and/or induced pluripotent stem cell that was reprogrammed by expression of Oct4, Sox2, Klf4, Myc, SV40 Large T antigen, or any combination thereof, optionally Oct4, Sox2, Klf4, Myc, and SV40 Large T antigen.

In some exemplary embodiments, culturing comprises passaging the cells one or more times. In some exemplary embodiments, culturing does not comprise passaging. In some exemplary embodiments, comprises culturing cells on feeder layer. In some exemplary embodiments, culturing does not include culturing cells on feeder layer. In some exemplary embodiments, culturing comprises culturing cells on a 3D matrix. In some exemplary embodiments, culturing comprises culturing the cells in suspension. In some exemplary embodiments, wherein culturing comprises culturing the cells adherently on a cell culture surface.

Described in certain example embodiments herein are methods of generating reprogrammed cells, optionally induced pluripotent stem cells, the method comprising reprogramming a somatic cell; and culturing the reprogrammed somatic cell using a method of maintaining pluripotency of and/or inhibiting or preventing differentiation of a cell or cell population described above and elsewhere herein. In some embodiments the somatic cell is a blood cell or a cell within blood.

In certain example embodiments, reprogramming comprises expressing SV40 Large T antigen, Oct4, Sox2, Klf4, Myc, or any combination thereof, optionally SV40 Large T antigen, Oct4, Sox2, Klf4, and Myc, in the somatic cell.

In certain example embodiments, the somatic cell is a non-human somatic cell.

In certain example embodiments, the somatic cell is a ruminant somatic cell.

In certain example embodiments, the somatic cell is a bovine somatic cell, an ovine somatic cell, a caprine somatic cell, a cervine somatic cell, a giraffe somatic cell, or a camel somatic cell.

In certain example embodiments, the somatic cell is a murine somatic cell, an equine somatic cell, a feline somatic cell, or a canine somatic cell.

Described in certain exemplary embodiments herein are methods comprising differentiating a cell generated by a method of generating reprogrammed cells, optionally induced pluripotent stem cells described above and elsewhere herein and/or cultured by a method of maintaining pluripotency of and/or inhibiting or preventing differentiation of a cell or cell population described above and elsewhere herein; and/or modifying the cell.

In certain example embodiments, modifying comprises genetic or genomic modification, RNA modification, or both.

Described in certain exemplary embodiments herein are cells produced by performing a method of maintaining pluripotency of and/or inhibiting or preventing differentiation of a cell or cell population, a method of generating reprogrammed cells, optionally induced pluripotent stem cells; methods of differentiating and/or modifying cells; or any combination thereof described above or elsewhere herein. In certain example embodiments, the cell is a reprogrammed cell, a pluripotent stem cell, an induced pluripotent stem cell, or a differentiated cell and/or a modified cell.

Described in certain exemplary embodiments herein are differentiated and/or modified cells produced by performing a method described above or elsewhere herein.

Described in certain exemplary embodiments engineered cells comprising a bovine induced pluripotent stem cell signature and/or program, wherein the bovine induced pluripotent stem cell signature and/or program comprises one or more genes and/or programs as set forth in one or more of FIGS. 2A-2E, 3B-3C, 3F-3G, 6, 7A-7D, 8A-8C and Tables 2-5.

In certain example embodiments, the engineered cell is a genetically edited or otherwise modified cell.

In certain example embodiments, the engineered cell is a non-human mammal cell.

In certain example embodiments, the engineered cell is a ruminant cell.

In certain example embodiments, the engineered cell is a bovine cell, ovine cell, caprine cell, a cervine cell, a giraffe cell, or a camel cell.

In certain example embodiments, the engineered cell is a murine cell, an equine cell, a feline cell, or a canine cell.

In certain example embodiments, the engineered cell is a pluripotent stem cell.

In certain example embodiments, the engineered cell is an induced pluripotent stem cell.

In certain example embodiments, the engineered cell expresses or has expressed SV40 large T antigen, Oct4, Sox2, Klf4, and Myc.

In certain example embodiments, the engineered cell has been cultured in a stem cell culture media as described above and elsewhere herein and/or cultured and/or generated via a method as described above and elsewhere herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—Use of OSKM+LT dramatically enhances the efficiency of inducing biPSCs. (FIG. 1A) Reprogramming method timeline showing procedures and culture conditions. (FIG. 1B) Representative images of alkaline phosphatase (ALP) stained plate from reprogramming experiments at Day 25 showing cultures of OSKM, OSKM+N and OSKM+LT conditions at 25 days. No colonies were recorded for the condition with just OSKM. Quantitative analysis show confirmed significant increase in reprogramming efficiency with OSKM-LT (p<0.05). Morphological examination of changes during the reprogramming timeline distinctively showed that OSKM+LT resulted in numerous compact colonies that contain rounded edges with individual cells not discernible. This is in contrast to the dense accumulation without rounded edges observed for OSKM+N. (FIG. 1C) Representative images showing ALP staining before and after passaging OSKM+LT-derived colonies in SC medium. Even after the first passage, colony morphology shifted to flattened colonies with demarcated cells and decrease in intensity of ALP staining. Colonies could not be maintained beyond the third passage.

FIG. 2A-2E—Functional analysis of signal transduction in the bovine inner cell mass. (FIG. 2A) Deducting transcriptome expression of bovine trophoblast stem cells/TSCs from Day 8 blastocysts to enrich for genes expressed in the inner cell mass. Of the 14,667 genes in the transcriptome, 2,394 genes were barcoded as upregulated in the bovine blastocyst (log 10 fold-change >1.0, and P>0.05) and were separated for subsequent signal transduction analysis. (FIG. 2B) Heatmap showing that expression of conserved transcripts associated with pluripotency across different species were among these 2,394 genes selected from the blastocyst transcriptome. (FIG. 2C) StemChecker identified transcriptional regulatory programs in the 2,394 genes that were associated with and significant for embryonal carcinoma cells [EC cells], iPSCs and ESCs. (FIG. 2D) Analysis of kinase-based signaling in the 2,394 upregulated genes indicated that TGFβ-mediated signaling was actively suppressed in the bovine inner cell mass. (FIG. 2D) Examination of transcripts in bovine trophoblast stem cells/TSCs showed that TGFB1, TGFB2, and TGFB3 are expressed, and could be part of the blastocoel fluid. (FIG. 2E) Together with TGFβ, TSCs also expressed high levels of latent TGFβ binding proteins (LTBPs), providing indirect evidence that TGFβ levels might be highly regulated around the developing inner cell mass.

FIG. 3A-3H—Inhibition TGFβ/activin/nodal in addition to GSK3β, MEK1/2 supports pluripotent self-renewal and long-term culture biPSCs. (FIG. 3A) Representative images of biPSCs passaged in GMTi medium over an extended period of time. Colony morphology aligned with naïve PSCs as described for mice and humans and was strictly preserved in GMTi medium without any change indicative of differentiation. (FIG. 3B) Reprogrammed biPSCs were consistently SSEA1 positive across different passages [Passage 20 shown]. (FIG. 3C) RT-PCR analysis of OSKM transgenes showed that expression was sustained in early passages [Passage 2] and silenced in later passages [Passage 10 and 20]. (FIG. 3D) RT-PCR analysis of LT gene expression in biPSC [passage 20] also showed complete silencing. (FIG. 3E) Representative chromosomal spreads from individual biPSCs from passage 22 showing 60 chromosomes. (FIG. 3F) Gene expression profiling of biPSCs [performed at Passage 8] indicated endogenous activation of pluripotency-associated genes compared to expression seen in primary trophoblast stem cells/TSCs. (FIG. 3G) Expression levels of genes defined in studies on other species as associated with naïve (L1) or primed (L2) pluripotency, compared between 16-CE and biPSCs, indicated a mostly similar pattern of expression. (FIG. 3H) Genes associated with trophectoderm [TE], mixed lineages [ML], endoderm, mesoderm and ectoderm were low to no expression in biPSCs indicating an undifferentiated state. TE-restricted gene expression was observed only in TSCs. Full gene list used for this panel is included in Table 3.

FIG. 4A-4B—Indispensable roles for GSK3β, MEK1/2, and TGFβ/activin/nodal inhibitors in biPSC pluripotency sustenance. (FIG. 4A) Use of GMTi medium allowed culture of biPSCs on different cell-free surfaces such as gelatin and Matrigel®, without any effect to pluripotency sustenance. Representative images and quantification of size indicative of self-renewal and expansion of biPSCs over a period of 4 days. Rapid proliferation could be documented by average colony size increase of 10-16-fold over a period of 4 days. (FIG. 4B) Including all three: GSK3β inhibitor (CHIR99021), MEK1/2 inhibitor (PD0325901) and TGFβ/activin/nodal inhibitor (A83-01) is essential for sustaining the biPSC morphology and pluripotency. Eliminating one of the above three inhibitors in biPSCs cultures resulted in differentiation. Representative images showing the effect of removing CHIR99021, PD0325901 or A83-01 in biPSC cultures; detrimental effects to colony morphology consistent with differentiation can be noted within 48 hours with removal.

FIG. 5A-5B—Pluripotent biPSCs readily differentiate to cells of the three germ layers. (FIG. 5A) Embryoid bodies could be generated from biPSC cultures with removal of inhibitors and supplementing serum. Representative images show embryoid bodies from 5 days of culture; outgrowths from these embryoid bodies when plated in gelatin-coated dishes presented a diversity of differentiated cell with distinctive morphologies. (FIG. 5B) Subcutaneous introduction of biPSCs in immunodeficient NSG mice resulted in teratoma formation, with significant growth observed by 6 weeks. Teratomas collected measured more than 1 cm in rough diameter. Representative images of hematoxylin and eosin stained histological sections of teratomas showing differentiation of biPSCs into the three different germ layers. Four panels show different regions within teratoma sections with insets pointing to: [1] Bone tissue (mesoderm), [2] Neural tube/crest (ectoderm), [3] Hyaline cartilage (mesoderm), [4] Ciliated respiratory epithelium (endoderm), [5] Cardiomyocytes (mesoderm), and [6] Adipocytes (mesoderm).

FIG. 6—Core transcription factors contributing to the phenotypic homeostasis of the bovid pluripotent state. Systems biology investigation into active transcription enrichment that accounts for the majority of gene expression in 16-cell stage embryos (16-CEs) and biPSCs indicated a consistent repertoire of significant factors between these two groups representing the bovid pluripotent state. Data represents expression levels for the identified transcription factors in 16-CEs and biPSCs (as counts per million/CPM, and fold-change compared to fibroblasts), the strength of association (odds ratio/OR), and the percent targets detected in the analysis.

FIG. 7A-7D—Transcription regulator network associated with sustenance of bovid pluripotency. (FIG. 7A) Comparison of transcription factors upregulated in the 16-cell stage embryos (16-CEs) and biPSCs (fold-change compared to fibroblasts), identified 312 common regulators. (FIG. 7B) Plotting the correlation of fold-change across these common regulators between biPSCs and 16-CEs revealed 77 factors as highly expressed in both groups (quadrant II, with a >3 fold-change upregulation cutoff). (FIG. 7C) Expression levels (as CPM) for the 77 factors identified in quadrant II. (FIG. 7D) Constructing an interaction network map of transcriptional regulators common for 16-CE and biPSCs disclosed the known regulatory mechanisms associated with the identified genes.

FIG. 8A-8C—Integrative expression-interaction analysis predicts cell surface receptors that regulate pluripotency signaling in bovids. (FIG. 8A) Analysis of gene ontology under molecular function indicated common systems between 16-cell stage embryos (16-CEs) and biPSCs in upregulated genes; comparisons to fibroblasts were evaluated independently and significant elements merged for 16-CE and biPSCs. (FIG. 8B) Integrative analysis combining gene expression and protein-protein interactions predicted 11 common upstream membrane receptors that represent signaling in 16-CE and biPSCs. (FIG. 8C) Expression levels for these 11 identified membrane receptors were highly expressed in both 16-CE and biPSCs. Heatmaps show expression as counts per million/CPM, and fold-change compared to fibroblasts.

FIG. 9—Murine induced pluripotent stem cells can be generated and cultured with the GMTi medium. These cells also exhibit robust morphological characteristics of pluripotency. They sustain these characteristics even under feeder-free conditions such as surface coating with gelatin and Matrigel.

FIG. 10—Murine iPSCs generated and sustained using GMTi medium could contribute robustly to both embryonic and extraembryonic chimeras. Stable expression of nuclear GFP and membrane-mCherry transgenes in murine iPSCs generated and cultured using GMTi medium. Images show miPSC colonies with GFP and mCherry fluorescence—dual transgene labeling. Injection of labeled miPSCs into murine blastocyst embryos resulted in incorporation of cells into both embryonic and extraembryonic (placental) tissues. GFP and mCherry transgene expression can be seen in both the embryo and placenta (asterisk; a dotted line has been used to demarcate bulk of the extraembryonic tissue).

FIG. 11A-11C—Derivation of adult blood-derived fibrocytes and reprogramming to induced pluripotent stem cells (iPSCs). (FIG. 11A) Blood sample collected aseptically can be used to grow fibrocytes. (FIG. 11B) Representative images showing attachment and growth of fibrocytes from a sheep blood sample. (FIG. 11C) Representative images showing morphological changes during the reprogramming timeline using the OSKM+LT and culture conditions. Similar to fibroblasts, fibrocytes form compact iPSC colonies that contain rounded edges with individual cells not discernible.

FIG. 12—Bovine PSCs on different growth substrates.

FIG. 13—Reprogramming sheep fibroblasts to induced pluripotent stem cells (oiPSCs) using OSKM+LT. (FIG. 13A) Reprogramming method timeline showing procedures and culture conditions. (FIG. 13B) Representative images showing morphological changes during the reprogramming timeline. Fibroblasts form compact colonies that contain rounded edges with individual cells not discernible. (FIG. 13C) Representative images showing passaged oiPSCs on MEFs and Matrigel®. Compact colonies are positive for alkaline phosphatase even with repeated passages.

FIG. 14—Pluripotency gene expression in ovine iPSCs. Selected expression of core pluripotency genes compiled in ovine iPSCs are similar to that observed in bovine iPSCs. Results presented are from mRNA-sequencing in both bovine and ovine iPSCs.

FIG. 15A-15B—Pluripotent ovine iPSCs readily differentiate to cells of the three germ layers. (FIG. 15A) Sub-cutaneous introduction of sheep oiPSCs in immunodeficient NSG mice resulted in teratoma formation, with significant growth observed by 6 weeks. Teratomas collected measured more than 1 cm in rough diameter. (FIG. 15B) Representative images of hematoxylin and eosin-stained histological sections of teratomas showing differentiation of oiPSCs into the three different germ layers: [1] Epidermis (ectoderm), [2]Bone marrow (mesoderm), [3] Hyaline cartilage (mesoderm), [4] Lung alveoli (endoderm), and [5] Cardiac muscle (mesoderm).

FIG. 16—Blastocyst embryos resulting from use of iPSCs as nuclear donors in SCNT procedure as described herein.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Barttlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids, as well as those from the environment that contain a biologic entity or part thereof. Samples may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals (e.g., horses, cattle, pigs, sheep, goats, bison, camels, oxen, and the like), sport animals (e.g., horses), wild animals (e.g., bears, deer, elk, moose, etc.) and pets (e.g., canines and felines). Tissues, cells and their progeny of a biological entity obtained in vivo or cultured in vitro are also encompassed.

As used herein, “naïve pluripotency” refers to the ground state of pluripotency. Naïve pluripotent cells are cells having a ground state of pluripotency similar to that observed in murine embryonic stem cells.

As used herein, “culturing” can refer to maintaining cells under conditions in which they can proliferate and avoid senescence as a group of cells. “Culturing” can also include conditions in which the cells also or alternatively differentiate, or conditions that maintain a pluripotent, totipotent, or multipotent state. Culturing conditions can include passaging. Culturing conditions can also exclude passaging. Culturing conditions can include supplementing cells with one or more nutrients, preservatives, anti-infectives, and/or other factors and/or agents. Culture conditions can include the type of media, support (e.g., 3D matrix), physical stress (e.g., temperature, pO₂, pCO₂, strain, etc.) applied to the cells.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” can refer to e.g., 1%, 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, a “population” of cells is any number of cells greater than 1, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more cells, such as at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein “reduced expression” or “underexpression” refers to a reduced or decreased expression of a gene, such as a gene relating to an antigen processing pathway, or a gene product thereof in sample as compared to the expression of said gene or gene product in a suitable control. The term “reduced expression” can refer to a can refer to e.g., 1%, 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more reduced expression relative to a suitable control.

As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Leading up to the 1980s, studies on ruminants were at the forefront of developments in reproductive physiology and assisted reproduction for mammalian species (Britt et al., 1981; Willett, 1956). Derivation of murine embryonic stem cells (mESCs) in 1981 (Evans and Kaufman, 1981; Martin, 1981), opened up new possibilities for advancement, and there was significant investment in deriving bovine ESCs (bESCs). Despite preliminary successes in isolating pluripotent cells from the bovine inner cell mass (Cherny et al., 1994; Saito et al., 1992; Sims and First, 1994a; Stice et al., 1996), it was not possible to effectively sustain these cells in culture (First et al., 1994; Talbot and Blomberg, 2008). After human ESCs (hESCs) were derived in 1998 (Thomson et al., 1998), it brought to light understanding that signaling mechanisms for sustaining ESCs in culture were quite divergent. Growth factor leukemia inhibitory factor (LIF) acting via STAT3 was found to support self-renewal in mESCs (Niwa et al., 1998; Williams et al., 1988), and fibroblast growth factor 2 (FGF2) acting via activin/nodal were identified for hESCs (James et al., 2005b; Vallier et al., 2005). Numerous attempts to derive and study bESCs have continued over the years without definitive methods to sustain pluripotency and self-renewal long-term (Cao et al., 2009; Cibelli et al., 1998; Cong et al., 2014; Iwasaki et al., 2000; Kim et al., 2012; Lim et al., 2011; Mitalipova et al., 2001; Pant and Keefer, 2009; Pashaiasl et al., 2010; Pashaiasl et al., 2013; Saito et al., 1992; Saito et al., 2003; Sims and First, 1994b; Stice, 1996; Talbot et al., 1995; Van Stekelenburg-Hamers et al., 1995; Wang et al., 2005); the most recent studies being demonstrations that a primed form of bESCs (Bogliotti et al., 2018) and other early transitional states (Huang et al., 2014) that express pluripotency markers and retain the ability to differentiate into the three germ layers, can be cultured from bovine blastocysts. Hitherto, these studies have not only highlighted serious challenges to sustenance of pluripotency, but also the paucity in understanding of signaling and regulation for self-renewal in bESCs (Ezashi et al., 2016; Keefer et al., 2007; Malaver-Ortega et al., 2012).

In 2006, discovery of induced pluripotent stem cells (iPSCs) that had characteristics identical to ESCs (Takahashi and Yamanaka, 2006; Takahashi et al., 2007), infused new enthusiasm for bovine pluripotency research. In the ensuing decade, we and others have made numerous attempts to generate biPSCs incorporating the four core reprogramming genes POU5F1 (OCT4), SOX2, KLF4 and MYC (OSKM), and other factors (Bai et al., 2016; Canizo et al., 2018; Cao et al., 2012; Cravero et al., 2015; Han et al., 2011; Heo et al., 2015; Huang et al., 2011; Kawaguchi et al., 2015; Lin et al., 2014; Pillai et al., 2019b; Sumer et al., 2011; Talluri et al., 2015; Wang et al., 2013; Zhao et al., 2017), but with limited success. Although several of these studies claim successful derivation of these cells, measures of quality have remained quite arbitrary (Pieri et al., 2019). In genome-integrating transgene based-approaches, the exogenous transgenes were not silenced (Cao et al., 2012; Cravero et al., 2015; Han et al., 2011; Heo et al., 2015; Huang et al., 2011; Sumer et al., 2011; Talluri et al., 2015; Zhao et al., 2017), or this was not evaluated (Canizo et al., 2018; Lin et al., 2014; Wang et al., 2013). In some studies, forced expression of the reprogramming genes induced trophoblast formation from bovine fibroblasts rather than pluripotent cells (Kawaguchi et al., 2016; Talbot et al., 2017). In the case of doxycycline-inducible reprogramming transgenes, continuous induction of exogenous expression was necessary to maintain bovine iPSCs (Kawaguchi et al., 2015). Supporting the lack in activation of the endogenous pluripotency network, some studies have concluded that bovine fibroblasts present an epigenetic block that prevents complete reprogramming (Canizo et al., 2018; Kawaguchi et al., 2015). In agreement, an extrapolation that progenitors can reprogram more readily than terminally differentiated cells was confirmed (Bai et al., 2016; Kawaguchi et al., 2015; Lin et al., 2014; Pillai et al., 2019b; Wang et al., 2013). This insufficiency led to additional testing for including Nanog (Pillai et al., 2019b; Sumer et al., 2011), knockdown of p53 (Pillai et al., 2019b), knockdown of Mbd3 (Pillai et al., 2019b), and overexpression of the microRNA 302/367 cluster (Bai et al., 2016; Pillai et al., 2019b), without success. For sustenance, these studies have attempted using LIF (Heo et al., 2015; Huang et al., 2011; Lin et al., 2014; Wang et al., 2013), FGF2 (Bai et al., 2016; Canizo et al., 2018), both (Cao et al., 2012; Cravero et al., 2015; Han et al., 2011; Kawaguchi et al., 2015; Pillai et al., 2019b; Sumer et al., 2011; Talluri et al., 2015; Zhao et al., 2017) or with added BMP4 (Zhao et al., 2017), without a clear functional consensus. Together with LIF, some studies have also tried pharmacological inhibition of mitogen-activated protein kinase kinase (MAP2K1/2 or MEK1/2), and glycogen synthase kinase 38 (GSK3β) to prevent differentiation and promote self-renewal (Heo et al., 2015; Huang et al., 2011). Despite these sustained efforts, a consistent and reproducible method for maintaining a pluripotent state remains to be deciphered for cattle or for any other ruminant species (Pieri et al., 2019; Su et al., 2020).

In investigating a variety of approaches to enhance efficiency in biPSC generation (Pillai et al., 2019b), Applicant has identified two compounding facets to the problem underpinning the lack of any true biPSC cell: (i) a stable epigenome in bovids resists iPSC reprogramming, exemplified by failures with methods that enhance permissiveness in murine and human somatic cells (Pillai et al., 2019b); (ii) there is dearth in understanding of signaling necessary for sustaining pluripotency and/or differentiation specific to bovids (Ezashi et al., 2016; Pillai et al., 2019b). Further, Applicant describes and demonstrates herein compositions and methods capable of reprogramming somatic cells, particularly bovine somatic cells and others that suffer from the same or similar problems and/or maintaining the pluripotency of reprogrammed and other pluripotent stem cells, such as those reprogrammed with large T antigen and OSKM expression and/or have the same or similar expression signatures or programs as the iPSCs Applicant has generated and/or a 16 cell bovine embryo. Applicant also describes and demonstrates herein reprogrammed and/or induced pluripotent cells in exemplary species. Applicant also demonstrates the use of the compositions, methods, and cells in somatic cell nuclear transfer (SCNT) for the generation of cloned and/or engineered non-human animals.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Cell Culture Compositions

Described in certain exemplary embodiments herein is a stem cell culture medium comprising an amount of a pluripotency composition comprising a glycogen synthase kinase 3 beta (GSK3beta) inhibitor; a mitogen-activated protein kinase kinase (MEK 1/2) inhibitor; and a transforming growth factor beta (TGF beta)/activin/nodal pathway inhibitor, wherein the pluripotency composition is effective to maintain pluripotency and/or inhibit and/or prevent differentiation of a cell.

Pluripotency and/or the ability to inhibit and/or prevent differentiation can be evaluated by culturing and observing morphological characteristics consistent with differentiated vs. pluripotent cells, measuring or detecting self-renewal, pluripotent, multi-potent, totipotent, differentiated cell markers or functions (such as via teratoma growth) and other assays set forth in e.g., the Working Examples herein.

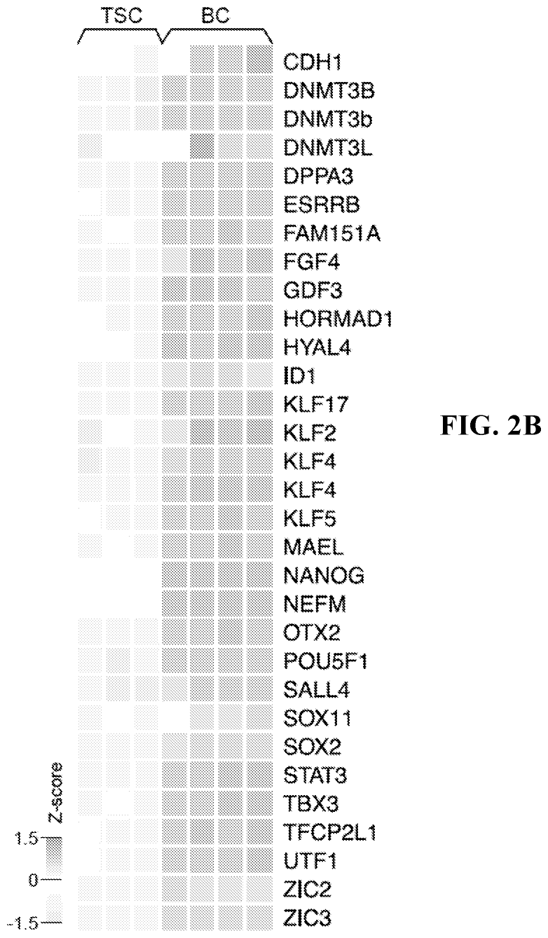

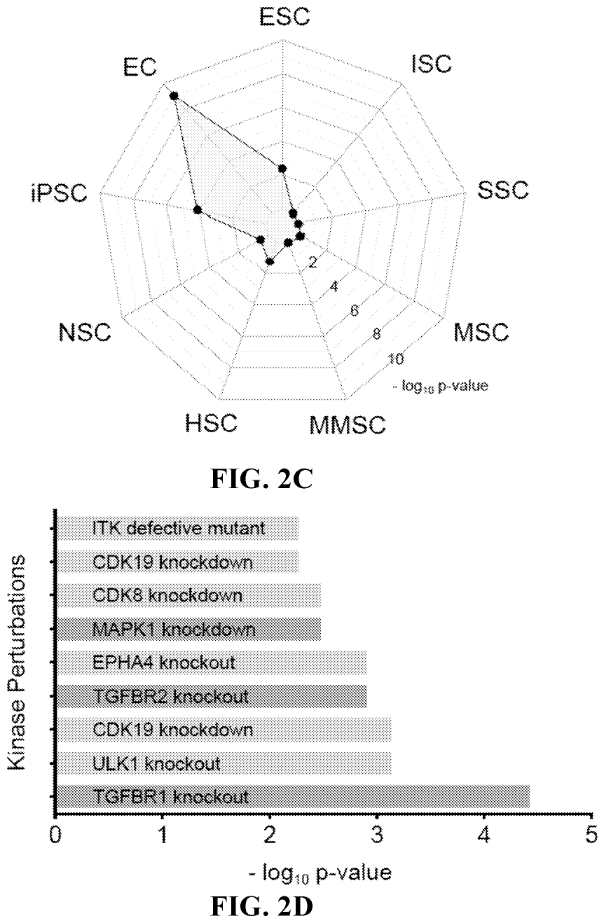

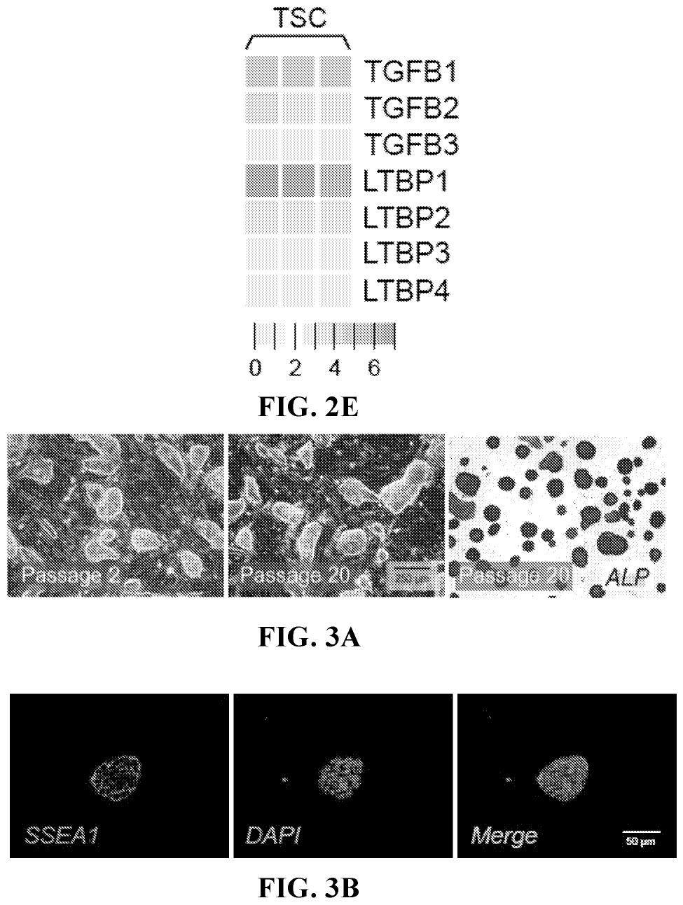

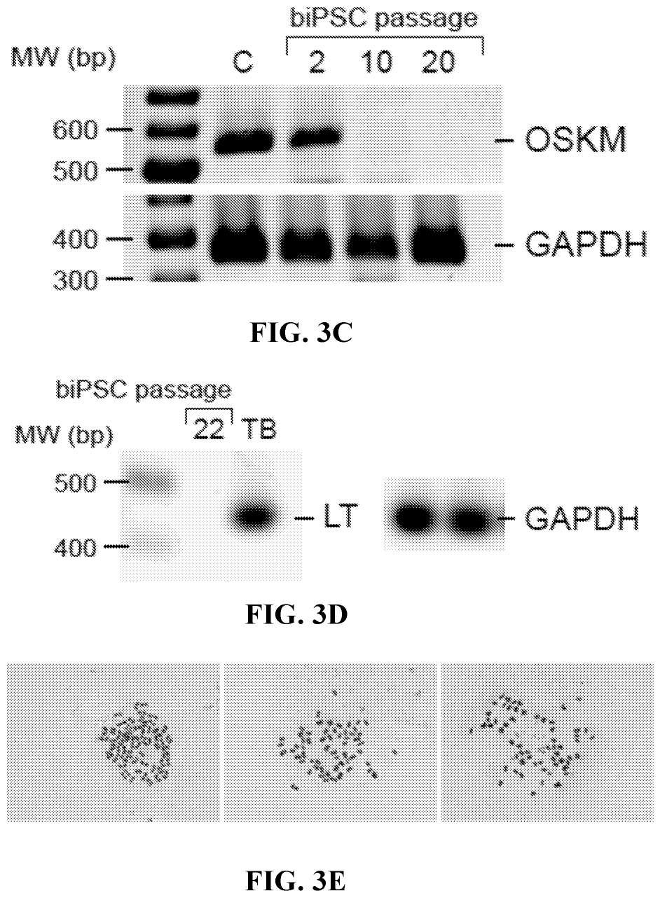

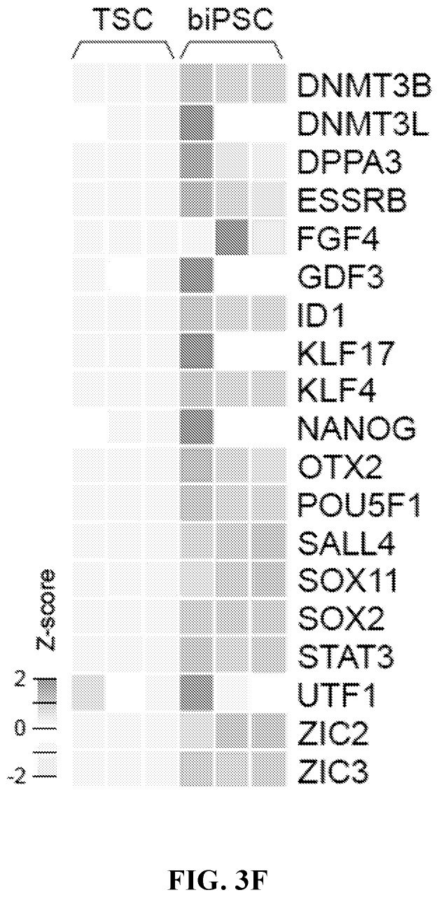

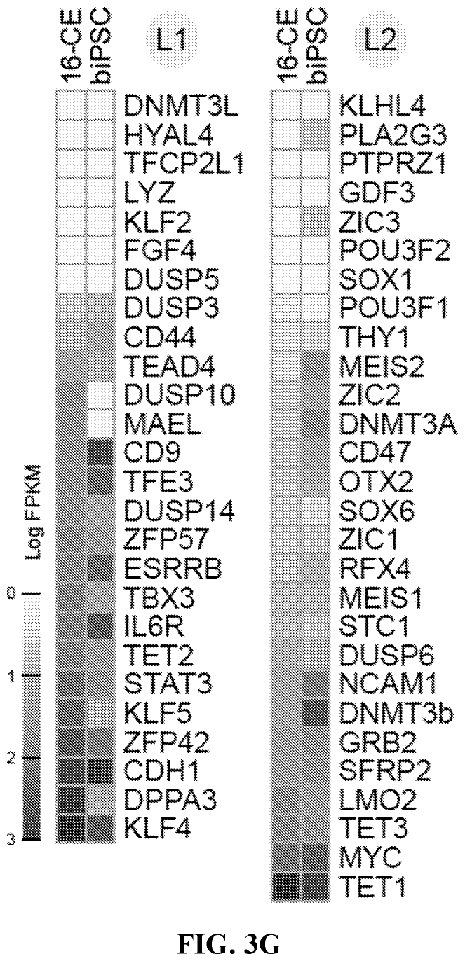

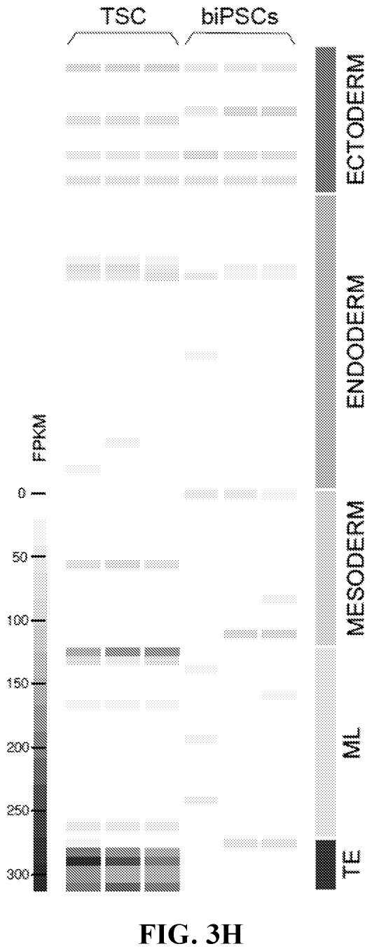

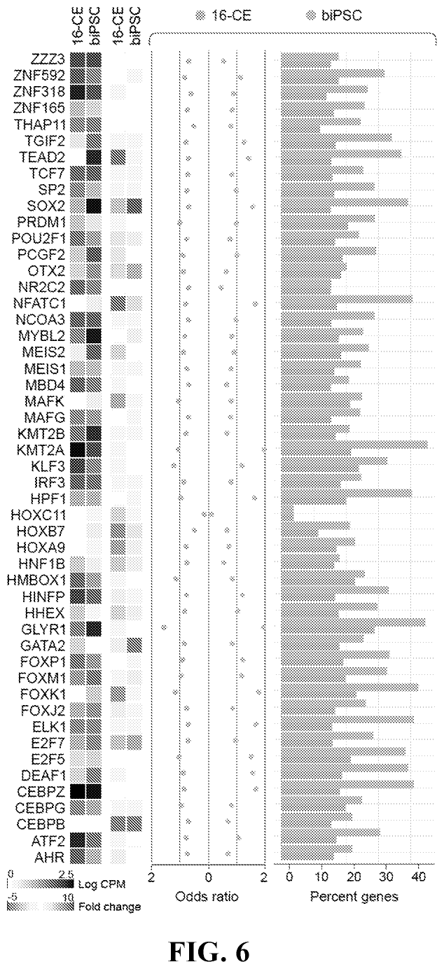

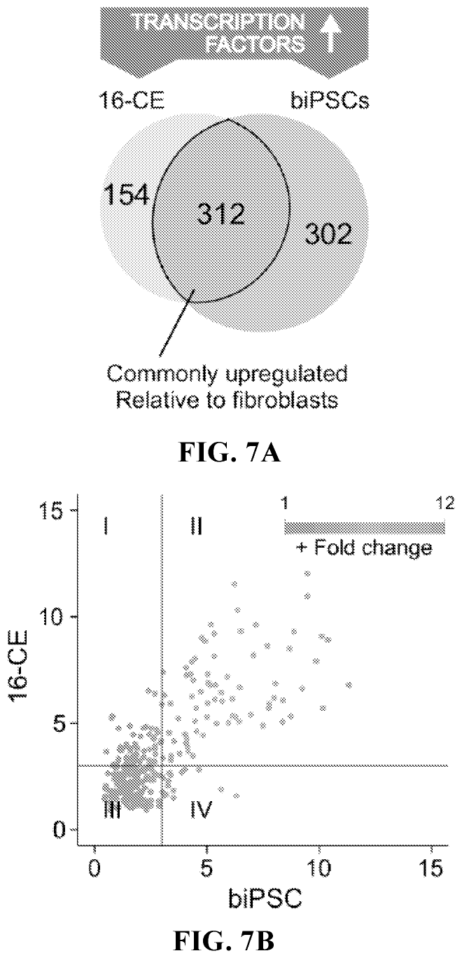

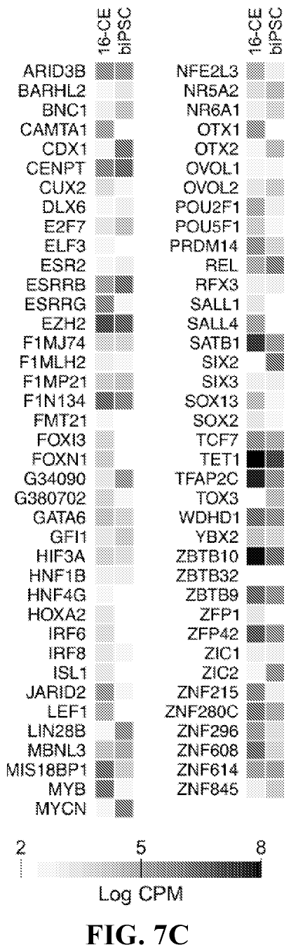

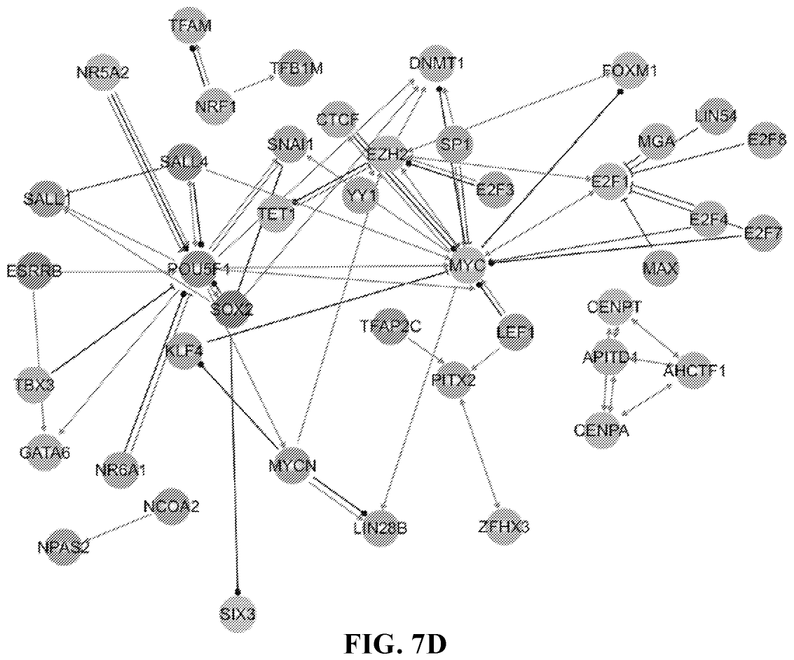

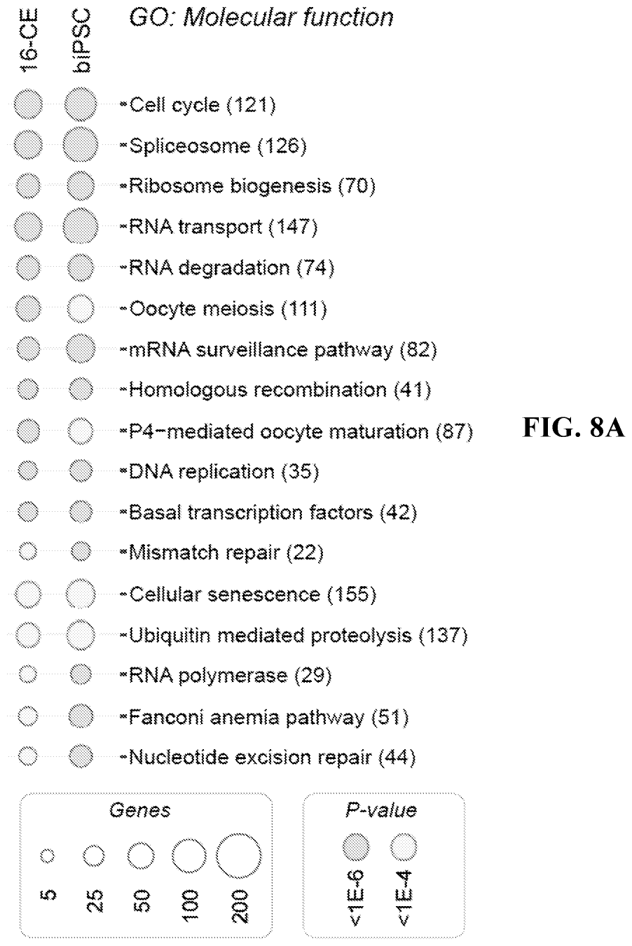

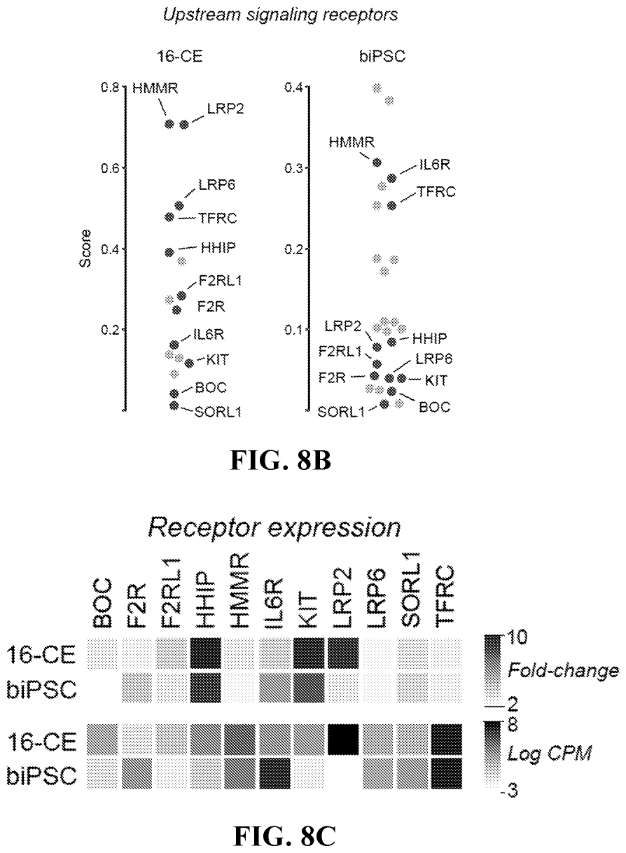

In some exemplary embodiments, the cell comprises a reprogrammed and/or a pluripotent stem cell signature and/or program. In some embodiments, the reprogrammed and/or a pluripotent stem cell signature and/or program comprises one or more genes and/or programs as set forth herein, particularly in the Working Examples, e.g., FIG. 2A-2E and related discussion therein, FIGS. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some embodiments, the reprogrammed and/or a pluripotent stem cell program comprises a TGFbeta signaling program. In some example embodiments, the TGFbeta signaling program comprises one or more genes and/or programs as set forth in the Working Examples herein, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof. In some embodiments, the reprogrammed and/or a pluripotent stem cell signature and/or program comprises a TGFbeta receptor 1, TGFbeta receptor 2, or both. In some embodiments, the TGFbeta program is a TGFbeta suppression or inhibition program. In some embodiments, the TGFbeta program is a TGFbeta signaling pathway suppression or inhibition program.

In some example embodiments, the cell comprises a 16-cell embryo signature and/or program. In some example embodiments, the 16-cell embryo signature and/or program comprises one or more genes and/or programs as set forth in the Working Examples herein, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, and expression analysis data deposited with NCBI by at least Aug. 27, 2021 with deposit ID GSE169624, or any combination thereof.

In some exemplary embodiments, cell does not comprise a trophectoderm cell signature and/or program; a trophoblast stem cell signature and/or program; a trophoblast signature and/or program; an endoderm cell signature and/or program; a mesoderm cell signature and/or program; an ectoderm cell signature and/or program; a differentiated cell signature or program; or any combination thereof.

In some example embodiments, the trophectoderm cell signature and/or program comprises one or more genes and/or programs as set forth herein, particularly in the Working Examples, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some example embodiments, the trophoblast signature and/or program comprises one or more genes and/or programs as set forth in as set forth herein, particularly in the Working Examples, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some example embodiments, the trophoblast stem cell signature and/or program comprises one or more genes and/or programs as set forth herein, particularly in the Working Examples, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some example embodiments, the endoderm cell signature and/or program comprises one or more genes and/or programs as set forth herein, particularly in the Working Examples, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some example embodiments, the ectoderm cell signature and/or program comprises one or more genes and/or programs as set forth herein, particularly in the Working Examples, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some example embodiments, the differentiated cell signature and/or program comprises one or more genes and/or programs as set forth herein particularly in the Working Examples, e.g., FIG. 2A-2E and related discussion therein, FIG. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

In some exemplary embodiments, the cell is not a trophectoderm cell, a trophoblast stem cell, a trophoblast, an endoderm cell, a mesoderm cell, or a differentiated cell.

As used herein the term “biological program”, “program”, and the like, can be used interchangeably with “expression program” and refers to a set of genes that share a role in a biological function (e.g., an activation program, suppression program, inhibition program, cell differentiation program, proliferation program, signaling program and/or the like). Biological programs can include a pattern of gene expression that result in a corresponding physiological event or phenotypic trait. Biological programs can include up to several hundred genes and/or proteins that are expressed in a spatially and temporally controlled fashion. Expression of individual genes and/or proteins can be shared between biological programs. Expression of individual genes and/or proteins can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor. As used herein, the term “topic” refers to a biological program. The biological program can be modeled as a distribution over (or across) expressed genes and/or proteins.

As used herein, the term “signature” may encompass any gene or genes, protein or proteins, or epigenetic element(s), whose expression profile or whose occurrence is associated with a specific microorganism type or subtype, state or life-stage of a microorganism type or subtype, or combination thereof. As used herein the term “replicating infectious agent signature” therefore refers to a signature that is unique to and thus can identify the replication stage of the life-cycle of an infectious agent. For ease of discussion, when discussing gene expression, any of gene or genes, protein or proteins, or epigenetic element(s) may be substituted. As used herein, the terms “signature”, “expression profile” may be used interchangeably. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. A signature can be composed of any number of genes, proteins epigenetic elements, and/or combinations thereof. For example, a gene signature may include a list of genes differentially expressed in a distinction of interest. In some embodiments signature can be composed completely of or contain 1, 2, 3, 4, 5, 6, 7, 8, 9, 10, 11, 12, 13, 14, 15, 16, 17, 18, 19, or 20 or more genes, proteins and/or epigenetic elements. In aspects, the signature can be composed completely of or contain 1-20 or more, 2-20 or more, 3-20 or more, 4-20 or more, 5-20 or more, 6-20 or more, 7-20 or more, 8-20 or more, 9-20 or more, 10-20 or more, 11-20 or more, 12-20 or more, 13-20 or more, 14-20 or more, 15-20 or more, 16-20 or more, 17-20 or more, 18-20 or more, 19-20 or more, or 20 or more genes, proteins and/or epigenetic elements. In some embodiments, a signature comprises or contains only 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more genes, proteins and/or epigenetic elements.

In certain example embodiments, the a signature and/or program described herein comprises one or more genes, proteins, transcripts, and/or epigenetic elements that have differential relative and/or absolute expression of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, to/or 200-fold or more.

The signature genes, proteins, and/or epigenetic elements may be detected or isolated by any suitable methods or technique. Exemplary suitable techniques include, but are not limited to, immunofluorescence, immunohistochemistry (IHC), fluorescence activated cell sorting (FACS), mass spectrometry (MS), mass cytometry (CyTOF), RNA-seq, single cell RNA-seq), quantitative RT-PCR, single cell qPCR, FISH, RNA-FISH, MERFISH (multiplex (in situ) RNA FISH), ATAC-seq, and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). Other suitable methods and techniques will be appreciated by one of ordinary skill in the art in view of this disclosure.

In some embodiments, the signature can be determined using an RNAseq technique. Exemplary techniques are described in e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012); Picelli, S. et al., 2014, “Full-length RNA-seq from single cells using Smart-seq2” Nature protocols 9, 171-181, doi:10.1038/nprot.2014.006; Macosko et al., 2015, “Highly Parallel Genome-wide Expression Profiling of Individual Cells Using Nanoliter Droplets” Cell 161, 1202-1214; WO2016/040476, WO2016168584A1, Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017); to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958, WO2017164936), which can be adapted for use with the present disclosure.

In some exemplary embodiments, the cell is a pluripotent stem cell, optionally an induced pluripotent stem cell. As the term is used herein, “reprogrammed” refers to a cell, such as somatic cell, partially or fully differentiated cell, that has been manipulated (e.g., chemically, biologically, and/or genetically) such that it goes to a multipotent, totipotent, pluripotent, naïve pluripotent, or primed pluripotent state and/or exhibits the ability to differentiate into one or more different types of cells and/or exhibits some or full self-renewal capabilities. As the term is used herein “induced pluripotent stem cell” is a pluripotent stem cell that has been generated from the reprogramming of a somatic cell and/or non-pluripotent stem cell.

In some exemplary embodiments, the cell is a pluripotent stem cell, optionally an induced pluripotent stem cell. In some exemplary embodiments, the cell is a reprogrammed cell, optionally an induced pluripotent stem cell, that was reprogrammed by expression of Oct4, Sox2, Klf4, Myc, SV40 Large T antigen, or any combination thereof. In some exemplary embodiments, the cell is a reprogrammed cell, optionally an induced pluripotent stem cell, that was reprogrammed by expression of Oct4, Sox2, Klf4, Myc, and SV40 Large T antigen. In some exemplary embodiments, the cell is a non-human mammalian cell. In some exemplary embodiments, the cell is a ruminant cell. In some exemplary embodiments, the cell is a bovine cell, ovine cell, caprine cell, a cervine cell, a giraffe cell, or a camel cell.

In some embodiments, the cell expresses or has expressed SV40 Large T antigen, Oct4, Sox2, Klf4, Myc, or any combination thereof. In some embodiments, the cell the pluripotent stem cell expresses or has expressed SV40 Large T antigen, Oct4, Sox2, Klf4, and Myc.

The stem cell culture medium can further contain additional compounds, compositions, and/or the like. Such additions can include, but are not limited to growth factors, preservatives, anti-infectives, pH indicators, inhibitory factors, nutrients, and/or the like. In some embodiments, the cell culture medium further comprises an amount of leukemia inhibitory factor (LIF), an amount of interleukin (IL-6), or both. In some embodiments, the cell culture medium comprises DMEM, MEM, Essential 6 medium, Neurobasal™, mTesR™, Leibovitz L-15, McCoy's 5A, a suitable whole or partial cell culture medium, or any combination thereof. In some embodiments, the cell culture medium comprises Ham's F12 nutrient composition, Ham's F10 nutrient composition, serum, Knockout serum replacement, N2 supplement, B-27 supplement, nutrient supplement (e.g., an amino acid(s), a vitamin(s), mineral(s), and/or the like), one or more anti-infectives (e.g., one or more antibiotics, one or more antifungals and/or the like), a reducing agent, a pH indicator, a buffer, or any combination thereof.

In some embodiments, the stem cell culture medium does not contain a growth factor.

In some embodiments, the GSK3beta inhibitor is selected from: a small molecule inhibitor, an antibody or fragment thereof, an aptamer, an RNAi molecule, a polypeptide ligand or antagonist, or any combination thereof. In some embodiments, the GSK3beta inhibitor is selected from: CHIR99021, SB-216763, GSK-3 inhibitor IX, kenpaullone or an analog thereof, lithium chloride, GSK-3beta inhibitor XII, GSK-3beta inhibitor VII, GSK-3inhibitor XVI, 10Z-hymenialdsine, indirubin, CHIR-98014, GSK-3beta inhibitor VI, indirubin-3′-monoxime, GSK-3 inhibitor X, SB-415268, TWS 119 ditrifluoroacetate, 5-iodo-indirubin-3′-monoxime, GSK-3beta inhibitor I, indirubin-5-sulfonic acid sodium salt, hymenidin, 3F8, Bisindlylmaleimide X HCl, indirubin-3′-monoxime-5-sulphonic acid, GSK-3 inhibitor II, GSK-3beta inhibitor VIII, GSK-3beta inhibitor XI, TCS 2002, alsterpaullone 2-cyanoethyl, A 1070722, MeBIO, AR-AO 14418-d3, 6-N-[2-[[4-(2,4-dichlorophenyl)-5-(1H-imidazol-2-yl)pyrimidin-2-yl]amino]ethyl]-3-nitropyridine-2,6-diamine, or any combination thereof.

In some embodiments, the MEK 1/2 inhibitor is selected from: a small molecule inhibitor, an antibody or fragment thereof, an aptamer, an RNAi molecule, a polypeptide ligand or antagonist, or any combination thereof. In some embodiments, the MEK 1/2 inhibitor is selected from: PD0325901, AS703988/MSC2015103B, PD184352, Selumetinib, MEK162, AZD8330, TAK-733, GDC-0623, Refametinib, RO4987655, WX-554, HL-085, Cobimetinib, Trametinib, binimetinib, Pimasertib, Mirdametinib, E6201, CH5126766, SHR7390, TQ-B3234, CS-3006, FCN-159, RO5126766, or any combination thereof.

In some embodiments, the TGF beta/activin/nodal pathway inhibitor is selected from: a small molecule inhibitor, an antibody or fragment thereof, an aptamer, an RNAi molecule, a polypeptide ligand or antagonist, or any combination thereof. In some embodiments, the TGF beta/activin/nodal pathway inhibitor is selected from: A83-01, SB431542, SB505124, LY2157299 (Galunisertib), LY550410, fresolimumab, AP12009, AP1104/AP15012, Lucanix™, ISTH0036, GC1008, 2G7, 1D11, LY2382770, CAT-192, Ki 26894, SD208, LY2109761, IN-1130, LY2157299, TEW-7197, PF-03446962, Gemogenovatucel-T, Trx-xFoxH1b aptamer, an inhibitor of myosins (e.g., pentachloropseudilin (PClP) and pentabromopseudilin (PBrP)), sorafenib, 6.3G9 antibody, 264RAD, lerdelimumab, soluble TbetaRII/III, 5,6-dihydro-4H-pyrrolo[1,2-b]pyrazole and analogs thereof, PF-03446962, KRC203, KRC360, LDN193189, ACE-041/Dalantercept, SB-525334, LY-364947, GW-6604, SD-208, or any combination thereof.

### Methods of Reprogramming Cells and Culturing Reprogrammed Cells

Described in several exemplary embodiments herein are methods of maintaining pluripotency of and/or inhibiting or preventing differentiation of a cell or cell population, the method comprising culturing a cell or cell population in the stem cell culture medium as described above and elsewhere herein.

In some exemplary embodiments, the cell comprises a reprogrammed cell and/or a pluripotent stem cell signature and/or program. In some exemplary embodiments, the reprogrammed cell and/or pluripotent stem cell program comprises a TGFbeta signaling program. In some exemplary embodiments, the reprogrammed cell and/or pluripotent stem cell signature comprises TGFbeta receptor 1, TGFbeta receptor 2, or both. In some exemplary embodiments, the cell comprises a 16-cell embryo signature and/or program. Such signatures and/or programs are described in greater detail elsewhere herein.

In some exemplary embodiments, the cell does not comprise a trophectoderm cell signature and/or program; a trophoblast stem cell signature and/or program; a trophoblast signature and/or program; an endoderm cell signature and/or program; a mesoderm cell signature and/or program; an ectoderm cell signature and/or program; a differentiated cell signature and/or program; or any combination thereof. Such signatures and/or programs are described above and elsewhere herein.

In some exemplary embodiments, the cell expresses or has expressed SV40 Large T antigen, Oct4, Sox2, Klf4, Myc, or any combination thereof. In some exemplary embodiments, cell expresses or has expressed SV40 Large T antigen, Oct4, Sox2, Klf4, and Myc.

In some exemplary embodiments, the cell is a non-human mammalian cell. In some exemplary embodiments, the cell is a ruminant cell. In some exemplary embodiments, the cell is a bovine cell, an ovine cell, a caprine cell, a cervine cell, a giraffe cell, or a camel cell. In some exemplary embodiments, the cell is a murine cell, an equine cell, a feline cell, or a cell. In some exemplary embodiments, the cell is a pluripotent stem cell, optionally an induced pluripotent stem cell. In some exemplary embodiments, the cell is a reprogrammed and/or induced pluripotent stem cell that was reprogrammed by expression of Oct4, Sox2, Klf4, Myc, and Large T antigen.

In some embodiments, culturing comprises passaging the cells 1 or more times. In some embodiments, culturing comprises passaging the cells 1, to/or 2, 3, 4, 5, 6, 7, 8, 9, 10, 11, 12, 13, 14, 15, 16, 17, 18, 19, 20, 21, 22, 23, 24, 25 or more times. In some embodiments, culturing does not comprise passaging. In some embodiments, culturing includes refreshing media 1 or more times, such as 1, to/or 2, 3, 4, 5, 6, 7, 8, 9, 10, 11, 12, 13, 14, 15, 16, 17, 18, 19, 20, 21, 22, 23, 24, 25 or more times. In some embodiments, culturing includes refreshing media but not passaging cells.

In some embodiments, culturing comprises culturing cells on feeder cell layer (also referred to herein as a “feeder layer”). Suitable feeder layers are generally known in the art and include, but are not limited to, mouse embryonic fibroblasts, irradiated mouse embryonic fibroblasts, and the like. In some embodiments, culturing does not include culturing cells on feeder layer.

In some embodiments, culturing comprises culturing cells adherently on a cell culture surface, such as on a scaffold or cell culture container surface.

In some embodiments, culturing comprises culturing cells in suspension.

In some embodiments, culturing comprises growing cells on a 3D matrix (also referred to in the art as a “scaffold”). Suitable 3D matrices include, but are not limited to, natural or synthetic extracellular matrix or one or more components thereof (e.g., one or more types of collagen, laminin, etc.), cartilage, alginate, calcium, laminin, a hydrogel, a polymer scaffold (such as polystyrene, polycarbonate, and/or the like), and/or the like, or any combination thereof. Other suitable 3D matrices are known in the art.

In some embodiments, culturing comprises a scaffold free method of culturing, such as a spheroid culturing technique, a hanging drop culturing technique, suspension cell culture, and/or the like.

In some embodiments, the method can include expanding cells, harvesting cells, and/or preserving and/or storing cells. In some embodiments, the starting culture is from a stored cell culture. In such embodiments, the method can include resuscitation of the stored cells. If the cells were frozen, this can include thawing.

A general guide to general cell culture can be found in Basic Science Methods for Clinical Researchers. 2017: 151-172, doi: 10.1016/B978-0-12-803077-6.00009-6.

In some embodiments, the method does not include differentiating cells.

Described in certain example embodiments herein are methods of generating reprogrammed cells, optionally induced pluripotent stem cells, the method comprising reprogramming a somatic cell; and culturing the reprogrammed somatic cell using a method of maintaining pluripotency of and/or inhibiting or preventing differentiation of a cell or cell population described above and elsewhere herein.

In some embodiments, reprogramming comprises expressing SV40 large T antigen, Oct4, Sox2, Klf4, and Myc in the somatic cell or non-pluripotent cell. In some embodiments, the somatic cell or non-pluripotent cell is a non-human somatic cell or non-pluripotent cell. In some embodiments, the somatic cell or non-pluripotent cell is a ruminant somatic cell or non-pluripotent cell. In some embodiments, the somatic cell or non-pluripotent cell is a bovine somatic cell or non-pluripotent cell, ovine somatic cell or non-pluripotent cell, caprine somatic cell or non-pluripotent cell, a cervine somatic cell or non-pluripotent cell, a giraffe somatic cell or non-pluripotent cell, or a camel somatic cell or non-pluripotent cell. In some embodiments, the somatic cell or non-pluripotent cell is a murine somatic cell or non-pluripotent cell, an equine somatic cell or non-pluripotent cell, a feline somatic cell or non-pluripotent cell, or a canine somatic cell or non-pluripotent cell. In some embodiments the somatic cell or non-pluripotent cell is a fibrocyte.

Also described herein are methods of differentiating and/or otherwise modifying a reprogrammed and/or pluripotent stem cell, such as an induced pluripotent stem cell.

In some embodiments, the method comprises differentiating a reprogrammed pluripotent stem cell optionally cultured by a method described above and elsewhere herein and/or modifying a pluripotent stem cell optionally generated by a method of described above or elsewhere herein, or both. In some embodiments, the reprogrammed and/or pluripotent stem cell is an induced pluripotent stem cell has a reprogrammed and/or pluripotent stem cell signature as described above and elsewhere herein. In some embodiments, the reprogrammed and/or pluripotent stem cell and/or reprogrammed and/or pluripotent stem cell program comprises a TGFbeta signaling program. In some embodiments, the reprogrammed and/or cell and/or reprogrammed and/or pluripotent stem cell signature comprises TGFbeta receptor 1, TGFbeta receptor 2, or both. Such signatures and/or programs are described above and elsewhere herein. In some embodiments, the reprogrammed and/or pluripotent stem cell and/or reprogrammed and/or pluripotent stem cell signature and/or program comprises a 16-cell embryo signature and/or program. Such signatures and/or programs are described above and elsewhere herein.

In some embodiments, the pluripotent stem cell does not comprise a trophectoderm cell signature and/or program; a trophoblast stem cell signature and/or program; a trophoblast signature and/or program; an endoderm cell signature and/or program; a mesoderm cell signature and/or program; an ectoderm cell signature and/or program; a differentiated cell signature and/or program; or any combination thereof. Such signatures and/or programs are described above and elsewhere herein.

In some embodiments, the reprogrammed and/or pluripotent stem cell is an induced pluripotent stem cell. In some embodiments, the reprogrammed and/or pluripotent stem cell, such as an induced pluripotent stem cell, was reprogrammed by expressing Large T antigen, Oct4, Sox2, Klf4, Myc or any combination thereof in a somatic or non-pluripotent stem cell. In some embodiments, the reprogrammed and/or pluripotent stem cell, such as an induced pluripotent stem cell, was reprogrammed by expressing Large T antigen, Oct4, Sox2, Klf4, and Myc in a somatic or non-pluripotent cell. In some embodiments, the reprogrammed and/or pluripotent stem cell, such as an induced pluripotent stem cell, expresses or has expressed Large T antigen, Oct4, Sox2, Klf4, Myc, or any combination thereof. In some embodiments, the reprogrammed and/or pluripotent stem cell, such as an induced pluripotent stem cell, expresses or has expressed Large T antigen, Oct4, Sox2, Klf4, and Myc.

In some embodiments, the reprogrammed and/or pluripotent stem cell, such as an induced pluripotent stem cell, is a non-human mammalian reprogrammed and/or pluripotent stem cell, such as an induced pluripotent stem cell. In some embodiments, the reprogrammed and/or pluripotent stem cell, such as an induced pluripotent stem cell, is a ruminant reprogrammed and/or pluripotent stem cell, such as an induced pluripotent stem cell. In some embodiments, the reprogrammed and/or pluripotent stem cell, such as an induced pluripotent stem cell, is a bovine reprogrammed and/or pluripotent stem cell, such as an induced pluripotent stem cell, ovine reprogrammed and/or pluripotent stem cell, such as an induced pluripotent stem cell, caprine reprogrammed and/or pluripotent stem cell, such as an induced pluripotent stem cell, a cervine reprogrammed and/or pluripotent stem cell, such as an induced pluripotent stem cell, a giraffe reprogrammed and/or pluripotent stem cell, such as an induced pluripotent stem cell, or a camel reprogrammed and/or pluripotent stem cell, such as an induced pluripotent stem cell. In some embodiments, the reprogrammed and/or pluripotent stem cell, such as an induced pluripotent stem cell, is a murine reprogrammed and/or pluripotent stem cell, such as an induced pluripotent stem cell, an equine reprogrammed and/or pluripotent stem cell, such as an induced pluripotent stem cell, a feline reprogrammed and/or pluripotent stem cell, such as an induced pluripotent stem cell, or a canine reprogrammed and/or pluripotent stem cell, such as an induced pluripotent stem cell. In some embodiments, the reprogrammed and/or pluripotent stem cell is an induced pluripotent stem cell. In some embodiments, the reprogrammed and/or pluripotent stem cell is an induced pluripotent stem cell that was reprogrammed by expression of Oct4, Sox2, Klf4, Myc, and Large T antigen.

In some embodiments, the reprogrammed and/or pluripotent stem cell (such as an induced pluripotent stem cell) was cultured using a method described above or elsewhere herein prior to differentiating and/or modifying.

In some embodiments, modifying comprises genetic and/or transcript modification. Such modification includes gene and RNA editing, RNA interference, transgene insertion, indels, deletions, and/or the like. Such modification can be carried out using any suitable methods. Such methods include random integration of exogenous DNA, RNA interference, programmable/guided nuclease modification (e.g., CRISPR-Cas systems, Zn finger nuclease systems, meganuclease systems, CRISPR-Associated transposase (CAST) systems, and/or the like), Cre-lox systems, transposon systems, recombinases, Base-editors, and/or the like. In some embodiments, the modification is DNA and/or RNA modification. In some embodiments, the modification is insertions, deletions, substitutions, mutations, indels, and/or the like. In some embodiments, 1 to 5,000 or more nucleotides are modified.

In some embodiments, the method further includes culturing the differentiated and/or modified cells in vitro. In some embodiments, the method further includes introducing the differentiated cells into a subject. In some embodiments, the method further includes introducing the differentiated and/or modified cells into a recipient non-human female such that the cells can implant into a uterus and generate a full non-human organism.

### Engineered Cells

Also described herein are engineered cells that comprise a bovine induced pluripotent stem cell signature, wherein the bovine induced pluripotent stem cell signature comprises one or more genes and/or programs as set forth herein, particularly in the Working Examples, e.g., FIG. 2A-2E and related discussion therein, FIGS. 3B-3D, 3F-3H and related discussion therein, FIG. 6 and related discussion therein, FIG. 7A-7D and related discussion therein, FIG. 8A-8C and related discussion therein, Tables 2-5 and related discussion, or any combination thereof.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man. In some embodiments, the engineered cells are similar to or identical in one or more aspects but not completely identical to a native bovine cell, such as a native bovine pluripotent, totipotent, multipotent, or other somatic cell. In some embodiments, the engineered cells may share a pluripotent signature as a native cell, but contain at least one difference (genotype, epigenetic, phenotype, etc. difference) as compared to the native cell.

In some embodiments, the engineered cell is a genetically edited or otherwise modified cell. In some embodiments, the engineered cell is a non-human mammal cell. In some embodiments, the engineered cell is a ruminant cell. In some embodiments, the engineered cell is a bovine cell, ovine cell, caprine cell, a cervine cell, a giraffe cell, or a camel cell. In some embodiments, the engineered cell is a murine cell, an equine cell, a feline cell, or a canine cell. In some embodiments, the engineered cell is a pluripotent stem cell. In some embodiments, the engineered cell is an induced pluripotent stem cell. In some embodiments, the engineered cell expresses or has expressed SV40 Large T antigen, Oct4, Sox2, Klf4, Myc, or any combination thereof. In some embodiments, the engineered cell expresses or has expressed SV40 large T antigen, Oct4, Sox2, Klf4, and Myc. In some embodiments, the iPSC cell is generated from a blood somatic cell. In some embodiments, the engineered cell has been cultured in a stem cell culture media as described above and elsewhere herein and/or cultured and/or generated via a method as described above and/or generated else wherein.

### Methods of Using the Reprogrammed/Engineered Cells

The reprogrammed and/or engineered cells described elsewhere herein can be used in a method of cloning, such as in non-human animal cloning. In some embodiments, the reprogrammed and/or engineered cells are used in somatic cell nuclear transfer. The compositions and methods described herein can increase the efficiency of somatic cell nuclear transfer over current techniques. As shown in e.g., working Example 6 herein, the reprogrammed cells developed using a method and/or stem cell culture of the present disclosure described herein resulted in a 35.3% blastocyst rate as compared to a 16.1% blastocyst rate with the use of conventional skin fibroblasts.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

Leading up to the 1980s, studies on ruminants were at the forefront of developments in reproductive physiology and assisted reproduction for mammalian species (Britt et al., 1981; Willett, 1956). Derivation of murine embryonic stem cells (mESCs) in 1981 (Evans and Kaufman, 1981; Martin, 1981), opened up new possibilities for advancement, and there was significant investment in deriving bovine ESCs (bESCs). Despite preliminary successes in isolating pluripotent cells from the bovine inner cell mass (Cherny et al., 1994; Saito et al., 1992; Sims and First, 1994a; Stice et al., 1996), it was not possible to effectively sustain these cells in culture (First et al., 1994; Talbot and Blomberg, 2008). After human ESCs (hESCs) were derived in 1998 (Thomson et al., 1998), it brought to light understanding that signaling mechanisms for sustaining ESCs in culture were quite divergent. Growth factor leukemia inhibitory factor (LIF) acting via STAT3 was found to support self-renewal in mESCs (Niwa et al., 1998; Williams et al., 1988), and fibroblast growth factor 2 (FGF2) acting via activin/nodal were identified for hESCs (James et al., 2005b; Vallier et al., 2005). Numerous attempts to derive and study bESCs have continued over the years without definitive methods to sustain pluripotency and self-renewal long-term (Cao et al., 2009; Cibelli et al., 1998; Cong et al., 2014; Iwasaki et al., 2000; Kim et al., 2012; Lim et al., 2011; Mitalipova et al., 2001; Pant and Keefer, 2009; Pashaiasl et al., 2010; Pashaiasl et al., 2013; Saito et al., 1992; Saito et al., 2003; Sims and First, 1994b; Stice, 1996; Talbot et al., 1995; Van Stekelenburg-Hamers et al., 1995; Wang et al., 2005); the most recent studies being demonstrations that a primed form of bESCs (Bogliotti et al., 2018) and other early transitional states (Huang et al., 2014) that express pluripotency markers and retain the ability to differentiate into the three germ layers, can be cultured from bovine blastocysts. Hitherto, these studies have not only highlighted serious challenges to sustenance of pluripotency, but also the paucity in understanding of signaling and regulation for self-renewal in bESCs (Ezashi et al., 2016; Keefer et al., 2007; Malaver-Ortega et al., 2012).

In 2006, discovery of induced pluripotent stem cells (iPSCs) that had characteristics identical to ESCs (Takahashi and Yamanaka, 2006; Takahashi et al., 2007), infused new enthusiasm for bovine pluripotency research. In the ensuing decade, Applicant and others have made numerous attempts to generate biPSCs incorporating the four core reprogramming genes POU5F1 (OCT4), SOX2, KLF4 and MYC (OSKM), and other factors (Bai et al., 2016; Canizo et al., 2018; Cao et al., 2012; Cravero et al., 2015; Han et al., 2011; Heo et al., 2015; Huang et al., 2011; Kawaguchi et al., 2015; Lin et al., 2014; Pillai et al., 2019b; Sumer et al., 2011; Talluri et al., 2015; Wang et al., 2013; Zhao et al., 2017), but with limited success. Although several of these studies claim successful derivation of these cells, measures of quality have remained quite arbitrary (Pieri et al., 2019). In genome-integrating transgene based-approaches, the exogenous transgenes were not silenced (Cao et al., 2012; Cravero et al., 2015; Han et al., 2011; Heo et al., 2015; Huang et al., 2011; Sumer et al., 2011; Talluri et al., 2015; Zhao et al., 2017), or this was not evaluated (Canizo et al., 2018; Lin et al., 2014; Wang et al., 2013). In some studies, forced expression of the reprogramming genes induced trophoblast formation from bovine fibroblasts rather than pluripotent cells (Kawaguchi et al., 2016; Talbot et al., 2017). In the case of doxycycline-inducible reprogramming transgenes, continuous induction of exogenous expression was necessary to maintain bovine iPSCs (Kawaguchi et al., 2015). Supporting the lack in activation of the endogenous pluripotency network, some studies have concluded that bovine fibroblasts present an epigenetic block that prevents complete reprogramming (Canizo et al., 2018; Kawaguchi et al., 2015). In agreement, an extrapolation that progenitors can reprogram more readily than terminally differentiated cells was confirmed (Bai et al., 2016; Kawaguchi et al., 2015; Lin et al., 2014; Pillai et al., 2019b; Wang et al., 2013). This insufficiency led to additional testing for including Nanog (Pillai et al., 2019b; Sumer et al., 2011), knockdown of p53 (Pillai et al., 2019b), knockdown of Mbd3 (Pillai et al., 2019b), and overexpression of the microRNA 302/367 cluster (Bai et al., 2016; Pillai et al., 2019b), without success. For sustenance, these studies have attempted using LIF (Heo et al., 2015; Huang et al., 2011; Lin et al., 2014; Wang et al., 2013), FGF2 (Bai et al., 2016; Canizo et al., 2018), both (Cao et al., 2012; Cravero et al., 2015; Han et al., 2011; Kawaguchi et al., 2015; Pillai et al., 2019b; Sumer et al., 2011; Talluri et al., 2015; Zhao et al., 2017) or with added BMP4 (Zhao et al., 2017), without a clear functional consensus. Together with LIF, some studies have also tried pharmacological inhibition of mitogen-activated protein kinase kinase (MAP2K1/2 or MEK1/2), and glycogen synthase kinase 38 (GSK3β) to prevent differentiation and promote self-renewal (Heo et al., 2015; Huang et al., 2011). Despite these sustained efforts, a consistent and reproducible method for maintaining a pluripotent state remains to be deciphered for cattle or for any other ruminant species (Pieri et al., 2019; Su et al., 2020).

In investigating a variety of approaches to enhance efficiency in biPSC generation (Pillai et al., 2019b), Applicant has identified two compounding facets to the problem: (i) a stable epigenome in bovids resists iPSC reprogramming, exemplified by failures with methods that enhance permissiveness in murine and human somatic cells (Pillai et al., 2019b); (ii) there is dearth in understanding of signaling necessary for sustaining pluripotency and/or differentiation specific to bovids (Ezashi et al., 2016; Pillai et al., 2019b).

To address these problems, Applicant first experimented an approach using the simian vacuolating virus 40 large T antigen (LT), that would likely overcome any epigenetic barriers to iPSC generation (Tan et al., 2017), and then used stage-specific transcriptomics datasets to delineate pluripotency [subtracting undifferentiated trophoblast stem cells/TSCs (Pillai et al., 2019a) from blastocyst embryos] to deduce intracellular signaling that could be preventing/suppressing post-induction long-term sustenance of biPSCs in culture. These trials culminated in a precise reproducible approach for induction and culture conditions for sustained self-renewal of naïve biPSCs. Transcriptome analysis of these biPSC in comparison to the 16-cell bovine embryos (16-CEs) revealed for the first time underlying constants (transcriptional networks and signaling pathways) that support the bovine pluripotent cell phenotype. See also Pillai et al. Biol Open. 2021 Oct. 15; 10(10):bio058756.doi: 10.1242/bio.058756, which is incorporated by reference as if expressed in its entirety herein.

### Materials and Methods

### Primary Cells and Culture

Bovine embryos (at 35-45 days in development) were collected from the abattoir (Cargill, Wyalusing, PA) for culturing embryonic fibroblasts (BEFs) as previously described (Pillai et al., 2019b). Embryos were first decapitated and eviscerated before mincing into small pieces less than 1 mm³, then plated for culture in fibroblast medium [Dulbecco's minimal essential medium with high glucose containing 10% fetal bovine serum, 1% non-essential amino acids supplement, and penicillin-streptomycin]. Cells were allowed to grow in a 37° C. humidified incubator under an atmosphere of 5% CO₂. Once cells were confluent, they were passaged twice for expansion and frozen aliquots prepared for experiments. Irradiated mouse embryonic fibroblast feeders (iMEFs) were prepared from cells cultured from embryonic day 13.5 mouse embryos as previously described (Pillai et al., 2019b).

### Viral Vectors and Induction of Reprogramming

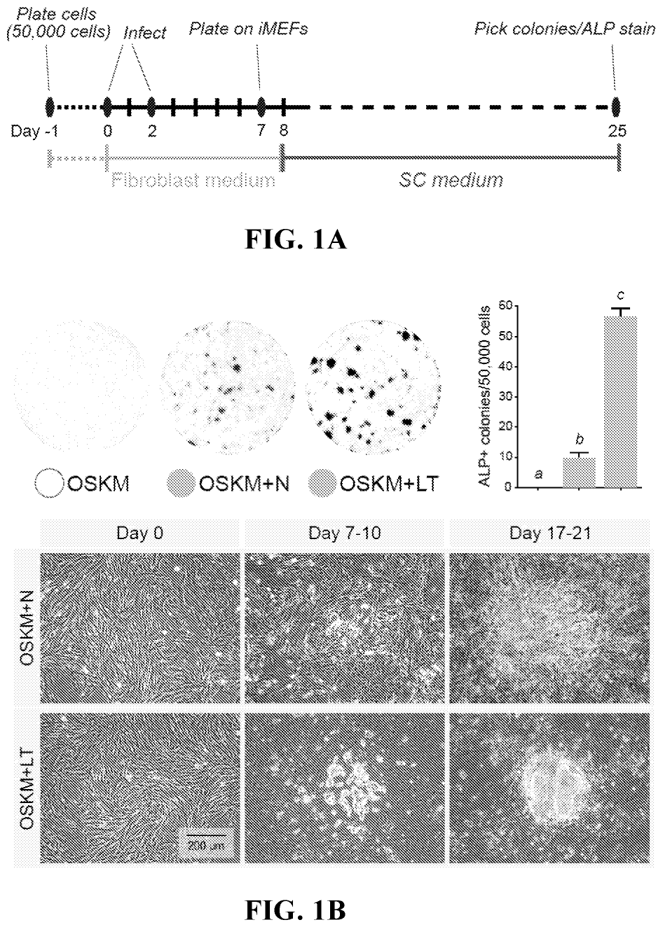

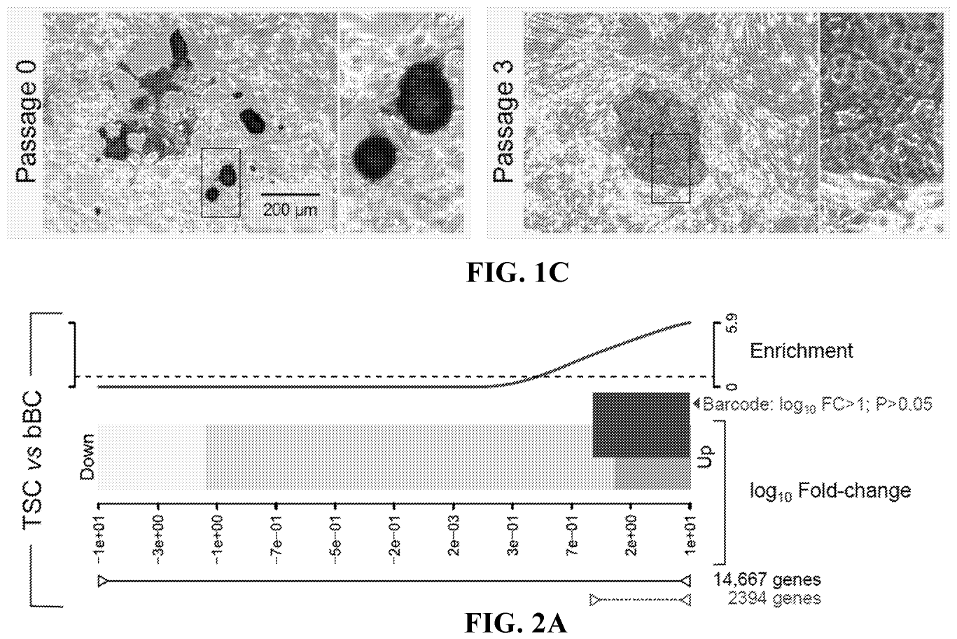

The human STEMCCA polycistronic lentiviral reprograming vector (Sommer et al., 2009), a lentiviral simian vacuolating virus 40 large T antigen vector (Mali et al., 2008a), and bovine Nanog previously generated by gene synthesis (Pillai et al., 2019b) and inserted into a lentiviral backbone (pLenti-EF1α) were used. In brief, 293T cells were co-transfected with gene inserts and helper plasmids that encode for lentiviral Gag, Pol, and Env proteins as previously described (Pillai et al., 2019b). Virus containing supernatants were collected at 48 and 72 hours, pooled, and passed through a 0.45 μm syringe filter before use in infecting BEFs. A green fluorescent protein (GFP) expressing pLenti-EF1α-GFP vector was used to package control lentiviruses to monitor packaging and infection efficiency. Method timeline is as indicated in FIG. 1A: BEFs were infected using STEMCCA in fibroblast medium supplemented with 6 μg/mL Polybrene (Sigma) for 24 hours. Seven days after infection, BEFs were collected by trypsinization (0.5% Trypsin EDTA, Millipore) and plated on irradiated mouse embryonic fibroblasts (iMEFs) at a density of 2.5×10⁴ cells/cm². After cell attachment the next day, medium was changed to stem cell (SC) medium [fibroblast medium containing 0.1 mM β-mercaptoethanol, 10 ng/mL of human leukemia inhibitory factor (LIF, Millipore) and 20 ng/ml of human fibroblast growth factor 2 (FGF2, Peprotech)]. To assess reprogramming efficiency, plates were collected at Day 25 for staining and quantification of colonies. For propagation, individual colonies were manually picked, dissociated into single cells using TrypLE™ (ThermoFisher) and plated on iMEFs in SC medium. Subsequent passages were performed by identical picking, dissociation, and plating of individual colonies on iMEFs. Passaging was continued as long as cells/colonies could be identified and propagated. All cultures were maintained in a humidified incubator at 37° C. under an atmosphere of 5% CO₂. Images were acquired using an inverted microscope (DMIL-LED, Leica) using a high-definition camera (MC190HD, Leica).

### Alkaline Phosphatase Staining

Labeling for alkaline phosphatase activity was performed using a kit (Vector Blue AP Substrate Kit) to visualize and quantify biPSC reprogramming progress. Reagents were added according to manufacturer instructions to 10 ml Tris-HCl, pH 8.5 buffer, and sufficient solution was added to wells containing cells and incubated for 30 minutes at 37° C. under an atmosphere of 5% CO₂. Entire plates were imaged for quantifying biPSC colonies for estimating reprogramming efficiency.

### Analysis of Signaling in the Bovine Inner Cell Mass

RNA-sequencing was performed using bovine blastocysts (day 7 after in vitro fertilization). In vitro embryo production was as previously described (Pillai et al., 2019a). Three independent batches of blastocysts produced were used for sequencing (˜120 blastocysts/group). In brief, total RNA was extracted from three independent groups of blastocysts by using RNAqueous Micro Total RNA Isolation Kit (ThermoFisher Scientific). Integrity of total RNA was checked using the Bioanalyzer 2100 (Agilent Technologies). Poly(A) capture was used to isolate mRNA. Fragmentation and cDNA library construction performed using TruSeq stranded total RNA sample preparation kit (Illumina). Three samples with unique bar code sequences were pooled for sequencing by synthesis to obtain short single reads on a HiSeq4000 (Illumina). Raw reads were subjected to quality control checks using FastQC tool (Babraham Bioinformatics). Reads were mapped to the bovine genome (ARS UCD 1.2) using bovine genome annotation file (Ensembl) using spliced transcripts alignment to a reference/STAR (Dobin and Gingeras, 2015). Comparisons were done to identify differentially expressed genes between blastocysts and undifferentiated TSCs [previously published by our group, GEO repository: GSE122418 (Pillai et al., 2019a)] using R package EdgeR (Robinson et al., 2010). Linear modeling, differential expression and a barcode plot visualization tool, Limma (Ritchie et al., 2015), was used for enrichment and examine genes that were significantly upregulated in the blastocysts compared to TSCs, to delineate gene expression associated with the inner cell mass/ICM. This gene list was then examined for known pluripotency-associated factors and subjected to analysis of overlap with gene signatures associated with stem cells using StemChecker [SysBiolab©] (Pinto et al., 2015). After the above validation, the gene list was subjected to enrichment analysis using the ESCAPE database with the Enrichr tool (Xu et al., 2013), to identify specific ‘Kinase Perturbations’ in the inner cell mass signaling pathways. Complete embryo RNA-seq datasets are available through NCBI GEO (GSE169674).

Sustenance of biPSCs Using Specific Pathway Inhibition

In iterative testing of methods compiled from previous published results (Pillai et al., 2019b) together with integrated data mining for signal transduction, Applicant established the GMTi medium that contained inhibitors for GSK3β, MEK1/2 and TGFβ/activin/nodal [DMEM/F12 containing N2 supplement, B-27 supplement, 1% non-essential amino acids supplement, 1% penicillin-streptomycin, 0.1 mM β-mercaptoethanol, 1.5 μM CHIR99021, 1 M PD0325901, 0.5 μM A83-01, and 20 ng/ml of hLIF or 20 ng/ml hIL6]. The biPSC colonies emerging from reprogramming trials were manually picked, dissociated into single cells using TrypLE™ (ThermoFisher) and plated on iMEFs in GMTi medium. For passaging, confluent cultures of biPSCs were rinsed once with PBS and incubated with TrypLE for 5 minutes. Cells were then collected in fibroblast medium, and centrifuged at 200×rcf for 5 minutes. The pellet was then resuspended in GMTi medium for plating on iMEFs. Passaging of biPSCs was performed repeatedly (every 3-4 days), with concurrent examination and imaging of morphology, growth characteristics and expression of PSC markers. Growth/expansion was indirectly estimated by measuring the surface area of biPSC colonies using ImageJ (Schneider et al., 2012). Base medium conditions lacking individual inhibitors for GSK3β, MEK1/2 or TGFβ/activin/nodal were also prepared and tested for their ability to sustain pluripotent cultures. All cultures were maintained in a humidified incubator at 37° C. under an atmosphere of 5% CO₂.

Feeder-Free Culture of biPSCs

Cell culture dishes coated with gelatin or Matrigel® were used for feeder-free cultures. For gelatin coating, culture dishes were incubated with 0.2% gelatin from porcine skin (Type A, MilliporeSigma) for 24 hours in a 37° C. humidified incubator, solution aspirated and the gelatin film allowed to air dry before use. For Matrigel® (Corning) coating, culture dishes were incubated with Matrigel® (diluted in cold DMEM/F12, 9 μg/cm²) for 2 hours in a 37° C. humidified incubator, rinsed once with DMEM/F12 before immediate use. To avoid iMEFs when transition to feeder-free cultures, biPSC colonies were picked and dissociated into single cells using TrypLE and plated in GMTi medium. Subsequent passages were as described for propagation on iMEFs. Cells were examined and imaged for morphology, growth characteristics and expression of PSC markers. All cultures were maintained in a humidified incubator at 37° C. under an atmosphere of 5% CO₂.

### Immunocytochemistry

Bovine iPSCs were grown on coverslips seeded with irradiated MEF feeders and fixed with 4% formaldehyde. Cells were then permeabilized with 0.1% Triton X-100 in PBS for 1 minute and blocked using 5% normal goat serum for 30 minutes. Coverslips were subsequently incubated with antibodies against SSEA1, 4 and 3 (1:200 dilution; Iowa Hybridoma Bank) for 1 hour. Coverslips were then washed three times using PBS and incubated with Alexa Fluor conjugated anti-mouse Fab′ fragments for 30 minutes, washed again with PBS, counterstained/mounted with 4′,6-diamidino-2-phenylindole (DAPI) containing Prolong Gold reagent (Life Technologies, Carlsbad, CA). Images were acquired using an inverted microscope (DMI 3000, Leica) using a cooled monochromatic camera (DFC365FX, Leica).

### Karyotyping

Bovine iPSCs were cultured under feeder-free and treated with 0.1 μg/ml mitotic arrestant, colcemid (Life Technologies) for 16 hours. The cells were then rinsed with PBS, and trypsinized to obtain a single cell suspension and pelleted by centrifugation at 100×g for 5 min. The cells were then resuspended in 5 ml of hypotonic solution (0.56% KCl) and incubated at 37° C. for 30 minutes and fixed with methanol:acetic acid solution (3:1, Carnoy's solution). Drops of the cells in suspension were collected with impact on glass slides that were pretreated with Carnoy's solution (1 minute) and washed with ice cold water. Slides were subsequently air-dried and stained with 5% Giemsa solution for 2 min and rinsed in water, air-dried again and mounted with coverslips using Permount (Sigma). The spreads were imaged using a light microscope (Leica DM750 LED) and high-definition camera (Leica ICC50W) and chromosome numbers in individual spreads were counted.

### Reverse Transcriptase Polymerase Chain Reaction

Total RNA was extracted from BEFs infected with STEMCCA lentivirus (Day 2), and biPSCs cultured on iMEFs in GMTi medium at passages 2 and 10 by sequential purification using Trizol™ (Life Technologies) and the RNeasy Mini Kit (Qiagen). Reverse transcription (cDNA synthesis) was carried out using 2 μg of total RNA with the Multiscribe™ reverse transcription kit (Life Technologies). Expression of the STEMCCA transgene was examined by performing polymerase chain reaction using primer pair: 5′-TTCACATGTCCCAGCACTACC-3′ (SEQ ID NO: 1) and 5′-GAAGCCGCTCCACATACAGT-3′ (SEQ ID NO: 2) that specifically amplifies a 560 bp region of the cDNA synthesized from the polycistronic STEMCCA mRNA. Expression of the LT transgene was examined by performing polymerase chain reaction using primer pair: 5′-GGCTACACTGTTTGTTGCCC-3′ (SEQ ID NO: 3) and 5′-GCCTGCAGTGTTTTAGGCAC-3′ (SEQ ID NO: 4) that specifically amplifies a 439 bp region of the cDNA synthesized from the LT mRNA.

Transcriptome of Bovine iPSCs

Colonies of biPSCs (passage 8) from three independent reprogramming events were selected for RNA-sequencing. Methods identical to that mentioned for blastocysts were used to extract mRNA, prepare libraries and sequence biPSC samples. After quality control and mapping, comparisons were performed to identify: (a) differentially expressed genes between biPSCs and undifferentiated TSCs, and (b) differentially expressed genes between biPSCs and fibroblasts, or 16-CE and fibroblasts, independently using R package EdgeR (Robinson et al., 2010). Transcriptome of primary bovine fibroblasts was from control samples in GEO repository: GSE61027 (Green et al., 2015). Gene expression associated with bovine trophectoderm have been previously defined (Pillai et al., 2019a); genes associated with formation of the ectoderm, mesoderm and endoderm were as previously compiled (Maguire et al., 2013), and confirmed using gene ontology definitions (GO: 0007492, 0007398, 0007498). Complete biPSC RNA-seq datasets are available through NCBI GEO (GSE169624).

### Embryoid Body Formation

Feeder-free cultures of biPSCs were dissociated into single cells using TrypLE, and resuspended in DMEM/F12 containing 10% FBS to achieve a concentration of 25,000 cells/ml. Rows of 20 μl hanging droplets for suspension culture of biPSCs were made on an up-turned lid (inner surface) of a 150 mm tissue culture dish. Inverting the droplets, the lid was placed on the dish that contained 10 ml PBS for maintaining humidity, and this setup was incubated at 37° C. under an atmosphere of 5% CO₂ for 2 days for embryoid body formation. Each embryoid body was then transferred to single wells of a low attachment 96 well plate and cultured for 3 more days. Images were acquired using stereo microscope (M80, Leica) using a high-definition camera (IC80HD, Leica).

### Teratoma Assay

Feeder-free cultures of biPSCs were dissociated into single cells using TrypLE, and resuspended in cold Matrigel® diluted in DMEM/F12 (80 μg/ml final) at a concentration of ˜10⁶ cells in 200 μl. Cell suspension was loaded into a chilled 1 ml syringe with a 30G needle and transported on ice. Immunodeficient NSG mice [NOD.Cg-Prkdc^{scid}Il2rg^{tm1Wjl}/SzJ, Jax® Mice (Shultz et al., 2005)], 6-8 weeks of age, were subcutaneously injected at 2-3 sites in the flank and/or back, introducing 50-100 μl of cell suspension at each site. NSG mice were maintained under standard care and monitored for physical appearance of teratomas. At 6 weeks after injections, mice were euthanized and teratomas collected and fixed in 4% formaldehyde and held for histological processing. Paraffin embedding, cutting thin sections (4 μm thickness), and staining using hematoxylin and eosin were as previously described for mouse tissues (Morohaku et al., 2013; Morohaku et al., 2014; Tu et al., 2014). Morphological assessment for differentiation was performed by identifying the diversity of tissue types using methods in histopathology. Images were acquired using an upright light microscope (DM1000LED, Leica) using a high-definition camera (ICC50HD, Leica).

### Analysis of Transcriptional Regulation and Pathways

Transcriptome of bovine 16-CEs were used as a reference to examine the equivalence of biPSC for both authentication and advancing understanding of pluripotency regulation and pathways. Transcriptome of bovine 16-CE was from GEO repository: GSE52415 (Graf et al., 2014). The bovine fibroblast transcriptome was used as a normalizing dataset to delineate genes upregulated in pluripotency. After gene expression analysis for the 16-CE and biPSC datasets, transcription factor enrichment analysis was performed using ChEA3 (Keenan et al., 2019), to identify factors responsible for gene expression in 16-CEs and biPSCs. ReMap transcription-factor target gene set library was used to process transcriptomics data from these groups yielding enrichment results/ranks. Distinct from this systems analysis, to precisely uncover the pluripotency-relevant transcriptional network, transcriptome of both 16-CE transcriptome and biPSC transcriptome were first compared to the transcriptome of primary bovine fibroblasts (Green et al., 2015) in order to reveal genes that are specifically upregulated in both. From this list, transcription factors were separated by using a comprehensive reference list of 1595 compiled from three databases (de Souza et al., 2018; Weirauch et al., 2014; Zhang et al., 2012). Transcription factors that were commonly upregulated in both 16-CEs and biPSCs were examined for relative expression levels to identify highly expressed transcription factors and their relevance to pluripotency was analyzed for functional association using the String database (Szklarczyk et al., 2017). Exploratory investigation and functional pathways associated with genes upregulated in biPSCs and 16-CEs compared to fibroblasts were analyzed using iDEP (Ge et al., 2018). Active receptor-mediated signaling in biPSCs were identified by integrated gene expression and protein interaction analysis using SPAGI (Kabir et al., 2018).

### Results

Inclusion of LT Considerably Enhances the Efficiency of Inducing biPSCs

Use of LT in addition to OSKM reprogramming factors resulted in a dramatic increase in biPSC colony formation (FIG. 1B). These colonies showed rounded edges with individual cells not being discernible. Use of OSKM alone did not result in biPSC induction, and addition of Nanog to OSKM resulted in partially reprogrammed biPSC-like cells that formed dense accumulations without rounded edges (FIG. 1B), also previously demonstrated (Pillai et al., 2019b). The compact colonies induced by OSKM+LT showed intense staining for alkaline phosphatase (ALP) (FIG. 1C; Passage 0). Nevertheless, these OSKM+LT induced biPSC colonies could not be sustained. With picking and passaging colonies using SC medium on MEF feeders, their morphology rapidly changed by Passage 2, and appeared flattened with individual cells being discernible and a weaker ALP positive intensity by Passage 3 (FIG. 1C; Passage 3). These colonies could not be maintained beyond Passage 3 or 4, and were lost without any structural aggregation and any residual cells in the locale became ALP negative.

### TGFβ Signaling is Prominently Disrupted in the Bovine Blastocyst Inner Cell Mass

In quantitative comparison of the transcriptome between early bovine blastocysts and undifferentiated TSCs, Applicant was able to delineate gene expression specific to the inner cell mass (FIG. 2A; Table 2). Within the cut-off value (log₁₀ fold-change >1 and p-value<0.05), Applicant identified a list of 2,394 genes possibly linked to the inner cell mass. Inspecting known pluripotency-specific markers across different species revealed that this delineated gene list was highly enriched in expression of genes restricted to pluripotent cells (FIG. 2B). Analysis of this gene list for overlap with the gene signature of stem cells also showed significant enrichment of transcripts associated with ESCs, iPSCs and embryonal carcinoma cells (FIG. 2C). In kinase perturbation analysis of this gene list to discover signaling pathways suppressed in the blastocyst inner cell mass, Applicant uncovered that transforming growth factor β (TGFβ) signaling via TGFβ receptors (TGFBR1 and TGFBR2) were actively suppressed and highly significant (FIG. 2D). Trophectoderm/TSCs that contribute to the blastocoel microenvironment were found to express TGFβ1, TGFβ2 and TGFβ3, concurrently with high levels of latent TGFβ binding proteins (LTBPs; FIG. 2E).

Inhibition of TGF Activin Nodal Pathway Supports Robust biPSC Sustenance

Use of GMTi medium that contains inhibitor A83-01 (Tojo et al., 2005), to block downstream TGFβ signaling via SMADs together with inhibitors for GSK3β and MEK1/2, supported robust self-renewal and repeated passages in sustaining cultures of biPSCs (FIG. 3A). Colonies showed a sustained morphology indicative of naïve pluripotency with little change from the time of dedifferentiation, with rounded edges and individual cells not being discernible (FIG. 3A). Colonies consistently stained positive for SSEA1 (FIG. 3B). After 22 repeated passages, clonal populations of biPSCs were found to have a normal karyotype (chromosomes 2n=60; FIG. 3C). Exogenous OSKM delivered by the STEMCCA vector was silenced in biPSC in the later passages (FIG. 3D). Expression of LT was also silenced in biPSCs examined at later passages (FIG. 3E). Gene expression profiling showed upregulation of endogenous pluripotency-associated genes in biPSCs (FIG. 3F). Comparing expression of genes previously associated with naïve or primed pluripotency between 16-CE and biPSCs indicated a generally similar pattern of expression (FIG. 3G). Gene expression specific to the trophectoderm (TE), and differentiation of the endoderm, mesoderm and ectoderm were not expressed in biPSCs (FIG. 3F; Table 3).

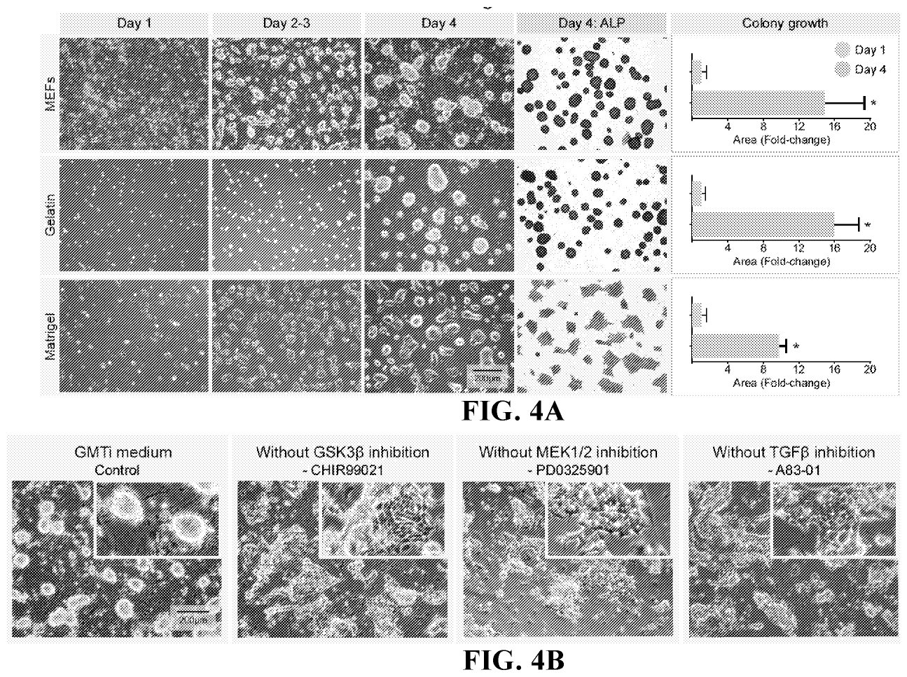

Culturing biPSC cells with GMTi medium allowed cells to grow in different cell-free substrates (FIG. 4A). They maintained a strong ALP positive characteristic with no tendency toward differentiation. Rapid proliferation could be detected by average colony size increases by approximately 15-fold, 16-fold and 10-fold in a period of 4 days when grown on iMEFs, gelatin and Matrigel® respectively. Experiments excluding single inhibitors to GSK3β (CHIR99021), MEK1/2 (PD0325901) or TGFβ/activin/nodal (A83-01) indicated that all three are necessary to sustain pluripotency with colony morphology indicative of naïve cells. Removal of even one of these three inhibitors in biPSC cultures resulted in differentiation based on shifts to cell morphology that could be noted within 48 hours (FIG. 4B). Beyond 48 hours, cells showed complete spontaneous differentiation (data not shown).

biPSCs Differentiate into Tissues of the Three Germ Layers

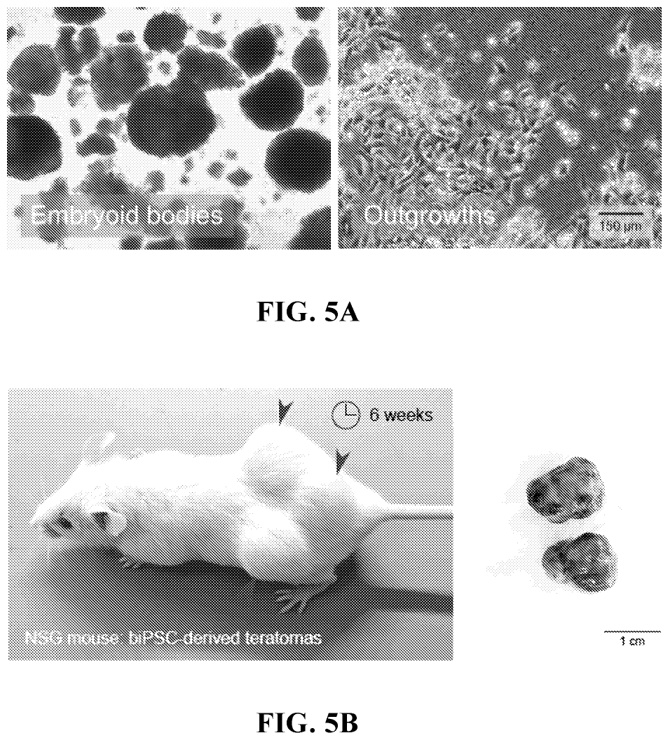

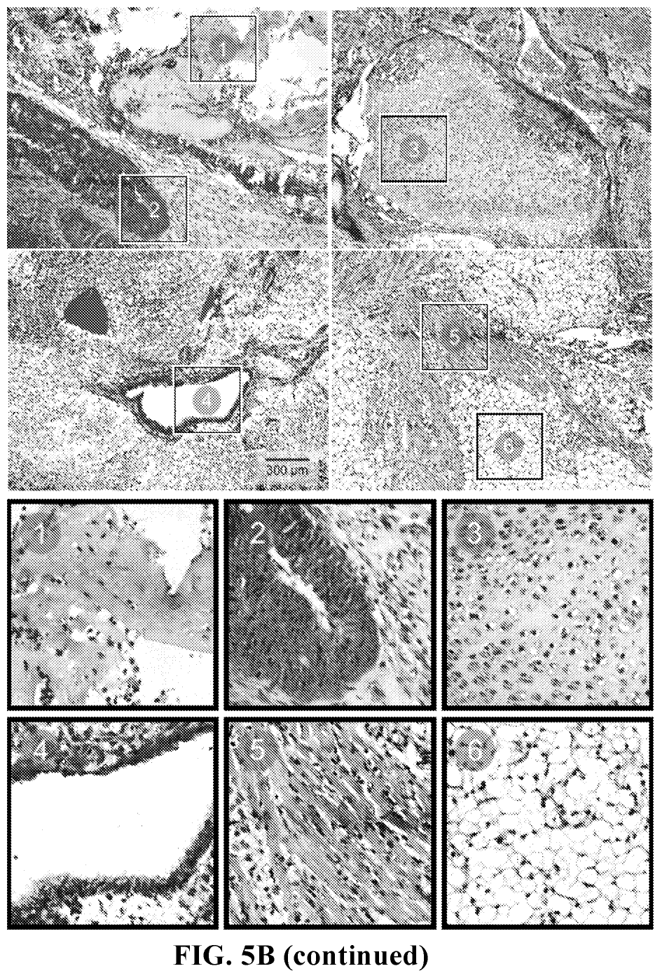

Differentiation of biPSCs to embryoid bodies could be attained in vitro with suspension cultures without providing the inhibitors for self-renewal (FIG. 5A). These embryoid bodies were viable and generated outgrowths of differentiated cells when plated in cell culture treated dishes. Subcutaneous grafting of biPSCs suspended in Matrigel® into immunodeficient NSG mice resulted in prominent teratoma growth by 6 weeks (FIG. 5B). Teratoma sizes per injection site were approximately 1 cm in diameter. Histological analysis of teratomas indicated the presence of different tissue types representing ectoderm, mesoderm and endoderm differentiation of biPSCs.

Transcriptome Uncovers Regulatory Machinery Associated with Bovine Pluripotency

Analysis of active transcription that captures the majority of gene expression seen in biPSCs indicated a repertoire of 50 prominent factors between 16-CEs and biPSCs that dictated the phenotypic state (FIG. 6). This ChEA analysis indicated a core level of commonality in gene regulation indirectly informing of the chromatin state (not strictly linked to pluripotency). Although all the predicted transcriptional regulators were expressed in both biPSCs and 16-CEs, in quantitative comparisons with fibroblasts, only 10 transcription factors (SOX2, E2F7, HNF1B, OTX2, POU2F1, FOXM1, HHEX, TCF7, HINFP and ZNF318) were prominently upregulated >2-fold in both indicating an exclusive phenotypic contribution of these factors to bovine pluripotent cells (available gene descriptions provided in Table 4).

In separate analysis, comparing the full list of transcription factors that were upregulated relative to fibroblasts (FDR <0.05) between biPSCs and 16-CEs indicated 312 common factors (FIG. 7A). This represented an exhaustive list of all the transcriptional regulators likely involved in pluripotency (Table 5). Plotting relative abundance (fold-change) between biPSCs and 16-CE to visualize the most prominent regulators revealed 77 common factors that were >3-fold higher in both biPSCs and 16-CEs (quadrant II, FIG. 7B). Examining expression levels (CPM) of these 77 common regulators indicated a consistent range in the levels of baseline expression in both 16-CEs and biPSCs (FIG. 7C). This list contained numerous factors that are not previously associated with pluripotency, and factors that remain uncharacterized genes. Mapping for functional association in the common regulators uncovered what is known of the transcriptional framework associated with bovine pluripotency, which also indicated that the biPSCs and 16-CEs had fundamental similarities (FIG. 7D). In addition to containing all four reprogramming-capable genes (OSKM), the map identified known interactions with other factors associated with specific functions such as epigenetic modification/chromatin remodeling, transcriptional repression/activation, cell cycle regulation, X chromosome inactivation, and estrogen signaling (Table 5). This overlap between biPSCs and 16-CE indicates similarities in transcriptional regulation and/or epigenetic state for existing or in preparation for future functional states.

### Integrated Algorithms Reveal Scope of Extrinsic Signal Perception in Bovine PSCs

Pathway analysis performed using genes upregulated in biPSCs and 16-CEs (in comparison to fibroblasts), indicated common enrichment to functional features (FIG. 8A). Beyond the basic cell maintenance and regulatory mechanisms, specific enrichment indicating rapid cell turnover and genome maintenance were uncovered: ribosome biogenesis, DNA replication, cellular senescence, oocyte meiosis, homologous recombination, mismatch repair and ribosome biogenesis. Examination for upstream receptor-mediated signaling revealed 11 surface membrane receptors common to both biPSCs and 16-CEs that capture signaling and active transcriptional mechanisms that represent the bovine pluripotent state (FIG. 8B). Receptor for the leukemia inhibitory factor family member IL6 was part of this list together with several other receptors previously unassociated with pluripotency. Expression levels examined for these receptors indicate that they were all significantly upregulated in biPSCs and 16-CEs compared to fibroblasts (FDR >0.001; FIG. 8C). This corroborated that the identified receptors are specific and relevant to bovine pluripotent cells.

### Discussion

In this report, Applicant presents the formula for generating naïve biPSCs with complete reprogramming to pluripotency, prolonged self-renewal capacity and silenced transgenes, a task that has remained a challenge despite numerous studies on this topic (Su et al., 2020). Using these cells, Applicant has uncovered core characteristics of transcriptional regulation and signaling that defines the bovine pluripotent state allowing comparative evaluation based on what is known in other species.

From previous work, Applicant concluded that bovine fibroblasts might have a stable epigenome that makes them refractory to complete reprogramming; OSKM did not induce colony formation in bovine fibroblasts (Pillai et al., 2019b). This meant that even if pluripotency genes are active/induced, cells are fixed in a differentiated phenotype or readily revert to a differentiated phenotype as large sections of the epigenome do not support the pluripotent self-renewal program. As reprogramming efficiency is positively correlated to the rate of complete reprogramming (Mikkelsen et al., 2008), Applicant investigated an extra factor LT together with OSKM. It has been shown that addition of LT with OSKM shows dramatic improvement to the pace and reprogramming efficiency in human cells (Mali et al., 2008b; Park et al., 2008a; Park et al., 2008b). Recently, it was shown that LT could help reprogram naked mole rat fibroblasts that were documented to be resistant to reprogramming by OSKM alone (Tan et al., 2017). Consistent with these reports, Applicant found that LT significantly enhances reprogramming efficiency in cattle.

Albeit not explained on the basis of complete reprogramming, such effect has also been indicated in certain ruminants such as sheep (Bao et al., 2011) and goats (Ren et al., 2011). Linked to a variety of influences encompassing transcription and epigenetics, LT affects a gamut of cellular targets/processes (Ahuja et al., 2005). One of the prominent effects of LT reported in the literature is its interaction and inactivation of TP53 (p53) functions, and the retinoblastoma family of proteins (particularly RB), dysregulating pivotal checkpoints in cell cycle control (Ali and DeCaprio, 2001). Although knockdown of TP53 has been shown to increase reprogramming efficiency in murine and human cells (Kawamura et al., 2009; Zhao et al., 2008), Applicant does not believe LT action in inducing biPSCs is via an effect on TP53, as similar knockdown of TP53 in BEFs did not enhance reprogramming efficiency (Pillai et al., 2019b). The equilibrium guiding TP53 and RB activities are known to vary between species; in the naked mole rat, reprogramming block released by LT was identified as being due to RB rather than TP53 (Tan et al., 2017). It has been shown that RB stabilizes heterochromatin via interactions with H3K9 methylases (Ait-Si-Ali et al., 2004; Nielsen et al., 2001). In association, RB was demonstrated to restrict reprogramming in murine fibroblasts by maintaining a more repressive chromatin state (Kareta et al., 2015). In somatic cell nuclear transfer experiments using BEFs, it was identified that failure of H3K9 demethylation presented a block to nuclear reprogramming (Liu et al., 2018). In this context, it can be interpreted that the LT induced increase in biPSC reprogramming could be at least in part due to an effect on RB. Nevertheless, cells derived using OSKM+LT could not be sustained or passaged without spontaneous differentiation in culture using SC medium. At this point, it was unclear whether this was due to incomplete reprogramming or solely the lack of appropriate medium conditions for sustenance.

Studies using small molecule inhibitors for MEK1/2 (PD0325901) and GSK3B (CHIR99021) have been previously shown to suspend biPSCs in a pluripotent state, but without the ability to proliferate (Huang et al., 2011). It has also been suggested that use of these same two inhibitors during the reprogramming process could yield biPSCs capable of self-renewal in culture, with added valproic acid (to inhibit histone deacetylation) and ascorbic acid (to promote histone and DNA demethylation) (Heo et al., 2015). Use of these inhibitors originated from studies that defined the naïve “ground state” of mESCs, which demonstrated that inhibitors for MEK1/2, GSK3B and FGFR released pluripotency from the dependence of exogenous growth conditions (Ying et al., 2008). Concurrent use of N2 and B27 supplements could support serum-free bulk cultures of naïve mESCs (Ying et al., 2008). In contrast FGFR inhibition has been shown not to be critical for sustaining naïve hESCs that could be maintained with inhibition of MEK1/2, GSK3B and PKC in the presence of hLIF (Guo et al., 2016). Notwithstanding, our attempts to use the above inhibitors with and without growth factors on OSKM+LT reprogrammed cells did not support self-renewal and/or ability to passage colonies (not shown). Therefore, Applicant turned to analysis of gene expression relevant to the bovine blastocyst inner cell mass to learn more about pathway targets that may be exclusive for biPSCs.

Specific enrichment for signaling perturbation indicated TGFBR1 signaling as a top presentation in the bovine inner cell mass gene expression. In miPSCs, use of TGFβ inhibitors have resulted in faster and more efficient induction of iPSCs; conversely addition of TGFβ has been shown to block reprogramming (Maherali and Hochedlinger, 2009). Subsequently it was demonstrated that TGFβ inhibition supports pluripotency by reducing ERK phosphorylation in miPSCs (Tan et al., 2015). In contrast, it was demonstrated that TGFβ signaling is necessary for the maintenance of pluripotency in hESCs (James et al., 2005a). Although these studies made it clear that TGFβ effects are not conserved in pluripotent cells across different species, following our analysis, Applicant discovered that TGFβ inhibition coupled with MEK1/2 and GSK3B inhibition could support robust cultures of naïve biPSCs. As pluripotency in in vivo blastocysts is critically shaped by the trophectodermal contributions to the blastocoel fluid (Pillai et al., 2019a), without being bound by theory Applicant hypothesizes that the LTBPs present minimize the levels of TGFβ available and restrict morphogenesis during pluripotent expansion. It is well known that TGFβ family of proteins (that include activin and nodal) are critical for specifying the body plan during metazoan development (Wu and Hill, 2009). During the period of our work, it was also revealed that a 6-small molecule cocktail that included a TGFβ inhibitor in combination with MAPK14, MAPK8, MAP2K1, GSK3A and BMP support naïve porcine iPSC lines in the presence of both FGF2 and LIF (Yuan et al., 2019). In comparison, biPSC cultures were liberated from dependence on growth factors in GMTi medium, a significant step forward towards uncovering pluripotency regulation in bovids and ruminants.

Complete reprogramming and long-term sustenance have not been reproducibly achieved in previous attempts to generate biPSCs using OSKM factors alone. Colony morphology coupled to gene expression analysis indicated naïve-type biPSCs. Applicant bases this definition on the fact that pluripotency gene expression in these biPSCs closely reflects that observed in 16-CE. In specifically examining genes defined in studies on other species as associated with naïve or primed pluripotency, Applicant found that expression in the 16-CE representative of bovine naïve cells, is novel and not confined to this bifurcated pattern. These results present the molecular signature of naïve cells in ruminant pluripotency that is also observed in the generated biPSCs in GMTi medium. Applicant did not encounter any aberrant reprogramming into the trophoblast lineage although this has been reported to occur in bovine cells transduced with OSKM factors (Kawaguchi et al., 2016; Talbot et al., 2017). This perhaps suggests that OSKM+LT resulted in complete reprogramming, followed by sustenance that ultimately silenced the exogenous OSKM and LT expression. The biPSCs cultured across multiple passages, and expanded under feeder-free conditions, were robust in generating embryoid bodies and readily differentiated into teratomas that were composed of ectodermal, mesodermal and endodermal lineages. The ability to sustain the biPSCs provided for the first time an opportunity to rigorously examine the bona fide transcriptional regulation and pathways associated with pluripotency in cattle.

In evaluating the transcriptional contribution to the en masse phenotype of bovine pluripotency, Applicant identified 10 enriched factors that were consistently upregulated in biPSCs and 16-CEs. Of these SOX2 has been well known for its role in pluripotency sustenance across different species (Avilion et al., 2003; Rodda et al., 2005), and is a component of the reprogramming factors used for iPSC generation (Takahashi and Yamanaka, 2006). Similarly, POU2F1/OCT1, a paralog of POU5F1/OCT4 that shares binding specificity by heterodimerization (Fletcher et al., 1987; Tomilin et al., 2000), was found to be a substantial contributor. POU2F1 is also known to interact with other cofactors suggesting a larger repertoire of targets and distinct specificities to this paralog (Shakya et al., 2011; Tomilin et al., 2000). Both POU2F1 and POU5F1 are known to interact with SOX2, albeit with differential activation properties (Ambrosetti et al., 1997). Involvement of OTX2 has been linked to maintenance of the metastable ESC state by opposing self-renewal and predisposing cells to differentiation (Acampora et al., 2013). Function of TCF7 as a binding partner of beta-catenin, the core factor involved in the transcriptional output of WNT signaling is well known (Cadigan and Waterman, 2012; Mao and Byers, 2011). The mechanism of GSK3B inhibition used to maintain pluripotency (Sato et al., 2004), as used in this study to maintain biPSCs, is via promoting beta-catenin targets. The atypical E2F7 responsible for transcriptional repression at E2F sites (Carvajal et al., 2012; Di Stefano et al., 2003; Liu et al., 2013), also regulated the expression landscape. The contribution of HNF1B and ZNF318 remains unknown with no prior studies to provide an interpretation of possible function.

In parallel, expression-based cut-off identifying 77 transcription regulators highly consistent between biPSCs and 16-CEs showed the specific transcriptional regulation underlying bovine pluripotency. This list included transcription regulators already known to be associated with pluripotency, together with several factors previously not associated with pluripotency, and few uncharacterized genes in cattle. This list forms a core resource for future investigations into divergent aspects of bovine pluripotency regulation. Mapping known elements from the commonly upregulated list showed specific nucleation in the transcriptional network indicating core influences of OCT4/POU5F1, SOX2, MYC, E2F land EZH2.

The algorithm identifying the 11 common membrane receptors from corresponding integration of gene expression and signaling could be considered as rigorous, as all these receptors were highly expressed in both biPSCs and 16-CEs indicating high relevance in pluripotency. This list included known elements such as IL6R, from which, downstream signaling via STAT3 is known to sustain pluripotency in murine ESCs (Nichols et al., 1994). LIF, an IL6 family member is long known to support pluripotency signaling in murine ESCs (Smith et al., 1988; Williams et al., 1988). Recently, IL6 treatment has been shown to increase cell numbers of the inner cell mass in bovine blastocysts (Wooldridge and Ealy, 2019), via a direct or indirect mitogenic effect on bovine pluripotent cells. However, excluding LIF or IL6 from the GMTi medium did not negatively affect biPSCs at least over a few passages, suggesting that with GSK3B, MEK1/2 and TGFβ/activin/nodal inhibition, IL6R-based signaling does not add to pluripotency sustenance as indicated by cell morphology. Not to be discounted yet is that biomimicry by providing IL6 in long-term cultures could buttress endogenous mechanisms in long-term sustenance as IL6R is indeed expressed in both biPSCs and 16-CE. Another element KIT, a receptor tyrosine kinase is known to be expressed in ESCs (Palmqvist et al., 2005). It has been demonstrated that KIT inhibition can affect both self-renewal and survival of differentiating cells (Bashamboo et al., 2006; Fraser et al., 2013). Receptor HMMR, and potential for signaling mediated by hyaluronan has not been previously dissected in pluripotency. However, it was reported that hyaluronan-gelatin hydrogels could maintain murine and human iPSCs (Liu et al., 2012). Recently, it has been observed that highly sulfated hyaluronic acid could maintain primed human iPSCs by promoting FGF2-ERK signaling, even in the absence of recombinant FGF2 (Miura et al., 2019). Although expressed, Sonic Hedgehog (SHH) signaling in human and mouse PSCs has not been linked to self-renewal but only in differentiation toward the neuroectodermal lineage by some studies (Lau et al., 2019; Wu et al., 2010). In contrast, one study in murine ESCs suggests that SHH-mediated GLI1 activation and phosphorylation of EGFR supports self-renewal (Heo et al., 2007). In subsequent studies, a more intricate relationship has been established balancing pluripotency and differentiation in that NANOG interacts with GLI1 providing negative feedback, permissive only to PSC-specific regulation of SHH signaling (Li et al., 2016). Our identification of HHIP (a member of the hedgehog interacting protein family), and BOC (a member of the immunoglobulin/fibronectin type III repeat family), both components of the cell surface SHH receptor complex (Izzi et al., 2011), supports a possible role for SHH signaling in bovine pluripotency. Members of the low-density lipoprotein receptor family widely known to be involved in receptor mediated endocytosis and associated endosomal sorting of lipoprotein and other protein ligands (LRP2, LRP6 and SORL1) were also identified. Of these LRP6 has been shown to be a component of the WNT complex that triggers beta-catenin signaling (Cselenyi et al., 2008); LRP2 has been shown to act as an auxiliary SHH receptor by increasing signaling capacity (Christ et al., 2012); SORL1 has been shown to be integrally involved in IL6 signaling, specifically promoting capacity for soluble IL6R or trans signaling as opposed to the classic cis signaling (Larsen and Petersen, 2017). Transferrin receptor (TFRC) widely known for iron acquisition by all mammalian cells (Dautry Varsat et al., 1983; Jandl et al., 1959), was also identified; iron uptake has been recently shown to promote WNT/W-catenin signaling (Mandala et al., 2020; Song et al., 2011). Protease-activated G-protein coupled receptors (F2R and F2RL1) have not been previously studied in PSCs. There are a variety of known signaling mechanisms supported by these receptors (Heuberger and Schuepbach, 2019), the relevance of which requires additional investigation. Collectively, similarities presented in these signaling receptors between the 16-CE and biPSCs not only indicate its authenticity, but also presents novel information regarding extracellular signaling mechanisms/mediators that might find critical roles in sustaining the bovine pluripotent state.

In developing these methods and mechanisms, Applicant finds it difficult to reconcile with recent evidence that inhibition of WNT signaling (using IWR1) is crucial for the derivation and propagation of a primed form of bovine ESCs (Bogliotti et al., 2018), and bovine expanded potential stem cells (EPSCs) (Zhao et al., 2021) [the latter was during the time this manuscript was under review]. However, the distinctions Applicant finds do support the notion that maturation through a continuum of pluripotent states in vivo can be captured in the form of different stable transitional states in vitro (Morgani et al., 2017). WNT/β-catenin signaling has been reported to be critically calibrated in early development and pluripotency (Zhang et al., 2013). In naïve murine PSCs, repressing WNT signaling induced differentiation towards a primed epiblast stem cell (EpiSC) state (Berge et al., 2011), and in the primed state, WNT activation (using CHIR99021) can result in intermediate pluripotent stem cells (intPSCs) that exhibit characteristics of both ESCs and EpiSCs (Tsukiyama and Ohinata, 2014). Although it is difficult to extrapolate these mechanisms to bovine PSCs, it is plausible that the primed form of bESCs captured using IWR1 (Bogliotti et al., 2018), and bovine EPSCs captured using both IWR1 and CHIR99021 (Zhao et al., 2021), both derived from blastocysts, represent stable transitional states.

In conclusion, Applicant has successfully established completely reprogrammed naïve bovine iPSC lines that show core parallels to 16-CEs. In addition to opening up possibilities for comparative studies on the basis of pluripotency regulation in a species that has baffled scientists for decades, Applicant presents a complete tool for advancing reproduction and biotechnology applications in an agriculturally important species.

### Example 1 Supplemental Information

TABLE 1
Exemplary Stem Cell Culture Medium

| Base medium: DMEM/F12 (1:1) generic composition | | |
|---|---|---|
| Supplement | Conc (μg/ml in base | |
| ingredient | medium or as indicated) | Function/Activity |

Part 1: Pathway Inhibitors

| | | |
|---|---|---|
| CHIR99021 | 1.5 μM | GSK3β inhibitor, and as a result a potent |
| | | Wnt/β-catenin signaling pathway activator |
| PD0325901 | 1 μM | MEK1/2 inhibitor |
| A83-01 | 0.5 μM | TGFβ/activin/nodal inhibitor, acts via ALK4 and |
| | | inactivating ALK5, ALK5 kinases respectively. |

Part 1: Minimum essential components - sufficient for sustenance with inhibitors

| | |
|---|---|
| Insulin | 8.125 |
| Putrescine | 32.21 |
| Progesterone | 0.0128 |
| Holo Transferin | 105 |

Part 2: Optional components - provided added benefits to pluripotency
sustenance, and could be important for long-term PSC health

| | |
|---|---|
| Sodium selenite | 0.0177 |
| Linoleic acid | 1 |
| Linolenic acid | 1 |
| Biotin | 2.5 |
| Oleic acid | 1 |
| DL-alpha tocopherol | 1 |
| acetate | |
| Corticosterone | 0.02 |
| BSA | 2500 |
| Heparin sodium salt | 0.16 |
| Folic acid | 132.3 |

Part 3: Optional components: Growth factors,
other components for sustaining endogenous signaling

| | |
|---|---|
| LIF or IL6 | 0.02 |
| SCF | 0.01 |
| Hyaluronic acid | 10 |
| (sulfated) | |
| Serum lipoproteins | 100 |
| (LDL) | |

TABLE 2
RNA Seq Data - Relative expression of genes identified as upregulated in comparison between bTSCs and bBC (for barcoded genes shown in FIG. 2A).

| ENSEMBL GENE IDs | Gene name | logFC | logCPM | PValue | FDR |
|---|---|---|---|---|---|
| ENSBTAG00000019173 | PAG12 | 12.42435603 | 8.431385277 | 0 | 0 |
| ENSBTAG00000020676 | MMP9 | 12.1523843 | 8.161358824 | 6.37884662301517e−316 | 7.39336804920813e−313 |
| ENSBTAG00000017020 | S100G | 11.94660203 | 7.956972369 | 6.71E−292 | 4.68E−289 |
| ENSBTAG00000045630 | G3MYT1 | 10.93733249 | 6.958532351 | 4.69E−189 | 1.35E−186 |
| ENSBTAG00000053661 | A0A3Q1M7A0 | 10.91707263 | 6.939636967 | 3.24E−187 | 8.96E−185 |
| ENSBTAG00000013950 | TKDP4 | 10.78503609 | 11.2299198 | 0 | 0 |
| ENSBTAG00000037929 | ADAM28 | 10.75998867 | 6.785138188 | 1.24E−173 | 2.94E−171 |
| ENSBTAG00000009080 | ITGB6 | 10.58919361 | 6.61726989 | 8.71E−160 | 1.77E−157 |
| ENSBTAG00000002335 | HAND1 | 10.27643801 | 6.311373128 | 3.77E−136 | 5.58E−134 |
| ENSBTAG00000008483 | CALCRL | 9.966487933 | 6.009998766 | 1.26E−115 | 1.50E−113 |
| ENSBTAG00000005198 | FGF1 | 9.949536135 | 5.993339807 | 1.53E−114 | 1.77E−112 |
| ENSBTAG00000038662 | GJB6 | 9.616004151 | 5.670665825 | 3.03E−95 | 2.61E−93 |
| ENSBTAG00000005250 | BGN | 9.502133757 | 5.561985102 | 3.04E−89 | 2.39E−87 |
| ENSBTAG00000015460 | TP63 | 9.398801191 | 5.462692456 | 4.07E−84 | 2.96E−82 |
| ENSBTAG00000002348 | SLC4A4 | 9.340976476 | 5.407715711 | 2.10E−81 | 1.45E−79 |
| ENSBTAG00000020060 | TXNIP | 9.10287118 | 5.18096249 | 1.17E−70 | 6.55E−69 |
| ENSBTAG00000031933 | ALOX12E | 9.09794692 | 5.176226651 | 1.17E−70 | 6.55E−69 |
| ENSBTAG00000018122 | TKDP1 | 9.070455318 | 14.24193908 | 0 | 0 |
| ENSBTAG00000004221 | ESM1 | 8.900570598 | 4.989104856 | 8.07E−63 | 3.89E−61 |
| ENSBTAG00000015059 | MMP13 | 8.865234193 | 4.957343174 | 1.90E−61 | 8.89E−60 |
| ENSBTAG00000009419 | TRIM29 | 8.729835149 | 4.830589808 | 1.40E−56 | 5.89E−55 |
| ENSBTAG00000011578 | CD44 | 8.410776553 | 8.283559546 | 6.5530636557992e−316 | 7.39336804920813e−313 |
| ENSBTAG00000007958 | TNS4 | 8.401286441 | 4.526821654 | 2.84E−46 | 9.03E−45 |
| ENSBTAG00000011810 | ANGPTL2 | 8.328755135 | 4.461013556 | 4.70E−44 | 1.41E−42 |
| ENSBTAG00000052866 | NA | 7.949431548 | 6.860117573 | 1.90E−173 | 4.43E−171 |
| ENSBTAG00000017531 | FETUB | 7.907376859 | 4.081032125 | 6.40E−34 | 1.42E−32 |
| ENSBTAG00000013688 | PGF | 7.884730335 | 4.061010671 | 2.12E−33 | 4.55E−32 |
| ENSBTAG00000013320 | TSPAN1 | 7.735469922 | 7.045878054 | 5.84E−188 | 1.65E−185 |
| ENSBTAG00000049869 | PRP1 | 7.731296047 | 3.926305688 | 2.92E−30 | 5.62E−29 |
| ENSBTAG00000000898 | F2RL2 | 7.729280998 | 7.063473251 | 1.37E−189 | 4.09E−187 |
| ENSBTAG00000006707 | ACSL5 | 7.689221043 | 3.889857916 | 1.81E−29 | 3.40E−28 |
| ENSBTAG00000000522 | AHSG | 7.625588882 | 3.834552817 | 3.82E−28 | 6.84E−27 |
| ENSBTAG00000009705 | SERPINF1 | 7.618919173 | 3.82949004 | 3.82E−28 | 6.84E−27 |
| ENSBTAG00000037858 | F1MIP0 | 7.608873095 | 3.820997946 | 7.06E−28 | 1.25E−26 |
| ENSBTAG00000019226 | PAG8 | 7.577902774 | 8.181418553 | 2.60E−295 | 2.00E−292 |
| ENSBTAG00000022759 | PAG11 | 7.516444904 | 10.45084065 | 0 | 0 |
| ENSBTAG00000000793 | LAMC2 | 7.489449979 | 7.175231514 | 1.09E−196 | 3.72E−194 |
| ENSBTAG00000017746 | SLIT3 | 7.467602078 | 3.699733233 | 1.81E−25 | 2.90E−24 |
| ENSBTAG00000033190 | PGM5 | 7.46506923 | 3.697552647 | 1.81E−25 | 2.90E−24 |
| ENSBTAG00000033961 | RNF217 | 7.453508116 | 3.687689396 | 3.37E−25 | 5.32E−24 |
| ENSBTAG00000026966 | RASSF9 | 7.443502169 | 3.679226003 | 3.37E−25 | 5.32E−24 |
| ENSBTAG00000019067 | TMC8 | 7.283219596 | 3.544306605 | 1.73E−22 | 2.42E−21 |
| ENSBTAG00000013481 | CYP4A22 | 7.23227348 | 3.501650446 | 6.10E−22 | 8.31E−21 |
| ENSBTAG00000003959 | ARHGAP24 | 7.223217106 | 3.494402019 | 1.14E−21 | 1.53E−20 |
| ENSBTAG00000017863 | SRGN | 7.189435483 | 5.944119576 | 3.45E−105 | 3.56E−103 |
| ENSBTAG00000037811 | CCL2 | 7.189206307 | 3.466406171 | 4.04E−21 | 5.27E−20 |
| ENSBTAG00000049075 | PRP14 | 7.156364662 | 3.438884363 | 7.61E−21 | 9.78E−20 |
| ENSBTAG00000000575 | TNC | 7.138416588 | 6.429527442 | 2.29E−136 | 3.43E−134 |
| ENSBTAG00000011343 | XAF1 | 7.133454502 | 3.420553811 | 1.43E−20 | 1.82E−19 |
| ENSBTAG00000013928 | WFDC2 | 7.09352088 | 3.387954997 | 5.10E−20 | 6.28E−19 |
| ENSBTAG00000018453 | ANKRD37 | 7.086574904 | 3.382408004 | 9.63E−20 | 1.17E−18 |
| ENSBTAG00000039509 | GPRC5A | 7.079060336 | 3.375960717 | 9.63E−20 | 1.17E−18 |
| ENSBTAG00000036078 | EMP1 | 7.073198319 | 6.529298845 | 2.53E−142 | 4.31E−140 |
| ENSBTAG00000004116 | PAG10 | 7.006020476 | 3.316620643 | 6.51E−19 | 7.56E−18 |
| ENSBTAG00000011847 | ASPN | 6.996941592 | 3.309767476 | 1.23E−18 | 1.42E−17 |
| ENSBTAG00000020638 | TIMP3 | 6.99491618 | 9.186976933 | 0 | 0 |
| ENSBTAG00000019437 | SLFN11 | 6.979759315 | 7.146693 | 1.95E−190 | 5.95E−188 |
| ENSBTAG00000001465 | P2RY1 | 6.976268935 | 3.29306931 | 2.34E−18 | 2.61E−17 |
| ENSBTAG00000017150 | EHF | 6.896732412 | 3.229506484 | 1.60E−17 | 1.70E−16 |
| ENSBTAG00000006039 | ARHGDIB | 6.880085121 | 7.011204544 | 5.95E−178 | 1.50E−175 |
| ENSBTAG00000017733 | CA2 | 6.829114812 | 7.165050632 | 3.54E−189 | 1.04E−186 |
| ENSBTAG00000030932 | IFI44L | 6.820482028 | 3.169424617 | 1.10E−16 | 1.12E−15 |
| ENSBTAG00000013662 | COL8A1 | 6.805847535 | 3.157974818 | 2.10E−16 | 2.10E−15 |
| ENSBTAG00000025302 | PRP6 | 6.798538974 | 3.151933352 | 2.10E−16 | 2.10E−15 |
| ENSBTAG00000025304 | PRP4 | 6.701549025 | 3.076666429 | 1.46E−15 | 1.39E−14 |
| ENSBTAG00000039970 | PAG16 | 6.686497818 | 3.065088406 | 2.80E−15 | 2.63E−14 |
| ENSBTAG00000001576 | TMEM140 | 6.684916988 | 3.064157616 | 2.80E−15 | 2.63E−14 |
| ENSBTAG00000013155 | COL2A1 | 6.658487543 | 3.043877049 | 5.35E−15 | 4.96E−14 |
| ENSBTAG00000021957 | LTBP2 | 6.608115948 | 3.005478885 | 1.96E−14 | 1.76E−13 |
| ENSBTAG00000001125 | ADAMDEC1 | 6.607882631 | 3.005320138 | 1.96E−14 | 1.76E−13 |
| ENSBTAG00000037649 | VIPR2 | 6.559404398 | 2.968652687 | 3.77E−14 | 3.33E−13 |
| ENSBTAG00000009086 | LOXL1 | 6.529262525 | 2.946083017 | 7.24E−14 | 6.26E−13 |
| ENSBTAG00000008682 | TLR3 | 6.527007328 | 2.94440955 | 7.24E−14 | 6.26E−13 |
| ENSBTAG00000052204 | NA | 6.514357167 | 2.934898603 | 1.39E−13 | 1.18E−12 |
| ENSBTAG00000012989 | UBE2L6 | 6.511740422 | 2.932951907 | 1.39E−13 | 1.18E−12 |
| ENSBTAG00000004754 | DSCAM | 6.511413393 | 2.932616575 | 1.39E−13 | 1.18E−12 |
| ENSBTAG00000054162 | HOXB8 | 6.458010719 | 2.89310107 | 5.15E−13 | 4.18E−12 |
| ENSBTAG00000030190 | COL6A3 | 6.446117705 | 2.88429122 | 5.15E−13 | 4.18E−12 |
| ENSBTAG00000018169 | ITGB4 | 6.435508804 | 8.373498125 | 1.96E−294 | 1.44E−291 |
| ENSBTAG00000011651 | HECTD2 | 6.433113781 | 2.874645912 | 5.15E−13 | 4.18E−12 |
| ENSBTAG00000019066 | FAM167A | 6.42776981 | 2.870753984 | 5.15E−13 | 4.18E−12 |
| ENSBTAG00000051382 | PRP2 | 6.425172571 | 2.868645929 | 5.15E−13 | 4.18E−12 |
| ENSBTAG00000004570 | PRRX1 | 6.422306055 | 2.86674818 | 9.92E−13 | 7.83E−12 |
| ENSBTAG00000012004 | TGFB3 | 6.393632807 | 2.84569117 | 9.92E−13 | 7.83E−12 |
| ENSBTAG00000018235 | SLC45A2 | 6.392511105 | 2.844935488 | 1.91E−12 | 1.46E−11 |
| ENSBTAG00000012370 | MGP | 6.389431244 | 2.842545116 | 1.91E−12 | 1.46E−11 |
| ENSBTAG00000017069 | GASK1B | 6.379410619 | 2.835390125 | 1.91E−12 | 1.46E−11 |
| ENSBTAG00000005260 | SPP1 | 6.373750696 | 7.938252492 | 8.26E−252 | 4.49E−249 |
| ENSBTAG00000026885 | ABCB11 | 6.343797454 | 2.809459166 | 3.69E−12 | 2.77E−11 |
| ENSBTAG00000019315 | PLA2G7 | 6.342629595 | 8.829366836 | 0 | 0 |
| ENSBTAG00000008021 | BST2 | 6.333235674 | 2.801910717 | 3.69E−12 | 2.77E−11 |
| ENSBTAG00000045567 | PRR15 | 6.332810488 | 2.801629235 | 3.69E−12 | 2.77E−11 |
| ENSBTAG00000014218 | DKK1 | 6.318100875 | 2.790773389 | 3.69E−12 | 2.77E−11 |
| ENSBTAG00000015801 | EFNB1 | 6.313120429 | 6.766689402 | 1.89E−153 | 3.69E−151 |
| ENSBTAG00000017425 | GJB2 | 6.307408504 | 5.816440815 | 9.42E−94 | 7.98E−92 |
| ENSBTAG00000003313 | TNFRSF9 | 6.302899658 | 2.779942057 | 7.13E−12 | 5.17E−11 |
| ENSBTAG00000013263 | HOXA1 | 6.296396712 | 2.775348783 | 7.13E−12 | 5.17E−11 |
| ENSBTAG00000053198 | A0A3Q1LJW9 | 6.295573087 | 2.774766936 | 7.13E−12 | 5.17E−11 |
| ENSBTAG00000012715 | KIF26B | 6.288551949 | 2.76977996 | 7.13E−12 | 5.17E−11 |
| ENSBTAG00000053027 | IFNT2 | 6.207001143 | 2.711692405 | 2.67E−11 | 1.84E−10 |
| ENSBTAG00000019672 | Q56JW3 | 6.201363753 | 2.707858805 | 5.17E−11 | 3.48E−10 |
| ENSBTAG00000020884 | CASP4 | 6.191298573 | 2.700710283 | 5.17E−11 | 3.48E−10 |
| ENSBTAG00000055295 | MUC16 | 6.168803912 | 2.684948919 | 5.17E−11 | 3.48E−10 |
| ENSBTAG00000001143 | GBP1 | 6.087258024 | 2.628502357 | 1.94E−10 | 1.25E−09 |
| ENSBTAG00000031279 | SELENOM | 6.082341201 | 2.625148719 | 3.77E−10 | 2.38E−09 |
| ENSBTAG00000002576 | GLDN | 6.033630949 | 2.591907577 | 7.33E−10 | 4.48E−09 |
| ENSBTAG00000031358 | DPYD | 6.032763257 | 2.591301176 | 7.33E−10 | 4.48E−09 |
| ENSBTAG00000021458 | DLX6 | 6.027639978 | 2.587866588 | 7.33E−10 | 4.48E−09 |
| ENSBTAG00000021029 | PRR5L | 6.014711392 | 2.579102869 | 7.33E−10 | 4.48E−09 |
| ENSBTAG00000018571 | IL1RL1 | 6.010328939 | 2.576157836 | 7.33E−10 | 4.48E−09 |
| ENSBTAG00000015854 | TCHHL1 | 6.004520862 | 2.572233702 | 7.33E−10 | 4.48E−09 |
| ENSBTAG00000030317 | PPP1R26 | 5.998619527 | 2.568243485 | 1.43E−09 | 8.51E−09 |
| ENSBTAG00000007581 | ADORA2B | 5.996888901 | 2.567065322 | 1.43E−09 | 8.51E−09 |
| ENSBTAG00000002006 | THBS1 | 5.988788759 | 8.418799276 | 1.46E−286 | 9.30E−284 |
| ENSBTAG00000019792 | SLC16A9 | 5.987992776 | 6.415439041 | 9.63E−126 | 1.24E−123 |
| ENSBTAG00000001060 | CXCR4 | 5.986755281 | 2.56029312 | 1.43E−09 | 8.51E−09 |
| ENSBTAG00000007705 | COLEC12 | 5.967838027 | 2.547542722 | 1.43E−09 | 8.51E−09 |
| ENSBTAG00000015562 | PLBD1 | 5.916384116 | 8.70535025 | 2.79327721842961e−310 | 2.73E−307 |
| ENSBTAG00000010023 | FAM107B | 5.910949904 | 2.509772869 | 2.77E−09 | 1.61E−08 |
| ENSBTAG00000054745 | lncRNA | 5.905606785 | 2.506167688 | 2.77E−09 | 1.61E−08 |
| ENSBTAG00000025494 | PAQR8 | 5.877070221 | 7.406106014 | 4.36E−196 | 1.42E−193 |
| ENSBTAG00000009443 | GCNT3 | 5.858819292 | 7.535408783 | 1.54E−205 | 5.51E−203 |
| ENSBTAG00000014278 | TBX2 | 5.857338703 | 2.474604946 | 5.41E−09 | 3.06E−08 |
| ENSBTAG00000016881 | ANPEP | 5.85373784 | 6.851738836 | 1.38E−153 | 2.74E−151 |
| ENSBTAG00000030518 | ECSCR | 5.834627508 | 2.459889111 | 1.05E−08 | 5.78E−08 |
| ENSBTAG00000039425 | KRT6A | 5.823884374 | 2.452930943 | 1.05E−08 | 5.78E−08 |
| ENSBTAG00000046468 | ACTBL2 | 5.816301923 | 2.44802759 | 1.05E−08 | 5.78E−08 |
| ENSBTAG00000032084 | GPR183 | 5.812165494 | 2.445346066 | 1.05E−08 | 5.78E−08 |
| ENSBTAG00000049392 | A0A3Q1M6W2 | 5.779081718 | 2.424112978 | 2.06E−08 | 1.09E−07 |
| ENSBTAG00000004903 | KRT15 | 5.755356756 | 4.744101377 | 1.44E−47 | 4.71E−46 |
| ENSBTAG00000007101 | F3 | 5.751435004 | 8.652558461 | 7.83E−300 | 6.75E−297 |
| ENSBTAG00000034712 | RAB33A | 5.750063884 | 2.405699046 | 4.02E−08 | 2.07E−07 |
| ENSBTAG00000005615 | CEACAM1 | 5.727949107 | 2.391660716 | 4.02E−08 | 2.07E−07 |
| ENSBTAG00000037558 | GRO1 | 5.681295056 | 2.362391235 | 7.86E−08 | 3.93E−07 |
| ENSBTAG00000008139 | HOXA3 | 5.636895065 | 2.334871496 | 1.54E−07 | 7.43E−07 |
| ENSBTAG00000005848 | PRTG | 5.633690044 | 2.332934678 | 1.54E−07 | 7.43E−07 |
| ENSBTAG00000019675 | ATXN1 | 5.629484265 | 2.330324591 | 1.54E−07 | 7.43E−07 |
| ENSBTAG00000009515 | CA6 | 5.616520213 | 2.322366597 | 1.54E−07 | 7.43E−07 |
| ENSBTAG00000019832 | TGFBR2 | 5.610881171 | 5.866625932 | 7.82E−91 | 6.41E−89 |
| ENSBTAG00000004303 | SLC27A2 | 5.584815972 | 7.22355203 | 7.20E−176 | 1.73E−173 |
| ENSBTAG00000008441 | SOCS3 | 5.579385225 | 2.299725311 | 3.01E−07 | 1.41E−06 |
| ENSBTAG00000033562 | LHX1 | 5.573045718 | 2.295875362 | 3.01E−07 | 1.41E−06 |
| ENSBTAG00000004885 | DDR2 | 5.56636622 | 2.291845779 | 3.01E−07 | 1.41E−06 |
| ENSBTAG00000005968 | TLL1 | 5.561149342 | 2.28867403 | 3.01E−07 | 1.41E−06 |
| ENSBTAG00000008096 | EDN1 | 5.555893838 | 2.285503771 | 3.01E−07 | 1.41E−06 |
| ENSBTAG00000019002 | SLC2A12 | 5.55423978 | 2.284507069 | 3.01E−07 | 1.41E−06 |
| ENSBTAG00000016542 | LAMB3 | 5.545679834 | 4.827237573 | 5.32E−49 | 1.83E−47 |
| ENSBTAG00000033268 | ZFHX4 | 5.53837187 | 2.274967737 | 3.01E−07 | 1.41E−06 |
| ENSBTAG00000004010 | PAPPA | 5.532872037 | 2.271695993 | 3.01E−07 | 1.41E−06 |
| ENSBTAG00000014847 | BBOX1 | 5.529520893 | 2.269680321 | 3.01E−07 | 1.41E−06 |
| ENSBTAG00000022360 | SOX5 | 5.515934499 | 2.261568573 | 5.90E−07 | 2.67E−06 |
| ENSBTAG00000003253 | NPPC | 5.468162378 | 2.233246167 | 5.90E−07 | 2.67E−06 |
| ENSBTAG00000001034 | IL18R1 | 5.467535584 | 2.232918112 | 5.90E−07 | 2.67E−06 |
| ENSBTAG00000044169 | PRELID2 | 5.461430267 | 4.885104312 | 4.75E−51 | 1.73E−49 |
| ENSBTAG00000049252 | GPX6 | 5.433118955 | 2.212778423 | 1.16E−06 | 5.06E−06 |
| ENSBTAG00000050440 | PAG20 | 5.414259469 | 2.201796915 | 1.16E−06 | 5.06E−06 |
| ENSBTAG00000052794 | SSLP1 | 5.400823492 | 11.06961349 | 0 | 0 |
| ENSBTAG00000010688 | CSTA | 5.398921235 | 2.192971706 | 1.16E−06 | 5.06E−06 |
| ENSBTAG00000012994 | LOX | 5.367582092 | 4.032292864 | 5.81E−29 | 1.07E−27 |
| ENSBTAG00000004351 | IL31RA | 5.366904815 | 2.174586182 | 2.27E−06 | 9.62E−06 |
| ENSBTAG00000001464 | GPR156 | 5.329658575 | 2.153451795 | 2.27E−06 | 9.62E−06 |
| ENSBTAG00000002788 | RAB27B | 5.320629724 | 2.14837903 | 4.47E−06 | 1.81E−05 |
| ENSBTAG00000020713 | BACH2 | 5.319974445 | 2.147994585 | 4.47E−06 | 1.81E−05 |
| ENSBTAG00000006096 | TKDP2 | 5.311425015 | 2.143176286 | 4.47E−06 | 1.81E−05 |
| ENSBTAG00000015872 | PROKR2 | 5.300888823 | 2.137280812 | 4.47E−06 | 1.81E−05 |
| ENSBTAG00000053183 | PAG4 | 5.300346155 | 2.136952763 | 4.47E−06 | 1.81E−05 |
| ENSBTAG00000039765 | SECTM1 | 5.292783099 | 2.132725795 | 4.47E−06 | 1.81E−05 |
| ENSBTAG00000026102 | PAG9 | 5.284455254 | 2.128082561 | 4.47E−06 | 1.81E−05 |
| ENSBTAG00000017827 | IVL | 5.281495952 | 2.126473118 | 4.47E−06 | 1.81E−05 |
| ENSBTAG00000014020 | RTKN2 | 5.280708645 | 4.676535209 | 1.10E−43 | 3.24E−42 |
| ENSBTAG00000018540 | NOX4 | 5.262036409 | 5.941312557 | 4.34E−91 | 3.57E−89 |
| ENSBTAG00000047482 | PRRT4 | 5.246204542 | 4.163958035 | 1.03E−30 | 2.02E−29 |
| ENSBTAG00000054510 | A0A3Q1M325 | 5.244414509 | 2.105948651 | 8.79E−06 | 3.40E−05 |
| ENSBTAG00000001495 | SPINLW1 | 5.24269503 | 2.105006258 | 8.79E−06 | 3.40E−05 |
| ENSBTAG00000051298 | PVR | 5.240793581 | 5.614050829 | 1.64E−76 | 1.01E−74 |
| ENSBTAG00000025471 | TNF | 5.234507211 | 2.100495825 | 8.79E−06 | 3.40E−05 |
| ENSBTAG00000045822 | C15H11orf96 | 5.232987366 | 4.001306468 | 3.38E−28 | 6.06E−27 |
| ENSBTAG00000050495 | GREM1 | 5.232632702 | 2.099476832 | 8.79E−06 | 3.40E−05 |
| ENSBTAG00000050058 | NA | 5.223072211 | 2.09423741 | 8.79E−06 | 3.40E−05 |
| ENSBTAG00000003014 | TRPV2 | 5.218688321 | 2.091851035 | 8.79E−06 | 3.40E−05 |
| ENSBTAG00000031383 | SMIM24 | 5.217096613 | 2.090975514 | 8.79E−06 | 3.40E−05 |
| ENSBTAG00000020554 | AIF1 | 5.215491403 | 5.564155996 | 8.84E−74 | 5.20E−72 |
| ENSBTAG00000000664 | SLC22A15 | 5.206192492 | 2.085042935 | 8.79E−06 | 3.40E−05 |
| ENSBTAG00000035995 | TNFAIP2 | 5.196096798 | 2.079572764 | 8.79E−06 | 3.40E−05 |
| ENSBTAG00000020406 | GPC3 | 5.185409942 | 2.073783983 | 8.79E−06 | 3.40E−05 |
| ENSBTAG00000027181 | LAMA3 | 5.184352986 | 6.319871626 | 1.01E−110 | 1.10E−108 |
| ENSBTAG00000020826 | CHIC1 | 5.170543398 | 2.065777366 | 8.79E−06 | 3.40E−05 |
| ENSBTAG00000027126 | MUC15 | 5.169216512 | 2.065061214 | 8.79E−06 | 3.40E−05 |
| ENSBTAG00000019289 | ITGA2 | 5.165384384 | 6.966454689 | 4.43E−150 | 8.44E−148 |
| ENSBTAG00000046142 | PRKAR1B | 5.156660802 | 2.058335617 | 1.73E−05 | 6.42E−05 |
| ENSBTAG00000031558 | Tmsb4x | 5.146346475 | 2.052823746 | 1.73E−05 | 6.42E−05 |
| ENSBTAG00000002714 | GNAI1 | 5.142537958 | 8.155619084 | 6.84E−236 | 3.35E−233 |
| ENSBTAG00000015844 | TFPI2 | 5.124126204 | 2.041015046 | 1.73E−05 | 6.42E−05 |
| ENSBTAG00000012307 | DTNA | 5.10023149 | 2.028419922 | 1.73E−05 | 6.42E−05 |
| ENSBTAG00000053050 | SAMD9 | 5.093512114 | 2.024894274 | 1.73E−05 | 6.42E−05 |
| ENSBTAG00000004775 | GRIA2 | 5.064828037 | 2.009930926 | 3.41E−05 | 0.000120389 |
| ENSBTAG00000027407 | TNFRSF6B | 5.051894934 | 2.003237686 | 3.41E−05 | 0.000120389 |
| ENSBTAG00000020590 | FZD2 | 5.05122231 | 2.002889307 | 3.41E−05 | 0.000120389 |
| ENSBTAG00000000088 | ALCAM | 5.026869174 | 6.039510351 | 2.05E−93 | 1.72E−91 |
| ENSBTAG00000014374 | CARD6 | 5.024523763 | 1.989155462 | 3.41E−05 | 0.000120389 |
| ENSBTAG00000014908 | CCL27 | 5.021217237 | 1.987462394 | 3.41E−05 | 0.000120389 |
| ENSBTAG00000045824 | SOX9 | 5.013804995 | 1.983675955 | 3.41E−05 | 0.000120389 |
| ENSBTAG00000020715 | PIK3C2G | 5.012722328 | 1.983120555 | 3.41E−05 | 0.000120389 |
| ENSBTAG00000000283 | CSF1 | 5.005381881 | 1.97938224 | 3.41E−05 | 0.000120389 |
| ENSBTAG00000026016 | PAG17 | 4.993387287 | 1.973290124 | 6.73E−05 | 0.000225789 |
| ENSBTAG00000049718 | NA | 4.976615422 | 1.964817828 | 6.73E−05 | 0.000225789 |
| ENSBTAG00000039861 | OAS1Y | 4.975469121 | 4.865624352 | 1.81E−47 | 5.91E−46 |
| ENSBTAG00000013918 | ADGRL3 | 4.965102386 | 7.192019524 | 1.08E−160 | 2.24E−158 |
| ENSBTAG00000021467 | IGFBP6 | 4.961626623 | 5.715550259 | 4.22E−78 | 2.67E−76 |
| ENSBTAG00000034680 | FAM131C | 4.957223039 | 1.955080165 | 6.73E−05 | 0.000225789 |
| ENSBTAG00000002792 | FUT11 | 4.955630331 | 1.954283304 | 6.73E−05 | 0.000225789 |
| ENSBTAG00000025442 | HSPA1L | 4.954903422 | 1.953919723 | 6.73E−05 | 0.000225789 |
| ENSBTAG00000018007 | NR2F2 | 4.951514391 | 4.410858963 | 1.26E−35 | 2.98E−34 |
| ENSBTAG00000006694 | CXCL14 | 4.950162926 | 1.951551134 | 6.73E−05 | 0.000225789 |
| ENSBTAG00000000945 | ATP2C2 | 4.943495446 | 8.044024462 | 1.02E−219 | 4.33E−217 |
| ENSBTAG00000054808 | MT1A | 4.938949072 | 4.17028566 | 2.66E−29 | 4.97E−28 |
| ENSBTAG00000031640 | PRP3 | 4.923231462 | 1.938170323 | 6.73E−05 | 0.000225789 |
| ENSBTAG00000016138 | CGA | 4.92297888 | 1.938043532 | 6.73E−05 | 0.000225789 |
| ENSBTAG00000019588 | BLA-DQB | 4.922542575 | 1.937826851 | 6.73E−05 | 0.000225789 |
| ENSBTAG00000046121 | B6VAE0 | 4.920469989 | 1.936803369 | 6.73E−05 | 0.000225789 |
| ENSBTAG00000012900 | TMEM229B | 4.920212625 | 1.936677194 | 6.73E−05 | 0.000225789 |
| ENSBTAG00000018920 | TRIM47 | 4.919086044 | 1.936119824 | 6.73E−05 | 0.000225789 |
| ENSBTAG00000015347 | WNT10B | 4.919022508 | 1.93608956 | 6.73E−05 | 0.000225789 |
| ENSBTAG00000017866 | CD36 | 4.916752488 | 6.284455285 | 1.06E−104 | 1.07E−102 |
| ENSBTAG00000001595 | MT1E | 4.913908911 | 8.059520203 | 1.03E−219 | 4.33E−217 |
| ENSBTAG00000000936 | SLC39A2 | 4.91065808 | 8.768002707 | 7.56E−272 | 4.62E−269 |
| ENSBTAG00000017158 | VSNL1 | 4.904298504 | 3.724332912 | 2.90E−22 | 4.01E−21 |
| ENSBTAG00000049393 | NA | 4.902536776 | 1.927970585 | 0.000132849 | 0.000425157 |
| ENSBTAG00000020536 | HERC6 | 4.895117336 | 4.699005509 | 8.49E−42 | 2.37E−40 |
| ENSBTAG00000047009 | ANKRD66 | 4.894359527 | 1.923960273 | 0.000132849 | 0.000425157 |
| ENSBTAG00000016414 | VDR | 4.878967168 | 4.977671169 | 2.48E−50 | 8.83E−49 |
| ENSBTAG00000004557 | DHRS9 | 4.877891525 | 4.995115273 | 5.45E−51 | 1.98E−49 |
| ENSBTAG00000014762 | ISG20 | 4.876230324 | 1.915114513 | 0.000132849 | 0.000425157 |
| ENSBTAG00000024608 | G3MYL1 | 4.872287817 | 1.913201998 | 0.000132849 | 0.000425157 |
| ENSBTAG00000004948 | LRATD1 | 4.871442099 | 5.151537413 | 6.44E−56 | 2.68E−54 |
| ENSBTAG00000004394 | PKIB | 4.869173148 | 7.306598521 | 2.22E−165 | 4.85E−163 |
| ENSBTAG00000002751 | VSTM5 | 4.862317527 | 1.908362221 | 0.000132849 | 0.000425157 |
| ENSBTAG00000004280 | SRPX | 4.850005901 | 1.902418565 | 0.000132849 | 0.000425157 |
| ENSBTAG00000021466 | COL3A1 | 4.835727549 | 5.826200733 | 1.68E−80 | 1.11E−78 |
| ENSBTAG00000005741 | DLX2 | 4.825200278 | 1.890517585 | 0.000132849 | 0.000425157 |
| ENSBTAG00000000735 | BCL2A1 | 4.822304353 | 1.889141357 | 0.000132849 | 0.000425157 |
| ENSBTAG00000013368 | ANKRD22 | 4.820207122 | 1.888134539 | 0.000132849 | 0.000425157 |
| ENSBTAG00000007383 | ZNF521 | 4.815896382 | 3.712705568 | 7.35E−21 | 9.47E−20 |
| ENSBTAG00000053587 | ST6GALNAC3 | 4.796766141 | 1.877006111 | 0.000262523 | 0.00079686 |
| ENSBTAG00000000394 | BACE2 | 4.796019116 | 4.73722622 | 6.10E−43 | 1.78E−41 |
| ENSBTAG00000050176 | HSP90AB1 | 4.776104511 | 1.867277295 | 0.000262523 | 0.00079686 |
| ENSBTAG00000009230 | FBLN7 | 4.762299201 | 7.095434733 | 8.80E−149 | 1.61E−146 |
| ENSBTAG00000050644 | A0A3Q1M2T3 | 4.744155465 | 5.542319488 | 6.41E−69 | 3.51E−67 |
| ENSBTAG00000016012 | WNT3 | 4.737156617 | 1.849142425 | 0.000262523 | 0.00079686 |
| ENSBTAG00000015052 | PROX2 | 4.735934541 | 1.848567802 | 0.000262523 | 0.00079686 |
| ENSBTAG00000008448 | MEIS1 | 4.733316586 | 1.847361947 | 0.000262523 | 0.00079686 |
| ENSBTAG00000015810 | C15H11orf34 | 4.732019633 | 4.80018844 | 6.90E−44 | 2.05E−42 |
| ENSBTAG00000006720 | GJA5 | 4.731392649 | 1.846476263 | 0.000262523 | 0.00079686 |
| ENSBTAG00000018645 | DLX5 | 4.728026358 | 4.966125328 | 1.39E−48 | 4.72E−47 |
| ENSBTAG00000006761 | CSH2 | 4.725600718 | 1.843815573 | 0.000262523 | 0.00079686 |
| ENSBTAG00000000859 | SLC38A1 | 4.722950511 | 3.8492711 | 2.05E−23 | 2.99E−22 |
| ENSBTAG00000006806 | KRT17 | 4.718331155 | 1.840467046 | 0.000262523 | 0.00079686 |
| ENSBTAG00000024340 | TRIL | 4.717148558 | 1.83991915 | 0.000262523 | 0.00079686 |
| ENSBTAG00000005230 | SHOX2 | 4.711557045 | 1.837358162 | 0.000262523 | 0.00079686 |
| ENSBTAG00000024582 | RPL10L | 4.711257225 | 1.837218633 | 0.000262523 | 0.00079686 |
| ENSBTAG00000014465 | SERPINE1 | 4.706860914 | 8.178133661 | 6.40E−219 | 2.61E−216 |
| ENSBTAG00000019519 | PWWP2B | 4.70229311 | 1.833117375 | 0.000262523 | 0.00079686 |
| ENSBTAG00000046648 | CPA4 | 4.70140402 | 3.7016497 | 7.35E−21 | 9.47E−20 |
| ENSBTAG00000013103 | COL1A1 | 4.698298765 | 7.319034084 | 5.95E−161 | 1.25E−158 |
| ENSBTAG00000010431 | LURAP1L | 4.691385751 | 1.828151277 | 0.000519245 | 0.001500643 |
| ENSBTAG00000009978 | TDRD6 | 4.688460193 | 1.826823754 | 0.000519245 | 0.001500643 |
| ENSBTAG00000015032 | CD14 | 4.686018916 | 3.888524166 | 3.54E−24 | 5.38E−23 |
| ENSBTAG00000031750 | PLAC8B | 4.685121978 | 1.825312377 | 0.000519245 | 0.001500643 |
| ENSBTAG00000011298 | EYA1 | 4.680614394 | 1.823269918 | 0.000519245 | 0.001500643 |
| ENSBTAG00000018661 | TMC6 | 4.679450926 | 5.254602718 | 9.41E−58 | 4.05E−56 |
| ENSBTAG00000017808 | ARSJ | 4.675216256 | 3.907814969 | 1.34E−23 | 1.97E−22 |
| ENSBTAG00000007196 | TAGLN | 4.673431545 | 6.305850009 | 1.36E−101 | 1.28E−99 |
| ENSBTAG00000008320 | SEPTIN5 | 4.671669991 | 5.76252802 | 7.61E−77 | 4.73E−75 |
| ENSBTAG00000054663 | PAG5 | 4.671654968 | 1.819230661 | 0.000519245 | 0.001500643 |
| ENSBTAG00000003165 | ADAMTS9 | 4.670202048 | 4.082621782 | 2.49E−27 | 4.30E−26 |
| ENSBTAG00000048460 | lncRNA | 4.664274471 | 1.815889073 | 0.000519245 | 0.001500643 |
| ENSBTAG00000016661 | USP18 | 4.654587505 | 6.952539612 | 3.26E−137 | 5.03E−135 |
| ENSBTAG00000012149 | HOXC8 | 4.653256194 | 1.810945558 | 0.000519245 | 0.001500643 |
| ENSBTAG00000052722 | NA | 4.632997302 | 1.801910851 | 0.000519245 | 0.001500643 |
| ENSBTAG00000002011 | KIAA0895 | 4.624616505 | 1.798188271 | 0.000519245 | 0.001500643 |
| ENSBTAG00000052560 | NA | 4.618211643 | 1.795350755 | 0.000519245 | 0.001500643 |
| ENSBTAG00000047998 | COL5A1 | 4.615356042 | 5.698900494 | 2.54E−73 | 1.47E−71 |
| ENSBTAG00000040323 | SECTM1 | 4.60285978 | 1.788602416 | 0.000519245 | 0.001500643 |
| ENSBTAG00000015065 | TMPRSS7 | 4.602097705 | 1.78823884 | 0.000519245 | 0.001500643 |
| ENSBTAG00000014831 | PPP1R3C | 4.596282032 | 3.808641863 | 1.19E−22 | 1.68E−21 |
| ENSBTAG00000014707 | ISG15 | 4.588086793 | 5.044835807 | 2.43E−49 | 8.43E−48 |
| ENSBTAG00000021609 | ADGRF1 | 4.583063028 | 1.77991808 | 0.001027951 | 0.002812865 |
| ENSBTAG00000051898 | FGF7 | 4.582988145 | 1.779891109 | 0.001027951 | 0.002812865 |
| ENSBTAG00000037735 | C5AR2 | 4.580613092 | 1.778850517 | 0.001027951 | 0.002812865 |
| ENSBTAG00000013235 | TINAGL1 | 4.571632758 | 6.348770593 | 2.14E−102 | 2.06E−100 |
| ENSBTAG00000007626 | IL2RG | 4.56410853 | 1.771700763 | 0.001027951 | 0.002812865 |
| ENSBTAG00000054438 | GPSM3 | 4.560518578 | 1.770136593 | 0.001027951 | 0.002812865 |
| ENSBTAG00000031548 | Slc23a4 | 4.559618751 | 4.749541382 | 7.93E−41 | 2.15E−39 |
| ENSBTAG00000000516 | OTOR | 4.553164978 | 1.766966969 | 0.001027951 | 0.002812865 |
| ENSBTAG00000017367 | IFIT5 | 4.550693634 | 5.279455295 | 1.49E−56 | 6.24E−55 |
| ENSBTAG00000015232 | RAPGEFL1 | 4.539718744 | 1.761182459 | 0.001027951 | 0.002812865 |
| ENSBTAG00000003669 | BNC2 | 4.531516044 | 1.757675733 | 0.001027951 | 0.002812865 |
| ENSBTAG00000049724 | lncRNA | 4.515741262 | 1.750956627 | 0.001027951 | 0.002812865 |
| ENSBTAG00000004848 | OLFML2A | 4.513460278 | 1.749984568 | 0.001027951 | 0.002812865 |
| ENSBTAG00000012406 | ZBP1 | 4.506103522 | 3.14308933 | 2.69E−13 | 2.23E−12 |
| ENSBTAG00000016208 | TGM2 | 4.50199979 | 9.056755484 | 1.46E−267 | 8.56E−265 |
| ENSBTAG00000025522 | UNC13C | 4.495415844 | 1.74236033 | 0.001027951 | 0.002812865 |
| ENSBTAG00000054543 | processed | 4.487692532 | 1.739117032 | 0.001027951 | 0.002812865 |
| | pseudogene | | | | |
| ENSBTAG00000055220 | A0A3Q1M115 | 4.483072938 | 1.737179936 | 0.001027951 | 0.002812865 |
| ENSBTAG00000000654 | ARMC4 | 4.47216535 | 1.732620336 | 0.001027951 | 0.002812865 |
| ENSBTAG00000015461 | A0A3Q1MEG6 | 4.470373852 | 1.731871152 | 0.001027951 | 0.002812865 |
| ENSBTAG00000010175 | COL8A2 | 4.466684172 | 1.730331138 | 0.001027951 | 0.002812865 |
| ENSBTAG00000048517 | pseudogene | 4.463991623 | 1.729207655 | 0.001027951 | 0.002812865 |
| ENSBTAG00000007851 | IL24 | 4.462141977 | 1.728457676 | 0.001027951 | 0.002812865 |
| ENSBTAG00000023659 | MT2A | 4.461670268 | 7.928224715 | 2.29E−192 | 7.31E−190 |
| ENSBTAG00000016776 | BAHCC1 | 4.453865709 | 1.725009296 | 0.002036901 | 0.005222028 |
| ENSBTAG00000024379 | LRRC17 | 4.447227213 | 1.722252411 | 0.002036901 | 0.005222028 |
| ENSBTAG00000012083 | IRX3 | 4.44259796 | 3.823718344 | 1.35E−21 | 1.81E−20 |
| ENSBTAG00000050611 | DEFB1 | 4.438386624 | 5.179964789 | 3.10E−52 | 1.18E−50 |
| ENSBTAG00000012646 | PLCXD2 | 4.431688843 | 4.044869725 | 4.61E−25 | 7.25E−24 |
| ENSBTAG00000008797 | ARHGEF38 | 4.418662003 | 1.710542049 | 0.002036901 | 0.005222028 |
| ENSBTAG00000027477 | B3GAT1 | 4.415053123 | 1.709054422 | 0.002036901 | 0.005222028 |
| ENSBTAG00000021009 | TRIM55 | 4.408471037 | 1.706387566 | 0.002036901 | 0.005222028 |
| ENSBTAG00000010820 | WNT11 | 4.406674183 | 4.030217428 | 8.10E−25 | 1.26E−23 |
| ENSBTAG00000004896 | ABLIM3 | 4.405472581 | 5.488173998 | 1.20E−62 | 5.71E−61 |
| ENSBTAG00000047141 | PAG7 | 4.404145397 | 1.704649924 | 0.002036901 | 0.005222028 |
| ENSBTAG00000046628 | LYZ3 | 4.402705908 | 3.892954725 | 4.23E−23 | 6.10E−22 |
| ENSBTAG00000009873 | NRN1 | 4.402512882 | 1.703975184 | 0.002036901 | 0.005222028 |
| ENSBTAG00000007273 | TF | 4.39053912 | 1.699144375 | 0.002036901 | 0.005222028 |
| ENSBTAG00000005092 | ROR2 | 4.386855209 | 1.697639353 | 0.002036901 | 0.005222028 |
| ENSBTAG00000003658 | RELN | 4.379968539 | 1.694872378 | 0.002036901 | 0.005222028 |
| ENSBTAG00000046803 | GAS1 | 4.373116578 | 1.692115221 | 0.002036901 | 0.005222028 |
| ENSBTAG00000051079 | TMSB4 | 4.369489088 | 1.690671816 | 0.002036901 | 0.005222028 |
| ENSBTAG00000015326 | GSDME | 4.367885632 | 1.69003343 | 0.002036901 | 0.005222028 |
| ENSBTAG00000048755 | S100A11 | 4.367523966 | 3.809577636 | 2.41E−21 | 3.18E−20 |
| ENSBTAG00000038111 | FTL | 4.362966203 | 1.688059947 | 0.002036901 | 0.005222028 |
| ENSBTAG00000038600 | LRRC15 | 4.361219799 | 1.687364414 | 0.002036901 | 0.005222028 |
| ENSBTAG00000005176 | GDNF | 4.354822483 | 1.684826321 | 0.002036901 | 0.005222028 |
| ENSBTAG00000014615 | SLC26A2 | 4.351649304 | 5.59264356 | 1.35E−65 | 6.95E−64 |
| ENSBTAG00000046814 | ACOT2 | 4.351141254 | 1.683353597 | 0.002036901 | 0.005222028 |
| ENSBTAG00000006686 | NPNT | 4.331132394 | 4.391023746 | 2.94E−31 | 5.84E−30 |
| ENSBTAG00000011178 | IL17RB | 4.330860158 | 1.675334855 | 0.002036901 | 0.005222028 |
| ENSBTAG00000050006 | HOXB6 | 4.325557611 | 1.673250223 | 0.002036901 | 0.005222028 |
| ENSBTAG00000002934 | ADGRF4 | 4.321256649 | 1.671556475 | 0.004039852 | 0.009669143 |
| ENSBTAG00000014628 | OAS2 | 4.318451863 | 1.670462062 | 0.004039852 | 0.009669143 |
| ENSBTAG00000018994 | TNFSF10 | 4.317309037 | 1.670020283 | 0.004039852 | 0.009669143 |
| ENSBTAG00000003329 | FST | 4.305188675 | 5.479203114 | 2.45E−61 | 1.14E−59 |
| ENSBTAG00000014988 | na | 4.299610148 | 1.663139251 | 0.004039852 | 0.009669143 |
| ENSBTAG00000040040 | TAS2R40 | 4.297875927 | 1.66244342 | 0.004039852 | 0.009669143 |
| ENSBTAG00000010833 | IL17RE | 4.295601454 | 3.918488021 | 2.32E−22 | 3.23E−21 |
| ENSBTAG00000012899 | IFNGR2 | 4.293527107 | 4.751961104 | 1.27E−39 | 3.34E−38 |
| ENSBTAG00000013125 | PLAUR | 4.288088191 | 5.302992886 | 2.89E−55 | 1.18E−53 |
| ENSBTAG00000044073 | CD248 | 4.28341251 | 1.656845805 | 0.004039852 | 0.009669143 |
| ENSBTAG00000021807 | TAC3 | 4.252850972 | 1.645143335 | 0.004039852 | 0.009669143 |
| ENSBTAG00000049807 | HS6ST2 | 4.249575437 | 1.643890178 | 0.004039852 | 0.009669143 |
| ENSBTAG00000010366 | HCRTR1 | 4.248570333 | 1.643519076 | 0.004039852 | 0.009669143 |
| ENSBTAG00000015145 | S100A11 | 4.248404604 | 8.704491292 | 2.48E−229 | 1.18E−226 |
| ENSBTAG00000030913 | MX1 | 4.247336739 | 7.244151179 | 1.25E−142 | 2.15E−140 |
| ENSBTAG00000010836 | PRRT3 | 4.24467949 | 1.642026263 | 0.004039852 | 0.009669143 |
| ENSBTAG00000016149 | C9 | 4.240583016 | 1.640471471 | 0.004039852 | 0.009669143 |
| ENSBTAG00000012237 | AEBP1 | 4.240312113 | 3.34563114 | 6.27E−15 | 5.79E−14 |
| ENSBTAG00000009948 | TRIM25 | 4.23455762 | 5.334447947 | 1.47E−55 | 6.07E−54 |
| ENSBTAG00000025847 | MORF4L1 | 4.231671911 | 1.637113126 | 0.004039852 | 0.009669143 |
| ENSBTAG00000002408 | FHAD1 | 4.221783566 | 1.633393634 | 0.004039852 | 0.009669143 |
| ENSBTAG00000004159 | SIX2 | 4.220781267 | 1.633013426 | 0.004039852 | 0.009669143 |
| ENSBTAG00000000853 | NA | 4.219758234 | 1.632626955 | 0.004039852 | 0.009669143 |
| ENSBTAG00000048539 | A0A3Q1N469 | 4.218448777 | 1.632134847 | 0.004039852 | 0.009669143 |
| ENSBTAG00000052194 | A0A3Q1N672 | 4.218210514 | 1.632030181 | 0.004039852 | 0.009669143 |
| ENSBTAG00000014665 | ADAMTS2 | 4.209728719 | 1.628871502 | 0.004039852 | 0.009669143 |
| ENSBTAG00000039036 | PAG18 | 4.207409602 | 1.628029589 | 0.004039852 | 0.009669143 |
| ENSBTAG00000030593 | RFTN1 | 4.206152139 | 1.627531533 | 0.004039852 | 0.009669143 |
| ENSBTAG00000055063 | NA | 4.205251664 | 1.627202629 | 0.004039852 | 0.009669143 |
| ENSBTAG00000005871 | MECOM | 4.201791493 | 1.625907582 | 0.004039852 | 0.009669143 |
| ENSBTAG00000018843 | SERPINA1 | 4.200456229 | 1.625436545 | 0.004039852 | 0.009669143 |
| ENSBTAG00000007079 | LCP1 | 4.199889128 | 7.171851686 | 6.55E−137 | 9.91E−135 |
| ENSBTAG00000009663 | YBX3 | 4.196214925 | 6.238490207 | 8.81E−90 | 6.98E−88 |
| ENSBTAG00000048671 | pseudogene | 4.192503645 | 1.622457486 | 0.004039852 | 0.009669143 |
| ENSBTAG00000002779 | GABRP | 4.187455348 | 4.757987988 | 1.09E−38 | 2.83E−37 |
| ENSBTAG00000007922 | RNF24 | 4.184311012 | 1.619426733 | 0.004039852 | 0.009669143 |
| ENSBTAG00000045678 | G3N0T8 | 4.179035098 | 1.617476045 | 0.004039852 | 0.009669143 |
| ENSBTAG00000008636 | PDE4B | 4.178530547 | 1.617283343 | 0.004039852 | 0.009669143 |
| ENSBTAG00000051443 | lncRNA | 4.177134601 | 1.6167783 | 0.004039852 | 0.009669143 |
| ENSBTAG00000018669 | PLB1 | 4.170227234 | 1.614230875 | 0.008019742 | 0.017816655 |
| ENSBTAG00000005670 | ARHGEF19 | 4.166622019 | 1.612913594 | 0.008019742 | 0.017816655 |
| ENSBTAG00000053191 | Gm19410 | 4.165483015 | 1.612494062 | 0.008019742 | 0.017816655 |
| ENSBTAG00000011952 | SULT1E1 | 4.159825972 | 5.652853879 | 1.45E−65 | 7.45E−64 |
| ENSBTAG00000006984 | CD55 | 4.149899366 | 6.574967548 | 7.54E−105 | 7.63E−103 |
| ENSBTAG00000018424 | ACKR3 | 4.148018384 | 1.606116731 | 0.008019742 | 0.017816655 |
| ENSBTAG00000052388 | HYAL3 | 4.143974947 | 1.604645539 | 0.008019742 | 0.017816655 |
| ENSBTAG00000035493 | NA | 4.140995223 | 1.603563568 | 0.008019742 | 0.017816655 |
| ENSBTAG00000054562 | NA | 4.130671554 | 1.599835896 | 0.008019742 | 0.017816655 |
| ENSBTAG00000022348 | PAG3 | 4.129522449 | 1.599440777 | 0.008019742 | 0.017816655 |
| ENSBTAG00000000987 | OTOG | 4.12595759 | 1.598125186 | 0.008019742 | 0.017816655 |
| ENSBTAG00000000161 | TMEM40 | 4.125215069 | 4.092977109 | 4.48E−25 | 7.05E−24 |
| ENSBTAG00000055051 | RNF125 | 4.123770775 | 5.251179975 | 6.04E−52 | 2.27E−50 |
| ENSBTAG00000019432 | ICAM2 | 4.117509942 | 1.595107252 | 0.008019742 | 0.017816655 |
| ENSBTAG00000012401 | PPIAL4A | 4.114623811 | 1.594061043 | 0.008019742 | 0.017816655 |
| ENSBTAG00000032092 | CDH11 | 4.114302864 | 4.923335379 | 2.51E−42 | 7.16E−41 |
| ENSBTAG00000050666 | NA | 4.109738074 | 3.894999191 | 7.22E−22 | 9.81E−21 |
| ENSBTAG00000034349 | IFI44 | 4.099756359 | 1.588740049 | 0.008019742 | 0.017816655 |
| ENSBTAG00000032808 | FASLG | 4.099372463 | 1.588626626 | 0.008019742 | 0.017816655 |
| ENSBTAG00000046580 | DHX58 | 4.096290912 | 4.718414824 | 2.33E−37 | 5.83E−36 |
| ENSBTAG00000053807 | OAS1X | 4.096220816 | 3.550624533 | 8.34E−17 | 8.54E−16 |
| ENSBTAG00000019942 | PABPC5 | 4.096109334 | 1.587455162 | 0.008019742 | 0.017816655 |
| ENSBTAG00000000900 | LRRC8C | 4.090891053 | 3.198737036 | 7.41E−13 | 5.93E−12 |
| ENSBTAG00000000711 | NDRG1 | 4.08295874 | 8.282836683 | 1.51E−196 | 5.04E−194 |
| ENSBTAG00000051732 | NA | 4.082681449 | 1.582689146 | 0.008019742 | 0.017816655 |
| ENSBTAG00000021485 | C3H1orf54 | 4.082506861 | 1.582628167 | 0.008019742 | 0.017816655 |
| ENSBTAG00000011494 | PYGL | 4.081460669 | 6.477224553 | 7.85E−98 | 6.98E−96 |
| ENSBTAG00000000986 | USH1C | 4.078359181 | 1.581165299 | 0.008019742 | 0.017816655 |
| ENSBTAG00000039695 | PPP1R18 | 4.076196348 | 4.811207882 | 2.53E−39 | 6.63E−38 |
| ENSBTAG00000015095 | TTC6 | 4.075865051 | 1.580284837 | 0.008019742 | 0.017816655 |
| ENSBTAG00000021059 | TNMD | 4.075641027 | 1.580203209 | 0.008019742 | 0.017816655 |
| ENSBTAG00000052702 | COLCA2 | 4.073250563 | 1.579368679 | 0.008019742 | 0.017816655 |
| ENSBTAG00000008575 | CGNL1 | 4.064046665 | 5.393585646 | 4.06E−55 | 1.64E−53 |
| ENSBTAG00000007755 | APOBEC3H | 4.06091283 | 1.57503425 | 0.008019742 | 0.017816655 |
| ENSBTAG00000037768 | MMP3 | 4.060408952 | 1.574873913 | 0.008019742 | 0.017816655 |
| ENSBTAG00000000540 | RNF112 | 4.057801885 | 1.573952274 | 0.008019742 | 0.017816655 |
| ENSBTAG00000054628 | PMEPA1 | 4.051327218 | 6.308838587 | 7.42E−90 | 5.91E−88 |
| ENSBTAG00000007794 | KRT20 | 4.047190617 | 1.570274231 | 0.008019742 | 0.017816655 |
| ENSBTAG00000012558 | ADAMTS12 | 4.042774325 | 1.568706869 | 0.008019742 | 0.017816655 |
| ENSBTAG00000025764 | A0A3Q1MGHO | 4.03874086 | 1.567315306 | 0.008019742 | 0.017816655 |
| ENSBTAG00000048354 | A0A3Q1M1G0 | 4.035925293 | 1.566335244 | 0.008019742 | 0.017816655 |
| ENSBTAG00000009924 | MR1 | 4.034195927 | 1.565731877 | 0.008019742 | 0.017816655 |
| ENSBTAG00000020223 | CASQ1 | 4.028305887 | 1.563709761 | 0.008019742 | 0.017816655 |
| ENSBTAG00000021191 | EHD2 | 4.026172204 | 4.14115068 | 6.93E−25 | 1.08E−23 |
| ENSBTAG00000053657 | A0A3Q1MEE4 | 4.009813406 | 1.557360978 | 0.008019742 | 0.017816655 |
| ENSBTAG00000029897 | MIR21 | 4.004104602 | 1.555412786 | 0.008019742 | 0.017816655 |
| ENSBTAG00000013205 | IL1RAP | 4.00335317 | 3.700947694 | 3.69E−18 | 4.08E−17 |
| ENSBTAG00000017461 | SLC16A3 | 4.003218185 | 3.624228716 | 8.12E−18 | 8.77E−17 |
| ENSBTAG00000002941 | FES | 4.001568276 | 4.254111394 | 1.33E−26 | 2.22E−25 |
| ENSBTAG00000038134 | ZDHHC1 | 4.000304534 | 1.554104291 | 0.015935136 | 0.032624321 |
| ENSBTAG00000016828 | TAPBP | 3.998176083 | 6.221369673 | 6.91E−85 | 5.15E−83 |
| ENSBTAG00000054270 | NA | 3.998118074 | 1.5533729 | 0.015935136 | 0.032624321 |
| ENSBTAG00000015313 | CEACAM19 | 3.992942445 | 3.993960813 | 1.17E−22 | 1.65E−21 |
| ENSBTAG00000037673 | GSTM1 | 3.991724122 | 1.551189643 | 0.015935136 | 0.032624321 |
| ENSBTAG00000053317 | YPEL4 | 3.987972582 | 1.549926084 | 0.015935136 | 0.032624321 |
| ENSBTAG00000019900 | LNPEP | 3.982746816 | 5.04805043 | 1.33E−44 | 4.08E−43 |
| ENSBTAG00000005162 | STX19 | 3.977411434 | 1.546358385 | 0.015935136 | 0.032624321 |
| ENSBTAG00000012630 | PAMR1 | 3.971163632 | 1.544258601 | 0.015935136 | 0.032624321 |
| ENSBTAG00000013976 | AGBL3 | 3.970475832 | 1.544023962 | 0.015935136 | 0.032624321 |
| ENSBTAG00000030973 | TUBA1D | 3.9690014 | 1.543530754 | 0.015935136 | 0.032624321 |
| ENSBTAG00000015345 | BNIPL | 3.968967135 | 1.54351666 | 0.015935136 | 0.032624321 |
| ENSBTAG00000037527 | OAS1Z | 3.966530459 | 1.542715626 | 0.015935136 | 0.032624321 |
| ENSBTAG00000031362 | NXPE2 | 3.965734714 | 1.54247158 | 0.015935136 | 0.032624321 |
| ENSBTAG00000000684 | ARHGEF10L | 3.96233315 | 4.086212688 | 1.07E−23 | 1.59E−22 |
| ENSBTAG00000007665 | NPR3 | 3.960773728 | 1.540789608 | 0.015935136 | 0.032624321 |
| ENSBTAG00000019625 | EHHADH | 3.954703218 | 1.538773844 | 0.015935136 | 0.032624321 |
| ENSBTAG00000013888 | FOXA1 | 3.954596367 | 4.191653654 | 3.36E−25 | 5.32E−24 |
| ENSBTAG00000015304 | ANXA9 | 3.954412585 | 1.538644687 | 0.015935136 | 0.032624321 |
| ENSBTAG00000003078 | COL10A1 | 3.946883824 | 1.536162701 | 0.015935136 | 0.032624321 |
| ENSBTAG00000017704 | ABCG2 | 3.943660724 | 5.745685013 | 1.88E−65 | 9.57E−64 |
| ENSBTAG00000033299 | IGFALS | 3.939312683 | 1.53364108 | 0.015935136 | 0.032624321 |
| ENSBTAG00000027020 | COL5A2 | 3.939121218 | 5.771377815 | 6.51E−66 | 3.39E−64 |
| ENSBTAG00000009625 | DMBX1 | 3.936892066 | 1.532836674 | 0.015935136 | 0.032624321 |
| ENSBTAG00000034147 | OLFML2B | 3.931494904 | 1.53105828 | 0.015935136 | 0.032624321 |
| ENSBTAG00000015718 | CASP8 | 3.930657059 | 3.724082889 | 1.18E−18 | 1.36E−17 |
| ENSBTAG00000026861 | RPL3 | 3.924482435 | 1.528743593 | 0.015935136 | 0.032624321 |
| ENSBTAG00000004211 | TNFRSF1A | 3.922316408 | 5.237638677 | 2.02E−48 | 6.81E−47 |
| ENSBTAG00000002978 | PRKG2 | 3.922275004 | 1.528035864 | 0.015935136 | 0.032624321 |
| ENSBTAG00000008172 | EGLN3 | 3.911704014 | 7.312617509 | 6.81E−135 | 9.70E−133 |
| ENSBTAG00000040340 | PAG21 | 3.908983182 | 1.523699063 | 0.015935136 | 0.032624321 |
| ENSBTAG00000052541 | NA | 3.904828567 | 1.522315101 | 0.015935136 | 0.032624321 |
| ENSBTAG00000016656 | PARP14 | 3.902708725 | 6.522757306 | 7.25E−96 | 6.33E−94 |
| ENSBTAG00000044062 | RBMS3 | 3.897312335 | 1.519871166 | 0.015935136 | 0.032624321 |
| ENSBTAG00000032485 | LHFPL1 | 3.896233741 | 1.519488644 | 0.015935136 | 0.032624321 |
| ENSBTAG00000002123 | MYO3A | 3.895693821 | 1.51934955 | 0.015935136 | 0.032624321 |
| ENSBTAG00000009664 | SP140 | 3.884612474 | 1.515758099 | 0.015935136 | 0.032624321 |
| ENSBTAG00000040406 | OBI1 | 3.882296326 | 1.515013086 | 0.015935136 | 0.032624321 |
| ENSBTAG00000053827 | MAN1B1 | 3.878069339 | 1.513635596 | 0.015935136 | 0.032624321 |
| ENSBTAG00000006232 | WDR86 | 3.869329942 | 1.51086061 | 0.015935136 | 0.032624321 |
| ENSBTAG00000005779 | FTMT | 3.867632023 | 1.510304242 | 0.015935136 | 0.032624321 |
| ENSBTAG00000031851 | SELPLG | 3.865847481 | 4.763927433 | 1.04E−36 | 2.56E−35 |
| ENSBTAG00000014324 | ANTXR2 | 3.864743788 | 5.384175603 | 1.64E−52 | 6.26E−51 |
| ENSBTAG00000015238 | DSC3 | 3.864352053 | 1.509242776 | 0.015935136 | 0.032624321 |
| ENSBTAG00000019300 | TPMT | 3.857074512 | 4.448039488 | 1.66E−29 | 3.14E−28 |
| ENSBTAG00000051240 | A0A3Q1LXB7 | 3.856247711 | 1.506666615 | 0.015935136 | 0.032624321 |
| ENSBTAG00000033222 | CALR3 | 3.855309911 | 1.506370984 | 0.015935136 | 0.032624321 |
| ENSBTAG00000013292 | SIDT1 | 3.843959444 | 1.502765155 | 0.015935136 | 0.032624321 |
| ENSBTAG00000034985 | PHLDA1 | 3.839799398 | 4.885492469 | 6.39E−39 | 1.66E−37 |
| ENSBTAG00000019143 | RPN1 | 3.839654066 | 1.50140723 | 0.015935136 | 0.032624321 |
| ENSBTAG00000001382 | SLC26A9 | 3.839480274 | 3.479525387 | 1.53E−15 | 1.46E−14 |
| ENSBTAG00000002190 | DOCK8 | 3.838671375 | 4.931129504 | 2.11E−40 | 5.69E−39 |
| ENSBTAG00000053595 | HOXA9 | 3.835078793 | 1.499973201 | 0.015935136 | 0.032624321 |
| ENSBTAG00000013210 | ADAMTS4 | 3.831467205 | 1.498831748 | 0.015935136 | 0.032624321 |
| ENSBTAG00000006287 | NEDD9 | 3.831181688 | 6.266985432 | 2.35E−83 | 1.66E−81 |
| ENSBTAG00000007687 | GDF6 | 3.829755462 | 1.498300961 | 0.015935136 | 0.032624321 |
| ENSBTAG00000011500 | CASQ2 | 3.82914136 | 1.498105831 | 0.015935136 | 0.032624321 |
| ENSBTAG00000006811 | EPHA3 | 3.828814525 | 1.497990007 | 0.015935136 | 0.032624321 |
| ENSBTAG00000054551 | MT1X | 3.826909119 | 1.497391475 | 0.015935136 | 0.032624321 |
| ENSBTAG00000015462 | BNC1 | 3.825770331 | 4.311485861 | 6.50E−27 | 1.10E−25 |
| ENSBTAG00000055302 | ICAM2 | 3.825739394 | 1.497052867 | 0.015935136 | 0.032624321 |
| ENSBTAG00000024915 | SECTM1A | 3.823834533 | 1.496458304 | 0.015935136 | 0.032624321 |
| ENSBTAG00000003290 | SLC9A7 | 3.819274569 | 1.4950164 | 0.015935136 | 0.032624321 |
| ENSBTAG00000022920 | RNF128 | 3.816518852 | 5.68275814 | 4.27E−61 | 1.98E−59 |
| ENSBTAG00000050471 | A0A3Q1LY62 | 3.81409087 | 1.493374518 | 0.015935136 | 0.032624321 |
| ENSBTAG00000010637 | ARSG | 3.811437553 | 1.49256811 | 0.015935136 | 0.032624321 |
| ENSBTAG00000034246 | ACRBP | 3.810943874 | 1.492402201 | 0.015935136 | 0.032624321 |
| ENSBTAG00000001333 | PPARG | 3.807884461 | 4.976512272 | 4.69E−41 | 1.28E−39 |
| ENSBTAG00000008593 | INCA1 | 3.807270344 | 1.491249839 | 0.031692221 | 0.059808262 |
| ENSBTAG00000053259 | NA | 3.806047672 | 1.490882021 | 0.031692221 | 0.059808262 |
| ENSBTAG00000007569 | TNFRSF11A | 3.805078487 | 1.490576955 | 0.031692221 | 0.059808262 |
| ENSBTAG00000034384 | VWA3B | 3.804818313 | 1.49048884 | 0.031692221 | 0.059808262 |
| ENSBTAG00000030523 | ZPBP2 | 3.803874392 | 1.490202009 | 0.031692221 | 0.059808262 |
| ENSBTAG00000010998 | CFLAR | 3.803735497 | 4.665351979 | 1.19E−33 | 2.59E−32 |
| ENSBTAG00000020944 | PIEZO1 | 3.802404357 | 6.959902847 | 1.16E−113 | 1.32E−111 |
| ENSBTAG00000002292 | SMARCA1 | 3.799939071 | 6.211333966 | 1.13E−80 | 7.55E−79 |
| ENSBTAG00000021504 | TIRAP | 3.799682342 | 1.488914593 | 0.031692221 | 0.059808262 |
| ENSBTAG00000054742 | NA | 3.790930651 | 1.486193423 | 0.031692221 | 0.059808262 |
| ENSBTAG00000005163 | S100A1 | 3.779637394 | 3.691441146 | 4.53E−17 | 4.70E−16 |
| ENSBTAG00000033381 | SLC35F2 | 3.777964221 | 1.482194094 | 0.031692221 | 0.059808262 |
| ENSBTAG00000006187 | MFAP4 | 3.776541371 | 1.481762673 | 0.031692221 | 0.059808262 |
| ENSBTAG00000022837 | ZNF75D | 3.775441196 | 1.481425122 | 0.031692221 | 0.059808262 |
| ENSBTAG00000010002 | IRF2 | 3.773783912 | 4.015916508 | 5.00E−22 | 6.84E−21 |
| ENSBTAG00000003994 | IGFBP3 | 3.772042062 | 9.013309014 | 7.23E−214 | 2.79E−211 |
| ENSBTAG00000019612 | RNASE4 | 3.759313568 | 1.476523191 | 0.031692221 | 0.059808262 |
| ENSBTAG00000033170 | LRRC58 | 3.758107038 | 3.097001567 | 2.65E−11 | 1.83E−10 |
| ENSBTAG00000000920 | DNAH17 | 3.755403257 | 3.842610011 | 9.56E−20 | 1.17E−18 |
| ENSBTAG00000019694 | ADAM23 | 3.75512693 | 1.475237658 | 0.031692221 | 0.059808262 |
| ENSBTAG00000015212 | IFNAR2 | 3.751666638 | 2.431155325 | 3.91E−06 | 1.61E−05 |
| ENSBTAG00000008505 | APOB | 3.747721603 | 1.473025264 | 0.031692221 | 0.059808262 |
| ENSBTAG00000049316 | A0A3Q1M5N2 | 3.744477522 | 1.472014583 | 0.031692221 | 0.059808262 |
| ENSBTAG00000048426 | TUBB | 3.742603122 | 1.471446746 | 0.031692221 | 0.059808262 |
| ENSBTAG00000050384 | NA | 3.740824037 | 1.470912304 | 0.031692221 | 0.059808262 |
| ENSBTAG00000002070 | KLC3 | 3.734396372 | 1.468983907 | 0.031692221 | 0.059808262 |
| ENSBTAG00000006599 | INHBE | 3.728478282 | 1.467219062 | 0.031692221 | 0.059808262 |
| ENSBTAG00000000009 | FOXF1 | 3.728057484 | 1.467079597 | 0.031692221 | 0.059808262 |
| ENSBTAG00000011824 | OGN | 3.726436462 | 1.466599645 | 0.031692221 | 0.059808262 |
| ENSBTAG00000009707 | MYL1 | 3.725925813 | 1.466447809 | 0.031692221 | 0.059808262 |
| ENSBTAG00000000648 | ADAMTS5 | 3.698704093 | 1.458346857 | 0.031692221 | 0.059808262 |
| ENSBTAG00000010606 | PPP1R3B | 3.696035928 | 3.541777466 | 1.97E−15 | 1.86E−14 |
| ENSBTAG00000048959 | | 3.695186123 | 1.457323762 | 0.031692221 | 0.059808262 |
| ENSBTAG00000048628 | HHAT | 3.69456107 | 1.457138645 | 0.031692221 | 0.059808262 |
| ENSBTAG00000049333 | TNFAIP2 | 3.693391142 | 1.456779451 | 0.031692221 | 0.059808262 |
| ENSBTAG00000010970 | NA | 3.693311716 | 1.456765267 | 0.031692221 | 0.059808262 |
| ENSBTAG00000049163 | TSPAN9 | 3.69134456 | 4.071515778 | 3.78E−22 | 5.20E−21 |
| ENSBTAG00000011734 | ANKRD1 | 3.688535132 | 5.52336598 | 3.54E−54 | 1.40E−52 |
| ENSBTAG00000046324 | Clec2f | 3.685307013 | 1.454442545 | 0.031692221 | 0.059808262 |
| ENSBTAG00000029237 | 5S_rRNA | 3.684057199 | 1.454050116 | 0.031692221 | 0.059808262 |
| ENSBTAG00000008739 | SAMD11 | 3.680413035 | 1.452996282 | 0.031692221 | 0.059808262 |
| ENSBTAG00000017797 | LY75 | 3.674823477 | 3.530662865 | 3.48E−15 | 3.26E−14 |
| ENSBTAG00000054914 | A0A3Q1M9K1 | 3.674728825 | 1.451320231 | 0.031692221 | 0.059808262 |
| ENSBTAG00000008661 | C15H11orf52 | 3.672818005 | 4.183847762 | 1.11E−23 | 1.65E−22 |
| ENSBTAG00000010457 | NUAK1 | 3.669318648 | 2.721979733 | 1.66E−08 | 8.90E−08 |
| ENSBTAG00000005479 | LIPC | 3.66903197 | 1.449662762 | 0.031692221 | 0.059808262 |
| ENSBTAG00000046654 | CERK | 3.666025357 | 3.379844178 | 5.06E−14 | 4.44E−13 |
| ENSBTAG00000010660 | CACNA1C | 3.661976412 | 1.447617895 | 0.031692221 | 0.059808262 |
| ENSBTAG00000007369 | PDLIM2 | 3.660750063 | 4.704656303 | 6.14E−33 | 1.29E−31 |
| ENSBTAG00000023411 | 1300017J02Rik | 3.660283844 | 5.533364332 | 8.14E−54 | 3.19E−52 |
| ENSBTAG00000044027 | ITGBL1 | 3.657973576 | 1.446449933 | 0.031692221 | 0.059808262 |
| ENSBTAG00000003752 | SLC25A24 | 3.657864187 | 7.546197933 | 4.68E−136 | 6.87E−134 |
| ENSBTAG00000012847 | NIBAN2 | 3.65532939 | 6.074959693 | 3.93E−72 | 2.26E−70 |
| ENSBTAG00000025233 | CBARP | 3.65078847 | 1.444389171 | 0.031692221 | 0.059808262 |
| ENSBTAG00000050189 | A0A3Q1LQP5 | 3.649088132 | 1.443896799 | 0.031692221 | 0.059808262 |
| ENSBTAG00000012216 | MLKL | 3.646909606 | 3.785467721 | 1.00E−17 | 1.08E−16 |
| ENSBTAG00000014543 | PLXNA4 | 3.643802242 | 1.44236247 | 0.031692221 | 0.059808262 |
| ENSBTAG00000008650 | CAMK1D | 3.642355098 | 1.441954921 | 0.031692221 | 0.059808262 |
| ENSBTAG00000052465 | Cd24a | 3.639648734 | 1.441175654 | 0.031692221 | 0.059808262 |
| ENSBTAG00000046277 | RGS4 | 3.638638016 | 1.440890861 | 0.031692221 | 0.059808262 |
| ENSBTAG00000012992 | CDH6 | 3.637917516 | 1.440693955 | 0.031692221 | 0.059808262 |
| ENSBTAG00000051077 | A0A3Q1NBR7 | 3.635666764 | 1.440032791 | 0.031692221 | 0.059808262 |
| ENSBTAG00000020704 | RAMP3 | 3.634404704 | 1.439664406 | 0.031692221 | 0.059808262 |
| ENSBTAG00000014053 | MAK | 3.63382235 | 1.43951545 | 0.031692221 | 0.059808262 |
| ENSBTAG00000016005 | PPP3CA | 3.629271437 | 5.525034577 | 1.90E−53 | 7.40E−52 |
| ENSBTAG00000003665 | NUP210 | 3.624477611 | 4.693056402 | 1.65E−32 | 3.44E−31 |
| ENSBTAG00000020263 | TIMP4 | 3.624147002 | 1.436773856 | 0.031692221 | 0.059808262 |
| ENSBTAG00000053867 | NA | 3.623392057 | 1.436526069 | 0.031692221 | 0.059808262 |
| ENSBTAG00000006606 | RFLNB | 3.622591925 | 1.436313585 | 0.031692221 | 0.059808262 |
| ENSBTAG00000011677 | H1-2 | 3.614358204 | 1.433973268 | 0.031692221 | 0.059808262 |
| ENSBTAG00000008342 | TMPRSS13 | 3.614102639 | 1.433894914 | 0.031692221 | 0.059808262 |
| ENSBTAG00000038865 | TCEA3 | 3.611842086 | 1.433234787 | 0.031692221 | 0.059808262 |
| ENSBTAG00000008380 | ITGA11 | 3.611659971 | 2.857149883 | 9.99E−09 | 5.52E−08 |
| ENSBTAG00000004046 | DTX4 | 3.608818633 | 4.720648973 | 2.28E−33 | 4.87E−32 |
| ENSBTAG00000012263 | ASAP3 | 3.607887589 | 4.59990955 | 1.14E−30 | 2.22E−29 |
| ENSBTAG00000019054 | EPSTI1 | 3.60251568 | 4.887450314 | 5.72E−36 | 1.38E−34 |
| ENSBTAG00000018123 | FBLN5 | 3.599171504 | 2.815396851 | 1.80E−08 | 9.64E−08 |
| ENSBTAG00000001509 | ELK3 | 3.597031853 | 3.621098045 | 7.46E−16 | 7.24E−15 |
| ENSBTAG00000053511 | SERPINB6 | 3.592301006 | 1.42777254 | 0.031692221 | 0.059808262 |
| ENSBTAG00000038020 | DTD2 | 3.583889818 | 1.425406482 | 0.0630888 | 0.107620776 |
| ENSBTAG00000013085 | CELA3B | 3.583757149 | 1.42535308 | 0.0630888 | 0.107620776 |
| ENSBTAG00000010742 | PKD2L1 | 3.579180739 | 4.219037047 | 1.37E−23 | 2.01E−22 |
| ENSBTAG00000045991 | G3N2X2 | 3.574793516 | 1.42287883 | 0.0630888 | 0.107620776 |
| ENSBTAG00000024934 | SECTM1 | 3.572354699 | 1.42222675 | 0.0630888 | 0.107620776 |
| ENSBTAG00000013066 | IGF2 | 3.569595957 | 3.703348852 | 2.73E−16 | 2.71E−15 |
| ENSBTAG00000046155 | RGN | 3.565814331 | 1.42040095 | 0.0630888 | 0.107620776 |
| ENSBTAG00000001085 | FAM20A | 3.564066081 | 1.419910705 | 0.0630888 | 0.107620776 |
| ENSBTAG00000001675 | ITPKA | 3.563819352 | 1.419832031 | 0.0630888 | 0.107620776 |
| ENSBTAG00000006703 | PTGDR | 3.556225435 | 1.41776307 | 0.0630888 | 0.107620776 |
| ENSBTAG00000015752 | SP110 | 3.555344853 | 1.417512168 | 0.0630888 | 0.107620776 |
| ENSBTAG00000020688 | ARHGEF15 | 3.553652072 | 1.417040807 | 0.0630888 | 0.107620776 |
| ENSBTAG00000021791 | PARP9 | 3.55074388 | 4.944446737 | 4.28E−37 | 1.06E−35 |
| ENSBTAG00000008703 | EIF2AK2 | 3.544831246 | 5.733220808 | 2.29E−58 | 1.00E−56 |
| ENSBTAG00000037508 | EBF1 | 3.544097878 | 1.414412003 | 0.0630888 | 0.107620776 |
| ENSBTAG00000047223 | GNAS | 3.543900802 | 5.366966381 | 5.82E−48 | 1.93E−46 |
| ENSBTAG00000009998 | GALNT16 | 3.541280432 | 4.117465038 | 2.73E−22 | 3.79E−21 |
| ENSBTAG00000055209 | A0A3Q1M2S3 | 3.540392497 | 1.413405783 | 0.0630888 | 0.107620776 |
| ENSBTAG00000012020 | TRAF5 | 3.536073488 | 3.874161567 | 2.20E−18 | 2.47E−17 |
| ENSBTAG00000031669 | CTNNA2 | 3.535472436 | 1.412076943 | 0.0630888 | 0.107620776 |
| ENSBTAG00000047383 | ABCC4 | 3.531647372 | 1.411037301 | 0.0630888 | 0.107620776 |
| ENSBTAG00000015297 | BANK1 | 3.52878777 | 1.410262035 | 0.0630888 | 0.107620776 |
| ENSBTAG00000047914 | CDHR5 | 3.528780205 | 1.410260323 | 0.0630888 | 0.107620776 |
| ENSBTAG00000007719 | MFSD6 | 3.528762166 | 5.520799784 | 1.75E−51 | 6.44E−50 |
| ENSBTAG00000012234 | RHBG | 3.523729642 | 5.227527836 | 5.27E−44 | 1.58E−42 |
| ENSBTAG00000044158 | LDLRAD3 | 3.522054551 | 4.051065908 | 6.73E−21 | 8.68E−20 |
| ENSBTAG00000008592 | FCGR1A | 3.521385098 | 1.408270657 | 0.0630888 | 0.107620776 |
| ENSBTAG00000037996 | ARMCX3 | 3.518998835 | 6.517196267 | 7.92E−86 | 5.96E−84 |
| ENSBTAG00000006645 | LPAR6 | 3.516121501 | 1.406824523 | 0.0630888 | 0.107620776 |
| ENSBTAG00000000177 | MSLN | 3.514924896 | 1.406505996 | 0.0630888 | 0.107620776 |
| ENSBTAG00000052513 | A0A3Q1MAY4 | 3.514887556 | 1.406495008 | 0.0630888 | 0.107620776 |
| ENSBTAG00000013213 | OSR2 | 3.513972462 | 1.406251698 | 0.0630888 | 0.107620776 |
| ENSBTAG00000020199 | F2R | 3.510772895 | 7.168150637 | 5.11E−113 | 5.77E−111 |
| ENSBTAG00000053373 | ABCC4 | 3.507560697 | 1.404530926 | 0.0630888 | 0.107620776 |
| ENSBTAG00000003018 | FSTL3 | 3.506485317 | 6.324475841 | 1.97E−77 | 1.24E−75 |
| ENSBTAG00000022120 | FGB | 3.499491759 | 1.402382309 | 0.0630888 | 0.107620776 |
| ENSBTAG00000010943 | SLC22A23 | 3.496006884 | 5.023342278 | 8.16E−39 | 2.11E−37 |
| ENSBTAG00000002940 | WNT16 | 3.495017073 | 3.226276978 | 5.32E−12 | 3.93E−11 |
| ENSBTAG00000053187 | | 3.493801208 | 1.400855417 | 0.0630888 | 0.107620776 |
| ENSBTAG00000045507 | ZNF469 | 3.491963557 | 1.400363804 | 0.0630888 | 0.107620776 |
| ENSBTAG00000038718 | Prl | 3.491383832 | 1.40022813 | 0.0630888 | 0.107620776 |
| ENSBTAG00000017457 | STK10 | 3.490622744 | 7.001639982 | 6.73E−105 | 6.90E−103 |
| ENSBTAG00000023026 | SERPINB2 | 3.48845609 | 3.932780671 | 1.40E−18 | 1.60E−17 |
| ENSBTAG00000050252 | NA | 3.487415294 | 1.399155906 | 0.0630888 | 0.107620776 |
| ENSBTAG00000004650 | ZNF516 | 3.48738683 | 1.399144042 | 0.0630888 | 0.107620776 |
| ENSBTAG00000000582 | LY6G6E | 3.486157285 | 6.030990674 | 4.52E−67 | 2.41E−65 |
| ENSBTAG00000032031 | ZBTB12 | 3.485286543 | 1.398576942 | 0.0630888 | 0.107620776 |
| ENSBTAG00000016879 | DENND11 | 3.48135973 | 1.397540557 | 0.0630888 | 0.107620776 |
| ENSBTAG00000005884 | WDR31 | 3.480143162 | 1.397214043 | 0.0630888 | 0.107620776 |
| ENSBTAG00000005390 | GMFG | 3.478293446 | 1.396722367 | 0.0630888 | 0.107620776 |
| ENSBTAG00000017674 | SCNN1D | 3.478124171 | 1.396693693 | 0.0630888 | 0.107620776 |
| ENSBTAG00000001638 | FGA | 3.477479879 | 1.396540387 | 0.0630888 | 0.107620776 |
| ENSBTAG00000009844 | CCN1 | 3.476414257 | 7.900674636 | 6.54E−144 | 1.17E−141 |
| ENSBTAG00000008530 | SLC36A4 | 3.474936636 | 4.680327926 | 4.05E−31 | 8.02E−30 |
| ENSBTAG00000051859 | KIF20B | 3.474283117 | 1.395660633 | 0.0630888 | 0.107620776 |
| ENSBTAG00000017266 | ITGA6 | 3.473224442 | 8.750919055 | 3.16E−181 | 8.27E−179 |
| ENSBTAG00000010085 | SLC7A2 | 3.472968139 | 1.395331883 | 0.0630888 | 0.107620776 |
| ENSBTAG00000033107 | OSMR | 3.470727626 | 1.39472835 | 0.0630888 | 0.107620776 |
| ENSBTAG00000054825 | PSMA7 | 3.470638717 | 1.394697647 | 0.0630888 | 0.107620776 |
| ENSBTAG00000048365 | WNT7B | 3.469855439 | 3.352102438 | 1.04E−12 | 8.15E−12 |
| ENSBTAG00000027625 | SFRP1 | 3.469161471 | 4.992313497 | 1.35E−37 | 3.40E−36 |
| ENSBTAG00000018252 | ARRDC4 | 3.466279244 | 4.409671329 | 2.32E−26 | 3.84E−25 |
| ENSBTAG00000053271 | A0A3Q1M7V3 | 3.462939715 | 1.392684223 | 0.0630888 | 0.107620776 |
| ENSBTAG00000008142 | IFIH1 | 3.453686641 | 4.683978796 | 2.12E−30 | 4.10E−29 |
| ENSBTAG00000054325 | A0A3Q1MJG4 | 3.444581043 | 3.431447004 | 6.07E−13 | 4.89E−12 |
| ENSBTAG00000006995 | SPTBN1 | 3.437313349 | 7.753656455 | 7.33E−136 | 1.06E−133 |
| ENSBTAG00000003054 | INSRR | 3.436484797 | 1.385813298 | 0.0630888 | 0.107620776 |
| ENSBTAG00000003877 | ZCCHC24 | 3.434304641 | 1.385230356 | 0.0630888 | 0.107620776 |
| ENSBTAG00000017709 | ATRNL1 | 3.425907472 | 1.383055746 | 0.0630888 | 0.107620776 |
| ENSBTAG00000016704 | SLC37A2 | 3.425682998 | 3.723674195 | 2.73E−16 | 2.71E−15 |
| ENSBTAG00000050661 | IFNT3 | 3.424044029 | 6.53004825 | 1.10E−83 | 7.82E−82 |
| ENSBTAG00000003152 | IFI27 | 3.42389214 | 4.283217151 | 3.03E−23 | 4.39E−22 |
| ENSBTAG00000009933 | DTX3L | 3.423663389 | 4.52528366 | 1.55E−27 | 2.69E−26 |
| ENSBTAG00000010704 | LRRC4 | 3.421925777 | 1.382041962 | 0.0630888 | 0.107620776 |
| ENSBTAG00000020520 | RASD1 | 3.421284923 | 1.381864941 | 0.0630888 | 0.107620776 |
| ENSBTAG00000014540 | PERM1 | 3.418974061 | 1.38128851 | 0.0630888 | 0.107620776 |
| ENSBTAG00000017834 | PRELP | 3.417607102 | 1.380917856 | 0.0630888 | 0.107620776 |
| ENSBTAG00000006367 | CCN2 | 3.41478359 | 7.629731395 | 9.02E−129 | 1.20E−126 |
| ENSBTAG00000051376 | SLC25A4 | 3.413499159 | 1.379877415 | 0.0630888 | 0.107620776 |
| ENSBTAG00000033096 | PAG2 | 3.413333896 | 10.71926706 | 6.07E−250 | 3.18E−247 |
| ENSBTAG00000001702 | TMEM107 | 3.409362168 | 1.378818563 | 0.0630888 | 0.107620776 |
| ENSBTAG00000021775 | SLC4A9 | 3.409027922 | 1.378758496 | 0.0630888 | 0.107620776 |
| ENSBTAG00000045588 | BST2 | 3.408326928 | 2.758833504 | 1.05E−07 | 5.19E−07 |
| ENSBTAG00000023407 | B3GALT5 | 3.408194275 | 4.211779388 | 2.16E−22 | 3.01E−21 |
| ENSBTAG00000004792 | CHMP4C | 3.404427038 | 1.377562871 | 0.0630888 | 0.107620776 |
| ENSBTAG00000021649 | Rpl27a | 3.403331386 | 1.377288726 | 0.0630888 | 0.107620776 |
| ENSBTAG00000047939 | ZNF75D | 3.403066228 | 1.3772191 | 0.0630888 | 0.107620776 |
| ENSBTAG00000021358 | BLNK | 3.402635316 | 3.407374089 | 1.85E−12 | 1.42E−11 |
| ENSBTAG00000009570 | ANGPTL8 | 3.397044016 | 1.375695055 | 0.0630888 | 0.107620776 |
| ENSBTAG00000009997 | ANO1 | 3.394188977 | 3.21059105 | 9.62E−11 | 6.35E−10 |
| ENSBTAG00000011088 | SLC6A6 | 3.392622656 | 5.31189298 | 6.40E−44 | 1.91E−42 |
| ENSBTAG00000048549 | IFNT | 3.392392865 | 5.467701621 | 4.62E−48 | 1.54E−46 |
| ENSBTAG00000048788 | ZNF333 | 3.391061012 | 1.374176403 | 0.0630888 | 0.107620776 |
| ENSBTAG00000053140 | NA | 3.390531606 | 1.374041983 | 0.0630888 | 0.107620776 |
| ENSBTAG00000047319 | Gm266 | 3.389527371 | 2.996179303 | 3.07E−09 | 1.77E−08 |
| ENSBTAG00000008394 | MYL3 | 3.385845081 | 1.372869876 | 0.0630888 | 0.107620776 |
| ENSBTAG00000049382 | TMEM229A | 3.382857646 | 1.372113282 | 0.0630888 | 0.107620776 |
| ENSBTAG00000021282 | SGCE | 3.382679251 | 2.204820314 | 2.46E−05 | 8.93E−05 |
| ENSBTAG00000044049 | ABHD12B | 3.382417786 | 1.372009619 | 0.0630888 | 0.107620776 |
| ENSBTAG00000004685 | SRGAP3 | 3.380009927 | 1.371388269 | 0.0630888 | 0.107620776 |
| ENSBTAG00000054422 | | 3.378265785 | 1.370949731 | 0.0630888 | 0.107620776 |
| ENSBTAG00000017741 | HACD4 | 3.371924922 | 1.369349819 | 0.0630888 | 0.107620776 |
| ENSBTAG00000019627 | THY1 | 3.367210338 | 4.085509603 | 1.28E−20 | 1.64E−19 |
| ENSBTAG00000014506 | ZNF503 | 3.366010279 | 1.367878005 | 0.0630888 | 0.107620776 |
| ENSBTAG00000010551 | ATP1A2 | 3.365619252 | 1.367782675 | 0.0630888 | 0.107620776 |
| ENSBTAG00000049667 | A0A3Q1M583 | 3.364275429 | 1.367445211 | 0.0630888 | 0.107620776 |
| ENSBTAG00000053625 | SKP2 | 3.360793668 | 1.366578041 | 0.0630888 | 0.107620776 |
| ENSBTAG00000050138 | NA | 3.357613672 | 1.365782728 | 0.0630888 | 0.107620776 |
| ENSBTAG00000050392 | FAM171B | 3.35545568 | 6.343837855 | 3.03E−74 | 1.81E−72 |
| ENSBTAG00000031654 | KCND3 | 3.35388791 | 1.364846785 | 0.0630888 | 0.107620776 |
| ENSBTAG00000010693 | LMO7 | 3.353819157 | 7.924334165 | 1.25E−138 | 1.98E−136 |
| ENSBTAG00000003155 | IFI27L2 | 3.34791501 | 1.363380903 | 0.0630888 | 0.107620776 |
| ENSBTAG00000052619 | NA | 3.346588727 | 1.363061078 | 0.0630888 | 0.107620776 |
| ENSBTAG00000008431 | MOGAT1 | 3.340396275 | 5.588321762 | 2.55E−50 | 9.07E−49 |
| ENSBTAG00000013148 | CLDN1 | 3.339390178 | 5.702756751 | 1.76E−53 | 6.88E−52 |
| ENSBTAG00000001028 | MAP6 | 3.337680745 | 1.360857481 | 0.0630888 | 0.107620776 |
| ENSBTAG00000048931 | A0A3Q1MRL6 | 3.332855116 | 3.099552891 | 3.07E−10 | 1.94E−09 |
| ENSBTAG00000039285 | CSNK1A1 | 3.332035473 | 1.359475206 | 0.0630888 | 0.107620776 |
| ENSBTAG00000037389 | TRIM44 | 3.331994031 | 1.359460863 | 0.0630888 | 0.107620776 |
| ENSBTAG00000024839 | Gm6133 | 3.331139455 | 1.359252286 | 0.0630888 | 0.107620776 |
| ENSBTAG00000013942 | DAGLA | 3.330146792 | 1.359013749 | 0.0630888 | 0.107620776 |
| ENSBTAG00000010030 | EPHA4 | 3.327729884 | 5.506560379 | 3.31E−48 | 1.11E−46 |
| ENSBTAG00000022580 | INKA2 | 3.326320412 | 1.35807604 | 0.0630888 | 0.107620776 |
| ENSBTAG00000013202 | PLCZ1 | 3.323118799 | 1.357300376 | 0.0630888 | 0.107620776 |
| ENSBTAG00000010793 | CCDC80 | 3.322936356 | 4.01917746 | 3.02E−19 | 3.60E−18 |
| ENSBTAG00000049765 | NA | 3.317923665 | 1.356027659 | 0.125705897 | 0.191775369 |
| ENSBTAG00000053410 | NA | 3.313444626 | 1.354949307 | 0.125705897 | 0.191775369 |
| ENSBTAG00000053103 | TCHH | 3.309017688 | 4.584948404 | 1.97E−27 | 3.41E−26 |
| ENSBTAG00000053725 | NA | 3.305099274 | 1.352916604 | 0.125705897 | 0.191775369 |
| ENSBTAG00000010821 | ACER2 | 3.297035803 | 2.963212771 | 5.45E−09 | 3.08E−08 |
| ENSBTAG00000000917 | BMP1 | 3.291662775 | 5.190967906 | 8.17E−40 | 2.17E−38 |
| ENSBTAG00000037899 | DLK1 | 3.281007101 | 1.34712596 | 0.125705897 | 0.191775369 |
| ENSBTAG00000005284 | SERPINE3 | 3.277945403 | 1.346400329 | 0.125705897 | 0.191775369 |
| ENSBTAG00000020018 | FRK | 3.277458709 | 3.896939576 | 1.18E−17 | 1.27E−16 |
| ENSBTAG00000001088 | SLC17A8 | 3.276609981 | 1.346102894 | 0.125705897 | 0.191775369 |
| ENSBTAG00000033304 | TCIM | 3.27573243 | 3.954741551 | 7.08E−18 | 7.66E−17 |
| ENSBTAG00000021818 | ADGRE5 | 3.271209253 | 4.80058381 | 2.91E−31 | 5.79E−30 |
| ENSBTAG00000031998 | CXCL16 | 3.270886186 | 6.11766876 | 1.12E−64 | 5.60E−63 |
| ENSBTAG00000016502 | Novel gene | 3.270337193 | 1.344590167 | 0.125705897 | 0.191775369 |
| ENSBTAG00000021048 | ADM | 3.26856894 | 5.697899521 | 2.44E−52 | 9.33E−51 |
| ENSBTAG00000003366 | DDX58 | 3.268274654 | 5.53473496 | 7.88E−48 | 2.60E−46 |
| ENSBTAG00000018785 | TUBB1 | 3.265932699 | 1.343554136 | 0.125705897 | 0.191775369 |
| ENSBTAG00000018059 | CD80 | 3.264148768 | 1.3431335 | 0.125705897 | 0.191775369 |
| ENSBTAG00000017248 | A0A0A0MPA4 | 3.261705147 | 1.342555247 | 0.125705897 | 0.191775369 |
| ENSBTAG00000016658 | ABHD4 | 3.250349498 | 6.687730122 | 5.90E−84 | 4.26E−82 |
| ENSBTAG00000051860 | NA | 3.247980239 | 1.339323379 | 0.125705897 | 0.191775369 |
| ENSBTAG00000051743 | NA | 3.247738313 | 1.339262242 | 0.125705897 | 0.191775369 |
| ENSBTAG00000052141 | A0A3Q1LSI5 | 3.247624098 | 1.339218991 | 0.125705897 | 0.191775369 |
| ENSBTAG00000017339 | RUNX1T1 | 3.246124655 | 1.338878385 | 0.125705897 | 0.191775369 |
| ENSBTAG00000033169 | LAMB4 | 3.244058621 | 1.338416117 | 0.125705897 | 0.191775369 |
| ENSBTAG00000010830 | LRP1 | 3.237136082 | 8.342258749 | 4.43E−149 | 8.33E−147 |
| ENSBTAG00000011374 | S1PR3 | 3.233131621 | 1.335852648 | 0.125705897 | 0.191775369 |
| ENSBTAG00000012969 | DKK2 | 3.232321582 | 1.335649246 | 0.125705897 | 0.191775369 |
| ENSBTAG00000000869 | OBI1 | 3.226926734 | 1.334402085 | 0.125705897 | 0.191775369 |
| ENSBTAG00000032077 | KCND2 | 3.22096193 | 1.333027625 | 0.125705897 | 0.191775369 |
| ENSBTAG00000045498 | SULT2B1 | 3.220715643 | 1.332984135 | 0.125705897 | 0.191775369 |
| ENSBTAG00000053885 | FAM47E | 3.218038525 | 1.332344029 | 0.125705897 | 0.191775369 |
| ENSBTAG00000016826 | GPR176 | 3.216200706 | 1.331921329 | 0.125705897 | 0.191775369 |
| ENSBTAG00000018543 | VIL1 | 3.214770326 | 1.331600461 | 0.125705897 | 0.191775369 |
| ENSBTAG00000020705 | PRSS12 | 3.213578725 | 5.734976009 | 4.77E−52 | 1.80E−50 |
| ENSBTAG00000015717 | HEY1 | 3.213282288 | 4.029730071 | 1.47E−18 | 1.67E−17 |
| ENSBTAG00000016713 | TMEM30B | 3.21275101 | 3.331356903 | 1.70E−11 | 1.19E−10 |
| ENSBTAG00000049289 | NA | 3.212444071 | 1.331079399 | 0.125705897 | 0.191775369 |
| ENSBTAG00000001324 | SLCO2A1 | 3.20640926 | 6.264952216 | 2.66E−68 | 1.44E−66 |
| ENSBTAG00000054572 | PEG3 | 3.20552765 | 8.240051544 | 1.94E−143 | 3.42E−141 |
| ENSBTAG00000055316 | SNRPD1 | 3.202924594 | 1.328871615 | 0.125705897 | 0.191775369 |
| ENSBTAG00000000928 | SPECC1 | 3.201607069 | 3.949664763 | 7.08E−18 | 7.66E−17 |
| ENSBTAG00000031420 | E1BN83 | 3.200905596 | 1.328415307 | 0.125705897 | 0.191775369 |
| ENSBTAG00000008752 | NME5 | 3.200201127 | 1.328245164 | 0.125705897 | 0.191775369 |
| ENSBTAG00000047613 | HTRA3 | 3.199975954 | 1.32820339 | 0.125705897 | 0.191775369 |
| ENSBTAG00000047294 | CUL1 | 3.198646137 | 1.327907177 | 0.125705897 | 0.191775369 |
| ENSBTAG00000016688 | LDHA | 3.198306918 | 5.034741895 | 7.86E−35 | 1.81E−33 |
| ENSBTAG00000037650 | RTL9 | 3.19828992 | 1.327805537 | 0.125705897 | 0.191775369 |
| ENSBTAG00000051630 | A0A3Q1MH01 | 3.198028327 | 1.327765939 | 0.125705897 | 0.191775369 |
| ENSBTAG00000013099 | ALDOC | 3.197009436 | 5.677141336 | 6.31E−50 | 2.22E−48 |
| ENSBTAG00000010303 | ICAM1 | 3.195445403 | 5.674318508 | 2.36E−50 | 8.44E−49 |
| ENSBTAG00000003191 | FSCN1 | 3.195073919 | 7.61726335 | 2.17E−118 | 2.70E−116 |
| ENSBTAG00000000707 | CCN4 | 3.192501922 | 1.326486018 | 0.125705897 | 0.191775369 |
| ENSBTAG00000007423 | TNFRSF11B | 3.188150098 | 1.325513476 | 0.125705897 | 0.191775369 |
| ENSBTAG00000026994 | C2H2orf88 | 3.187120697 | 1.325271526 | 0.125705897 | 0.191775369 |
| ENSBTAG00000018343 | NCR3 | 3.184732337 | 1.32472215 | 0.125705897 | 0.191775369 |
| ENSBTAG00000020914 | CPNE8 | 3.183681172 | 3.104056526 | 1.60E−09 | 9.51E−09 |
| ENSBTAG00000046391 | GRAMD2A | 3.182239682 | 3.408823125 | 1.54E−11 | 1.08E−10 |
| ENSBTAG00000002604 | SP8 | 3.18210834 | 1.324130011 | 0.125705897 | 0.191775369 |
| ENSBTAG00000001209 | PHLDB2 | 3.181492308 | 6.079011217 | 1.70E−61 | 8.01E−60 |
| ENSBTAG00000001687 | STC1 | 3.180436196 | 4.516011396 | 1.61E−25 | 2.59E−24 |
| ENSBTAG00000049271 | NA | 3.177635997 | 1.323126822 | 0.125705897 | 0.191775369 |
| ENSBTAG00000052130 | Atxn3 | 3.176616444 | 1.322903002 | 0.125705897 | 0.191775369 |
| ENSBTAG00000016334 | SATB2 | 3.17597037 | 4.738566925 | 3.32E−29 | 6.18E−28 |
| ENSBTAG00000004511 | VAV2 | 3.174546611 | 5.550068256 | 5.98E−47 | 1.92E−45 |
| ENSBTAG00000024869 | CX3CL1 | 3.173355735 | 1.322169556 | 0.125705897 | 0.191775369 |
| ENSBTAG00000045998 | MAGEL2 | 3.171333614 | 1.321716947 | 0.125705897 | 0.191775369 |
| ENSBTAG00000005958 | PTK2B | 3.168912698 | 5.794394037 | 2.26E−53 | 8.76E−52 |
| ENSBTAG00000053871 | APELA | 3.166908436 | 1.320734801 | 0.125705897 | 0.191775369 |
| ENSBTAG00000005108 | SLIT2 | 3.166697857 | 2.914555603 | 3.03E−08 | 1.58E−07 |
| ENSBTAG00000001414 | KCTD12 | 3.165701729 | 1.320431167 | 0.125705897 | 0.191775369 |
| ENSBTAG00000019966 | TICAM1 | 3.16258214 | 3.330503678 | 2.95E−11 | 2.02E−10 |
| ENSBTAG00000004291 | SEPTIN6 | 3.161751977 | 4.21970092 | 8.62E−21 | 1.10E−19 |
| ENSBTAG00000019249 | HS3ST6 | 3.159040597 | 1.318948968 | 0.125705897 | 0.191775369 |
| ENSBTAG00000018203 | CD101 | 3.15567263 | 1.318214544 | 0.125705897 | 0.191775369 |
| ENSBTAG00000001609 | MAP2K6 | 3.153180395 | 2.681817522 | 6.06E−07 | 2.74E−06 |
| ENSBTAG00000031214 | A0A3Q1N580 | 3.151031232 | 1.317165362 | 0.125705897 | 0.191775369 |
| ENSBTAG00000000585 | LY6G6C | 3.145980177 | 6.251328793 | 3.58E−66 | 1.87E−64 |
| ENSBTAG00000053806 | BST2 | 3.144560808 | 1.315726412 | 0.125705897 | 0.191775369 |
| ENSBTAG00000009784 | RAB32 | 3.141949658 | 4.728735236 | 2.31E−28 | 4.19E−27 |
| ENSBTAG00000002390 | CCDC184 | 3.139035225 | 1.314500637 | 0.125705897 | 0.191775369 |
| ENSBTAG00000005685 | HSD11B2 | 3.133775584 | 1.313339359 | 0.125705897 | 0.191775369 |
| ENSBTAG00000013799 | processed | 3.133141678 | 4.132259548 | 1.11E−19 | 1.34E−18 |
| | pseudogene | | | | |
| ENSBTAG00000049814 | TNIK | 3.132002829 | 4.320909913 | 2.55E−22 | 3.54E−21 |
| ENSBTAG00000021218 | MYLPF | 3.130649034 | 1.312648745 | 0.125705897 | 0.191775369 |
| ENSBTAG00000011373 | MAML3 | 3.130169219 | 3.179915199 | 2.97E−10 | 1.88E−09 |
| ENSBTAG00000009359 | A0A3Q1NGZ9 | 3.129955254 | 1.312483494 | 0.125705897 | 0.191775369 |
| ENSBTAG00000049552 | NA | 3.129840136 | 1.312466893 | 0.125705897 | 0.191775369 |
| ENSBTAG00000050457 | A0A3Q1LFX8 | 3.128051012 | 1.312078044 | 0.125705897 | 0.191775369 |
| ENSBTAG00000048619 | PRRT3 | 3.127812119 | 3.372450048 | 4.53E−11 | 3.07E−10 |
| ENSBTAG00000027442 | NFIB | 3.125533494 | 1.311519665 | 0.125705897 | 0.191775369 |
| ENSBTAG00000050554 | A0A3Q1LM41 | 3.124217904 | 1.311240358 | 0.125705897 | 0.191775369 |
| ENSBTAG00000000745 | AQP1 | 3.122618612 | 1.310871775 | 0.125705897 | 0.191775369 |
| ENSBTAG00000016844 | ADGB | 3.118306829 | 1.309949899 | 0.125705897 | 0.191775369 |
| ENSBTAG00000044179 | CERS6 | 3.11410226 | 1.309010529 | 0.125705897 | 0.191775369 |
| ENSBTAG00000003336 | TPD52L1 | 3.112862211 | 4.814140295 | 5.75E−30 | 1.10E−28 |
| ENSBTAG00000046328 | XG | 3.111023364 | 1.308339038 | 0.125705897 | 0.191775369 |
| ENSBTAG00000054671 | GPC6 | 3.110651201 | 1.308255395 | 0.125705897 | 0.191775369 |
| ENSBTAG00000050968 | NA | 3.109967954 | 1.308116713 | 0.125705897 | 0.191775369 |
| ENSBTAG00000053958 | A0A3Q1M5J4 | 3.107763852 | 1.307638435 | 0.125705897 | 0.191775369 |
| ENSBTAG00000008416 | ABHD5 | 3.1029177 | 5.442017752 | 6.28E−43 | 1.84E−41 |
| ENSBTAG00000016541 | CAMK1G | 3.102696181 | 1.306539967 | 0.125705897 | 0.191775369 |
| ENSBTAG00000010349 | IL1A | 3.099929433 | 1.305958029 | 0.125705897 | 0.191775369 |
| ENSBTAG00000006335 | STAT6 | 3.099021934 | 4.294254769 | 4.96E−21 | 6.45E−20 |
| ENSBTAG00000046587 | CST6 | 3.098664597 | 3.935575102 | 9.24E−17 | 9.45E−16 |
| ENSBTAG00000008721 | MANSC1 | 3.097079947 | 4.308889995 | 3.03E−21 | 3.97E−20 |
| ENSBTAG00000036106 | PRP9 | 3.095883296 | 1.305083897 | 0.125705897 | 0.191775369 |
| ENSBTAG00000018073 | TSPO | 3.094004805 | 4.55599267 | 4.44E−25 | 7.00E−24 |
| ENSBTAG00000020267 | WNT9A | 3.092049541 | 1.304225002 | 0.125705897 | 0.191775369 |
| ENSBTAG00000004515 | KCNK1 | 3.090481113 | 5.175684544 | 3.55E−36 | 8.64E−35 |
| ENSBTAG00000004782 | PTPRCAP | 3.090254848 | 1.303847833 | 0.125705897 | 0.191775369 |
| ENSBTAG00000040242 | NA | 3.089063511 | 1.303581204 | 0.125705897 | 0.191775369 |
| ENSBTAG00000008665 | DIXDC1 | 3.079902794 | 1.301634073 | 0.125705897 | 0.191775369 |
| ENSBTAG00000008779 | ALOX15B | 3.079842062 | 1.301609014 | 0.125705897 | 0.191775369 |
| ENSBTAG00000000671 | PARP3 | 3.07812234 | 2.993886264 | 2.59E−08 | 1.36E−07 |
| ENSBTAG00000038783 | pseudogene | 3.077140666 | 1.301030621 | 0.125705897 | 0.191775369 |
| ENSBTAG00000016347 | NPM1 | 3.076955673 | 1.300988068 | 0.125705897 | 0.191775369 |
| ENSBTAG00000049982 | bta-mir-10181 | 3.076824175 | 1.300961383 | 0.125705897 | 0.191775369 |
| ENSBTAG00000015759 | A0A3Q1LQE8 | 3.076735296 | 1.300940741 | 0.125705897 | 0.191775369 |
| ENSBTAG00000053314 | NA | 3.074575975 | 1.300481986 | 0.125705897 | 0.191775369 |
| ENSBTAG00000004564 | MBNL1 | 3.073169552 | 5.159518523 | 1.30E−35 | 3.07E−34 |
| ENSBTAG00000016505 | TMC7 | 3.072857055 | 3.1764497 | 1.38E−09 | 8.29E−09 |
| ENSBTAG00000051675 | C15orf65 | 3.072701858 | 1.300071444 | 0.125705897 | 0.191775369 |
| ENSBTAG00000017678 | SPRED3 | 3.072356109 | 1.300006572 | 0.125705897 | 0.191775369 |
| ENSBTAG00000000027 | IGFL1 | 3.071790949 | 3.081261626 | 2.80E−09 | 1.62E−08 |
| ENSBTAG00000055110 | A0A3Q1LU32 | 3.069472133 | 1.29938951 | 0.125705897 | 0.191775369 |
| ENSBTAG00000050013 | A0A3Q1MAK0 | 3.06565132 | 1.298576519 | 0.125705897 | 0.191775369 |
| ENSBTAG00000021444 | TWIST2 | 3.061716525 | 1.297746362 | 0.125705897 | 0.191775369 |
| ENSBTAG00000033160 | FAM114A1 | 3.061327881 | 2.823564317 | 1.66E−07 | 7.99E−07 |
| ENSBTAG00000038462 | TMEM182 | 3.059452073 | 1.297241868 | 0.125705897 | 0.191775369 |
| ENSBTAG00000054445 | NA | 3.059149824 | 1.29718355 | 0.125705897 | 0.191775369 |
| ENSBTAG00000021557 | FUT2 | 3.057726326 | 3.92005754 | 2.60E−16 | 2.58E−15 |
| ENSBTAG00000015919 | PARM1 | 3.057498823 | 6.352023574 | 6.43E−67 | 3.40E−65 |
| ENSBTAG00000000454 | TMEM106C | 3.052344587 | 6.67383971 | 3.21E−77 | 2.01E−75 |
| ENSBTAG00000049010 | RNF150 | 3.05034687 | 2.409065559 | 1.30E−05 | 4.92E−05 |
| ENSBTAG00000002473 | ANGPTL4 | 3.050327748 | 1.295334182 | 0.125705897 | 0.191775369 |
| ENSBTAG00000001911 | PCDH11X | 3.047807957 | 1.294823488 | 0.125705897 | 0.191775369 |
| ENSBTAG00000007601 | NA | 3.043821701 | 1.293957335 | 0.125705897 | 0.191775369 |
| ENSBTAG00000054796 | NA | 3.043625524 | 1.29391983 | 0.125705897 | 0.191775369 |
| ENSBTAG00000014737 | VEGFC | 3.042023318 | 2.980450918 | 4.50E−08 | 2.30E−07 |
| ENSBTAG00000001094 | MTUS2 | 3.039553583 | 4.123670572 | 1.35E−18 | 1.55E−17 |
| ENSBTAG00000019988 | GNA15 | 3.039425058 | 4.822039281 | 2.40E−29 | 4.51E−28 |
| ENSBTAG00000004190 | ARHGAP29 | 3.037985 | 6.383340636 | 4.75E−67 | 2.52E−65 |
| ENSBTAG00000009863 | BHLHE40 | 3.037433291 | 7.809431857 | 2.99E−117 | 3.62E−115 |
| ENSBTAG00000040133 | C22H3orf20 | 3.037377063 | 1.292614336 | 0.125705897 | 0.191775369 |
| ENSBTAG00000053735 | PEG10 | 3.03715185 | 5.056555208 | 2.21E−33 | 4.74E−32 |
| ENSBTAG00000048507 | A0A3Q1LZ64 | 3.035878408 | 1.292301206 | 0.125705897 | 0.191775369 |
| ENSBTAG00000002169 | NA | 3.03447706 | 1.292010043 | 0.125705897 | 0.191775369 |
| ENSBTAG00000004413 | RHOBTB3 | 3.032029726 | 7.886823503 | 7.02E−120 | 8.79E−118 |
| ENSBTAG00000055045 | HSPB7 | 3.03174007 | 1.291436567 | 0.125705897 | 0.191775369 |
| ENSBTAG00000008275 | GREBIL | 3.030833744 | 7.393053965 | 1.30E−101 | 1.23E−99 |
| ENSBTAG00000017753 | APP | 3.029881189 | 9.592414978 | 3.51E−173 | 8.05E−171 |
| ENSBTAG00000016197 | PHLDB1 | 3.027674504 | 5.638411577 | 2.21E−46 | 7.04E−45 |
| ENSBTAG00000015260 | CLC | 3.027592044 | 1.290552699 | 0.125705897 | 0.191775369 |
| ENSBTAG00000017490 | PLD1 | 3.025890548 | 3.121539435 | 7.08E−09 | 3.97E−08 |
| ENSBTAG00000003027 | EMX2 | 3.024617834 | 1.289958839 | 0.125705897 | 0.191775369 |
| ENSBTAG00000043951 | IMPA2 | 3.022853108 | 4.001134678 | 5.10E−17 | 5.29E−16 |
| ENSBTAG00000026523 | PLAGL1 | 3.019687868 | 2.820405757 | 1.66E−07 | 7.99E−07 |
| ENSBTAG00000019782 | TPI1 | 3.0196592 | 4.590601716 | 4.39E−25 | 6.93E−24 |
| ENSBTAG00000008462 | FAM43A | 3.018590976 | 1.288705778 | 0.125705897 | 0.191775369 |
| ENSBTAG00000008683 | LDHA | 3.015984659 | 7.177228609 | 4.46E−93 | 3.71E−91 |
| ENSBTAG00000002683 | PFKP | 3.015133144 | 5.622998314 | 1.06E−45 | 3.31E−44 |
| ENSBTAG00000009206 | FOXS1 | 3.01465863 | 1.287889663 | 0.125705897 | 0.191775369 |
| ENSBTAG00000011420 | CA9 | 3.010322114 | 4.537805107 | 1.87E−24 | 2.87E−23 |
| ENSBTAG00000005111 | BMP2 | 3.00919161 | 5.094328938 | 9.57E−34 | 2.11E−32 |
| ENSBTAG00000047647 | G3N267 | 3.009156584 | 1.286746666 | 0.125705897 | 0.191775369 |
| ENSBTAG00000002082 | MARCKS | 3.008713463 | 8.478266822 | 4.31E−139 | 6.87E−137 |
| ENSBTAG00000012211 | HOXA5 | 3.008051622 | 1.286530855 | 0.125705897 | 0.191775369 |
| ENSBTAG00000044046 | SGCD | 3.0050793 | 1.285905688 | 0.125705897 | 0.191775369 |
| ENSBTAG00000014857 | GBP6 | 3.004774746 | 1.285856624 | 0.125705897 | 0.191775369 |
| ENSBTAG00000016567 | HBQ1 | 3.00352479 | 1.285585855 | 0.125705897 | 0.191775369 |
| ENSBTAG00000051495 | A0A3Q1MCZ5 | 2.995562103 | 1.283958447 | 0.250705566 | 0.339685777 |
| ENSBTAG00000022498 | Rpl23a | 2.995520246 | 1.283952914 | 0.250705566 | 0.339685777 |
| ENSBTAG00000020928 | ADPRHL1 | 2.994946354 | 1.283825852 | 0.250705566 | 0.339685777 |
| ENSBTAG00000018403 | ARHGAP9 | 2.991672099 | 1.283181747 | 0.250705566 | 0.339685777 |
| ENSBTAG00000005021 | SEMA5A | 2.99164081 | 2.599561292 | 3.45E−06 | 1.43E−05 |
| ENSBTAG00000017242 | FADS6 | 2.989800269 | 2.364270909 | 2.36E−05 | 8.59E−05 |
| ENSBTAG00000046614 | B6VAE4 | 2.987875845 | 4.707766275 | 9.87E−27 | 1.66E−25 |
| ENSBTAG00000048609 | NA | 2.987354952 | 1.282285492 | 0.250705566 | 0.339685777 |
| ENSBTAG00000011525 | SLC5A8 | 2.98505705 | 1.281837034 | 0.250705566 | 0.339685777 |
| ENSBTAG00000004795 | RIPPLY2 | 2.98394861 | 1.281587397 | 0.250705566 | 0.339685777 |
| ENSBTAG00000019517 | ELN | 2.980554548 | 1.280902067 | 0.250705566 | 0.339685777 |
| ENSBTAG00000032733 | NA | 2.97988625 | 1.280763011 | 0.250705566 | 0.339685777 |
| ENSBTAG00000003856 | | 2.978185418 | 1.28042019 | 0.250705566 | 0.339685777 |
| ENSBTAG00000053742 | NA | 2.978050094 | 1.280402219 | 0.250705566 | 0.339685777 |
| ENSBTAG00000018946 | NIBAN3 | 2.976718752 | 1.280125297 | 0.250705566 | 0.339685777 |
| ENSBTAG00000054134 | ATP11C | 2.97629093 | 1.280026996 | 0.250705566 | 0.339685777 |
| ENSBTAG00000051412 | HBA | 2.972651672 | 1.279302664 | 0.250705566 | 0.339685777 |
| ENSBTAG00000049045 | NA | 2.971933105 | 1.279157245 | 0.250705566 | 0.339685777 |
| ENSBTAG00000035379 | NPM1 | 2.971264817 | 1.279018676 | 0.250705566 | 0.339685777 |
| ENSBTAG00000001822 | SNX10 | 2.970966302 | 7.704581074 | 6.50E−110 | 7.06E−108 |
| ENSBTAG00000005238 | NCKAP5 | 2.969980587 | 3.055599481 | 8.53E−09 | 4.74E−08 |
| ENSBTAG00000006716 | PTGS1 | 2.965792902 | 4.123211279 | 5.56E−18 | 6.07E−17 |
| ENSBTAG00000010610 | DDIT4L | 2.964122994 | 3.983963342 | 1.41E−16 | 1.43E−15 |
| ENSBTAG00000015235 | HMCN1 | 2.956031824 | 3.681811528 | 4.10E−13 | 3.36E−12 |
| ENSBTAG00000009070 | NPFFR2 | 2.955548546 | 1.275833732 | 0.250705566 | 0.339685777 |
| ENSBTAG00000052290 | NA | 2.953697062 | 1.275499009 | 0.250705566 | 0.339685777 |
| ENSBTAG00000021420 | EPHA7 | 2.953025904 | 1.275369222 | 0.250705566 | 0.339685777 |
| ENSBTAG00000026236 | AZGP1 | 2.952685461 | 1.275299439 | 0.250705566 | 0.339685777 |
| ENSBTAG00000001078 | SRL | 2.95225111 | 1.275200873 | 0.250705566 | 0.339685777 |
| ENSBTAG00000048508 | INHBA | 2.950516936 | 4.292790567 | 5.39E−20 | 6.64E−19 |
| ENSBTAG00000049011 | LLPH | 2.95032569 | 1.274823245 | 0.250705566 | 0.339685777 |
| ENSBTAG00000053998 | A0A3Q1MAW1 | 2.950033595 | 1.274768155 | 0.250705566 | 0.339685777 |
| ENSBTAG00000020535 | PYCARD | 2.949289996 | 1.274587113 | 0.250705566 | 0.339685777 |
| ENSBTAG00000004061 | PP2D1 | 2.947813813 | 1.274319019 | 0.250705566 | 0.339685777 |
| ENSBTAG00000015296 | PTPRB | 2.946859166 | 1.27413596 | 0.250705566 | 0.339685777 |
| ENSBTAG00000016515 | EFNA5 | 2.944117638 | 3.777531525 | 1.82E−14 | 1.64E−13 |
| ENSBTAG00000050914 | CYYR1 | 2.942784044 | 1.273339012 | 0.250705566 | 0.339685777 |
| ENSBTAG00000003668 | RADX | 2.942661038 | 5.284330293 | 1.93E−36 | 4.73E−35 |
| ENSBTAG00000005052 | GLP2R | 2.942215112 | 1.273212116 | 0.250705566 | 0.339685777 |
| ENSBTAG00000005077 | CXCL12 | 2.941554261 | 1.273073335 | 0.250705566 | 0.339685777 |
| ENSBTAG00000052317 | MTURN | 2.940215919 | 3.500768965 | 7.81E−12 | 5.62E−11 |
| ENSBTAG00000011742 | TNIK | 2.938032097 | 5.740265524 | 4.26E−47 | 1.38E−45 |
| ENSBTAG00000032369 | NMI | 2.93587996 | 3.349786141 | 1.33E−10 | 8.69E−10 |
| ENSBTAG00000013476 | CPA5 | 2.933799023 | 1.271548263 | 0.250705566 | 0.339685777 |
| ENSBTAG00000050991 | TTC21A | 2.932731872 | 1.27132333 | 0.250705566 | 0.339685777 |
| ENSBTAG00000036257 | ZNF75D | 2.932581943 | 1.271295385 | 0.250705566 | 0.339685777 |
| ENSBTAG00000046176 | SPEG | 2.932435794 | 2.94286873 | 7.82E−08 | 3.92E−07 |
| ENSBTAG00000018347 | IL33 | 2.932250133 | 4.571837533 | 1.15E−23 | 1.69E−22 |
| ENSBTAG00000055143 | WDR93 | 2.932093793 | 1.27119032 | 0.250705566 | 0.339685777 |
| ENSBTAG00000012164 | CP | 2.930743788 | 1.270940334 | 0.250705566 | 0.339685777 |
| ENSBTAG00000001156 | ST3GAL1 | 2.930243954 | 1.270853113 | 0.250705566 | 0.339685777 |
| ENSBTAG00000053165 | processed | 2.928823134 | 1.270548972 | 0.250705566 | 0.339685777 |
| | pseudogene | | | | |
| ENSBTAG00000010447 | LSP1 | 2.927025533 | 1.270207817 | 0.250705566 | 0.339685777 |
| ENSBTAG00000011441 | pseudogene | 2.926156839 | 1.270017613 | 0.250705566 | 0.339685777 |
| ENSBTAG00000006243 | GCNT7 | 2.924224868 | 1.269661426 | 0.250705566 | 0.339685777 |
| ENSBTAG00000031267 | SERPINB9 | 2.923765633 | 4.689101238 | 1.54E−25 | 2.48E−24 |
| ENSBTAG00000034674 | CLCNKA | 2.922949323 | 1.269396422 | 0.250705566 | 0.339685777 |
| ENSBTAG00000027080 | SLC8A3 | 2.920436835 | 1.268921301 | 0.250705566 | 0.339685777 |
| ENSBTAG00000017284 | RHBDL1 | 2.919822427 | 1.268781306 | 0.250705566 | 0.339685777 |
| ENSBTAG00000005439 | FAM102B | 2.918318842 | 3.033164947 | 1.49E−08 | 8.02E−08 |
| ENSBTAG00000013769 | NECTIN3 | 2.915858726 | 5.943846417 | 7.89E−52 | 2.95E−50 |
| ENSBTAG00000019332 | SLC9C1 | 2.913741663 | 1.267579803 | 0.250705566 | 0.339685777 |
| ENSBTAG00000017851 | RXRA | 2.91335875 | 6.432843475 | 3.69E−65 | 1.86E−63 |
| ENSBTAG00000032933 | TTC23L | 2.912662116 | 1.267390998 | 0.250705566 | 0.339685777 |
| ENSBTAG00000039046 | CD24 | 2.911530777 | 6.766950309 | 1.43E−75 | 8.69E−74 |
| ENSBTAG00000049784 | BLMH | 2.911180874 | 1.267108803 | 0.250705566 | 0.339685777 |
| ENSBTAG00000010229 | LAMA2 | 2.907752557 | 1.266417901 | 0.250705566 | 0.339685777 |
| ENSBTAG00000054180 | PAG1 | 2.901331862 | 1.265197585 | 0.250705566 | 0.339685777 |
| ENSBTAG00000031252 | CD82 | 2.900147632 | 2.708065453 | 2.75E−06 | 1.15E−05 |
| ENSBTAG00000052352 | NA | 2.89179823 | 1.263332513 | 0.250705566 | 0.339685777 |
| ENSBTAG00000003977 | SLC52A3 | 2.884252032 | 5.67990748 | 3.88E−44 | 1.17E−42 |
| ENSBTAG00000040304 | FIN5G0 | 2.88182116 | 1.261414034 | 0.250705566 | 0.339685777 |
| ENSBTAG00000054238 | A0A3Q1MA01 | 2.880908804 | 1.261239965 | 0.250705566 | 0.339685777 |
| ENSBTAG00000026919 | PXDC1 | 2.877794771 | 4.921474723 | 8.83E−29 | 1.61E−27 |
| ENSBTAG00000054098 | SERPINB6 | 2.87721558 | 1.260522257 | 0.250705566 | 0.339685777 |
| ENSBTAG00000044010 | EMB | 2.871486608 | 2.631424053 | 8.32E−06 | 3.26E−05 |
| ENSBTAG00000051540 | FNDC5 | 2.869463464 | 1.259040909 | 0.250705566 | 0.339685777 |
| ENSBTAG00000021576 | LMOD1 | 2.866955955 | 1.258558094 | 0.250705566 | 0.339685777 |
| ENSBTAG00000006205 | DNAJB7 | 2.866441886 | 1.25846838 | 0.250705566 | 0.339685777 |
| ENSBTAG00000044311 | SNORD2 | 2.865171066 | 1.258233768 | 0.250705566 | 0.339685777 |
| ENSBTAG00000021583 | TPGS2 | 2.860264177 | 4.693962423 | 9.04E−25 | 1.40E−23 |
| ENSBTAG00000053753 | Pabpn1 | 2.858907293 | 1.257038354 | 0.250705566 | 0.339685777 |
| ENSBTAG00000017943 | GALNT6 | 2.858039992 | 1.256879876 | 0.250705566 | 0.339685777 |
| ENSBTAG00000038411 | CBX1 | 2.849036586 | 1.255167798 | 0.250705566 | 0.339685777 |
| ENSBTAG00000005745 | HPSE | 2.841607576 | 2.677662792 | 4.79E−06 | 1.93E−05 |
| ENSBTAG00000007331 | PLOD2 | 2.840889351 | 7.188804483 | 6.80E−86 | 5.16E−84 |
| ENSBTAG00000010935 | EML4 | 2.840725238 | 7.54060112 | 5.85E−98 | 5.24E−96 |
| ENSBTAG00000047330 | FABP5 | 2.839826494 | 8.688604271 | 7.11E−134 | 9.93E−132 |
| ENSBTAG00000003039 | PSMB8 | 2.839441645 | 1.253373604 | 0.250705566 | 0.339685777 |
| ENSBTAG00000020061 | KCNJ12 | 2.838878709 | 1.253270697 | 0.250705566 | 0.339685777 |
| ENSBTAG00000046022 | PYY | 2.833676951 | 1.252272545 | 0.250705566 | 0.339685777 |
| ENSBTAG00000001258 | TMEM50B | 2.833596794 | 6.952005006 | 1.93E−78 | 1.23E−76 |
| ENSBTAG00000010663 | ADAM15 | 2.83234763 | 3.817962892 | 1.02E−13 | 8.75E−13 |
| ENSBTAG00000011337 | ANKRD33B | 2.824449903 | 1.250574669 | 0.250705566 | 0.339685777 |
| ENSBTAG00000020054 | TNFRSF21 | 2.818922174 | 8.112591547 | 5.27E−115 | 6.14E−113 |
| ENSBTAG00000021230 | SFTPB | 2.818477456 | 1.24945551 | 0.250705566 | 0.339685777 |
| ENSBTAG00000013214 | Pga5 | 2.818150689 | 1.249388299 | 0.250705566 | 0.339685777 |
| ENSBTAG00000038461 | Pzp | 2.815536492 | 1.24891156 | 0.250705566 | 0.339685777 |
| ENSBTAG00000018869 | IGSF6 | 2.81021417 | 1.247933842 | 0.250705566 | 0.339685777 |
| ENSBTAG00000025782 | TNFSF8 | 2.808492839 | 1.247612626 | 0.250705566 | 0.339685777 |
| ENSBTAG00000037702 | SP140L | 2.804772572 | 1.246933459 | 0.250705566 | 0.339685777 |
| ENSBTAG00000013401 | ARHGEF40 | 2.801772647 | 3.9723555 | 5.70E−15 | 5.28E−14 |
| ENSBTAG00000003751 | MACC1 | 2.801562014 | 5.113343375 | 1.68E−31 | 3.37E−30 |
| ENSBTAG00000017549 | KITLG | 2.794360678 | 3.388784045 | 1.87E−10 | 1.21E−09 |
| ENSBTAG00000053665 | NA | 2.791127536 | 1.244420554 | 0.250705566 | 0.339685777 |
| ENSBTAG00000038141 | ADGRG1 | 2.789396318 | 6.77189234 | 1.68E−71 | 9.58E−70 |
| ENSBTAG00000021693 | RPP25 | 2.788975079 | 1.244017826 | 0.250705566 | 0.339685777 |
| ENSBTAG00000039171 | ADAM7 | 2.784537146 | 1.243222768 | 0.250705566 | 0.339685777 |
| ENSBTAG00000038523 | ATP1A4 | 2.780694845 | 1.242534323 | 0.250705566 | 0.339685777 |
| ENSBTAG00000013281 | SPATA21 | 2.778421672 | 1.242118722 | 0.250705566 | 0.339685777 |
| ENSBTAG00000043077 | | 2.77820571 | 1.242075954 | 0.250705566 | 0.339685777 |
| ENSBTAG00000003802 | TEKT3 | 2.777977849 | 1.242022818 | 0.250705566 | 0.339685777 |
| ENSBTAG00000046922 | TWIST1 | 2.77672731 | 2.428621376 | 5.98E−05 | 0.000203386 |
| ENSBTAG00000014574 | CFAP43 | 2.776239305 | 1.241722379 | 0.250705566 | 0.339685777 |
| ENSBTAG00000022799 | NOTCH1 | 2.775500456 | 3.604684459 | 9.01E−12 | 6.46E−11 |
| ENSBTAG00000005434 | PLVAP | 2.775370173 | 1.241567957 | 0.250705566 | 0.339685777 |
| ENSBTAG00000016127 | SPINT3 | 2.774274113 | 1.241352017 | 0.250705566 | 0.339685777 |
| ENSBTAG00000046820 | G3MX45 | 2.77412256 | 1.241334467 | 0.250705566 | 0.339685777 |
| ENSBTAG00000016411 | RNF122 | 2.772753261 | 4.878073117 | 1.05E−26 | 1.75E−25 |
| ENSBTAG00000053945 | A0A3Q1LNX3 | 2.771557757 | 1.240867859 | 0.250705566 | 0.339685777 |
| ENSBTAG00000010408 | IKBKE | 2.770411057 | 3.592549803 | 9.01E−12 | 6.46E−11 |
| ENSBTAG00000049560 | ICAM1 | 2.7697301 | 1.240537897 | 0.250705566 | 0.339685777 |
| ENSBTAG00000021543 | MDFIC | 2.766582799 | 6.710965368 | 7.52E−69 | 4.08E−67 |
| ENSBTAG00000015163 | TM4SF1 | 2.760451285 | 6.03944568 | 1.56E−50 | 5.62E−49 |
| ENSBTAG00000013070 | EAF2 | 2.757790236 | 2.878934639 | 4.05E−07 | 1.87E−06 |
| ENSBTAG00000014932 | ARSE | 2.757122068 | 5.603352212 | 5.31E−40 | 1.41E−38 |
| ENSBTAG00000044023 | CDK6 | 2.752430623 | 3.664349712 | 4.32E−12 | 3.22E−11 |
| ENSBTAG00000021416 | PRXL2A | 2.752405967 | 8.671490719 | 6.86E−127 | 8.98E−125 |
| ENSBTAG00000048485 | TUBB2A | 2.746030262 | 3.890440333 | 3.07E−14 | 2.73E−13 |
| ENSBTAG00000051798 | lncRNA | 2.745757996 | 1.236255434 | 0.250705566 | 0.339685777 |
| ENSBTAG00000016519 | MBOAT1 | 2.74518145 | 5.976226698 | 1.70E−48 | 5.77E−47 |
| ENSBTAG00000047147 | CRYBG3 | 2.743235564 | 4.822212512 | 1.61E−25 | 2.59E−24 |
| ENSBTAG00000008401 | PFKFB3 | 2.742914531 | 6.661191697 | 1.47E−66 | 7.78E−65 |
| ENSBTAG00000053896 | TOP1MT | 2.742173178 | 1.23561184 | 0.250705566 | 0.339685777 |
| ENSBTAG00000038549 | CYP4F2 | 2.741456663 | 1.235504797 | 0.250705566 | 0.339685777 |
| ENSBTAG00000054737 | NA | 2.732157944 | 1.233847051 | 0.250705566 | 0.339685777 |
| ENSBTAG00000018270 | NFATC2 | 2.729117411 | 1.233326465 | 0.250705566 | 0.339685777 |
| ENSBTAG00000013479 | SLC9A3R2 | 2.727737482 | 2.956728068 | 5.18E−07 | 2.36E−06 |
| ENSBTAG00000004838 | IRX5 | 2.727167519 | 1.232960507 | 0.250705566 | 0.339685777 |
| ENSBTAG00000023600 | APOD | 2.726325354 | 1.232811712 | 0.250705566 | 0.339685777 |
| ENSBTAG00000051083 | WNT4 | 2.726179704 | 4.56809878 | 2.10E−21 | 2.79E−20 |
| ENSBTAG00000025314 | PRP8 | 2.724277874 | 1.232475334 | 0.250705566 | 0.339685777 |
| ENSBTAG00000007235 | NTNG2 | 2.723367053 | 1.232287361 | 0.250705566 | 0.339685777 |
| ENSBTAG00000030575 | BHLHE41 | 2.721856788 | 2.56041613 | 4.29E−05 | 0.000149263 |
| ENSBTAG00000010361 | DLL4 | 2.71999693 | 1.231712483 | 0.250705566 | 0.339685777 |
| ENSBTAG00000016240 | RNF157 | 2.719023987 | 5.040734165 | 6.43E−29 | 1.18E−27 |
| ENSBTAG00000039462 | PCLAF | 2.717291065 | 4.729198203 | 9.78E−24 | 1.46E−22 |
| ENSBTAG00000040564 | COX7C | 2.715213687 | 1.230895846 | 0.250705566 | 0.339685777 |
| ENSBTAG00000012411 | HAPLN1 | 2.71230045 | 1.230373313 | 0.250705566 | 0.339685777 |
| ENSBTAG00000008471 | MX2 | 2.709917387 | 3.3813712 | 1.17E−09 | 7.03E−09 |
| ENSBTAG00000019234 | BMP6 | 2.708370472 | 1.229691121 | 0.250705566 | 0.339685777 |
| ENSBTAG00000047191 | KCNA6 | 2.707304835 | 3.406601005 | 6.99E−10 | 4.29E−09 |
| ENSBTAG00000003585 | CD47 | 2.707240264 | 6.758140404 | 4.94E−68 | 2.66E−66 |
| ENSBTAG00000053087 | NA | 2.707069486 | 1.229435647 | 0.250705566 | 0.339685777 |
| ENSBTAG00000011034 | ANGPT2 | 2.706276761 | 1.229311085 | 0.250705566 | 0.339685777 |
| ENSBTAG00000051401 | NA | 2.705771816 | 1.229227929 | 0.250705566 | 0.339685777 |
| ENSBTAG00000009354 | EVI2A | 2.703188709 | 1.228760335 | 0.250705566 | 0.339685777 |
| ENSBTAG00000046768 | IGFBP1 | 2.700536695 | 1.228332389 | 0.250705566 | 0.339685777 |
| ENSBTAG00000012456 | HEPACAM2 | 2.697637645 | 1.227819003 | 0.250705566 | 0.339685777 |
| ENSBTAG00000038415 | SLC6A12 | 2.694732595 | 1.227324725 | 0.250705566 | 0.339685777 |
| ENSBTAG00000012642 | TRADD | 2.686617306 | 2.715976344 | 1.03E−05 | 3.93E−05 |
| ENSBTAG00000021245 | SPRY1 | 2.68658338 | 3.065988158 | 2.22E−07 | 1.05E−06 |
| ENSBTAG00000052330 | NA | 2.677922816 | 1.22442319 | 0.250705566 | 0.339685777 |
| ENSBTAG00000005386 | SLC2A2 | 2.676398327 | 1.224177517 | 0.250705566 | 0.339685777 |
| ENSBTAG00000005359 | TGFB2 | 2.675654573 | 3.642946474 | 1.18E−11 | 8.43E−11 |
| ENSBTAG00000021960 | GPX8 | 2.671818828 | 3.833803698 | 3.66E−13 | 3.01E−12 |
| ENSBTAG00000015716 | ERO1A | 2.668445521 | 8.402853336 | 3.40E−114 | 3.89E−112 |
| ENSBTAG00000031532 | DACT2 | 2.666434293 | 2.444915251 | 5.98E−05 | 0.000203386 |
| ENSBTAG00000002997 | ADAMTSL1 | 2.664745867 | 3.256985521 | 7.26E−09 | 4.07E−08 |
| ENSBTAG00000008092 | RAB37 | 2.657088454 | 3.458353129 | 8.74E−10 | 5.32E−09 |
| ENSBTAG00000017071 | C1QTNF3 | 2.656830432 | 2.673206444 | 1.74E−05 | 6.46E−05 |
| ENSBTAG00000040116 | H1-10 | 2.65411437 | 3.989686284 | 1.99E−14 | 1.79E−13 |
| ENSBTAG00000044012 | TMEM196 | 2.653166713 | 1.220222721 | 0.250705566 | 0.339685777 |
| ENSBTAG00000015609 | PREX1 | 2.650561723 | 4.355363788 | 2.20E−18 | 2.47E−17 |
| ENSBTAG00000011490 | TLCD2 | 2.648696404 | 1.219456956 | 0.250705566 | 0.339685777 |
| ENSBTAG00000017662 | EEF2K | 2.647261781 | 3.913928865 | 6.63E−14 | 5.76E−13 |
| ENSBTAG00000010178 | PTPRD | 2.646166611 | 2.226736757 | 0.000957727 | 0.002654881 |
| ENSBTAG00000021378 | S100A13 | 2.645082753 | 2.966253402 | 5.18E−07 | 2.36E−06 |
| ENSBTAG00000012335 | UBA7 | 2.644802759 | 4.253951923 | 7.01E−17 | 7.20E−16 |
| ENSBTAG00000021210 | HES2 | 2.641639114 | 3.592546347 | 5.34E−11 | 3.59E−10 |
| ENSBTAG00000009014 | UPK1B | 2.640558681 | 1.218102942 | 0.250705566 | 0.339685777 |
| ENSBTAG00000003806 | ECM1 | 2.638232706 | 5.620376285 | 3.05E−38 | 7.84E−37 |
| ENSBTAG00000002783 | PCYOX1 | 2.633910137 | 3.410397017 | 6.99E−10 | 4.29E−09 |
| ENSBTAG00000023186 | AC092647.5 | 2.633516936 | 1.216901556 | 0.250705566 | 0.339685777 |
| ENSBTAG00000015988 | MYH11 | 2.630747136 | 2.812564215 | 2.06E−06 | 8.80E−06 |
| ENSBTAG00000004558 | C10H15orf48 | 2.630742577 | 1.216468216 | 0.250705566 | 0.339685777 |
| ENSBTAG00000013469 | SRCIN1 | 2.630609685 | 1.216447961 | 0.250705566 | 0.339685777 |
| ENSBTAG00000002278 | FBN1 | 2.63045105 | 5.073143239 | 5.92E−28 | 1.05E−26 |
| ENSBTAG00000052616 | Gm27855 | 2.628698023 | 1.216123076 | 0.250705566 | 0.339685777 |
| ENSBTAG00000005793 | PEA15 | 2.627535537 | 5.15550307 | 1.75E−29 | 3.31E−28 |
| ENSBTAG00000002113 | ZNF41 | 2.626899498 | 1.21581374 | 0.250705566 | 0.339685777 |
| ENSBTAG00000044712 | bta-mir-2383 | 2.625197569 | 1.215541207 | 0.250705566 | 0.339685777 |
| ENSBTAG00000012280 | PGLYRP2 | 2.624748304 | 3.072303282 | 1.32E−07 | 6.46E−07 |
| ENSBTAG00000018513 | FHL1 | 2.621555941 | 2.028537318 | 0.002529621 | 0.006372717 |
| ENSBTAG00000017508 | CYSLTR2 | 2.621089883 | 1.214859359 | 0.250705566 | 0.339685777 |
| ENSBTAG00000003440 | FRRS1 | 2.620234524 | 5.255987211 | 3.41E−31 | 6.75E−30 |
| ENSBTAG00000021636 | GPR155 | 2.619782242 | 4.84037615 | 3.56E−24 | 5.41E−23 |
| ENSBTAG00000014920 | SEMA3E | 2.616489738 | 1.214108083 | 0.250705566 | 0.339685777 |
| ENSBTAG00000054533 | GNG12 | 2.612453216 | 7.087280518 | 1.51E−73 | 8.81E−72 |
| ENSBTAG00000051704 | TNFRSF10D | 2.608345799 | 5.550150349 | 2.07E−36 | 5.06E−35 |
| ENSBTAG00000001299 | CYP7B1 | 2.604676348 | 1.212158016 | 0.250705566 | 0.339685777 |
| ENSBTAG00000017166 | C9H6orf58 | 2.603871718 | 6.45793818 | 1.79E−56 | 7.49E−55 |
| ENSBTAG00000019587 | PI15 | 2.602842441 | 1.211856112 | 0.250705566 | 0.339685777 |
| ENSBTAG00000017514 | CCND1 | 2.598250339 | 3.242832037 | 1.22E−08 | 6.64E−08 |
| ENSBTAG00000021144 | PSTPIP1 | 2.598162767 | 1.21109154 | 0.250705566 | 0.339685777 |
| ENSBTAG00000047088 | Slc23a4 | 2.590509095 | 1.209835595 | 0.250705566 | 0.339685777 |
| ENSBTAG00000007554 | IFI6 | 2.588201826 | 3.179291872 | 5.69E−08 | 2.90E−07 |
| ENSBTAG00000019958 | NXNL2 | 2.58636855 | 2.550606755 | 4.29E−05 | 0.000149263 |
| ENSBTAG00000001602 | IL4R | 2.584800862 | 4.398270602 | 5.77E−18 | 6.28E−17 |
| ENSBTAG00000018704 | BTG3 | 2.581080145 | 4.449904071 | 1.20E−18 | 1.38E−17 |
| ENSBTAG00000017738 | EMID1 | 2.580741489 | 1.208251468 | 0.500470377 | 0.600982399 |
| ENSBTAG00000010529 | FZD6 | 2.580371969 | 6.908731675 | 1.12E−67 | 5.97E−66 |
| ENSBTAG00000010050 | COL16A1 | 2.576511502 | 1.843883314 | 0.023254718 | 0.045985836 |
| ENSBTAG00000001903 | C11H2orf50 | 2.576413861 | 2.807601036 | 2.06E−06 | 8.80E−06 |
| ENSBTAG00000021272 | ABCG1 | 2.57561496 | 1.207409864 | 0.500470377 | 0.600982399 |
| ENSBTAG00000004414 | SLC30A10 | 2.575591807 | 1.207437369 | 0.500470377 | 0.600982399 |
| ENSBTAG00000000580 | LY6G6F | 2.572664623 | 1.206958345 | 0.500470377 | 0.600982399 |
| ENSBTAG00000008842 | JPH1 | 2.564956736 | 2.574759562 | 4.29E−05 | 0.000149263 |
| ENSBTAG00000048655 | NT5E | 2.562567925 | 5.355831518 | 9.49E−32 | 1.93E−30 |
| ENSBTAG00000021026 | PJA1 | 2.561711945 | 5.911847791 | 8.45E−43 | 2.45E−41 |
| ENSBTAG00000009019 | SH3PXD2B | 2.560780594 | 3.439472284 | 1.44E−09 | 8.62E−09 |
| ENSBTAG00000002767 | GNB4 | 2.560557971 | 7.27545297 | 8.49E−77 | 5.26E−75 |
| ENSBTAG00000001390 | B4GALT4 | 2.559714991 | 5.060930737 | 1.85E−26 | 3.07E−25 |
| ENSBTAG00000013391 | ANKH | 2.5594524 | 8.741477204 | 3.04E−115 | 3.57E−113 |
| ENSBTAG00000002052 | PLOD1 | 2.559217635 | 4.036929144 | 5.05E−14 | 4.43E−13 |
| ENSBTAG00000017183 | PDLIM3 | 2.556615337 | 1.204385688 | 0.500470377 | 0.600982399 |
| ENSBTAG00000043963 | FHDC1 | 2.556544915 | 2.455035471 | 0.000214938 | 0.000667478 |
| ENSBTAG00000048125 | ENTPD2 | 2.554483321 | 4.140667405 | 3.73E−15 | 3.49E−14 |
| ENSBTAG00000015905 | ARHGAP32 | 2.553463897 | 6.430852853 | 2.04E−54 | 8.09E−53 |
| ENSBTAG00000019322 | HABP2 | 2.549249936 | 1.203221815 | 0.500470377 | 0.600982399 |
| ENSBTAG00000050685 | A0A3Q1MEX0 | 2.548500792 | 1.203087643 | 0.500470377 | 0.600982399 |
| ENSBTAG00000021697 | PDGFB | 2.547555828 | 4.830163011 | 1.29E−23 | 1.90E−22 |
| ENSBTAG00000016805 | SGMS2 | 2.545753326 | 5.057508274 | 9.42E−27 | 1.58E−25 |
| ENSBTAG00000015074 | PTGDS | 2.543489554 | 3.193632987 | 5.69E−08 | 2.90E−07 |
| ENSBTAG00000019230 | PTGER3 | 2.542315892 | 1.202111019 | 0.500470377 | 0.600982399 |
| ENSBTAG00000050127 | Z82206.1 | 2.541937414 | 1.2020453 | 0.500470377 | 0.600982399 |
| ENSBTAG00000011613 | PLS3 | 2.540833525 | 6.11774263 | 3.12E−46 | 9.87E−45 |
| ENSBTAG00000007909 | NOTCH2 | 2.5386713 | 5.465861998 | 2.02E−33 | 4.34E−32 |
| ENSBTAG00000010069 | EGR1 | 2.53755832 | 5.593206576 | 1.01E−35 | 2.42E−34 |
| ENSBTAG00000018313 | MBNL2 | 2.534279127 | 4.856630617 | 7.15E−24 | 1.07E−22 |
| ENSBTAG00000008115 | TNFAIP1 | 2.531413339 | 4.483587369 | 1.51E−18 | 1.71E−17 |
| ENSBTAG00000019885 | ZNF280C | 2.531038238 | 2.585290199 | 2.49E−05 | 9.03E−05 |
| ENSBTAG00000012771 | CSF1R | 2.526810542 | 1.199682588 | 0.500470377 | 0.600982399 |
| ENSBTAG00000007592 | RARG | 2.522052899 | 3.751853682 | 6.98E−12 | 5.08E−11 |
| ENSBTAG00000011624 | FGF21 | 2.522031807 | 1.198933452 | 0.500470377 | 0.600982399 |
| ENSBTAG00000013537 | FER1L6 | 2.521941543 | 2.777117466 | 6.03E−06 | 2.40E−05 |
| ENSBTAG00000001368 | LGALS3BP | 2.521398917 | 6.690948753 | 7.47E−60 | 3.34E−58 |
| ENSBTAG00000020513 | HNRNPF | 2.519655376 | 1.198530764 | 0.500470377 | 0.600982399 |
| ENSBTAG00000051565 | ITGB3BP | 2.5145065 | 1.19773095 | 0.500470377 | 0.600982399 |
| ENSBTAG00000009975 | PBX4 | 2.514465799 | 2.986257999 | 1.04E−06 | 4.58E−06 |
| ENSBTAG00000039337 | CYP4F2 | 2.514099795 | 1.197680739 | 0.500470377 | 0.600982399 |
| ENSBTAG00000005090 | CEMIP2 | 2.509637859 | 6.739709374 | 1.16E−60 | 5.33E−59 |
| ENSBTAG00000002028 | DIPK1A | 2.504877207 | 5.789420652 | 1.10E−38 | 2.83E−37 |
| ENSBTAG00000017363 | SAT1 | 2.503044108 | 8.211639655 | 4.90E−98 | 4.41E−96 |
| ENSBTAG00000009418 | FAM131B | 2.50113567 | 4.482604487 | 1.51E−18 | 1.71E−17 |
| ENSBTAG00000020895 | LOXL4 | 2.50012564 | 5.391325777 | 1.13E−31 | 2.28E−30 |
| ENSBTAG00000054692 | A0A3Q1MGV3 | 2.499136675 | 1.195345444 | 0.500470377 | 0.600982399 |
| ENSBTAG00000016165 | KRT7 | 2.497742605 | 6.736491051 | 1.96E−60 | 8.96E−59 |
| ENSBTAG00000015828 | FKBP11 | 2.496593644 | 4.922800564 | 6.49E−24 | 9.76E−23 |
| ENSBTAG00000020035 | RCAN1 | 2.494069298 | 5.227135659 | 3.04E−28 | 5.49E−27 |
| ENSBTAG00000012972 | CYP2U1 | 2.48844268 | 3.289197503 | 2.46E−08 | 1.29E−07 |
| ENSBTAG00000008718 | GALNT17 | 2.487457501 | 5.641590532 | 5.50E−36 | 1.33E−34 |
| ENSBTAG00000002939 | FURIN | 2.486320415 | 10.21191432 | 4.64E−140 | 7.57E−138 |
| ENSBTAG00000051913 | UST | 2.486038809 | 4.789401115 | 7.69E−22 | 1.04E−20 |
| ENSBTAG00000011833 | GRIA3 | 2.47732672 | 2.184490364 | 0.001644903 | 0.004348556 |
| ENSBTAG00000017228 | TP53I11 | 2.477058596 | 4.531684203 | 4.96E−19 | 5.81E−18 |
| ENSBTAG00000050197 | C10orf99 | 2.474633303 | 3.631551621 | 3.37E−10 | 2.13E−09 |
| ENSBTAG00000015419 | ARHGEF37 | 2.470296059 | 4.360048979 | 5.57E−17 | 5.75E−16 |
| ENSBTAG00000015412 | TGM7 | 2.469788137 | 1.190877644 | 0.500470377 | 0.600982399 |
| ENSBTAG00000037452 | PNP | 2.469004307 | 2.230659437 | 0.000957727 | 0.002654881 |
| ENSBTAG00000051578 | lncRNA | 2.468287877 | 1.190660907 | 0.500470377 | 0.600982399 |
| ENSBTAG00000018909 | CREB5 | 2.466830198 | 2.998209901 | 1.04E−06 | 4.58E−06 |
| ENSBTAG00000051245 | SOX4 | 2.46379277 | 2.850478236 | 4.22E−06 | 1.72E−05 |
| ENSBTAG00000013909 | BOC | 2.461354816 | 2.41133092 | 0.000364708 | 0.001085684 |
| ENSBTAG00000032515 | PLA2R1 | 2.460824871 | 7.394353886 | 1.70E−75 | 1.03E−73 |
| ENSBTAG00000002596 | VSTM1 | 2.460806576 | 1.189525933 | 0.500470377 | 0.600982399 |
| ENSBTAG00000008817 | LAMA4 | 2.458990973 | 2.548317484 | 7.37E−05 | 0.000245528 |
| ENSBTAG00000053469 | IFN-tau-c1 | 2.458016069 | 3.577517981 | 4.68E−10 | 2.92E−09 |
| ENSBTAG00000032893 | SPATS2 | 2.457232882 | 1.18897762 | 0.500470377 | 0.600982399 |
| ENSBTAG00000009148 | GPA33 | 2.456128168 | 2.267527683 | 0.000957727 | 0.002654881 |
| ENSBTAG00000012139 | SIX1 | 2.454309855 | 1.188547701 | 0.500470377 | 0.600982399 |
| ENSBTAG00000018364 | TMEM132A | 2.45402804 | 5.366131371 | 3.12E−30 | 6.01E−29 |
| ENSBTAG00000003741 | NEURL1 | 2.453769036 | 1.188458647 | 0.500470377 | 0.600982399 |
| ENSBTAG00000016057 | CSRP1 | 2.452051973 | 6.125926661 | 9.60E−45 | 2.95E−43 |
| ENSBTAG00000018799 | GPSM1 | 2.450036043 | 4.0873229 | 3.85E−14 | 3.40E−13 |
| ENSBTAG00000011104 | RTN4 | 2.446153382 | 8.763517589 | 2.73E−107 | 2.88E−105 |
| ENSBTAG00000024772 | TRIP6 | 2.445649165 | 4.84804479 | 3.36E−22 | 4.64E−21 |
| ENSBTAG00000019734 | CD276 | 2.445625142 | 3.130501051 | 2.62E−07 | 1.23E−06 |
| ENSBTAG00000046503 | NCOA7 | 2.440363864 | 3.372784247 | 1.06E−08 | 5.82E−08 |
| ENSBTAG00000054191 | PAG15 | 2.439135092 | 1.18628659 | 0.500470377 | 0.600982399 |
| ENSBTAG00000021617 | ZC3HAV1 | 2.438915762 | 5.704993532 | 7.15E−36 | 1.72E−34 |
| ENSBTAG00000021464 | SPRYD3 | 2.438168148 | 5.365452499 | 1.24E−29 | 2.36E−28 |
| ENSBTAG00000003369 | SYT12 | 2.436876573 | 1.185926953 | 0.500470377 | 0.600982399 |
| ENSBTAG00000001864 | NR4A3 | 2.43260629 | 1.185308938 | 0.500470377 | 0.600982399 |
| ENSBTAG00000004272 | ISG12(B) | 2.429314117 | 2.208771046 | 0.001644903 | 0.004348556 |
| ENSBTAG00000001405 | GLCE | 2.428615698 | 6.179116162 | 4.23E−45 | 1.30E−43 |
| ENSBTAG00000010850 | SERTAD4 | 2.427840978 | 6.102103108 | 1.67E−43 | 4.93E−42 |
| ENSBTAG00000013215 | MREG | 2.426339706 | 2.857378085 | 4.22E−06 | 1.72E−05 |
| ENSBTAG00000019929 | ITGAV | 2.41957659 | 7.693314273 | 6.20E−81 | 4.17E−79 |
| ENSBTAG00000009292 | TMEM100 | 2.419102849 | 1.1833155 | 0.500470377 | 0.600982399 |
| ENSBTAG00000019652 | STK26 | 2.417474842 | 5.777880031 | 1.54E−36 | 3.80E−35 |
| ENSBTAG00000015874 | CASP3 | 2.415474317 | 7.078465872 | 2.02E−65 | 1.03E−63 |
| ENSBTAG00000024929 | PPP1R27 | 2.414400707 | 1.182591087 | 0.500470377 | 0.600982399 |
| ENSBTAG00000053007 | ITPKB | 2.409510438 | 6.988340024 | 4.06E−63 | 1.99E−61 |
| ENSBTAG00000054623 | HOXB9 | 2.407502452 | 4.051717118 | 1.55E−13 | 1.31E−12 |
| ENSBTAG00000034918 | IFIT2 | 2.406661091 | 1.181492153 | 0.500470377 | 0.600982399 |
| ENSBTAG00000014064 | FGFR2 | 2.405967508 | 6.298098986 | 4.31E−47 | 1.39E−45 |
| ENSBTAG00000018837 | SLC39A11 | 2.405627698 | 6.742146047 | 4.84E−57 | 2.05E−55 |
| ENSBTAG00000006282 | POU2AF1 | 2.402226243 | 1.180844749 | 0.500470377 | 0.600982399 |
| ENSBTAG00000016762 | SLC25A12 | 2.401572699 | 5.564719658 | 1.11E−32 | 2.33E−31 |
| ENSBTAG00000009156 | CAMK2N1 | 2.389250271 | 2.811123755 | 1.18E−05 | 4.51E−05 |
| ENSBTAG00000017263 | MXI1 | 2.389094687 | 4.563432561 | 5.10E−18 | 5.58E−17 |
| ENSBTAG00000013004 | ITIH5 | 2.388788679 | 1.178896601 | 0.500470377 | 0.600982399 |
| ENSBTAG00000019554 | FBP2 | 2.385888693 | 2.544276229 | 0.000233821 | 0.000721231 |
| ENSBTAG00000017122 | HSPG2 | 2.384699854 | 8.185735736 | 4.25E−91 | 3.52E−89 |
| ENSBTAG00000054516 | CYP17A1 | 2.38312458 | 2.800167967 | 1.18E−05 | 4.51E−05 |
| ENSBTAG00000017063 | EPB41L4B | 2.381663121 | 4.958417291 | 4.28E−23 | 6.16E−22 |
| ENSBTAG00000003601 | ZMAT1 | 2.381029765 | 1.17777533 | 0.500470377 | 0.600982399 |
| ENSBTAG00000009020 | CRIM1 | 2.372671325 | 6.822838745 | 1.26E−57 | 5.39E−56 |
| ENSBTAG00000053098 | A0A3Q1LV70 | 2.371775883 | 2.189000785 | 0.001644903 | 0.004348556 |
| ENSBTAG00000010562 | CD34 | 2.369773656 | 1.176153016 | 0.500470377 | 0.600982399 |
| ENSBTAG00000021118 | CYP26A1 | 2.364181547 | 2.102275272 | 0.004766113 | 0.011242294 |
| ENSBTAG00000033008 | MYOZ1 | 2.362155604 | 4.847517274 | 3.93E−21 | 5.13E−20 |
| ENSBTAG00000046339 | VASN | 2.360100974 | 2.623922851 | 8.37E−05 | 0.000277266 |
| ENSBTAG00000053330 | NA | 2.360056179 | 1.174739418 | 0.500470377 | 0.600982399 |
| ENSBTAG00000004851 | NCKAP5L | 2.357616962 | 3.301803239 | 7.62E−08 | 3.82E−07 |
| ENSBTAG00000012409 | POSTN | 2.356668151 | 5.183175928 | 4.26E−25 | 6.72E−24 |
| ENSBTAG00000008353 | CDKN1A | 2.352026989 | 5.418624303 | 3.00E−29 | 5.60E−28 |
| ENSBTAG00000014907 | IL11RA | 2.350091723 | 5.918224234 | 6.04E−38 | 1.54E−36 |
| ENSBTAG00000045751 | ABCC4 | 2.345300689 | 1.172661894 | 0.500470377 | 0.600982399 |
| ENSBTAG00000019733 | ADGRL4 | 2.342121279 | 1.17224385 | 0.500470377 | 0.600982399 |
| ENSBTAG00000019595 | EGFLAM | 2.339126893 | 3.804744795 | 4.17E−11 | 2.83E−10 |
| ENSBTAG00000013222 | CD109 | 2.33780166 | 5.240290408 | 2.40E−26 | 3.96E−25 |
| ENSBTAG00000001476 | A0A3Q1MD45 | 2.335808677 | 1.171358049 | 0.500470377 | 0.600982399 |
| ENSBTAG00000051910 | FGF10 | 2.333810328 | 1.17107554 | 0.500470377 | 0.600982399 |
| ENSBTAG00000026501 | MGC127055 | 2.333797804 | 1.171083633 | 0.500470377 | 0.600982399 |
| ENSBTAG00000053984 | A0A3S5ZPE7 | 2.332849647 | 1.170942588 | 0.500470377 | 0.600982399 |
| ENSBTAG00000047036 | GPR39 | 2.332751622 | 1.170933317 | 0.500470377 | 0.600982399 |
| ENSBTAG00000014921 | IL6 | 2.332721452 | 3.991020157 | 2.45E−12 | 1.86E−11 |
| ENSBTAG00000023832 | ADAM8 | 2.331464386 | 4.740451425 | 2.60E−19 | 3.11E−18 |
| ENSBTAG00000004954 | TOX | 2.331216329 | 1.170711768 | 0.500470377 | 0.600982399 |
| ENSBTAG00000013491 | EML1 | 2.324636433 | 4.832683973 | 1.34E−20 | 1.71E−19 |
| ENSBTAG00000006434 | SYNPO2 | 2.323811536 | 3.835385464 | 4.76E−11 | 3.22E−10 |
| ENSBTAG00000004772 | THEM4 | 2.323234728 | 5.86219401 | 4.91E−36 | 1.19E−34 |
| ENSBTAG00000044208 | DUSP4 | 2.319739877 | 3.463242865 | 1.37E−08 | 7.41E−08 |
| ENSBTAG00000039335 | ARRDC2 | 2.31951246 | 4.665455293 | 1.53E−18 | 1.73E−17 |
| ENSBTAG00000034435 | NKD2 | 2.319464407 | 2.538662857 | 0.000233821 | 0.000721231 |
| ENSBTAG00000001207 | SERPINB8 | 2.31455443 | 5.840155503 | 1.50E−35 | 3.53E−34 |
| ENSBTAG00000002080 | CCN3 | 2.314064731 | 2.868555766 | 1.30E−05 | 4.92E−05 |
| ENSBTAG00000009051 | MMP19 | 2.312973966 | 1.988287005 | 0.007782663 | 0.017599935 |
| ENSBTAG00000013926 | FCGRT | 2.311980938 | 4.096954809 | 5.20E−13 | 4.21E−12 |
| ENSBTAG00000054774 | processed | 2.308770132 | 8.104240911 | 2.99E−84 | 2.18E−82 |
| | pseudogene | | | | |
| ENSBTAG00000020661 | ABCA1 | 2.30874002 | 7.520532089 | 1.97E−71 | 1.11E−69 |
| ENSBTAG00000014401 | SORBS3 | 2.307148905 | 4.640113618 | 5.35E−18 | 5.86E−17 |
| ENSBTAG00000017595 | AC093155.3 | 2.306402735 | 4.571985357 | 2.15E−17 | 2.28E−16 |
| ENSBTAG00000019065 | EEPD1 | 2.305720814 | 2.378086535 | 0.000615949 | 0.001760693 |
| ENSBTAG00000006894 | NOS2 | 2.30361485 | 6.222970107 | 1.90E−42 | 5.47E−41 |
| ENSBTAG00000015978 | ANXA1 | 2.300749775 | 10.13983149 | 3.63E−121 | 4.59E−119 |
| ENSBTAG00000022005 | TLDC2 | 2.295643821 | 1.165801858 | 0.500470377 | 0.600982399 |
| ENSBTAG00000030174 | ACOX1 | 2.293888763 | 7.976036515 | 9.64E−81 | 6.46E−79 |
| ENSBTAG00000016486 | SORBS2 | 2.290308433 | 2.395875655 | 0.000615949 | 0.001760693 |
| ENSBTAG00000040034 | Stra6l | 2.289837814 | 1.164980466 | 0.500470377 | 0.600982399 |
| ENSBTAG00000010423 | LIFR | 2.288648012 | 2.843101249 | 2.14E−05 | 7.83E−05 |
| ENSBTAG00000003837 | RSPO1 | 2.287651238 | 1.164717245 | 0.500470377 | 0.600982399 |
| ENSBTAG00000019251 | EPB41L3 | 2.285738178 | 6.804914964 | 7.11E−54 | 2.79E−52 |
| ENSBTAG00000002554 | TCAF1 | 2.285345109 | 7.201519302 | 4.74E−63 | 2.31E−61 |
| ENSBTAG00000008923 | GPR83 | 2.284611824 | 3.688517736 | 3.86E−10 | 2.43E−09 |
| ENSBTAG00000008289 | ZBTB4 | 2.284574799 | 4.947281882 | 1.53E−21 | 2.04E−20 |
| ENSBTAG00000014191 | QSOX1 | 2.284180746 | 6.895149239 | 4.04E−56 | 1.68E−54 |
| ENSBTAG00000001839 | OCIAD2 | 2.282853026 | 3.234244241 | 5.29E−07 | 2.41E−06 |
| ENSBTAG00000014400 | E2F2 | 2.281435132 | 2.421293881 | 0.000364708 | 0.001085684 |
| ENSBTAG00000006801 | TMEM106A | 2.278868351 | 2.790509645 | 1.98E−05 | 7.26E−05 |
| ENSBTAG00000005353 | DES | 2.27773942 | 3.509891382 | 5.26E−09 | 2.99E−08 |
| ENSBTAG00000045754 | SMIM1 | 2.272822592 | 5.897545398 | 1.56E−35 | 3.66E−34 |
| ENSBTAG00000032521 | PLEKHH2 | 2.270517241 | 3.228679397 | 5.29E−07 | 2.41E−06 |
| ENSBTAG00000010662 | LIMK2 | 2.266318892 | 9.021999876 | 5.02E−99 | 4.60E−97 |
| ENSBTAG00000002224 | UHRF1 | 2.264416052 | 5.076416427 | 5.69E−23 | 8.14E−22 |
| ENSBTAG00000020465 | FERMT1 | 2.258140584 | 6.036482957 | 1.16E−37 | 2.94E−36 |
| ENSBTAG00000014329 | WFDC12 | 2.256886986 | 1.160534745 | 0.500470377 | 0.600982399 |
| ENSBTAG00000005377 | SOSTDC1 | 2.249761433 | 2.679397608 | 0.000147851 | 0.000469379 |
| ENSBTAG00000012817 | JAG1 | 2.249712497 | 4.748327957 | 1.78E−18 | 2.01E−17 |
| ENSBTAG00000018774 | NUAK2 | 2.24823633 | 6.471417572 | 4.47E−46 | 1.41E−44 |
| ENSBTAG00000017719 | AKAP6 | 2.244869762 | 3.029232922 | 3.18E−06 | 1.32E−05 |
| ENSBTAG00000000770 | PGM2L1 | 2.243319443 | 3.504918871 | 2.39E−08 | 1.26E−07 |
| ENSBTAG00000013819 | URAD | 2.24229257 | 3.449863622 | 3.54E−08 | 1.84E−07 |
| ENSBTAG00000000460 | SYTL2 | 2.239295254 | 3.74357447 | 7.53E−10 | 4.60E−09 |
| ENSBTAG00000039556 | WIPI1 | 2.235980899 | 2.419285361 | 0.000615949 | 0.001760693 |
| ENSBTAG00000032538 | TPRG1 | 2.234397186 | 1.157572748 | 0.500470377 | 0.600982399 |
| ENSBTAG00000017616 | ADSS1 | 2.233397622 | 2.63926136 | 0.000242042 | 0.000744709 |
| ENSBTAG00000021752 | DNAJB4 | 2.228862927 | 4.631027082 | 3.22E−17 | 3.38E−16 |
| ENSBTAG00000003598 | P3H3 | 2.226409736 | 5.021506055 | 1.87E−21 | 2.48E−20 |
| ENSBTAG00000020614 | PRKCE | 2.225698446 | 3.863646387 | 2.27E−10 | 1.45E−09 |
| ENSBTAG00000012945 | PDE6A | 2.225332444 | 1.156392613 | 0.500470377 | 0.600982399 |
| ENSBTAG00000008793 | RNASE1 | 2.224441463 | 6.894613075 | 4.32E−54 | 1.70E−52 |
| ENSBTAG00000050933 | SHISA4 | 2.222136095 | 5.50360489 | 3.90E−28 | 6.97E−27 |
| ENSBTAG00000008330 | RNF19B | 2.218341333 | 5.939766947 | 5.53E−35 | 1.28E−33 |
| ENSBTAG00000017446 | E2F8 | 2.217157554 | 3.758408218 | 7.53E−10 | 4.60E−09 |
| ENSBTAG00000023374 | FUT1 | 2.215377319 | 3.772417842 | 4.77E−10 | 2.97E−09 |
| ENSBTAG00000024407 | CNN3 | 2.214751268 | 4.357351728 | 2.03E−14 | 1.82E−13 |
| ENSBTAG00000012834 | ARSI | 2.214723926 | 2.358537277 | 0.001034963 | 0.002827306 |
| ENSBTAG00000008105 | RBM38 | 2.209609986 | 3.590847423 | 1.01E−08 | 5.60E−08 |
| ENSBTAG00000021322 | TM4SF20 | 2.208922062 | 1.154249843 | 0.500470377 | 0.600982399 |
| ENSBTAG00000001830 | LRRC46 | 2.204756151 | 1.153705061 | 0.500470377 | 0.600982399 |
| ENSBTAG00000007608 | BMT2 | 2.20321131 | 4.693753052 | 1.80E−17 | 1.91E−16 |
| ENSBTAG00000032097 | FBXO21 | 2.203003857 | 7.369930274 | 6.02E−63 | 2.92E−61 |
| ENSBTAG00000006938 | PPM1F | 2.202944401 | 5.065114848 | 1.14E−21 | 1.53E−20 |
| ENSBTAG00000009112 | TRAM2 | 2.201382966 | 6.872894132 | 1.48E−52 | 5.69E−51 |
| ENSBTAG00000013204 | ATP13A3 | 2.200156989 | 7.88309197 | 1.08E−73 | 6.31E−72 |
| ENSBTAG00000012186 | DKKL1 | 2.199774335 | 4.343606481 | 3.10E−14 | 2.75E−13 |
| ENSBTAG00000005738 | ATP8B1 | 2.196214349 | 6.661737584 | 5.30E−48 | 1.76E−46 |
| ENSBTAG00000000296 | IMPDH1 | 2.193407548 | 5.075753877 | 5.28E−22 | 7.22E−21 |
| ENSBTAG00000047605 | Slc23a4 | 2.187302966 | 2.155939 | 0.004766113 | 0.011242294 |
| ENSBTAG00000012454 | SLC35A3 | 2.1870913 | 9.122752408 | 5.33E−95 | 4.57E−93 |
| ENSBTAG00000021535 | CROT | 2.187030162 | 4.081630332 | 5.36E−12 | 3.95E−11 |
| ENSBTAG00000009217 | WNT10A | 2.186245651 | 2.250536652 | 0.004737196 | 0.011188479 |
| ENSBTAG00000030301 | MAP7D1 | 2.18621971 | 5.129939883 | 3.68E−22 | 5.07E−21 |
| ENSBTAG00000003699 | ZFP3 | 2.186197057 | 4.601949656 | 2.51E−16 | 2.50E−15 |
| ENSBTAG00000050435 | Tmsb4x | 2.184476207 | 8.067451939 | 2.51E−76 | 1.54E−74 |
| ENSBTAG00000012751 | BTBD6 | 2.183209529 | 4.745009104 | 1.00E−17 | 1.08E−16 |
| ENSBTAG00000004869 | CXADR | 2.183140785 | 7.716461713 | 4.06E−69 | 2.23E−67 |
| ENSBTAG00000011885 | NNT | 2.182474931 | 4.558291126 | 5.15E−16 | 5.03E−15 |
| ENSBTAG00000017369 | MAMDC2 | 2.176540611 | 2.545657384 | 0.000388159 | 0.001150825 |
| ENSBTAG00000012338 | FAH | 2.174924228 | 3.758679988 | 1.99E−09 | 1.17E−08 |
| ENSBTAG00000026156 | VGLL3 | 2.172674973 | 3.582052012 | 1.01E−08 | 5.60E−08 |
| ENSBTAG00000055120 | OPHN1 | 2.170057075 | 2.340582224 | 0.001729326 | 0.004546339 |
| ENSBTAG00000027201 | RAPH1 | 2.165242848 | 6.42938264 | 1.08E−42 | 3.10E−41 |
| ENSBTAG00000008319 | DUSP9 | 2.164460634 | 1.14855498 | 0.500470377 | 0.600982399 |
| ENSBTAG00000000163 | DDIT4 | 2.159627674 | 6.820142994 | 1.49E−49 | 5.20E−48 |
| ENSBTAG00000008509 | SLC38A3 | 2.151712188 | 2.276024507 | 0.002871868 | 0.007144113 |
| ENSBTAG00000050814 | CFAP70 | 2.148685313 | 3.348224254 | 3.67E−07 | 1.70E−06 |
| ENSBTAG00000004019 | PMEL | 2.148363066 | 3.013533707 | 1.42E−05 | 5.34E−05 |
| ENSBTAG00000015907 | IRX4 | 2.148090976 | 3.599841587 | 2.67E−08 | 1.40E−07 |
| ENSBTAG00000010487 | SMIM3 | 2.146168037 | 3.236978256 | 1.43E−06 | 6.20E−06 |
| ENSBTAG00000001608 | STAR | 2.144935053 | 1.146074536 | 0.500470377 | 0.600982399 |
| ENSBTAG00000011820 | DAPK2 | 2.143485952 | 2.417305867 | 0.001721408 | 0.004528771 |
| ENSBTAG00000040298 | GSTT1 | 2.143223689 | 2.897886044 | 3.61E−05 | 0.000126775 |
| ENSBTAG00000016221 | AMOT | 2.141239642 | 9.358360962 | 2.87E−99 | 2.66E−97 |
| ENSBTAG00000005654 | TMSB10 | 2.139802431 | 10.93082246 | 2.35E−111 | 2.59E−109 |
| ENSBTAG00000004720 | SULF1 | 2.139332023 | 4.098075528 | 8.99E−12 | 6.45E−11 |
| ENSBTAG00000033669 | IL17RC | 2.139297558 | 4.566610501 | 1.29E−15 | 1.24E−14 |
| ENSBTAG00000016093 | PLP2 | 2.136832223 | 5.984233786 | 8.53E−34 | 1.88E−32 |
| ENSBTAG00000004349 | DAZAP2 | 2.135715724 | 6.74366723 | 1.68E−47 | 5.51E−46 |
| ENSBTAG00000051259 | bta-mir-2315 | 2.134031822 | 2.449352412 | 0.001053648 | 0.002872856 |
| ENSBTAG00000054755 | A0A3Q1MZS8 | 2.132505673 | 2.530436246 | 0.000388159 | 0.001150825 |
| ENSBTAG00000008814 | ADGRA2 | 2.132371535 | 3.010005168 | 1.42E−05 | 5.34E−05 |
| ENSBTAG00000049397 | CAV2 | 2.132012584 | 5.012347319 | 3.50E−20 | 4.34E−19 |
| ENSBTAG00000005373 | TPM1 | 2.12946245 | 8.817420666 | 2.95E−86 | 2.25E−84 |
| ENSBTAG00000038156 | DYNLT3 | 2.128705637 | 6.355081714 | 2.97E−40 | 7.97E−39 |
| ENSBTAG00000033766 | KRT16 | 2.127104442 | 1.143891901 | 0.500470377 | 0.600982399 |
| ENSBTAG00000007578 | SHTN1 | 2.122906762 | 7.914522575 | 9.57E−70 | 5.30E−68 |
| ENSBTAG00000031793 | NUDT2 | 2.122793944 | 3.453004594 | 9.60E−08 | 4.77E−07 |
| ENSBTAG00000015249 | B4GALT1 | 2.122604765 | 9.134318534 | 1.77E−90 | 1.45E−88 |
| ENSBTAG00000027569 | APBB2 | 2.121739948 | 5.582867651 | 3.53E−27 | 6.05E−26 |
| ENSBTAG00000014382 | KANK4 | 2.120866423 | 1.143121903 | 0.500470377 | 0.600982399 |
| ENSBTAG00000017665 | DHRS4 | 2.1201067 | 4.596431692 | 9.37E−16 | 9.03E−15 |
| ENSBTAG00000009091 | RNASEL | 2.118139492 | 2.474261339 | 0.001053648 | 0.002872856 |
| ENSBTAG00000007878 | ARHGAP22 | 2.114207443 | 2.918188817 | 3.61E−05 | 0.000126775 |
| ENSBTAG00000052575 | lncRNA | 2.113662641 | 2.571252128 | 0.000639459 | 0.001819741 |
| ENSBTAG00000005574 | CLU | 2.106294626 | 7.262012901 | 2.61E−56 | 1.09E−54 |
| ENSBTAG00000052904 | MEX3B | 2.101943171 | 4.50350959 | 9.11E−15 | 8.34E−14 |
| ENSBTAG00000011207 | CNN1 | 2.101416969 | 2.18021074 | 0.007756131 | 0.017553493 |
| ENSBTAG00000012036 | HR | 2.099354597 | 1.833594907 | 0.03963978 | 0.072977711 |
| ENSBTAG00000020843 | PCSK1 | 2.09261815 | 1.13967586 | 0.500470377 | 0.600982399 |
| ENSBTAG00000003166 | AXL | 2.092448021 | 3.37142909 | 9.34E−07 | 4.13E−06 |
| ENSBTAG00000019347 | PLXDC1 | 2.092345283 | 1.139644983 | 0.500470377 | 0.600982399 |
| ENSBTAG00000011634 | ACP3 | 2.08865429 | 4.700944573 | 1.63E−16 | 1.65E−15 |
| ENSBTAG00000013856 | SSBP2 | 2.088421028 | 3.774853337 | 5.01E−09 | 2.86E−08 |
| ENSBTAG00000015607 | ERMP1 | 2.088135063 | 5.69503494 | 3.10E−28 | 5.58E−27 |
| ENSBTAG00000048862 | TBX1 | 2.087163246 | 2.774671026 | 9.28E−05 | 0.000305452 |
| ENSBTAG00000008150 | PKIA | 2.085167498 | 7.648137836 | 1.07E−62 | 5.14E−61 |
| ENSBTAG00000007417 | GDF11 | 2.084043843 | 2.478054581 | 0.001053648 | 0.002872856 |
| ENSBTAG00000048924 | A0A3Q1LPH5 | 2.083692179 | 5.544109279 | 4.68E−26 | 7.66E−25 |
| ENSBTAG00000000571 | ARHGAP27 | 2.082199017 | 4.741004435 | 5.42E−17 | 5.60E−16 |
| ENSBTAG00000015197 | KLHDC8A | 2.080987818 | 5.024423433 | 8.27E−20 | 1.01E−18 |
| ENSBTAG00000026369 | ENC1 | 2.079129577 | 6.091941688 | 2.50E−34 | 5.67E−33 |
| ENSBTAG00000004322 | FOS | 2.078573303 | 5.987295243 | 1.52E−32 | 3.18E−31 |
| ENSBTAG00000007867 | STAT1 | 2.075281106 | 6.53679042 | 8.75E−42 | 2.43E−40 |
| ENSBTAG00000021457 | EFEMP2 | 2.07508009 | 3.378437953 | 5.95E−07 | 2.69E−06 |
| ENSBTAG00000024426 | PPP1R9A | 2.069862273 | 3.348170877 | 5.83E−07 | 2.65E−06 |
| ENSBTAG00000015604 | ZNF385A | 2.069420365 | 5.133095437 | 1.43E−20 | 1.82E−19 |
| ENSBTAG00000021338 | OAF | 2.067120872 | 3.504541748 | 9.98E−08 | 4.94E−07 |
| ENSBTAG00000005186 | DBNDD2 | 2.066985184 | 5.38540732 | 1.04E−23 | 1.54E−22 |
| ENSBTAG00000021068 | SUFU | 2.065870413 | 1.967609923 | 0.022219621 | 0.044135318 |
| ENSBTAG00000054779 | NA | 2.063858589 | 1.136234724 | 0.500470377 | 0.600982399 |
| ENSBTAG00000021768 | CCNG2 | 2.06360218 | 4.193887788 | 7.05E−12 | 5.13E−11 |
| ENSBTAG00000020049 | CABP1 | 2.060232957 | 1.135795919 | 0.500470377 | 0.600982399 |
| ENSBTAG00000016751 | MYO6 | 2.059832809 | 7.54142346 | 1.07E−59 | 4.76E−58 |
| ENSBTAG00000007584 | INPP1 | 2.059349266 | 3.832070608 | 3.34E−09 | 1.92E−08 |
| ENSBTAG00000008062 | DMRT2 | 2.057453861 | 1.135469543 | 0.500470377 | 0.600982399 |
| ENSBTAG00000001250 | TFAP2A | 2.051979751 | 6.910600903 | 7.43E−48 | 2.45E−46 |
| ENSBTAG00000000434 | CRYAB | 2.048471219 | 2.789470598 | 9.28E−05 | 0.000305452 |
| ENSBTAG00000011112 | KCNK5 | 2.048280783 | 6.233630095 | 1.14E−35 | 2.72E−34 |
| ENSBTAG00000024888 | PDE5A | 2.047834621 | 3.701433589 | 1.82E−08 | 9.71E−08 |
| ENSBTAG00000026758 | AKR7L | 2.047425274 | 2.217602443 | 0.007756131 | 0.017553493 |
| ENSBTAG00000052650 | lncRNA | 2.046506894 | 2.045629707 | 0.013417755 | 0.028312217 |
| ENSBTAG00000006990 | MYRF | 2.045617101 | 4.701955909 | 5.75E−16 | 5.61E−15 |
| ENSBTAG00000000310 | MFAP5 | 2.041274757 | 2.46244109 | 0.001053648 | 0.002872856 |
| ENSBTAG00000007863 | GLS | 2.041239087 | 5.188507268 | 9.04E−21 | 1.16E−19 |
| ENSBTAG00000038488 | TMSB4X | 2.039606597 | 6.654315665 | 9.34E−43 | 2.70E−41 |
| ENSBTAG00000013879 | DGKH | 2.036977482 | 2.562797657 | 0.001031235 | 0.002819747 |
| ENSBTAG00000007071 | RAI14 | 2.035516957 | 5.812388963 | 7.03E−29 | 1.29E−27 |
| ENSBTAG00000048903 | PRNP | 2.034743931 | 6.134278669 | 1.01E−33 | 2.22E−32 |
| ENSBTAG00000000706 | ADAMTS1 | 2.033371081 | 4.930701595 | 6.89E−18 | 7.46E−17 |
| ENSBTAG00000018013 | EMP3 | 2.032718432 | 2.309640965 | 0.002871868 | 0.007144113 |
| ENSBTAG00000012818 | PDLIM5 | 2.032348979 | 6.479127504 | 2.50E−39 | 6.56E−38 |
| ENSBTAG00000003829 | HPN | 2.032119661 | 2.285258283 | 0.002871868 | 0.007144113 |
| ENSBTAG00000016991 | EFNB2 | 2.028558022 | 6.925423202 | 2.17E−47 | 7.07E−46 |
| ENSBTAG00000006676 | FIBIN | 2.027189683 | 3.001823162 | 2.27E−05 | 8.28E−05 |
| ENSBTAG00000010956 | SCARB2 | 2.02690883 | 9.2207722 | 2.64E−88 | 2.04E−86 |
| ENSBTAG00000015893 | EPN3 | 2.024331372 | 2.1969175 | 0.007756131 | 0.017553493 |
| ENSBTAG00000007214 | ELL2 | 2.023188402 | 7.200254659 | 7.94E−52 | 2.97E−50 |
| ENSBTAG00000007490 | SULF2 | 2.022841041 | 3.558060079 | 1.02E−07 | 5.05E−07 |
| ENSBTAG00000031503 | NDUFA4L2 | 2.021697856 | 1.131281997 | 0.500470377 | 0.600982399 |
| ENSBTAG00000005807 | MICU1 | 2.01981888 | 6.846001773 | 2.88E−45 | 8.96E−44 |
| ENSBTAG00000008023 | RHBDL2 | 2.019241508 | 3.306597806 | 1.46E−06 | 6.31E−06 |
| ENSBTAG00000048525 | SERF1A | 2.017661403 | 1.130809147 | 0.500470377 | 0.600982399 |
| ENSBTAG00000037651 | S100A2 | 2.017483859 | 8.508651261 | 1.05E−73 | 6.17E−72 |
| ENSBTAG00000014620 | FOXI1 | 2.016672733 | 2.072165892 | 0.013417755 | 0.028312217 |
| ENSBTAG00000030910 | F1MLR1 | 2.012787395 | 2.947321536 | 5.75E−05 | 0.000195977 |
| ENSBTAG00000006988 | M1AP | 2.012241542 | 2.523401356 | 0.001653454 | 0.004365654 |
| ENSBTAG00000014906 | VCAN | 2.012039087 | 3.602509617 | 6.67E−08 | 3.36E−07 |
| ENSBTAG00000047582 | CSNK1E | 2.011198129 | 5.091270393 | 2.64E−19 | 3.15E−18 |
| ENSBTAG00000017287 | DRAM1 | 2.010882256 | 5.468379884 | 9.46E−24 | 1.41E−22 |
| ENSBTAG00000044061 | FAXC | 2.009517532 | 3.510959365 | 2.48E−07 | 1.17E−06 |
| ENSBTAG00000020989 | SUSD4 | 2.009442749 | 5.350084544 | 3.68E−22 | 5.07E−21 |
| ENSBTAG00000018499 | ANXA8L1 | 2.009202784 | 10.66771853 | 4.70E−98 | 4.25E−96 |
| ENSBTAG00000009125 | ALDH1A3 | 2.00414108 | 8.944395357 | 2.16E−79 | 1.39E−77 |
| ENSBTAG00000009543 | ESYT1 | 2.000649844 | 6.739798715 | 1.01E−42 | 2.92E−41 |
| ENSBTAG00000004018 | DGKA | 2.000631859 | 6.265474197 | 7.56E−35 | 1.74E−33 |
| ENSBTAG00000051588 | A0A3Q1LU09 | 1.997713397 | 2.218700563 | 0.007756131 | 0.017553493 |
| ENSBTAG00000016352 | STX10 | 1.997404581 | 2.991043457 | 3.62E−05 | 0.00012713 |
| ENSBTAG00000008036 | CELSR1 | 1.996591529 | 6.031736095 | 2.21E−31 | 4.44E−30 |
| ENSBTAG00000014059 | ATP2B4 | 1.996190779 | 6.918433066 | 6.06E−46 | 1.90E−44 |
| ENSBTAG00000033345 | STAR | 1.993599426 | 4.788680748 | 4.99E−16 | 4.88E−15 |
| ENSBTAG00000010667 | PIK3IP1 | 1.992425327 | 2.658500229 | 0.000618805 | 0.00176748 |
| ENSBTAG00000018316 | ZMYND15 | 1.99208767 | 2.719240809 | 0.000241864 | 0.000744475 |
| ENSBTAG00000019679 | SYNE4 | 1.988714224 | 2.95727224 | 5.75E−05 | 0.000195977 |
| ENSBTAG00000006194 | FOSL1 | 1.986495972 | 2.830318345 | 0.000148238 | 0.000470302 |
| ENSBTAG00000014608 | KDM1B | 1.982860203 | 3.966218722 | 1.50E−09 | 8.95E−09 |
| ENSBTAG00000016841 | ATP10B | 1.980784393 | 2.303845905 | 0.002871868 | 0.007144113 |
| ENSBTAG00000025400 | PARP4 | 1.978331484 | 5.172939698 | 8.32E−20 | 1.02E−18 |
| ENSBTAG00000049058 | INHBB | 1.978313641 | 4.486845574 | 2.54E−13 | 2.11E−12 |
| ENSBTAG00000034222 | CAB39L | 1.977787852 | 6.121164638 | 3.03E−32 | 6.25E−31 |
| ENSBTAG00000012615 | ZEB2 | 1.976837378 | 2.558116239 | 0.001031235 | 0.002819747 |
| ENSBTAG00000024381 | MAN1A1 | 1.971754319 | 4.346936425 | 5.58E−12 | 4.10E−11 |
| ENSBTAG00000034776 | DDAH1 | 1.970833952 | 2.583596264 | 0.000639459 | 0.001819741 |
| ENSBTAG00000021217 | COL11A1 | 1.969055838 | 2.896482276 | 0.000143291 | 0.000455495 |
| ENSBTAG00000006140 | PEX11A | 1.968398228 | 3.205217931 | 1.37E−05 | 5.17E−05 |
| ENSBTAG00000037804 | IKZF2 | 1.968393091 | 2.205844398 | 0.007756131 | 0.017553493 |
| ENSBTAG00000005410 | RGS7 | 1.964914966 | 2.040048845 | 0.013417755 | 0.028312217 |
| ENSBTAG00000019855 | MAP3K15 | 1.963871638 | 3.712390988 | 4.36E−08 | 2.24E−07 |
| ENSBTAG00000016290 | MOB1B | 1.962841765 | 7.340879102 | 1.31E−51 | 4.85E−50 |
| ENSBTAG00000011802 | COL6A1 | 1.95940295 | 3.055665825 | 3.56E−05 | 0.000125275 |
| ENSBTAG00000020119 | RNF43 | 1.957038176 | 3.869742046 | 3.41E−09 | 1.96E−08 |
| ENSBTAG00000004653 | ADGRA3 | 1.95662213 | 7.119227691 | 8.97E−48 | 2.95E−46 |
| ENSBTAG00000005681 | ME1 | 1.956049039 | 5.162462818 | 5.18E−19 | 6.05E−18 |
| ENSBTAG00000000686 | NANP | 1.953744323 | 3.107345864 | 3.44E−05 | 0.000121424 |
| ENSBTAG00000047426 | LYPD6 | 1.950927896 | 2.364392633 | 0.004506777 | 0.010680384 |
| ENSBTAG00000031217 | MYL6B | 1.950501853 | 3.163524345 | 8.97E−06 | 3.46E−05 |
| ENSBTAG00000051808 | OR52I1 | 1.950190413 | 1.123161463 | 1 | 1 |
| ENSBTAG00000007624 | FRMD4B | 1.948320193 | 3.855449613 | 5.21E−09 | 2.97E−08 |
| ENSBTAG00000000894 | PGK1 | 1.947861433 | 9.385641209 | 1.25E−83 | 8.82E−82 |
| ENSBTAG00000052734 | PAPOLB | 1.947151725 | 2.920660582 | 9.09E−05 | 0.000299776 |
| ENSBTAG00000009336 | LXN | 1.94646369 | 4.311764754 | 8.15E−12 | 5.86E−11 |
| ENSBTAG00000048737 | DEFB10 | 1.945427036 | 1.122661175 | 1 | 1 |
| ENSBTAG00000047293 | NRIP1 | 1.942793537 | 5.023343443 | 9.32E−18 | 1.00E−16 |
| ENSBTAG00000003708 | SEC23A | 1.942350561 | 5.923863517 | 2.41E−28 | 4.36E−27 |
| ENSBTAG00000053105 | TMEM263 | 1.941693997 | 3.297668073 | 5.51E−06 | 2.21E−05 |
| ENSBTAG00000007741 | CRLF1 | 1.941390995 | 2.406253825 | 0.002794825 | 0.006976124 |
| ENSBTAG00000021427 | HOXB3 | 1.9407723 | 3.411085518 | 1.45E−06 | 6.27E−06 |
| ENSBTAG00000048493 | NAALADL2 | 1.939523996 | 3.445703053 | 5.99E−07 | 2.71E−06 |
| ENSBTAG00000004587 | DUSP6 | 1.939223129 | 5.154061593 | 3.44E−19 | 4.07E−18 |
| ENSBTAG00000019390 | CA12 | 1.938378407 | 2.420244424 | 0.001721408 | 0.004528771 |
| ENSBTAG00000016091 | KLHL25 | 1.935397901 | 3.24140684 | 5.64E−06 | 2.25E−05 |
| ENSBTAG00000020632 | NQO1 | 1.932251097 | 5.537228689 | 4.48E−23 | 6.45E−22 |
| ENSBTAG00000025329 | IRF2BPL | 1.931465102 | 3.534058043 | 5.85E−07 | 2.66E−06 |
| ENSBTAG00000014731 | GAPDH | 1.929184909 | 10.47966703 | 4.81E−90 | 3.85E−88 |
| ENSBTAG00000017626 | GFPT1 | 1.928290105 | 7.147928816 | 3.77E−47 | 1.22E−45 |
| ENSBTAG00000009653 | TCL1B | 1.927546482 | 1.120693867 | 1 | 1 |
| ENSBTAG00000011748 | SGMS1 | 1.92749149 | 7.045586365 | 3.85E−45 | 1.19E−43 |
| ENSBTAG00000054306 | NA | 1.925169488 | 4.263587398 | 2.67E−11 | 1.84E−10 |
| ENSBTAG00000019596 | GPRC5B | 1.923254484 | 3.883074896 | 7.97E−09 | 4.45E−08 |
| ENSBTAG00000011100 | CTSC | 1.920934529 | 6.001696124 | 3.95E−29 | 7.35E−28 |
| ENSBTAG00000000815 | EPHA2 | 1.917631803 | 5.303529767 | 1.61E−20 | 2.04E−19 |
| ENSBTAG00000000786 | NQO2 | 1.917412128 | 1.929003647 | 0.036417288 | 0.067740313 |
| ENSBTAG00000031194 | PHLDA2 | 1.915458503 | 2.032977401 | 0.013417755 | 0.028312217 |
| ENSBTAG00000011108 | USP54 | 1.914941375 | 5.454544615 | 6.56E−22 | 8.93E−21 |
| ENSBTAG00000018554 | processed | 1.913542085 | 3.523430741 | 8.97E−07 | 3.98E−06 |
| | pseudogene | | | | |
| ENSBTAG00000017060 | ITGB2 | 1.911283825 | 2.653201995 | 0.000982063 | 0.00271516 |
| ENSBTAG00000012464 | AFAP1 | 1.910646675 | 6.782629822 | 2.94E−40 | 7.92E−39 |
| ENSBTAG00000003804 | SCCPDH | 1.908087463 | 5.100024204 | 5.52E−18 | 6.03E−17 |
| ENSBTAG00000011590 | GLIPR1 | 1.907658488 | 5.74059488 | 2.58E−25 | 4.10E−24 |
| ENSBTAG00000011421 | CD37 | 1.906295213 | 2.375917386 | 0.004506777 | 0.010680384 |
| ENSBTAG00000009093 | GAL3ST2 | 1.904544176 | 3.470414243 | 9.19E−07 | 4.07E−06 |
| ENSBTAG00000053653 | A0A3Q1MLA9 | 1.902335254 | 1.117927217 | 1 | 1 |
| ENSBTAG00000008078 | TRIM59 | 1.901358034 | 3.514883931 | 8.97E−07 | 3.98E−06 |
| ENSBTAG00000004676 | TMEM184A | 1.899311868 | 2.706696431 | 0.000387836 | 0.001150825 |
| ENSBTAG00000008714 | CLSTN2 | 1.896214916 | 4.387183119 | 1.26E−11 | 8.93E−11 |
| ENSBTAG00000024291 | FAM71F2 | 1.893321549 | 2.061168072 | 0.013417755 | 0.028312217 |
| ENSBTAG00000052304 | Prss43 | 1.891491821 | 1.116793158 | 1 | |
| ENSBTAG00000005668 | SLC39A8 | 1.891105331 | 7.050107135 | 6.61E−44 | 1.97E−42 |
| ENSBTAG00000039195 | NHS | 1.884259148 | 2.871395015 | 0.00022482 | 0.000695516 |
| ENSBTAG00000003632 | CYP39A1 | 1.884112235 | 2.601529787 | 0.001549681 | 0.00412358 |
| ENSBTAG00000054881 | RNLS | 1.880668818 | 3.225264237 | 8.80E−06 | 3.40E−05 |
| ENSBTAG00000021487 | CIART | 1.877606846 | 2.105618625 | 0.020265727 | 0.040650631 |
| ENSBTAG00000006082 | DNASE2 | 1.873936322 | 6.719783787 | 3.28E−38 | 8.38E−37 |
| ENSBTAG00000000097 | EFCAB14 | 1.872957175 | 7.76212072 | 1.99E−54 | 7.92E−53 |
| ENSBTAG00000002127 | TCP11L2 | 1.872810292 | 3.441275009 | 2.18E−06 | 9.27E−06 |
| ENSBTAG00000013023 | MAP4K4 | 1.872803191 | 7.162147663 | 4.23E−45 | 1.30E−43 |
| ENSBTAG00000025762 | CNP | 1.871151131 | 4.23184067 | 1.97E−10 | 1.27E−09 |
| ENSBTAG00000013562 | SKP2 | 1.869957056 | 5.464577243 | 2.73E−21 | 3.60E−20 |
| ENSBTAG00000005710 | NCAM1 | 1.866780443 | 3.95176817 | 7.81E−09 | 4.37E−08 |
| ENSBTAG00000010304 | CHKA | 1.866194669 | 5.871551986 | 9.72E−26 | 1.57E−24 |
| ENSBTAG00000008600 | TRAF3IP2 | 1.865708048 | 3.619172942 | 5.50E−07 | 2.51E−06 |
| ENSBTAG00000004028 | POLK | 1.862910706 | 5.010485441 | 1.76E−16 | 1.77E−15 |
| ENSBTAG00000021176 | CRISPLD2 | 1.861415384 | 4.577577917 | 8.87E−13 | 7.05E−12 |
| ENSBTAG00000023607 | HACD2 | 1.859234517 | 6.695343254 | 1.69E−37 | 4.26E−36 |
| ENSBTAG00000010293 | PARD3B | 1.859154303 | 4.924993165 | 1.39E−15 | 1.32E−14 |
| ENSBTAG00000002081 | BMPR1B | 1.858866128 | 2.723579134 | 0.000916982 | 0.002552064 |
| ENSBTAG00000021215 | VWA5A | 1.858220343 | 3.525416375 | 2.00E−06 | 8.55E−06 |
| ENSBTAG00000046547 | CIDEA | 1.857777282 | 3.382723441 | 3.46E−06 | 1.43E−05 |
| ENSBTAG00000006789 | FGD5 | 1.856056007 | 2.90649565 | 0.000143291 | 0.000455495 |
| ENSBTAG00000013093 | ALDH3B1 | 1.854524581 | 2.657624771 | 0.000982063 | 0.00271516 |
| ENSBTAG00000002996 | SHROOM4 | 1.85353955 | 2.277727089 | 0.011452377 | 0.024593268 |
| ENSBTAG00000021286 | SLC37A1 | 1.851801415 | 6.219518592 | 1.71E−30 | 3.31E−29 |
| ENSBTAG00000050527 | NA | 1.851146865 | 2.036584394 | 0.022219621 | 0.044135318 |
| ENSBTAG00000035926 | E1BED8 | 1.850667872 | 3.005350682 | 8.74E−05 | 0.000288938 |
| ENSBTAG00000008931 | CREG1 | 1.848045711 | 4.476051701 | 5.86E−12 | 4.30E−11 |
| ENSBTAG00000015824 | RNF141 | 1.847331506 | 4.949371945 | 9.90E−16 | 9.54E−15 |
| ENSBTAG00000020105 | AQP7 | 1.843330211 | 1.111725715 | 1 | 1 |
| ENSBTAG00000053826 | BORCS7 | 1.838510268 | 2.648619516 | 0.000982063 | 0.00271516 |
| ENSBTAG00000012180 | BMF | 1.835250709 | 4.341415457 | 8.74E−11 | 5.78E−10 |
| ENSBTAG00000003489 | IPCEF1 | 1.834897184 | 2.670400083 | 0.000618805 | 0.00176748 |
| ENSBTAG00000005714 | ACTC1 | 1.834275108 | 4.508044446 | 4.00E−12 | 2.99E−11 |
| ENSBTAG00000000436 | TNFAIP3 | 1.833741065 | 3.848703491 | 2.77E−08 | 1.45E−07 |
| ENSBTAG00000002804 | PDGFRB | 1.831489359 | 2.689844279 | 0.001427196 | 0.003823322 |
| ENSBTAG00000048114 | AL355312.6 | 1.83124469 | 3.749921724 | 1.48E−07 | 7.19E−07 |
| ENSBTAG00000008143 | STK17A | 1.830602523 | 6.484230252 | 2.94E−33 | 6.24E−32 |
| ENSBTAG00000005633 | ARHGEF28 | 1.830076434 | 5.522999878 | 3.45E−21 | 4.52E−20 |
| ENSBTAG00000014890 | CYP19A1 | 1.829791767 | 6.070311201 | 6.93E−28 | 1.23E−26 |
| ENSBTAG00000021518 | TMPRSS4 | 1.829587928 | 7.237430642 | 2.35E−44 | 7.14E−43 |
| ENSBTAG00000021134 | DPP9 | 1.82838473 | 5.147223702 | 3.67E−17 | 3.84E−16 |
| ENSBTAG00000021904 | KIF26A | 1.827196256 | 3.282292324 | 1.32E−05 | 4.97E−05 |
| ENSBTAG00000004139 | BACH1 | 1.824904257 | 5.956208009 | 4.99E−26 | 8.15E−25 |
| ENSBTAG00000015611 | TBC1D9B | 1.824131223 | 6.892932797 | 5.20E−39 | 1.35E−37 |
| ENSBTAG00000049656 | processed | 1.823554873 | 4.426835143 | 1.85E−11 | 1.29E−10 |
| | pseudogene | | | | |
| ENSBTAG00000030817 | LMBR1 | 1.821970833 | 5.860034469 | 8.02E−25 | 1.25E−23 |
| ENSBTAG00000001568 | PPIC | 1.820761879 | 4.475233829 | 1.24E−11 | 8.81E−11 |
| ENSBTAG00000006948 | GARNL3 | 1.820710002 | 2.998281335 | 8.74E−05 | 0.000288938 |
| ENSBTAG00000021396 | HEG1 | 1.820059962 | 3.802201715 | 9.49E−08 | 4.72E−07 |
| ENSBTAG00000046533 | RAB31 | 1.819673601 | 6.100727269 | 4.88E−28 | 8.68E−27 |
| ENSBTAG00000005403 | ADI1 | 1.818424142 | 4.602114591 | 1.29E−12 | 1.01E−11 |
| ENSBTAG00000000599 | CCNI | 1.817223455 | 8.229685928 | 2.79E−58 | 1.21E−56 |
| ENSBTAG00000038706 | MT1E | 1.817039588 | 1.930684076 | 0.036417288 | 0.067740313 |
| ENSBTAG00000008541 | MGST1 | 1.814713356 | 6.183537675 | 9.50E−29 | 1.73E−27 |
| ENSBTAG00000000892 | CGAS | 1.814689252 | 3.805880783 | 9.49E−08 | 4.72E−07 |
| ENSBTAG00000000720 | CTSL | 1.813408766 | 10.20420297 | 5.11E−79 | 3.27E−77 |
| ENSBTAG00000012500 | RARA | 1.810360854 | 5.003683306 | 7.33E−16 | 7.12E−15 |
| ENSBTAG00000003405 | TP53I3 | 1.809373083 | 4.616279668 | 1.29E−12 | 1.00E−11 |
| ENSBTAG00000048577 | lncRNA | 1.806418025 | 2.074343935 | 0.032277735 | 0.060756871 |
| ENSBTAG00000005810 | PDZK1 | 1.804868169 | 6.25563363 | 1.41E−29 | 2.67E−28 |
| ENSBTAG00000019267 | MMP2 | 1.804114825 | 3.900353288 | 3.94E−08 | 2.04E−07 |
| ENSBTAG00000021934 | RETSAT | 1.801483447 | 3.062023097 | 8.29E−05 | 0.000274768 |
| ENSBTAG00000012444 | ADAM12 | 1.800278381 | 1.515721235 | 0.126234735 | 0.192462044 |
| ENSBTAG00000012305 | ADGRL2 | 1.799533784 | 7.854046211 | 2.96E−52 | 1.13E−50 |
| ENSBTAG00000053460 | VWC2L | 1.799303931 | 1.107220654 | 1 | 1 |
| ENSBTAG00000010256 | TMEM170B | 1.798689565 | 2.643501558 | 0.000982063 | 0.00271516 |
| ENSBTAG00000023073 | FAM89A | 1.795085162 | 3.824525568 | 6.31E−08 | 3.20E−07 |
| ENSBTAG00000013749 | RHOQ | 1.793872105 | 4.629095654 | 1.84E−12 | 1.41E−11 |
| ENSBTAG00000001139 | ACHE | 1.792738513 | 2.23816379 | 0.018020507 | 0.036471199 |
| ENSBTAG00000001706 | SLC9A2 | 1.791621714 | 4.10489178 | 3.20E−09 | 1.85E−08 |
| ENSBTAG00000020935 | HIF1A | 1.790786973 | 8.108908541 | 3.84E−55 | 1.56E−53 |
| ENSBTAG00000000369 | EPS8 | 1.784536667 | 4.636922674 | 1.84E−12 | 1.41E−11 |
| ENSBTAG00000016255 | PLEK2 | 1.783173837 | 2.176668213 | 0.012594782 | 0.026779888 |
| ENSBTAG00000009677 | PARP10 | 1.783071355 | 4.636880355 | 1.84E−12 | 1.41E−11 |
| ENSBTAG00000003438 | ZBTB43 | 1.782306248 | 4.717385689 | 6.01E−13 | 4.84E−12 |
| ENSBTAG00000019916 | CLMN | 1.781073915 | 4.250059166 | 4.24E−10 | 2.65E−09 |
| ENSBTAG00000012976 | WLS | 1.77882968 | 7.931676227 | 4.75E−52 | 1.80E−50 |
| ENSBTAG00000011935 | RASSF6 | 1.777683147 | 2.989099237 | 0.000136241 | 0.000435442 |
| ENSBTAG00000053833 | A0A3Q1MLM0 | 1.777492569 | 4.333091304 | 1.28E−10 | 8.37E−10 |
| ENSBTAG00000037456 | AHDC1 | 1.776359872 | 4.032424622 | 1.59E−08 | 8.54E−08 |
| ENSBTAG00000008609 | SEPTIN8 | 1.776334134 | 6.338730119 | 1.03E−29 | 1.95E−28 |
| ENSBTAG00000012371 | CPD | 1.772702469 | 7.450127453 | 2.21E−45 | 6.87E−44 |
| ENSBTAG00000007870 | FKBP14 | 1.772471914 | 4.20570975 | 1.97E−09 | 1.16E−08 |
| ENSBTAG00000004368 | NFATC3 | 1.769516226 | 5.766181785 | 9.76E−23 | 1.38E−21 |
| ENSBTAG00000012762 | C7H5orf24 | 1.769451175 | 4.359320139 | 1.26E−10 | 8.22E−10 |
| ENSBTAG00000007238 | AL355312.6 | 1.768813425 | 2.304749685 | 0.011452377 | 0.024593268 |
| ENSBTAG00000013493 | BNIP2 | 1.767375941 | 4.004443307 | 1.65E−08 | 8.86E−08 |
| ENSBTAG00000031707 | FRMD6 | 1.767125513 | 5.430604468 | 5.15E−19 | 6.02E−18 |
| ENSBTAG00000018309 | SDCBP | 1.766415226 | 3.709800718 | 5.02E−07 | 2.30E−06 |
| ENSBTAG00000010351 | SNX25 | 1.764351499 | 4.743032188 | 5.94E−13 | 4.79E−12 |
| ENSBTAG00000005816 | IRF9 | 1.763927619 | 4.259966472 | 8.89E−10 | 5.40E−09 |
| ENSBTAG00000007881 | IFIT1 | 1.761268636 | 2.509618257 | 0.005858484 | 0.013565897 |
| ENSBTAG00000009075 | RNF207 | 1.758627566 | 4.923694491 | 3.33E−14 | 2.95E−13 |
| ENSBTAG00000017604 | RAB13 | 1.755802378 | 6.025131265 | 3.33E−25 | 5.28E−24 |
| ENSBTAG00000002076 | STAU2 | 1.755373063 | 4.578580557 | 1.16E−11 | 8.25E−11 |
| ENSBTAG00000007147 | CUEDC1 | 1.752188759 | 4.843742128 | 1.38E−13 | 1.17E−12 |
| ENSBTAG00000015779 | PML | 1.752007771 | 4.900370332 | 4.74E−14 | 4.17E−13 |
| ENSBTAG00000013274 | CENPVL1 | 1.751371684 | 4.159166209 | 4.41E−09 | 2.52E−08 |
| ENSBTAG00000009637 | SLC12A2 | 1.750477094 | 6.273956233 | 2.68E−28 | 4.84E−27 |
| ENSBTAG00000016933 | IL27RA | 1.750144498 | 1.786065001 | 0.0667965 | 0.113234427 |
| ENSBTAG00000008585 | ARHGEF10 | 1.748812793 | 2.492639556 | 0.002634628 | 0.00661453 |
| ENSBTAG00000031483 | CALM1 | 1.74859926 | 5.146191924 | 3.97E−16 | 3.90E−15 |
| ENSBTAG00000009191 | TUSC3 | 1.747655204 | 5.186126594 | 2.06E−16 | 2.06E−15 |
| ENSBTAG00000000536 | ZNF395 | 1.745285644 | 5.621956539 | 1.63E−20 | 2.06E−19 |
| ENSBTAG00000009391 | ADPRH | 1.744875691 | 2.076521636 | 0.032277735 | 0.060756871 |
| ENSBTAG00000013901 | PTDSS1 | 1.744564122 | 7.324848668 | 2.74E−42 | 7.79E−41 |
| ENSBTAG00000001359 | B3GNT7 | 1.744481817 | 6.501229281 | 3.16E−31 | 6.28E−30 |
| ENSBTAG00000000598 | CST3 | 1.744431694 | 6.118460635 | 3.19E−26 | 5.25E−25 |
| ENSBTAG00000012505 | ARHGEF17 | 1.743987883 | 4.3754984 | 1.79E−10 | 1.16E−09 |
| ENSBTAG00000004112 | GAMT | 1.743915014 | 3.72831579 | 3.35E−07 | 1.56E−06 |
| ENSBTAG00000048432 | BTBD3 | 1.741345323 | 6.083287165 | 8.13E−26 | 1.32E−24 |
| ENSBTAG00000031575 | VAV3 | 1.74040464 | 3.866653567 | 1.30E−07 | 6.37E−07 |
| ENSBTAG00000020893 | MATN3 | 1.736466056 | 2.399499574 | 0.010213283 | 0.022156498 |
| ENSBTAG00000047664 | GNAZ | 1.735493634 | 2.508065738 | 0.005858484 | 0.013565897 |
| ENSBTAG00000014124 | MLLT3 | 1.735151376 | 2.0569165 | 0.032277735 | 0.060756871 |
| ENSBTAG00000008091 | SELENBP1 | 1.731779242 | 2.757263041 | 0.00129759 | 0.003504331 |
| ENSBTAG00000003619 | SEC24D | 1.731623797 | 7.349481858 | 7.26E−42 | 2.04E−40 |
| ENSBTAG00000003857 | SUSD6 | 1.731344992 | 5.712395529 | 2.34E−21 | 3.09E−20 |
| ENSBTAG00000000501 | KLHL13 | 1.730402064 | 3.91248553 | 8.33E−08 | 4.16E−07 |
| ENSBTAG00000001343 | DEPDC1 | 1.728238615 | 5.570748334 | 6.97E−20 | 8.54E−19 |
| ENSBTAG00000009569 | DOCK6 | 1.727919 | 7.176169152 | 1.10E−39 | 2.92E−38 |
| ENSBTAG00000013773 | PKP4 | 1.726840173 | 6.608497035 | 6.08E−32 | 1.25E−30 |
| ENSBTAG00000021808 | CACNB3 | 1.726776629 | 5.131789435 | 1.08E−15 | 1.04E−14 |
| ENSBTAG00000011913 | CKAP4 | 1.726483753 | 6.369626081 | 8.66E−29 | 1.58E−27 |
| ENSBTAG00000046104 | B3GALNT1 | 1.723047702 | 5.137072339 | 1.49E−15 | 1.42E−14 |
| ENSBTAG00000002527 | ZSWIM4 | 1.721960599 | 5.493492179 | 5.58E−19 | 6.50E−18 |
| ENSBTAG00000048273 | C6H4orf3 | 1.721724272 | 7.291855089 | 7.27E−41 | 1.98E−39 |
| ENSBTAG00000031476 | DLL1 | 1.719693586 | 3.717734386 | 1.05E−06 | 4.63E−06 |
| ENSBTAG00000009698 | ABCD3 | 1.718968217 | 7.399556641 | 4.97E−42 | 1.40E−40 |
| ENSBTAG00000004603 | PHC2 | 1.718000099 | 5.918553015 | 2.80E−23 | 4.07E−22 |
| ENSBTAG00000019915 | GSN | 1.717870617 | 7.561049235 | 3.35E−44 | 1.02E−42 |
| ENSBTAG00000026097 | LIMD1 | 1.71733033 | 4.596019945 | 1.62E−11 | 1.14E−10 |
| ENSBTAG00000009238 | HOXC6 | 1.715875442 | 3.088027727 | 0.000180779 | 0.000566677 |
| ENSBTAG00000004008 | FAM199X | 1.714754418 | 3.169790939 | 0.000109347 | 0.000355846 |
| ENSBTAG00000005124 | JADE3 | 1.711136327 | 6.01173694 | 4.00E−24 | 6.06E−23 |
| ENSBTAG00000018831 | DAO | 1.710768045 | 1.098526465 | 1 | 1 |
| ENSBTAG00000049845 | HOXD8 | 1.7100643 | 2.4249088 | 0.006551784 | 0.015021888 |
| ENSBTAG00000006615 | CASP7 | 1.709465249 | 4.100718964 | 1.45E−08 | 7.85E−08 |
| ENSBTAG00000020869 | DDC | 1.70883555 | 1.098339135 | 1 | 1 |
| ENSBTAG00000009132 | TMPRSS2 | 1.706664185 | 3.737339042 | 7.09E−07 | 3.18E−06 |
| ENSBTAG00000002977 | ECE1 | 1.70587053 | 6.684351433 | 2.56E−32 | 5.28E−31 |
| ENSBTAG00000019722 | GUCY2C | 1.705615544 | 2.345381335 | 0.015784905 | 0.032624321 |
| ENSBTAG00000025644 | CALM1 | 1.703979272 | 8.791601017 | 2.04E−58 | 8.95E−57 |
| ENSBTAG00000005129 | CEP55 | 1.703024875 | 6.339009261 | 7.22E−28 | 1.27E−26 |
| ENSBTAG00000002341 | ETS1 | 1.701595 | 4.895760962 | 1.85E−13 | 1.56E−12 |
| ENSBTAG00000054330 | TUBB2B | 1.700681208 | 4.721581737 | 4.83E−12 | 3.58E−11 |
| ENSBTAG00000053086 | PHLDA3 | 1.698694915 | 3.231488798 | 1.00E−04 | 0.00032768 |
| ENSBTAG00000011975 | SERPINB1 | 1.698660761 | 4.50893577 | 1.05E−10 | 6.89E−10 |
| ENSBTAG00000007522 | RAPIGDS1 | 1.69815125 | 6.890921183 | 1.17E−34 | 2.69E−33 |
| ENSBTAG00000009495 | BCAM | 1.697720719 | 9.012857174 | 3.88E−62 | 1.84E−60 |
| ENSBTAG00000024162 | SLC19A1 | 1.697710878 | 2.939897339 | 0.000692924 | 0.001960857 |
| ENSBTAG00000015350 | PLIN1 | 1.694706486 | 2.310803282 | 0.011452377 | 0.024593268 |
| ENSBTAG00000025219 | SLFN12 | 1.694635795 | 3.355149265 | 1.79E−05 | 6.61E−05 |
| ENSBTAG00000039326 | GCNT2 | 1.693124143 | 5.554578396 | 5.86E−19 | 6.82E−18 |
| ENSBTAG00000007534 | GGH | 1.692894426 | 4.540622622 | 7.05E−11 | 4.71E−10 |
| ENSBTAG00000005205 | DSE | 1.689967383 | 5.029015902 | 2.20E−14 | 1.97E−13 |
| ENSBTAG00000002471 | CHL1 | 1.689906447 | 5.694914634 | 2.49E−20 | 3.11E−19 |
| ENSBTAG00000013790 | MAP3K1 | 1.686135112 | 7.839146984 | 1.46E−46 | 4.66E−45 |
| ENSBTAG00000011879 | B4GALT2 | 1.686085951 | 4.696863192 | 7.07E−12 | 5.14E−11 |
| ENSBTAG00000008893 | ANO10 | 1.68435664 | 5.390777972 | 3.03E−17 | 3.19E−16 |
| ENSBTAG00000032089 | CDC42EP2 | 1.681408989 | 2.914417087 | 0.000502019 | 0.001466172 |
| ENSBTAG00000020681 | FNDC3A | 1.679846643 | 7.767142014 | 3.25E−45 | 1.01E−43 |
| ENSBTAG00000000828 | CAPN6 | 1.677949707 | 5.204349398 | 1.04E−15 | 1.00E−14 |
| ENSBTAG00000006860 | VWA2 | 1.677344134 | 3.92696552 | 1.71E−07 | 8.22E−07 |
| ENSBTAG00000019343 | MDFI | 1.675753638 | 4.007331409 | 1.03E−07 | 5.08E−07 |
| ENSBTAG00000030521 | IER5L | 1.674001315 | 2.102974953 | 0.032277735 | 0.060756871 |
| ENSBTAG00000017804 | BNIP3 | 1.672775316 | 5.711819424 | 3.40E−20 | 4.22E−19 |
| ENSBTAG00000048965 | TVP23B | 1.672558536 | 6.092932911 | 4.09E−24 | 6.18E−23 |
| ENSBTAG00000015909 | PDE8A | 1.672410419 | 5.592984993 | 4.44E−19 | 5.23E−18 |
| ENSBTAG00000005909 | LEPROT | 1.672111302 | 7.67353828 | 8.97E−44 | 2.66E−42 |
| ENSBTAG00000049558 | CYP17A1 | 1.670233677 | 2.041366492 | 0.050825686 | 0.091310673 |
| ENSBTAG00000007703 | NMT2 | 1.668743836 | 5.494972269 | 4.79E−18 | 5.26E−17 |
| ENSBTAG00000014291 | WNT2B | 1.666183752 | 4.717069536 | 6.85E−12 | 4.99E−11 |
| ENSBTAG00000014636 | ZFHX3 | 1.664892615 | 5.435641954 | 2.21E−17 | 2.34E−16 |
| ENSBTAG00000015303 | MPP6 | 1.664724495 | 3.329752854 | 4.06E−05 | 0.000141828 |
| ENSBTAG00000000287 | GNPDA2 | 1.663935813 | 4.966656697 | 1.69E−13 | 1.43E−12 |
| ENSBTAG00000005893 | TMEM68 | 1.663695746 | 5.286790916 | 3.81E−16 | 3.75E−15 |
| ENSBTAG00000001112 | SMOX | 1.661575899 | 3.814842575 | 6.21E−07 | 2.81E−06 |
| ENSBTAG00000003935 | TMBIM1 | 1.6607939 | 9.208384115 | 4.20E−61 | 1.95E−59 |
| ENSBTAG00000006162 | RAB11FIP5 | 1.658357204 | 5.935269029 | 3.74E−22 | 5.14E−21 |
| ENSBTAG00000035230 | TOR1AIP2 | 1.656653384 | 5.585141371 | 1.45E−18 | 1.65E−17 |
| ENSBTAG00000020589 | ENTPD1 | 1.656181558 | 3.660658331 | 2.48E−06 | 1.05E−05 |
| ENSBTAG00000001805 | TWSG1 | 1.655919327 | 5.875376974 | 1.36E−21 | 1.81E−20 |
| ENSBTAG00000005843 | REXO2 | 1.655742466 | 7.658510756 | 6.57E−43 | 1.92E−41 |
| ENSBTAG00000017747 | PLSCR3 | 1.654907424 | 5.579624056 | 1.08E−18 | 1.25E−17 |
| ENSBTAG00000013744 | SYNPO | 1.65412421 | 4.206428645 | 8.53E−09 | 4.74E−08 |
| ENSBTAG00000011059 | CDKN2C | 1.653323915 | 2.253733762 | 0.018020507 | 0.036471199 |
| ENSBTAG00000017691 | KIAA0040 | 1.653207374 | 2.959253642 | 0.000692924 | 0.001960857 |
| ENSBTAG00000024482 | RABGAP1L | 1.653172163 | 4.159639135 | 1.31E−08 | 7.12E−08 |
| ENSBTAG00000011446 | SEMA7A | 1.652064848 | 5.328219878 | 2.00E−16 | 2.00E−15 |
| ENSBTAG00000046979 | LGMN | 1.651130028 | 6.875232874 | 8.08E−33 | 1.70E−31 |
| ENSBTAG00000021506 | ARL6IP5 | 1.650017829 | 5.991031838 | 1.39E−22 | 1.95E−21 |
| ENSBTAG00000001658 | AKR1D1 | 1.649799581 | 2.129515566 | 0.020265727 | 0.040650631 |
| ENSBTAG00000049730 | TCEAL1 | 1.647970097 | 2.628763472 | 0.003394439 | 0.008343596 |
| ENSBTAG00000011966 | LAMC1 | 1.647542937 | 8.871498041 | 1.29E−57 | 5.49E−56 |
| ENSBTAG00000021393 | FLRT2 | 1.645163527 | 1.922374833 | 0.05898686 | 0.104110743 |
| ENSBTAG00000003953 | ZNF316 | 1.642706088 | 2.259896317 | 0.018020507 | 0.036471199 |
| ENSBTAG00000020762 | HUNK | 1.642463241 | 2.630038647 | 0.003394439 | 0.008343596 |
| ENSBTAG00000023445 | SIAH2 | 1.639651514 | 4.872713831 | 1.38E−12 | 1.07E−11 |
| ENSBTAG00000020684 | ARPC1B | 1.638311661 | 3.212631512 | 0.00014986 | 0.000474729 |
| ENSBTAG00000001042 | MXD1 | 1.636941993 | 5.152565379 | 1.00E−14 | 9.16E−14 |
| ENSBTAG00000000497 | AKR1A1 | 1.635880726 | 6.412776851 | 6.60E−27 | 1.11E−25 |
| ENSBTAG00000021746 | ANXA5 | 1.63510206 | 7.907350951 | 7.77E−45 | 2.39E−43 |
| ENSBTAG00000008886 | STOX2 | 1.634543508 | 2.807698456 | 0.000843798 | 0.002356883 |
| ENSBTAG00000000687 | POC1B | 1.63263255 | 4.671147181 | 5.45E−11 | 3.65E−10 |
| ENSBTAG00000004283 | PPFIBP1 | 1.632384854 | 6.166971821 | 3.91E−24 | 5.93E−23 |
| ENSBTAG00000012694 | UHMK1 | 1.631624148 | 3.65232691 | 3.68E−06 | 1.51E−05 |
| ENSBTAG00000012112 | SLC7A6 | 1.631367394 | 5.199055591 | 5.09E−15 | 4.74E−14 |
| ENSBTAG00000006839 | JOSD2 | 1.627981158 | 2.220618615 | 0.028078466 | 0.054664984 |
| ENSBTAG00000004131 | RAP2C | 1.625803194 | 6.83396729 | 2.21E−31 | 4.44E−30 |
| ENSBTAG00000007389 | IFI35 | 1.622608795 | 4.103049725 | 8.35E−08 | 4.17E−07 |
| ENSBTAG00000024485 | KLHL20 | 1.62125418 | 6.072603129 | 7.28E−23 | 1.04E−21 |
| ENSBTAG00000018395 | KIAA1217 | 1.618870671 | 5.763402933 | 1.45E−19 | 1.76E−18 |
| ENSBTAG00000009048 | EFHD2 | 1.618342767 | 7.760066193 | 3.21E−42 | 9.08E−41 |
| ENSBTAG00000006659 | TMEM116 | 1.617308695 | 2.267440375 | 0.018020507 | 0.036471199 |
| ENSBTAG00000004852 | PLEKHO1 | 1.616861092 | 3.77833544 | 1.96E−06 | 8.41E−06 |
| ENSBTAG00000019076 | PAK6 | 1.616854077 | 4.961003765 | 6.16E−13 | 4.96E−12 |
| ENSBTAG00000017486 | ADARB1 | 1.613689749 | 3.239719888 | 1.00E−04 | 0.00032768 |
| ENSBTAG00000008167 | TTC3 | 1.609259846 | 5.672846275 | 1.41E−18 | 1.60E−17 |
| ENSBTAG00000012330 | B2M | 1.608520931 | 6.447115544 | 2.04E−26 | 3.38E−25 |
| ENSBTAG00000010244 | CLIC5 | 1.608392671 | 1.089058444 | 1 | 1 |
| ENSBTAG00000012858 | NT5C2 | 1.603940753 | 4.990017483 | 5.73E−13 | 4.63E−12 |
| ENSBTAG00000052230 | A0A3Q1MHP0 | 1.603576806 | 1.707596463 | 0.111028651 | 0.178057673 |
| ENSBTAG00000008913 | TMEM98 | 1.603173321 | 3.421266744 | 3.40E−05 | 0.000120389 |
| ENSBTAG00000021182 | DOCK4 | 1.60291642 | 6.060539057 | 4.46E−22 | 6.12E−21 |
| ENSBTAG00000019910 | SDCBP | 1.602132025 | 7.110955232 | 5.03E−34 | 1.12E−32 |
| ENSBTAG00000007036 | SLC17A7 | 1.60114717 | 3.3177753 | 0.000121684 | 0.000393463 |
| ENSBTAG00000003509 | PLEKHA2 | 1.599115214 | 4.814981023 | 1.06E−11 | 7.59E−11 |
| ENSBTAG00000039954 | COPZ2 | 1.598829037 | 2.775930461 | 0.001983888 | 0.005161909 |
| ENSBTAG00000021237 | DST | 1.597788454 | 5.669992526 | 1.87E−18 | 2.11E−17 |
| ENSBTAG00000009596 | C8H9orf43 | 1.596156256 | 2.171653921 | 0.043278561 | 0.078980546 |
| ENSBTAG00000015024 | GXYLT1 | 1.595598777 | 5.138235363 | 6.57E−14 | 5.71E−13 |
| ENSBTAG00000005946 | USP13 | 1.59505079 | 1.729123559 | 0.111028651 | 0.178057673 |
| ENSBTAG00000010899 | TIMP2 | 1.594736806 | 5.140484389 | 6.57E−14 | 5.71E−13 |
| ENSBTAG00000015637 | IMPAD1 | 1.593254698 | 6.602273906 | 9.90E−28 | 1.74E−26 |
| ENSBTAG00000016194 | FBXO32 | 1.590876152 | 3.096799743 | 0.000371659 | 0.00110548 |
| ENSBTAG00000000546 | TOB1 | 1.587476854 | 6.12946249 | 1.71E−22 | 2.39E−21 |
| ENSBTAG00000038241 | REPIN1 | 1.587135095 | 3.999292388 | 4.23E−07 | 1.94E−06 |
| ENSBTAG00000033137 | PSD3 | 1.585444561 | 4.433705208 | 2.57E−09 | 1.50E−08 |
| ENSBTAG00000024219 | TRPV6 | 1.584800837 | 2.880792868 | 0.001168419 | 0.003171208 |
| ENSBTAG00000021969 | KLHL29 | 1.58324975 | 2.043234259 | 0.050825686 | 0.091310673 |
| ENSBTAG00000002820 | RBBP7 | 1.581138491 | 8.61456679 | 1.65E−49 | 5.73E−48 |
| ENSBTAG00000032366 | SLC23A2 | 1.580076812 | 4.622023094 | 2.26E−10 | 1.45E−09 |
| ENSBTAG00000015352 | MTMR4 | 1.580070717 | 7.205995796 | 2.65E−34 | 5.99E−33 |
| ENSBTAG00000015178 | ECI2 | 1.579669315 | 5.409644884 | 8.00E−16 | 7.75E−15 |
| ENSBTAG00000043996 | SAMD12 | 1.579157297 | 3.043917647 | 0.000413547 | 0.001220666 |
| ENSBTAG00000000843 | NDRG2 | 1.578803884 | 6.905016054 | 6.09E−31 | 1.20E−29 |
| ENSBTAG00000050110 | A0A3Q1N924 | 1.577980507 | 3.493318811 | 2.09E−05 | 7.66E−05 |
| ENSBTAG00000010498 | SLC40A1 | 1.57716196 | 6.493995234 | 3.08E−26 | 5.06E−25 |
| ENSBTAG00000000442 | RBP4 | 1.575918716 | 1.960078933 | 0.079010289 | 0.131567201 |
| ENSBTAG00000012708 | SERPINI1 | 1.575360035 | 4.131627744 | 1.11E−07 | 5.48E−07 |
| ENSBTAG00000014249 | IVNS1ABP | 1.575357131 | 9.071179488 | 1.89E−54 | 7.55E−53 |
| ENSBTAG00000007723 | KDSR | 1.572006806 | 5.748477847 | 1.33E−18 | 1.52E−17 |
| ENSBTAG00000044150 | UCK2 | 1.570590791 | 4.787164595 | 2.17E−11 | 1.51E−10 |
| ENSBTAG00000001173 | PLXNA2 | 1.570234016 | 3.025633139 | 0.000621113 | 0.001772693 |
| ENSBTAG00000011744 | USP31 | 1.569811348 | 5.253234076 | 2.05E−14 | 1.84E−13 |
| ENSBTAG00000004177 | TANC1 | 1.569543564 | 4.347415493 | 1.20E−08 | 6.54E−08 |
| ENSBTAG00000020835 | KIAA0513 | 1.569367966 | 4.236660249 | 4.31E−08 | 2.21E−07 |
| ENSBTAG00000003650 | NR4A2 | 1.567420942 | 2.445947119 | 0.013697545 | 0.028852777 |
| ENSBTAG00000000185 | SLC6A14 | 1.564912685 | 2.102245374 | 0.032277735 | 0.060756871 |
| ENSBTAG00000019368 | IGFBP7 | 1.563475752 | 4.144623603 | 1.47E−07 | 7.15E−07 |
| ENSBTAG00000048843 | UBE2W | 1.562983065 | 5.951506342 | 2.75E−20 | 3.44E−19 |
| ENSBTAG00000007952 | MVP | 1.561333278 | 5.76111437 | 2.24E−18 | 2.51E−17 |
| ENSBTAG00000021955 | NPC2 | 1.558021016 | 7.587728346 | 1.23E−37 | 3.10E−36 |
| ENSBTAG00000026307 | ZNF629 | 1.557470408 | 2.331939821 | 0.024168926 | 0.04761392 |
| ENSBTAG00000033221 | CCDC152 | 1.554293907 | 3.829478707 | 3.46E−06 | 1.43E−05 |
| ENSBTAG00000020269 | MISP | 1.552679947 | 3.149731597 | 0.000491785 | 0.001438001 |
| ENSBTAG00000018810 | THBS2 | 1.550831639 | 5.264498766 | 6.38E−14 | 5.56E−13 |
| ENSBTAG00000014883 | GABARAP | 1.547541306 | 7.026778278 | 4.64E−31 | 9.15E−30 |
| ENSBTAG00000006574 | ARL8A | 1.546828324 | 4.180064196 | 1.35E−07 | 6.60E−07 |
| ENSBTAG00000018431 | NAV2 | 1.545377677 | 6.310881106 | 1.11E−23 | 1.65E−22 |
| ENSBTAG00000021660 | IKBIP | 1.544427571 | 4.560705673 | 1.31E−09 | 7.89E−09 |
| ENSBTAG00000025398 | UBD | 1.543886855 | 3.571879681 | 2.52E−05 | 9.13E−05 |
| ENSBTAG00000012046 | JUNB | 1.54312622 | 4.645424991 | 5.49E−10 | 3.40E−09 |
| ENSBTAG00000016473 | NAT1 | 1.541895517 | 2.631281038 | 0.005182791 | 0.012131503 |
| ENSBTAG00000013145 | DDHD2 | 1.541117827 | 5.776550469 | 2.85E−18 | 3.17E−17 |
| ENSBTAG00000019204 | RTN3 | 1.539407246 | 7.802901661 | 3.74E−39 | 9.78E−38 |
| ENSBTAG00000006366 | NFATC4 | 1.536144872 | 3.632852169 | 1.56E−05 | 5.86E−05 |
| ENSBTAG00000007288 | CDK19 | 1.535549666 | 3.487584485 | 5.97E−05 | 0.000203016 |
| ENSBTAG00000001446 | ABRACL | 1.533783891 | 5.577909745 | 2.40E−16 | 2.39E−15 |
| ENSBTAG00000001987 | SWAP70 | 1.533394394 | 8.172068369 | 1.63E−42 | 4.69E−41 |
| ENSBTAG00000006824 | APPL1 | 1.532235545 | 5.88833382 | 4.99E−19 | 5.85E−18 |
| ENSBTAG00000031802 | SPATA16 | 1.530926422 | 2.84556686 | 0.001767854 | 0.004635994 |
| ENSBTAG00000011762 | ANKRD52 | 1.528479892 | 4.823301438 | 4.94E−11 | 3.33E−10 |
| ENSBTAG00000009037 | GALNT12 | 1.528417619 | 6.607821865 | 5.46E−26 | 8.90E−25 |
| ENSBTAG00000004799 | DENND2A | 1.528309453 | 3.559401374 | 3.68E−05 | 0.000129084 |
| ENSBTAG00000020345 | CNN3 | 1.527233075 | 5.69370909 | 3.62E−17 | 3.79E−16 |
| ENSBTAG00000008733 | MAGED1 | 1.526320433 | 7.746732516 | 4.29E−38 | 1.10E−36 |
| ENSBTAG00000050873 | RAB18 | 1.52556945 | 2.767809541 | 0.003974151 | 0.009650475 |
| ENSBTAG00000054239 | KIF1B | 1.523149043 | 5.333513978 | 2.23E−14 | 1.99E−13 |
| ENSBTAG00000016554 | C1H3orf52 | 1.52271186 | 4.522557945 | 2.77E−09 | 1.61E−08 |
| ENSBTAG00000014083 | AMIGO1 | 1.521367134 | 3.281458752 | 0.000262959 | 0.000798018 |
| ENSBTAG00000039968 | PIP4P2 | 1.520360078 | 1.583599817 | 0.290910266 | 0.385192821 |
| ENSBTAG00000018517 | VLDLR | 1.51853545 | 4.134802034 | 2.10E−07 | 9.99E−07 |
| ENSBTAG00000003298 | LZTS2 | 1.516849374 | 3.48639728 | 5.97E−05 | 0.000203016 |
| ENSBTAG00000019026 | EXTL2 | 1.514642868 | 4.877509024 | 4.12E−11 | 2.80E−10 |
| ENSBTAG00000004066 | PARP8 | 1.514067436 | 2.965037591 | 0.001379936 | 0.00370823 |
| ENSBTAG00000011224 | CITED2 | 1.513237765 | 6.359343185 | 3.75E−23 | 5.42E−22 |
| ENSBTAG00000031572 | IFT80 | 1.511550886 | 3.119490231 | 0.000724218 | 0.002041527 |
| ENSBTAG00000013689 | MCTP2 | 1.509368755 | 2.82950445 | 0.002658966 | 0.006666504 |
| ENSBTAG00000005217 | E1BA06 | 1.509326471 | 2.18636431 | 0.043278561 | 0.078980546 |
| ENSBTAG00000031171 | SH3GLB1 | 1.509162341 | 6.153923665 | 5.38E−21 | 6.98E−20 |
| ENSBTAG00000019686 | NCKAP1L | 1.505597782 | 2.320643677 | 0.024168926 | 0.04761392 |
| ENSBTAG00000003876 | MCU | 1.504677476 | 6.456385637 | 5.25E−24 | 7.90E−23 |
| ENSBTAG00000001511 | BCL6 | 1.503352826 | 3.48814491 | 5.97E−05 | 0.000203016 |
| ENSBTAG00000014399 | QSER1 | 1.501858795 | 6.190462433 | 3.25E−21 | 4.26E−20 |
| ENSBTAG00000008688 | HEYL | 1.501829589 | 4.983922104 | 1.19E−11 | 8.45E−11 |
| ENSBTAG00000020983 | RRAS | 1.501352648 | 3.442122655 | 6.66E−05 | 0.000225545 |
| ENSBTAG00000010065 | TRPC5 | 1.501216399 | 2.375867041 | 0.015784905 | 0.032624321 |
| ENSBTAG00000023416 | PPP2R3A | 1.499004515 | 4.999279284 | 1.53E−11 | 1.07E−10 |
| ENSBTAG00000015904 | RORA | 1.498895608 | 2.751915158 | 0.005900325 | 0.0136477 |
| ENSBTAG00000001919 | ZNF652 | 1.498863637 | 3.309287749 | 0.00017924 | 0.000562335 |
| ENSBTAG00000048664 | CDK14 | 1.498819346 | 6.830687408 | 1.37E−27 | 2.38E−26 |
| ENSBTAG00000008607 | ARID3A | 1.498681516 | 7.009469261 | 2.33E−29 | 4.39E−28 |
| ENSBTAG00000015802 | CREB3L2 | 1.498326663 | 5.049709528 | 5.38E−12 | 3.97E−11 |
| ENSBTAG00000006609 | ERRFI1 | 1.497569581 | 6.963310285 | 8.54E−29 | 1.56E−27 |
| ENSBTAG00000012544 | IFNGR1 | 1.494919333 | 5.743447509 | 4.18E−17 | 4.35E−16 |
| ENSBTAG00000040061 | ZBTB38 | 1.494142114 | 3.776697482 | 7.94E−06 | 3.11E−05 |
| ENSBTAG00000009055 | RNF144B | 1.493061277 | 4.672429919 | 9.25E−10 | 5.61E−09 |
| ENSBTAG00000008805 | USP3 | 1.492322828 | 4.730866717 | 4.19E−10 | 2.62E−09 |
| ENSBTAG00000045925 | CITED1 | 1.490832059 | 6.140355708 | 1.80E−20 | 2.27E−19 |
| ENSBTAG00000022288 | AMMECR1 | 1.489215358 | 2.350037153 | 0.015784905 | 0.032624321 |
| ENSBTAG00000051248 | NACAD | 1.488893694 | 2.070103283 | 0.050825686 | 0.091310673 |
| ENSBTAG00000014548 | NIBAN1 | 1.48190998 | 1.728413894 | 0.228981052 | 0.323302376 |
| ENSBTAG00000011828 | ARID3B | 1.481022466 | 5.943001402 | 1.24E−18 | 1.43E−17 |
| ENSBTAG00000020214 | LRRC8B | 1.480169551 | 3.730042143 | 1.64E−05 | 6.14E−05 |
| ENSBTAG00000014685 | HPRT1 | 1.476960833 | 6.115979059 | 4.83E−20 | 5.96E−19 |
| ENSBTAG00000004021 | CDK2 | 1.475896624 | 5.79902789 | 2.80E−17 | 2.96E−16 |
| ENSBTAG00000002914 | GALNT18 | 1.474312258 | 2.086969962 | 0.099050358 | 0.160864976 |
| ENSBTAG00000023929 | FOSL2 | 1.47381334 | 5.823224197 | 3.35E−17 | 3.51E−16 |
| ENSBTAG00000025667 | ASTN2 | 1.47158698 | 3.437966551 | 0.000181646 | 0.000569274 |
| ENSBTAG00000044129 | ST6GALNAC6 | 1.471551405 | 5.02897488 | 1.83E−11 | 1.28E−10 |
| ENSBTAG00000051316 | pseudogene | 1.469904983 | 4.999392037 | 1.53E−11 | 1.07E−10 |
| ENSBTAG00000012387 | PAM | 1.468773566 | 4.899538723 | 1.61E−10 | 1.05E−09 |
| ENSBTAG00000026283 | ASPH | 1.46861986 | 6.458731307 | 5.91E−23 | 8.43E−22 |
| ENSBTAG00000017574 | LMNA | 1.467109309 | 7.952457365 | 1.43E−37 | 3.61E−36 |
| ENSBTAG00000005724 | SYTL4 | 1.46570162 | 3.980130255 | 2.32E−06 | 9.79E−06 |
| ENSBTAG00000006324 | NAB2 | 1.464009832 | 2.058752446 | 0.050825686 | 0.091310673 |
| ENSBTAG00000002688 | ATP1B1 | 1.463315538 | 5.615414986 | 1.80E−15 | 1.70E−14 |
| ENSBTAG00000017629 | BRMS1L | 1.46280276 | 2.14499493 | 0.065911358 | 0.111940932 |
| ENSBTAG00000022471 | TTPA | 1.462268539 | 4.198923125 | 4.49E−07 | 2.06E−06 |
| ENSBTAG00000001460 | LIN7C | 1.459483989 | 7.684859409 | 1.38E−34 | 3.17E−33 |
| ENSBTAG00000037457 | MBD2 | 1.459284316 | 5.091238823 | 6.13E−12 | 4.49E−11 |
| ENSBTAG00000018334 | PLAGL2 | 1.458458381 | 4.28520536 | 1.34E−07 | 6.55E−07 |
| ENSBTAG00000001050 | LDLRAP1 | 1.458283681 | 3.682361838 | 2.63E−05 | 9.50E−05 |
| ENSBTAG00000012405 | PEAR1 | 1.455739123 | 3.042636641 | 0.00121188 | 0.003283697 |
| ENSBTAG00000047739 | TSC22D1 | 1.453736194 | 6.501467666 | 8.85E−23 | 1.25E−21 |
| ENSBTAG00000046869 | RNH1 | 1.45324583 | 7.60288637 | 9.78E−34 | 2.15E−32 |
| ENSBTAG00000010018 | ATP7A | 1.451939178 | 5.500893527 | 2.16E−14 | 1.93E−13 |
| ENSBTAG00000034269 | RCBTB1 | 1.451398004 | 4.416093202 | 3.41E−08 | 1.77E−07 |
| ENSBTAG00000018402 | TSC22D2 | 1.450018869 | 5.784692047 | 1.68E−16 | 1.70E−15 |
| ENSBTAG00000021107 | STXBP5 | 1.449498853 | 6.233410001 | 2.43E−20 | 3.05E−19 |
| ENSBTAG00000005397 | CD46 | 1.44881874 | 10.08780993 | 1.01E−51 | 3.77E−50 |
| ENSBTAG00000006620 | SLC24A4 | 1.447626739 | 2.071927975 | 0.099050358 | 0.160864976 |
| ENSBTAG00000015910 | ITGB1 | 1.446717181 | 10.26693791 | 3.02E−52 | 1.15E−50 |
| ENSBTAG00000000569 | HES1 | 1.443941666 | 4.949726947 | 1.03E−10 | 6.76E−10 |
| ENSBTAG00000000838 | CCDC88C | 1.442915171 | 2.602682602 | 0.014983817 | 0.031395378 |
| ENSBTAG00000008361 | PON2 | 1.442268115 | 5.265758689 | 7.84E−13 | 6.26E−12 |
| ENSBTAG00000008969 | SLC9A8 | 1.441547345 | 3.541488373 | 5.34E−05 | 0.000183081 |
| ENSBTAG00000017529 | CA8 | 1.440813326 | 3.805961108 | 1.86E−05 | 6.87E−05 |
| ENSBTAG00000020418 | CA5B | 1.440585196 | 3.950856719 | 4.66E−06 | 1.88E−05 |
| ENSBTAG00000049490 | RABGAP1L | 1.439511348 | 3.37753335 | 0.000297514 | 0.000897684 |
| ENSBTAG00000014205 | PRKAR2A | 1.438615385 | 6.604256725 | 1.59E−23 | 2.32E−22 |
| ENSBTAG00000037962 | MAML2 | 1.437871485 | 3.941345273 | 3.66E−06 | 1.51E−05 |
| ENSBTAG00000006515 | ESPN | 1.436047068 | 3.294213344 | 0.000489492 | 0.001432153 |
| ENSBTAG00000012219 | CSPG4 | 1.432976793 | 2.010992711 | 0.079010289 | 0.131567201 |
| ENSBTAG00000011284 | SLC39A1 | 1.432516767 | 7.497557354 | 1.07E−31 | 2.18E−30 |
| ENSBTAG00000017124 | GPSM2 | 1.431531203 | 5.175607614 | 7.63E−12 | 5.50E−11 |
| ENSBTAG00000011190 | FLNA | 1.431071273 | 9.554190053 | 2.46E−48 | 8.28E−47 |
| ENSBTAG00000016549 | CARMIL1 | 1.430395873 | 7.290194258 | 8.01E−30 | 1.53E−28 |
| ENSBTAG00000012482 | CDCP1 | 1.428434524 | 5.666867121 | 1.92E−15 | 1.82E−14 |
| ENSBTAG00000017571 | PLEKHA5 | 1.428201965 | 6.715684091 | 3.79E−24 | 5.75E−23 |
| ENSBTAG00000014127 | PTGS2 | 1.427743462 | 12.67728519 | 3.16E−55 | 1.29E−53 |
| ENSBTAG00000050016 | COX7B2 | 1.426666327 | 3.036424171 | 0.001778683 | 0.004661891 |
| ENSBTAG00000033315 | DNAJC1 | 1.426073241 | 5.991963809 | 9.32E−18 | 1.00E−16 |
| ENSBTAG00000017215 | WDR76 | 1.425859284 | 3.857182821 | 1.17E−05 | 4.45E−05 |
| ENSBTAG00000001956 | HINT3 | 1.425551949 | 4.172577521 | 8.82E−07 | 3.92E−06 |
| ENSBTAG00000017639 | RPS6KA3 | 1.424138952 | 5.178815216 | 5.70E−12 | 4.19E−11 |
| ENSBTAG00000047856 | PAK2 | 1.423561146 | 7.779912543 | 4.76E−34 | 1.06E−32 |
| ENSBTAG00000052611 | GUSB | 1.423069844 | 2.751467041 | 0.005900325 | 0.0136477 |
| ENSBTAG00000021308 | IRS1 | 1.423067903 | 4.120438122 | 1.09E−06 | 4.77E−06 |
| ENSBTAG00000016823 | CSNK1G1 | 1.422778608 | 4.487521861 | 3.62E−08 | 1.88E−07 |
| ENSBTAG00000016963 | ADAMTS6 | 1.422515133 | 2.781279188 | 0.003974151 | 0.009650475 |
| ENSBTAG00000005534 | ENO3 | 1.422411277 | 5.232215422 | 2.65E−12 | 2.01E−11 |
| ENSBTAG00000017530 | PLGRKT | 1.41998893 | 5.202692879 | 6.70E−12 | 4.89E−11 |
| ENSBTAG00000020272 | SIK2 | 1.417571511 | 5.57930797 | 2.03E−14 | 1.82E−13 |
| ENSBTAG00000012090 | GGTA1 | 1.417302828 | 7.773686869 | 5.85E−34 | 1.30E−32 |
| ENSBTAG00000019866 | NRP1 | 1.414540582 | 2.635027758 | 0.010150436 | 0.022045017 |
| ENSBTAG00000013018 | AMPD2 | 1.412846662 | 4.668677313 | 5.42E−09 | 3.06E−08 |
| ENSBTAG00000010152 | MID1 | 1.412216298 | 5.165187077 | 1.36E−11 | 9.66E−11 |
| ENSBTAG00000016415 | LTA4H | 1.410689593 | 6.487727679 | 1.35E−21 | 1.81E−20 |
| ENSBTAG00000010395 | DOCK9 | 1.410312708 | 8.587373914 | 4.03E−40 | 1.08E−38 |
| ENSBTAG00000010843 | PGRMC2 | 1.409724292 | 8.038896233 | 1.01E−35 | 2.41E−34 |
| ENSBTAG00000021100 | PPP1R3F | 1.409464187 | 2.399641449 | 0.030907879 | 0.05970313 |
| ENSBTAG00000020958 | LRRC8D | 1.408194253 | 6.775496507 | 4.49E−24 | 6.77E−23 |
| ENSBTAG00000005788 | VANGL1 | 1.408183402 | 4.650373718 | 5.90E−09 | 3.33E−08 |
| ENSBTAG00000010786 | TACC2 | 1.406990508 | 5.481944452 | 1.12E−13 | 9.56E−13 |
| ENSBTAG00000003530 | DDX31 | 1.405908693 | 6.763040955 | 6.61E−24 | 9.94E−23 |
| ENSBTAG00000022681 | EOGT | 1.404847936 | 5.223398256 | 7.82E−12 | 5.63E−11 |
| ENSBTAG00000050161 | LY6K | 1.404190969 | 2.672973849 | 0.006821752 | 0.015584835 |
| ENSBTAG00000016484 | ATP11C | 1.403744482 | 4.848921741 | 1.09E−09 | 6.60E−09 |
| ENSBTAG00000048459 | CARHSP1 | 1.402218056 | 5.204004218 | 6.70E−12 | 4.89E−11 |
| ENSBTAG00000051765 | | 1.39953412 | 2.654694619 | 0.010150436 | 0.022045017 |
| ENSBTAG00000031569 | GOLM1 | 1.398329584 | 5.109561363 | 3.00E−11 | 2.06E−10 |
| ENSBTAG00000004216 | MAP3K2 | 1.397661603 | 3.845937018 | 1.66E−05 | 6.19E−05 |
| ENSBTAG00000014670 | BEX5 | 1.39757779 | 3.856516144 | 1.17E−05 | 4.45E−05 |
| ENSBTAG00000006372 | KLHL42 | 1.395455032 | 3.553303852 | 9.85E−05 | 0.000322955 |
| ENSBTAG00000007415 | SLC7A8 | 1.393511823 | 8.620683868 | 1.54E−39 | 4.04E−38 |
| ENSBTAG00000011540 | SPG21 | 1.392372992 | 4.711849058 | 6.23E−09 | 3.51E−08 |
| ENSBTAG00000019342 | OSBPL3 | 1.391228604 | 6.410014854 | 2.37E−20 | 2.98E−19 |
| ENSBTAG00000055208 | AKR1B1 | 1.391065694 | 4.678464773 | 9.29E−09 | 5.15E−08 |
| ENSBTAG00000031295 | HSDL2 | 1.38986675 | 5.594913943 | 4.07E−14 | 3.59E−13 |
| ENSBTAG00000015808 | ZNF609 | 1.388672683 | 5.471736257 | 1.93E−13 | 1.62E−12 |
| ENSBTAG00000054434 | lncRNA | 1.386494914 | 1.802160516 | 0.148266661 | 0.221448789 |
| ENSBTAG00000002566 | PGPEP1 | 1.386290003 | 4.399546295 | 1.61E−07 | 7.75E−07 |
| ENSBTAG00000002539 | TRIOBP | 1.38544288 | 6.59897358 | 5.85E−22 | 7.99E−21 |
| ENSBTAG00000050550 | FAM110B | 1.385046044 | 2.405222344 | 0.030907879 | 0.05970313 |
| ENSBTAG00000010444 | ERBB3 | 1.38339651 | 8.271267416 | 1.70E−36 | 4.20E−35 |
| ENSBTAG00000002715 | GMPR2 | 1.383198516 | 3.622664048 | 0.000108865 | 0.000354592 |
| ENSBTAG00000019472 | NR3C1 | 1.381801508 | 4.334953164 | 3.77E−07 | 1.74E−06 |
| ENSBTAG00000039711 | ZFP62 | 1.381729436 | 2.191941456 | 0.081278796 | 0.13479377 |
| ENSBTAG00000000855 | NXN | 1.381687262 | 6.341907459 | 1.26E−19 | 1.53E−18 |
| ENSBTAG00000020166 | ZNFX1 | 1.381456226 | 6.123840718 | 5.75E−18 | 6.26E−17 |
| ENSBTAG00000039466 | CDR2L | 1.380195721 | 4.340410036 | 2.72E−07 | 1.28E−06 |
| ENSBTAG00000015930 | DNAL1 | 1.379263045 | 2.456779825 | 0.038117617 | 0.070474107 |
| ENSBTAG00000016723 | GFI1 | 1.378609963 | 1.686027859 | 0.346056464 | 0.44594652 |
| ENSBTAG00000037819 | SFT2D2 | 1.377580293 | 5.06136107 | 1.39E−10 | 9.04E−10 |
| ENSBTAG00000054222 | SVIP | 1.37723527 | 3.636074655 | 0.000108865 | 0.000354592 |
| ENSBTAG00000019956 | CMPK1 | 1.37711926 | 6.46443499 | 1.34E−20 | 1.71E−19 |
| ENSBTAG00000003989 | GSTO1 | 1.376559001 | 4.085190017 | 3.00E−06 | 1.25E−05 |
| ENSBTAG00000025822 | CALML4 | 1.376272161 | 2.179409683 | 0.043278561 | 0.078980546 |
| ENSBTAG00000047274 | KIF21B | 1.372217964 | 3.775453956 | 3.73E−05 | 0.000130692 |
| ENSBTAG00000003661 | SLC35F2 | 1.370420663 | 5.484173979 | 6.22E−13 | 5.00E−12 |
| ENSBTAG00000031810 | WDR88 | 1.370137988 | 2.332134831 | 0.045727836 | 0.083016484 |
| ENSBTAG00000011698 | TYK2 | 1.36915829 | 5.985787734 | 1.50E−16 | 1.52E−15 |
| ENSBTAG00000043971 | NOTCH3 | 1.368915447 | 5.038378097 | 2.68E−10 | 1.71E−09 |
| ENSBTAG00000008613 | TCP11L1 | 1.368063448 | 3.544395178 | 0.000140875 | 0.000448787 |
| ENSBTAG00000012804 | REEP5 | 1.36665714 | 8.50932948 | 2.11E−37 | 5.28E−36 |
| ENSBTAG00000012253 | AGO1 | 1.366111631 | 7.819892682 | 4.32E−32 | 8.86E−31 |
| ENSBTAG00000020126 | MYO10 | 1.36559603 | 5.535738571 | 2.34E−13 | 1.96E−12 |
| ENSBTAG00000000357 | PRAG1 | 1.364649703 | 5.192709279 | 2.61E−11 | 1.81E−10 |
| ENSBTAG00000039153 | RPS6KA4 | 1.364139278 | 4.091373471 | 3.00E−06 | 1.25E−05 |
| ENSBTAG00000003581 | SETD7 | 1.361257801 | 5.747327038 | 1.18E−14 | 1.07E−13 |
| ENSBTAG00000048271 | MICAL3 | 1.359768765 | 6.003459195 | 1.39E−16 | 1.41E−15 |
| ENSBTAG00000001306 | FXN | 1.358653953 | 5.504950964 | 4.48E−13 | 3.67E−12 |
| ENSBTAG00000008958 | NSMAF | 1.356751758 | 4.59237243 | 5.28E−08 | 2.69E−07 |
| ENSBTAG00000045757 | TNNC1 | 1.355986181 | 2.246579067 | 0.05489969 | 0.097808395 |
| ENSBTAG00000013801 | PBX1 | 1.354957115 | 2.653247376 | 0.010150436 | 0.022045017 |
| ENSBTAG00000032996 | P4HA1 | 1.354183664 | 7.08334077 | 2.30E−25 | 3.67E−24 |
| ENSBTAG00000018955 | SOHLH2 | 1.353413209 | 3.094149485 | 0.001547391 | 0.004118981 |
| ENSBTAG00000004995 | C1GALT1 | 1.353334972 | 5.847035279 | 2.82E−15 | 2.65E−14 |
| ENSBTAG00000013957 | GALNT3 | 1.352866098 | 8.082190529 | 1.27E−33 | 2.77E−32 |
| ENSBTAG00000021341 | BTBD1 | 1.352467547 | 5.083408648 | 2.11E−10 | 1.35E−09 |
| ENSBTAG00000046936 | MRFAP1L1 | 1.351733979 | 8.192188026 | 2.20E−34 | 5.01E−33 |
| ENSBTAG00000014027 | TRIM36 | 1.351663171 | 4.342011658 | 6.45E−07 | 2.91E−06 |
| ENSBTAG00000010909 | SLC30A1 | 1.351254824 | 7.321183721 | 2.58E−27 | 4.44E−26 |
| ENSBTAG00000007809 | PPP1R36 | 1.350403276 | 2.607421318 | 0.014983817 | 0.031395378 |
| ENSBTAG00000015354 | ZDHHC9 | 1.34974674 | 7.496073709 | 1.06E−28 | 1.93E−27 |
| ENSBTAG00000044063 | B4GALT6 | 1.349200928 | 6.592649677 | 6.05E−21 | 7.83E−20 |
| ENSBTAG00000003072 | ACADVL | 1.344222907 | 6.305563852 | 1.42E−18 | 1.62E−17 |
| ENSBTAG00000014377 | CHD3 | 1.3441991 | 6.079141021 | 7.75E−17 | 7.95E−16 |
| ENSBTAG00000004023 | KIAA1324L | 1.343412862 | 2.958692981 | 0.005432627 | 0.012669796 |
| ENSBTAG00000013472 | COL1A2 | 1.342475344 | 6.838906672 | 6.40E−23 | 9.13E−22 |
| ENSBTAG00000000656 | NFATC1 | 1.340338115 | 4.172976353 | 2.96E−06 | 1.24E−05 |
| ENSBTAG00000030319 | GBGT1 | 1.33918939 | 5.650880351 | 1.01E−13 | 8.68E−13 |
| ENSBTAG00000002736 | DNMT1 | 1.338865755 | 5.299407663 | 1.20E−11 | 8.52E−11 |
| ENSBTAG00000015457 | FGFR1 | 1.33836416 | 3.789256864 | 6.47E−05 | 0.000219078 |
| ENSBTAG00000053854 | ABHD14A | 1.338301921 | 1.6949577 | 0.346056464 | 0.44594652 |
| ENSBTAG00000026913 | HHIPL1 | 1.337947146 | 2.778363298 | 0.010803314 | 0.023346428 |
| ENSBTAG00000021224 | ZSWIM6 | 1.33570385 | 3.859681181 | 2.91E−05 | 0.00010437 |
| ENSBTAG00000009181 | INPP5A | 1.33541663 | 6.596939935 | 1.28E−20 | 1.63E−19 |
| ENSBTAG00000001895 | PGGT1B | 1.335371679 | 5.390847953 | 7.30E−12 | 5.28E−11 |
| ENSBTAG00000051479 | GLS | 1.333549454 | 3.916265746 | 3.15E−05 | 0.000112483 |
| ENSBTAG00000038929 | BCAS4 | 1.330033126 | 4.1609649 | 4.10E−06 | 1.68E−05 |
| ENSBTAG00000009552 | ATP2B1 | 1.32909715 | 8.204768902 | 1.81E−33 | 3.90E−32 |
| ENSBTAG00000010397 | B3GNT5 | 1.327926803 | 5.067192708 | 4.95E−10 | 3.08E−09 |
| ENSBTAG00000020412 | RAP2A | 1.326893485 | 5.411017425 | 5.21E−12 | 3.86E−11 |
| ENSBTAG00000018784 | CTSZ | 1.323899665 | 7.792259574 | 5.65E−30 | 1.08E−28 |
| ENSBTAG00000021343 | ARHGEF12 | 1.323781788 | 6.132697847 | 1.11E−16 | 1.13E−15 |
| ENSBTAG00000007678 | MKX | 1.323470752 | 4.287777997 | 1.87E−06 | 8.02E−06 |
| ENSBTAG00000009965 | ECI1 | 1.322931882 | 3.909174789 | 3.15E−05 | 0.000112483 |
| ENSBTAG00000005709 | ABHD3 | 1.322792715 | 2.007235844 | 0.079010289 | 0.131567201 |
| ENSBTAG00000005278 | WEE1 | 1.318229365 | 6.977135814 | 3.28E−23 | 4.74E−22 |
| ENSBTAG00000010745 | THRA | 1.317762816 | 3.284227906 | 0.001758254 | 0.004617243 |
| ENSBTAG00000009902 | AKR1B1 | 1.317385242 | 8.562230256 | 2.11E−35 | 4.92E−34 |
| ENSBTAG00000014135 | WDR17 | 1.316878525 | 2.473793753 | 0.026042006 | 0.051077575 |
| ENSBTAG00000034626 | FBXO16 | 1.316558331 | 1.807812016 | 0.148266661 | 0.221448789 |
| ENSBTAG00000038062 | FUT4 | 1.316060744 | 4.368885733 | 5.78E−07 | 2.63E−06 |
| ENSBTAG00000014486 | ATP12A | 1.315079488 | 7.54939936 | 8.21E−28 | 1.45E−26 |
| ENSBTAG00000003012 | TRAF1 | 1.31463511 | 3.514694662 | 0.000285207 | 0.000863391 |
| ENSBTAG00000016936 | MISP3 | 1.314280688 | 3.020580045 | 0.004639589 | 0.010970312 |
| ENSBTAG00000022242 | DPP3 | 1.309602136 | 6.798163869 | 1.44E−21 | 1.92E−20 |
| ENSBTAG00000051440 | lncRNA | 1.308799895 | 2.409318781 | 0.030907879 | 0.05970313 |
| ENSBTAG00000014250 | ZC3HAV1L | 1.307521968 | 4.491536067 | 3.36E−07 | 1.56E−06 |
| ENSBTAG00000018229 | NFIX | 1.307090894 | 1.993971897 | 0.079010289 | 0.131567201 |
| ENSBTAG00000012295 | NKD1 | 1.306891545 | 2.537357149 | 0.031810919 | 0.060001382 |
| ENSBTAG00000006165 | PRDX4 | 1.305663351 | 6.021503608 | 1.38E−15 | 1.32E−14 |
| ENSBTAG00000006757 | CA11 | 1.304967005 | 1.684687186 | 0.346056464 | 0.44594652 |
| ENSBTAG00000016910 | EEF1AKMT3 | 1.304870017 | 3.290939481 | 0.001243815 | 0.003366495 |
| ENSBTAG00000043565 | Mt tRNA | 1.304233162 | 3.518913567 | 0.000492341 | 0.001438765 |
| ENSBTAG00000010964 | STARD5 | 1.303834951 | 3.102547255 | 0.002778785 | 0.006941993 |
| ENSBTAG00000020931 | CHN2 | 1.302200198 | 3.18686716 | 0.001672133 | 0.004411797 |
| ENSBTAG00000011424 | TPM2 | 1.301253049 | 3.753997272 | 0.000125529 | 0.000405448 |
| ENSBTAG00000051809 | STMP1 | 1.300305178 | 6.796402943 | 2.90E−21 | 3.81E−20 |
| ENSBTAG00000025434 | ZFP36L1 | 1.296560897 | 6.972852009 | 1.46E−22 | 2.05E−21 |
| ENSBTAG00000010866 | BMPER | 1.296126876 | 1.885902334 | 0.182431139 | 0.26547662 |
| ENSBTAG00000040585 | NR6A1 | 1.295602475 | 5.743030392 | 1.52E−13 | 1.28E−12 |
| ENSBTAG00000019338 | IL13RA1 | 1.294685124 | 6.700706873 | 2.09E−20 | 2.63E−19 |
| ENSBTAG00000019376 | FNDC3B | 1.294032946 | 7.804799054 | 4.66E−29 | 8.65E−28 |
| ENSBTAG00000019404 | IL10RB | 1.293525576 | 6.051141345 | 1.42E−15 | 1.36E−14 |
| ENSBTAG00000004949 | FDXACB1 | 1.292322874 | 1.872914293 | 0.182431139 | 0.26547662 |
| ENSBTAG00000047591 | C23H6orf141 | 1.291177379 | 3.629367327 | 0.000264867 | 0.000803311 |
| ENSBTAG00000020824 | KRT10 | 1.290649599 | 1.962654693 | 0.121053183 | 0.191775369 |
| ENSBTAG00000013060 | IQGAP1 | 1.289862383 | 9.138924028 | 5.10E−38 | 1.30E−36 |
| ENSBTAG00000003864 | SLC13A4 | 1.287249536 | 7.733788404 | 4.69E−28 | 8.36E−27 |
| ENSBTAG00000004115 | MYLIP | 1.287048869 | 5.244609784 | 1.51E−10 | 9.78E−10 |
| ENSBTAG00000010394 | MCF2L2 | 1.286837586 | 2.013793572 | 0.146653336 | 0.219631365 |
| ENSBTAG00000014530 | NTNG1 | 1.286601492 | 2.719669084 | 0.022248279 | 0.044174294 |
| ENSBTAG00000027809 | RABGAP1L | 1.286249056 | 3.057447584 | 0.005617268 | 0.013056809 |
| ENSBTAG00000008860 | RIPOR1 | 1.286189845 | 6.091007398 | 1.12E−15 | 1.08E−14 |
| ENSBTAG00000023372 | MYCN | 1.283942342 | 6.140050457 | 4.58E−16 | 4.49E−15 |
| ENSBTAG00000005936 | DMC1 | 1.282642811 | 2.233201066 | 0.05489969 | 0.097808395 |
| ENSBTAG00000013736 | PROM1 | 1.2786993 | 5.510557931 | 9.32E−12 | 6.68E−11 |
| ENSBTAG00000016204 | C1RL | 1.278457939 | 1.923161603 | 0.182431139 | 0.26547662 |
| ENSBTAG00000006413 | KATNBL1 | 1.277750849 | 3.948945534 | 3.83E−05 | 0.00013432 |
| ENSBTAG00000037907 | DSEL | 1.277538507 | 5.074717877 | 1.68E−09 | 9.98E−09 |
| ENSBTAG00000015334 | ZHX1 | 1.276665167 | 4.420036948 | 1.05E−06 | 4.63E−06 |
| ENSBTAG00000011011 | SSH2 | 1.276212465 | 4.221456488 | 5.40E−06 | 2.17E−05 |
| ENSBTAG00000003199 | SLC35C1 | 1.274501176 | 6.870636298 | 4.18E−21 | 5.44E−20 |
| ENSBTAG00000005165 | CSGALNACT2 | 1.272893185 | 4.852778152 | 2.59E−08 | 1.36E−07 |
| ENSBTAG00000018902 | ZC2HC1A | 1.272228217 | 4.526610902 | 4.94E−07 | 2.26E−06 |
| ENSBTAG00000001826 | SASH1 | 1.270636205 | 5.799400467 | 1.34E−13 | 1.14E−12 |
| ENSBTAG00000002175 | SRPK2 | 1.269598238 | 5.427259069 | 3.15E−11 | 2.16E−10 |
| ENSBTAG00000054072 | ADM2 | 1.269532861 | 4.326819347 | 2.76E−06 | 1.16E−05 |
| ENSBTAG00000002998 | MELTF | 1.269352973 | 2.274785099 | 0.05489969 | 0.097808395 |
| ENSBTAG00000004423 | ARHGAP42 | 1.267031813 | 4.867373047 | 2.34E−08 | 1.24E−07 |
| ENSBTAG00000051782 | ATAD2 | 1.266926508 | 3.388829718 | 0.001297258 | 0.003504331 |
| ENSBTAG00000021632 | MSRA | 1.265967835 | 5.105058283 | 1.84E−09 | 1.09E−08 |
| ENSBTAG00000006663 | CYTH1 | 1.26496523 | 4.536110168 | 5.94E−07 | 2.69E−06 |
| ENSBTAG00000002626 | ARHGAP6 | 1.264478329 | 1.838793444 | 0.269789176 | 0.361699985 |
| ENSBTAG00000020458 | STX6 | 1.264056301 | 4.71284651 | 1.14E−07 | 5.62E−07 |
| ENSBTAG00000007622 | CTSD | 1.263678116 | 11.00318369 | 3.12E−42 | 8.86E−41 |
| ENSBTAG00000015182 | STARD10 | 1.263585094 | 5.072125436 | 2.22E−09 | 1.30E−08 |
| ENSBTAG00000008704 | FIGN | 1.262871893 | 2.858122476 | 0.009169522 | 0.02013314 |
| ENSBTAG00000005514 | TRIO | 1.262612474 | 6.796112652 | 3.13E−20 | 3.88E−19 |
| ENSBTAG00000032122 | CD19 | 1.262567277 | 3.413430767 | 0.000925026 | 0.002571035 |
| ENSBTAG00000013910 | POLR3GL | 1.262447955 | 1.976693533 | 0.121053183 | 0.191775369 |
| ENSBTAG00000034113 | GALNT10 | 1.262178845 | 5.651921877 | 1.58E−12 | 1.22E−11 |
| ENSBTAG00000012677 | ARHGAP12 | 1.261182053 | 6.668476075 | 3.41E−19 | 4.05E−18 |
| ENSBTAG00000001457 | TMEM267 | 1.261110103 | 3.39208577 | 0.001297258 | 0.003504331 |
| ENSBTAG00000005929 | SPTBN2 | 1.261044008 | 5.184492412 | 8.97E−10 | 5.45E−09 |
| ENSBTAG00000025868 | TLN1 | 1.258885729 | 8.520904972 | 2.34E−32 | 4.85E−31 |
| ENSBTAG00000034496 | Sem1 | 1.258814949 | 3.424359468 | 0.000925026 | 0.002571035 |
| ENSBTAG00000005814 | PSME2 | 1.25798306 | 5.543657117 | 9.60E−12 | 6.88E−11 |
| ENSBTAG00000016462 | TCF4 | 1.257187611 | 3.643944259 | 0.000443323 | 0.00130305 |
| ENSBTAG00000020244 | EFNA1 | 1.256796206 | 6.204629799 | 6.84E−16 | 6.65E−15 |
| ENSBTAG00000024663 | LAD1 | 1.256602919 | 6.056552462 | 7.28E−15 | 6.70E−14 |
| ENSBTAG00000001668 | WNT7A | 1.256316065 | 2.32036911 | 0.066893327 | 0.113319985 |
| ENSBTAG00000003245 | BCAR3 | 1.253185115 | 5.146250636 | 1.40E−09 | 8.41E−09 |
| ENSBTAG00000004440 | SLC15A1 | 1.250684801 | 4.950018608 | 1.19E−08 | 6.48E−08 |
| ENSBTAG00000002614 | RUFY2 | 1.250680964 | 4.748705431 | 1.23E−07 | 6.03E−07 |
| ENSBTAG00000013329 | AP1M1 | 1.250364448 | 4.337077906 | 3.32E−06 | 1.38E−05 |
| ENSBTAG00000015309 | CFAP300 | 1.250269272 | 2.76951454 | 0.015565713 | 0.032526329 |
| ENSBTAG00000001932 | CRAT | 1.249379819 | 6.456161667 | 2.97E−17 | 3.13E−16 |
| ENSBTAG00000020330 | BTG1 | 1.248182917 | 7.855625446 | 1.46E−27 | 2.54E−26 |
| ENSBTAG00000046553 | SECISBP2L | 1.246093021 | 6.392767753 | 5.41E−17 | 5.59E−16 |
| ENSBTAG00000000899 | TMEM17 | 1.2449099 | 2.632785658 | 0.026583932 | 0.052001404 |
| ENSBTAG00000017306 | AKNA | 1.2445563 | 3.719581744 | 0.000200163 | 0.000624239 |
| ENSBTAG00000013007 | FAM210B | 1.24450728 | 6.710150819 | 4.49E−19 | 5.29E−18 |
| ENSBTAG00000023074 | C28H1orf198 | 1.243155746 | 4.034196241 | 3.56E−05 | 0.00012525 |
| ENSBTAG00000003498 | FAT2 | 1.243151185 | 2.010761505 | 0.146653336 | 0.219631365 |
| ENSBTAG00000016103 | CBFB | 1.241382671 | 5.895493575 | 1.62E−13 | 1.37E−12 |
| ENSBTAG00000002178 | PPL | 1.238421319 | 5.881626763 | 1.84E−13 | 1.55E−12 |
| ENSBTAG00000013990 | WNT6 | 1.237450449 | 5.869417735 | 2.46E−13 | 2.06E−12 |
| ENSBTAG00000046337 | TUBB6 | 1.236758082 | 5.214842699 | 1.16E−09 | 7.01E−09 |
| ENSBTAG00000012289 | AKR7A2 | 1.236662648 | 4.31224387 | 3.75E−06 | 1.54E−05 |
| ENSBTAG00000048308 | ICMT | 1.236626752 | 6.001664633 | 4.27E−14 | 3.77E−13 |
| ENSBTAG00000050091 | GCNT4 | 1.236063557 | 6.318897989 | 3.26E−16 | 3.23E−15 |
| ENSBTAG00000051373 | AKR1B1 | 1.235659703 | 4.755021634 | 9.23E−08 | 4.59E−07 |
| ENSBTAG00000039599 | HOXB4 | 1.234313546 | 3.543997044 | 0.000824939 | 0.002309042 |
| ENSBTAG00000008502 | AC093227.2 | 1.233437046 | 1.802820402 | 0.148266661 | 0.221448789 |
| ENSBTAG00000004194 | RALGDS | 1.232134344 | 5.882157663 | 2.73E−13 | 2.26E−12 |
| ENSBTAG00000003183 | NTN4 | 1.231563694 | 1.716277635 | 0.346056464 | 0.44594652 |
| ENSBTAG00000033621 | SMCO4 | 1.231039387 | 5.197639118 | 1.66E−09 | 9.85E−09 |
| ENSBTAG00000049546 | NA | 1.230945297 | 2.126790955 | 0.099050358 | 0.160864976 |
| ENSBTAG00000049835 | lncRNA | 1.230774274 | 1.649514963 | 0.181536995 | 0.264698588 |
| ENSBTAG00000016506 | ST3GAL4 | 1.229992391 | 4.486543693 | 1.62E−06 | 6.97E−06 |
| ENSBTAG00000007567 | TMX3 | 1.229206519 | 6.93498557 | 2.87E−20 | 3.58E−19 |
| ENSBTAG00000005546 | HOXB7 | 1.228800806 | 3.508658681 | 0.000688479 | 0.001949783 |
| ENSBTAG00000019345 | RFK | 1.228502554 | 5.066557788 | 6.00E−09 | 3.38E−08 |
| ENSBTAG00000010389 | STBD1 | 1.227850844 | 4.377104609 | 3.52E−06 | 1.45E−05 |
| ENSBTAG00000001117 | ANKRD50 | 1.225315699 | 4.564684575 | 8.49E−07 | 3.78E−06 |
| ENSBTAG00000019436 | SLC39A13 | 1.223839775 | 3.492384376 | 0.000958794 | 0.002656836 |
| ENSBTAG00000001700 | CDC42 | 1.223137927 | 9.128378333 | 2.32E−34 | 5.28E−33 |
| ENSBTAG00000019269 | COL6A2 | 1.222967677 | 3.222917839 | 0.004816978 | 0.011349496 |
| ENSBTAG00000013017 | GNAT2 | 1.222716524 | 3.562535962 | 0.000594263 | 0.001703352 |
| ENSBTAG00000012724 | AJUBA | 1.222361147 | 3.541392341 | 0.000824939 | 0.002309042 |
| ENSBTAG00000000061 | PCDH7 | 1.22214539 | 2.135789491 | 0.118721218 | 0.188818489 |
| ENSBTAG00000008732 | ZC3H12C | 1.220688718 | 2.488001122 | 0.038117617 | 0.070474107 |
| ENSBTAG00000003947 | SSBP4 | 1.219559897 | 3.151465486 | 0.003384965 | 0.008324495 |
| ENSBTAG00000013127 | LPCAT3 | 1.219201489 | 7.11321442 | 2.80E−21 | 3.68E−20 |
| ENSBTAG00000018400 | BRMS1L | 1.218844056 | 4.08224628 | 3.72E−05 | 0.000130412 |
| ENSBTAG00000020266 | SLC19A2 | 1.217449946 | 5.640825267 | 1.21E−11 | 8.57E−11 |
| ENSBTAG00000020311 | USP20 | 1.217217782 | 5.09239872 | 6.47E−09 | 3.64E−08 |
| ENSBTAG00000007213 | SIRPA | 1.214121176 | 4.01519664 | 6.62E−05 | 0.000224183 |
| ENSBTAG00000003345 | FAT4 | 1.213752238 | 1.572884709 | 0.290910266 | 0.385192821 |
| ENSBTAG00000002391 | TGFB1I1 | 1.212516704 | 1.855633968 | 0.269789176 | 0.361699985 |
| ENSBTAG00000008705 | CABLES1 | 1.211779609 | 3.238309623 | 0.00346083 | 0.008493975 |
| ENSBTAG00000001186 | SH2D4A | 1.210524696 | 4.112153192 | 3.25E−05 | 0.000115927 |
| ENSBTAG00000006197 | ZDHHC21 | 1.210416256 | 4.149309042 | 2.85E−05 | 0.000102419 |
| ENSBTAG00000019533 | ADTRP | 1.210194287 | 1.958124412 | 0.121053183 | 0.191775369 |
| ENSBTAG00000004051 | SBDS | 1.208477431 | 7.171430297 | 2.73E−21 | 3.60E−20 |
| ENSBTAG00000016722 | PGGHG | 1.207603817 | 2.659797723 | 0.026583932 | 0.052001404 |
| ENSBTAG00000038536 | AC104389.5 | 1.206810197 | 2.400773473 | 0.05521166 | 0.098251567 |
| ENSBTAG00000018189 | CSTB | 1.205534334 | 6.920081416 | 1.48E−19 | 1.79E−18 |
| ENSBTAG00000032711 | HTATSF1 | 1.205487993 | 3.070127925 | 0.005617268 | 0.013056809 |
| ENSBTAG00000049591 | ITSN2 | 1.204356483 | 1.947814892 | 0.121053183 | 0.191775369 |
| ENSBTAG00000017053 | ABCB10 | 1.203684746 | 6.247528431 | 5.00E−15 | 4.66E−14 |
| ENSBTAG00000008429 | LRP4 | 1.202909691 | 4.595958923 | 7.57E−07 | 3.39E−06 |
| ENSBTAG00000003330 | ATL3 | 1.201533431 | 6.711248329 | 5.45E−18 | 5.96E−17 |
| ENSBTAG00000012442 | CTSB | 1.200959111 | 9.236248202 | 1.05E−33 | 2.30E−32 |
| ENSBTAG00000046332 | ACTA1 | 1.200461319 | 3.788623396 | 0.000339125 | 0.001013851 |
| ENSBTAG00000003458 | CDCA7 | 1.200176391 | 5.957143801 | 4.36E−13 | 3.57E−12 |
| ENSBTAG00000027713 | LYSMD3 | 1.199024873 | 3.744579715 | 0.000392621 | 0.001163818 |
| ENSBTAG00000016685 | FMO4 | 1.198895908 | 2.997891378 | 0.006610755 | 0.015147624 |
| ENSBTAG00000008966 | TSPAN7 | 1.198885571 | 3.707597947 | 0.00045487 | 0.001335384 |
| ENSBTAG00000000819 | RFX3 | 1.198620713 | 1.529962218 | 0.454664085 | 0.560901517 |
| ENSBTAG00000017594 | ZHX3 | 1.197862836 | 2.069313202 | 0.146653336 | 0.219631365 |
| ENSBTAG00000016494 | RELT | 1.197099245 | 2.479739592 | 0.064952308 | 0.110487105 |
| ENSBTAG00000013173 | SEPTIN3 | 1.196665628 | 2.426326681 | 0.05521166 | 0.098251567 |
| ENSBTAG00000005318 | RNF6 | 1.196441583 | 4.580922826 | 1.34E−06 | 5.85E−06 |
| ENSBTAG00000031441 | FXYD5 | 1.196193026 | 1.704150171 | 0.346056464 | 0.44594652 |
| ENSBTAG00000009830 | PLEKHB1 | 1.195565414 | 2.672264863 | 0.018472145 | 0.037313173 |
| ENSBTAG00000017592 | GPAT3 | 1.194536913 | 7.681941564 | 2.67E−24 | 4.07E−23 |
| ENSBTAG00000006768 | ENTPD4 | 1.194172581 | 7.462917446 | 6.52E−23 | 9.30E−22 |
| ENSBTAG00000013301 | GGT7 | 1.193706535 | 2.111816444 | 0.099050358 | 0.160864976 |
| ENSBTAG00000038195 | CD302 | 1.193662808 | 3.416088794 | 0.001551149 | 0.004126738 |
| ENSBTAG00000012673 | CDK18 | 1.19327328 | 4.405913447 | 6.62E−06 | 2.62E−05 |
| ENSBTAG00000016043 | GNB3 | 1.192952474 | 1.504028955 | 0.454664085 | 0.560901517 |
| ENSBTAG00000002578 | ZMYND11 | 1.192498316 | 6.970562606 | 1.95E−19 | 2.34E−18 |
| ENSBTAG00000013109 | STIM1 | 1.192416756 | 5.184816465 | 7.16E−09 | 4.02E−08 |
| ENSBTAG00000019938 | N4BP1 | 1.192340015 | 5.609405616 | 4.29E−11 | 2.91E−10 |
| ENSBTAG00000004783 | CORO1B | 1.192219452 | 5.698630563 | 1.23E−11 | 8.72E−11 |
| ENSBTAG00000020164 | BAZ1A | 1.191364918 | 6.517387221 | 2.25E−16 | 2.25E−15 |
| ENSBTAG00000009871 | RAB18 | 1.191122374 | 6.753435256 | 5.59E−18 | 6.09E−17 |
| ENSBTAG00000005344 | GNPNAT1 | 1.190890563 | 4.748357107 | 4.00E−07 | 1.85E−06 |
| ENSBTAG00000007507 | FAAH | 1.190838572 | 3.817242034 | 0.000248277 | 0.000762934 |
| ENSBTAG00000000639 | APRT | 1.190350785 | 4.454788492 | 5.15E−06 | 2.07E−05 |
| ENSBTAG00000010756 | ST7 | 1.188581521 | 3.919199781 | 0.000161741 | 0.000510163 |
| ENSBTAG00000053201 | A0A3Q1LMC3 | 1.187971175 | 1.707593105 | 0.346056464 | 0.44594652 |
| ENSBTAG00000013724 | ATG4A | 1.187217983 | 4.232839526 | 1.92E−05 | 7.07E−05 |
| ENSBTAG00000018133 | SEMA3A | 1.185569547 | 1.631122636 | 0.50965681 | 0.608774039 |
| ENSBTAG00000010120 | HERC3 | 1.185454136 | 5.926771057 | 1.19E−12 | 9.36E−12 |
| ENSBTAG00000003586 | MAN1A2 | 1.183335376 | 5.68450994 | 1.67E−11 | 1.17E−10 |
| ENSBTAG00000012450 | RAPGEF2 | 1.183021489 | 7.647082419 | 8.65E−24 | 1.29E−22 |
| ENSBTAG00000003378 | ACAP2 | 1.179967636 | 3.328327796 | 0.003478384 | 0.00852422 |
| ENSBTAG00000021813 | AMACR | 1.179598048 | 5.181552534 | 7.16E−09 | 4.02E−08 |
| ENSBTAG00000002232 | TRPC1 | 1.179430328 | 2.850078191 | 0.021951686 | 0.043668164 |
| ENSBTAG00000017355 | ERCC1 | 1.177893813 | 3.960062475 | 0.000140389 | 0.000447433 |
| ENSBTAG00000013166 | SMYD2 | 1.175190123 | 4.485025925 | 4.55E−06 | 1.84E−05 |
| ENSBTAG00000030503 | H2AFJ | 1.172673689 | 5.414916982 | 7.55E−10 | 4.61E−09 |
| ENSBTAG00000013411 | ENO1 | 1.172463629 | 10.76821131 | 2.41E−36 | 5.89E−35 |
| ENSBTAG00000017380 | MYO18A | 1.172160153 | 5.875106277 | 3.27E−12 | 2.47E−11 |
| ENSBTAG00000017095 | DENND4A | 1.171858652 | 5.279757793 | 4.67E−09 | 2.67E−08 |
| ENSBTAG00000044033 | EDIL3 | 1.171559667 | 1.782141591 | 0.228981052 | 0.323302376 |
| ENSBTAG00000016442 | MAP4K3 | 1.171426924 | 6.787313519 | 1.11E−17 | 1.19E−16 |
| ENSBTAG00000011723 | GRIK5 | 1.17136734 | 1.478092801 | 0.454664085 | 0.560901517 |
| ENSBTAG00000017482 | ISYNA1 | 1.16953652 | 7.276612266 | 5.88E−21 | 7.61E−20 |
| ENSBTAG00000013706 | MEGF9 | 1.169331654 | 3.195977097 | 0.004064062 | 0.009717574 |
| ENSBTAG00000048098 | PKP3 | 1.168771087 | 5.381546713 | 1.48E−09 | 8.80E−09 |
| ENSBTAG00000002313 | ITPR2 | 1.165287184 | 5.069658071 | 3.68E−08 | 1.91E−07 |
| ENSBTAG00000019496 | CDKN3 | 1.164120927 | 2.545712161 | 0.045693318 | 0.082984632 |
| ENSBTAG00000051267 | CRADD | 1.163904943 | 2.249908385 | 0.137805452 | 0.207856085 |
| ENSBTAG00000046185 | BTBD7 | 1.161820517 | 6.045644389 | 5.78E−13 | 4.67E−12 |
| ENSBTAG00000007356 | ELF1 | 1.161402774 | 6.699557249 | 6.57E−17 | 6.75E−16 |
| ENSBTAG00000012966 | SCRN3 | 1.159674822 | 2.975667181 | 0.009358679 | 0.020487125 |
| ENSBTAG00000031697 | KLHDC1 | 1.159646963 | 2.313687905 | 0.066893327 | 0.113319985 |
| ENSBTAG00000018463 | VIM | 1.158867105 | 6.317655788 | 2.06E−14 | 1.85E−13 |
| ENSBTAG00000003936 | PNKD | 1.158238293 | 2.405919955 | 0.079072739 | 0.131618602 |
| ENSBTAG00000005275 | PKIG | 1.157360528 | 1.709933746 | 0.346056464 | 0.44594652 |
| ENSBTAG00000024928 | TNFRSF1B | 1.15709731 | 4.814267204 | 2.85E−07 | 1.34E−06 |
| ENSBTAG00000014575 | COL4A5 | 1.156885176 | 4.236838078 | 2.58E−05 | 9.33E−05 |
| ENSBTAG00000019712 | ST14 | 1.156516128 | 7.963987492 | 9.45E−25 | 1.46E−23 |
| ENSBTAG00000000880 | GINM1 | 1.155685321 | 4.106087616 | 9.37E−05 | 0.000308304 |
| ENSBTAG00000013938 | PHC3 | 1.15465341 | 4.2208134 | 3.46E−05 | 0.000122056 |
| ENSBTAG00000026586 | PPP1R14C | 1.154266001 | 1.924058365 | 0.182431139 | 0.26547662 |
| ENSBTAG00000012433 | DENND1B | 1.153980555 | 4.897993239 | 2.13E−07 | 1.01E−06 |
| ENSBTAG00000018362 | TMEM109 | 1.152650585 | 6.22983682 | 7.43E−14 | 6.41E−13 |
| ENSBTAG00000021573 | DCAF12 | 1.152405673 | 6.270542429 | 5.21E−14 | 4.57E−13 |
| ENSBTAG00000010579 | ACADL | 1.152227607 | 3.486343892 | 0.002151683 | 0.005496122 |
| ENSBTAG00000055207 | SCD | 1.149679052 | 9.861507764 | 4.67E−33 | 9.86E−32 |
| ENSBTAG00000001097 | FKBP7 | 1.149598277 | 2.763467747 | 0.026281109 | 0.051463956 |
| ENSBTAG00000001692 | NDST1 | 1.147951033 | 5.415508456 | 1.43E−09 | 8.52E−09 |
| ENSBTAG00000010913 | SRXN1 | 1.147203573 | 3.954258949 | 0.000222042 | 0.000687792 |
| ENSBTAG00000021538 | MYO1E | 1.146602978 | 7.60311332 | 3.43E−22 | 4.74E−21 |
| ENSBTAG00000000919 | HEY2 | 1.146328173 | 1.950856446 | 0.182431139 | 0.26547662 |
| ENSBTAG00000008465 | ACCS | 1.145775166 | 2.217538269 | 0.081278796 | 0.13479377 |
| ENSBTAG00000009214 | ETS2 | 1.144465455 | 6.240973011 | 1.12E−13 | 9.56E−13 |
| ENSBTAG00000052901 | CD46 | 1.143624265 | 1.947096986 | 0.182431139 | 0.26547662 |
| ENSBTAG00000005180 | RBMS1 | 1.138338081 | 6.417881606 | 9.09E−15 | 8.32E−14 |
| ENSBTAG00000000783 | TGFA | 1.137708787 | 1.720808508 | 0.346056464 | 0.44594652 |
| ENSBTAG00000014207 | ADAMTS10 | 1.137698176 | 1.450419285 | 0.688379477 | 0.77593466 |
| ENSBTAG00000001141 | ADAM17 | 1.137424776 | 6.229080056 | 1.76E−13 | 1.48E−12 |
| ENSBTAG00000024490 | RAET1G | 1.136241303 | 1.742794312 | 0.228981052 | 0.323302376 |
| ENSBTAG00000015142 | MAPKBP1 | 1.135432602 | 4.810852176 | 5.71E−07 | 2.60E−06 |
| ENSBTAG00000021151 | MYH10 | 1.135092217 | 9.253971454 | 1.80E−30 | 3.48E−29 |
| ENSBTAG00000052055 | PRSS35 | 1.13501071 | 1.66528036 | 0.346056464 | 0.44594652 |
| ENSBTAG00000016757 | ZKSCAN1 | 1.134935958 | 4.328631474 | 2.03E−05 | 7.45E−05 |
| ENSBTAG00000005650 | SKAP2 | 1.134708438 | 6.157352021 | 3.30E−13 | 2.72E−12 |
| ENSBTAG00000005617 | SLC41A3 | 1.132236119 | 2.909540771 | 0.025457164 | 0.050017444 |
| ENSBTAG00000010681 | NR1H3 | 1.132100595 | 3.315857233 | 0.004784234 | 0.011281409 |
| ENSBTAG00000050728 | NCL | 1.131827033 | 2.025316995 | 0.146653336 | 0.219631365 |
| ENSBTAG00000007346 | COMMD4 | 1.130999059 | 5.090304957 | 6.43E−08 | 3.26E−07 |
| ENSBTAG00000017745 | IL6ST | 1.130708046 | 6.995081163 | 4.64E−18 | 5.09E−17 |
| ENSBTAG00000021037 | ARNT | 1.129120794 | 5.458632711 | 1.59E−09 | 9.49E−09 |
| ENSBTAG00000013545 | MAVS | 1.127859639 | 4.273643454 | 3.53E−05 | 0.000124518 |
| ENSBTAG00000013855 | ORMDL3 | 1.127362339 | 5.282960443 | 1.31E−08 | 7.15E−08 |
| ENSBTAG00000050443 | NA | 1.127085882 | 2.026405662 | 0.146653336 | 0.219631365 |
| ENSBTAG00000015255 | ST6GALNAC2 | 1.12691881 | 6.117650367 | 9.57E−13 | 7.59E−12 |
| ENSBTAG00000013455 | SPOPL | 1.126649406 | 4.8555357 | 6.84E−07 | 3.07E−06 |
| ENSBTAG00000001529 | NSD3 | 1.12630321 | 6.126592698 | 1.02E−12 | 8.06E−12 |
| ENSBTAG00000016481 | CALU | 1.126251156 | 8.089973374 | 3.08E−24 | 4.68E−23 |
| ENSBTAG00000001773 | CYREN | 1.125851682 | 3.415796212 | 0.003450459 | 0.008472773 |
| ENSBTAG00000016683 | NFKBIA | 1.124880874 | 3.767431584 | 0.000626257 | 0.001785635 |
| ENSBTAG00000012738 | ZNF827 | 1.123694902 | 2.683260257 | 0.044282377 | 0.080651884 |
| ENSBTAG00000012537 | CASP6 | 1.122924618 | 7.207888189 | 4.86E−19 | 5.71E−18 |
| ENSBTAG00000016101 | VHL | 1.122107842 | 3.071312623 | 0.012938808 | 0.027451685 |
| ENSBTAG00000017362 | CNOT6 | 1.119389316 | 6.096557022 | 1.64E−12 | 1.27E−11 |
| ENSBTAG00000050748 | lncRNA | 1.116851153 | 4.485681403 | 9.32E−06 | 3.59E−05 |
| ENSBTAG00000015549 | PCDH18 | 1.116595074 | 1.523861598 | 0.454664085 | 0.560901517 |
| ENSBTAG00000016420 | CTNNB1 | 1.114153908 | 9.565437823 | 2.16E−30 | 4.17E−29 |
| ENSBTAG00000004899 | ABLIM1 | 1.113887109 | 3.50448515 | 0.002151683 | 0.005496122 |
| ENSBTAG00000021996 | GATAD1 | 1.113882743 | 4.1229578 | 0.000108902 | 0.000354632 |
| ENSBTAG00000033398 | HKDC1 | 1.113203833 | 8.282357616 | 7.17E−25 | 1.12E−23 |
| ENSBTAG00000014676 | SHROOM1 | 1.112770821 | 5.090348352 | 9.57E−08 | 4.75E−07 |
| ENSBTAG00000047030 | CLC | 1.112640188 | 2.163762805 | 0.118721218 | 0.188818489 |
| ENSBTAG00000005206 | TMEM14A | 1.111389065 | 5.26700962 | 1.85E−08 | 9.89E−08 |
| ENSBTAG00000020865 | PELI1 | 1.111201745 | 5.436379961 | 5.15E−09 | 2.93E−08 |
| ENSBTAG00000013284 | SGK3 | 1.109498922 | 4.56995592 | 7.36E−06 | 2.90E−05 |
| ENSBTAG00000047078 | NTF4 | 1.109381467 | 2.438813099 | 0.05521166 | 0.098251567 |
| ENSBTAG00000022022 | GUCD1 | 1.10931176 | 5.859443311 | 4.37E−11 | 2.96E−10 |
| ENSBTAG00000044159 | C17H4orf33 | 1.107614654 | 2.918002755 | 0.018371623 | 0.03712546 |
| ENSBTAG00000001110 | SLC16A5 | 1.107059891 | 3.028963896 | 0.011078732 | 0.023871274 |
| ENSBTAG00000002317 | PTN | 1.106680035 | 4.002369469 | 0.000192258 | 0.000600607 |
| ENSBTAG00000013591 | CYB5R4 | 1.105743645 | 6.156187755 | 1.62E−12 | 1.25E−11 |
| ENSBTAG00000054367 | WNT5B | 1.105447747 | 3.449616846 | 0.003986321 | 0.009669143 |
| ENSBTAG00000011832 | ALDH18A1 | 1.105252119 | 5.197056923 | 4.55E−08 | 2.33E−07 |
| ENSBTAG00000014807 | DPP8 | 1.104980174 | 6.205081232 | 8.92E−13 | 7.09E−12 |
| ENSBTAG00000040334 | MEX3D | 1.104665385 | 3.826741077 | 0.00062515 | 0.00178352 |
| ENSBTAG00000053344 | A0A3Q1LNV3 | 1.104127876 | 2.827855588 | 0.030540633 | 0.059071537 |
| ENSBTAG00000023172 | ITSN2 | 1.103922795 | 5.098073465 | 1.41E−07 | 6.88E−07 |
| ENSBTAG00000015939 | NECTIN1 | 1.103706193 | 6.23024496 | 6.04E−13 | 4.87E−12 |
| ENSBTAG00000016267 | SERPING1 | 1.103031675 | 5.802808275 | 8.73E−11 | 5.78E−10 |
| ENSBTAG00000015434 | DSTN | 1.102128338 | 10.40616014 | 8.60E−32 | 1.76E−30 |
| ENSBTAG00000012961 | PAPSS1 | 1.102029611 | 6.198826723 | 1.31E−12 | 1.02E−11 |
| ENSBTAG00000011327 | OLFML3 | 1.10194955 | 1.969420697 | 0.21354755 | 0.304442255 |
| ENSBTAG00000016724 | NPEPL1 | 1.101538697 | 4.479292186 | 1.23E−05 | 4.67E−05 |
| ENSBTAG00000032674 | SENP5 | 1.101064519 | 4.644316194 | 3.86E−06 | 1.58E−05 |
| ENSBTAG00000021395 | PSME1 | 1.100342445 | 6.073491949 | 4.80E−12 | 3.57E−11 |
| ENSBTAG00000054095 | GMFB | 1.09985228 | 4.505573245 | 1.63E−05 | 6.12E−05 |
| ENSBTAG00000001535 | LIPH | 1.098305106 | 6.068753927 | 7.17E−12 | 5.19E−11 |
| ENSBTAG00000018035 | TGFBR1 | 1.097637972 | 6.107094881 | 3.62E−12 | 2.73E−11 |
| ENSBTAG00000015441 | ACTG2 | 1.097616951 | 5.257792364 | 4.38E−08 | 2.25E−07 |
| ENSBTAG00000018706 | PCTP | 1.096622392 | 5.313272124 | 2.00E−08 | 1.07E−07 |
| ENSBTAG00000011770 | TJP2 | 1.096406354 | 8.740993178 | 1.28E−26 | 2.14E−25 |
| ENSBTAG00000020745 | HIVEP3 | 1.095756536 | 2.085873263 | 0.241859291 | 0.338713856 |
| ENSBTAG00000004110 | pseudogene | 1.094209317 | 2.073222605 | 0.146653336 | 0.219631365 |
| ENSBTAG00000003276 | PRKCH | 1.093462277 | 3.426818204 | 0.003450459 | 0.008472773 |
| ENSBTAG00000005903 | LRP5 | 1.092973187 | 5.76187326 | 2.14E−10 | 1.37E−09 |
| ENSBTAG00000054545 | RNF11 | 1.092905299 | 6.66560662 | 4.90E−15 | 4.57E−14 |
| ENSBTAG00000005464 | ADCY6 | 1.092800083 | 4.073778766 | 0.000222664 | 0.000689427 |
| ENSBTAG00000011435 | NPEPPS | 1.091187952 | 8.181726544 | 2.21E−23 | 3.22E−22 |
| ENSBTAG00000002444 | MKI67 | 1.091032176 | 7.778278714 | 2.68E−21 | 3.54E−20 |
| ENSBTAG00000018125 | KIF5C | 1.090587461 | 6.332077029 | 3.85E−13 | 3.17E−12 |
| ENSBTAG00000039035 | HSPA6 | 1.090285923 | 2.467325857 | 0.091299084 | 0.149752144 |
| ENSBTAG00000001036 | HIBADH | 1.088344364 | 5.729161342 | 4.84E−10 | 3.02E−09 |
| ENSBTAG00000017298 | AAMDC | 1.087898908 | 3.281362487 | 0.008905401 | 0.019591347 |
| ENSBTAG00000001857 | DOK4 | 1.086603029 | 3.581000052 | 0.002132325 | 0.005449522 |
| ENSBTAG00000002425 | SCN9A | 1.083974209 | 3.433390522 | 0.003450459 | 0.008472773 |
| ENSBTAG00000047532 | TRIQK | 1.083310697 | 2.511726314 | 0.064952308 | 0.110487105 |
| ENSBTAG00000002464 | PLCH2 | 1.083221915 | 1.783812525 | 0.390409834 | 0.493420167 |
| ENSBTAG00000046273 | TLCD3A | 1.082366926 | 5.577407192 | 2.99E−09 | 1.73E−08 |
| ENSBTAG00000019052 | ANK3 | 1.08218272 | 4.433521927 | 2.43E−05 | 8.81E−05 |
| ENSBTAG00000008341 | ARSB | 1.081539424 | 6.857828837 | 7.81E−16 | 7.57E−15 |
| ENSBTAG00000027444 | SVIL | 1.081170832 | 5.696351486 | 7.81E−10 | 4.76E−09 |
| ENSBTAG00000015046 | MST1R | 1.080559901 | 3.110506235 | 0.014932701 | 0.031306164 |
| ENSBTAG00000024493 | DHRS3 | 1.08036933 | 4.624304974 | 8.58E−06 | 3.35E−05 |
| ENSBTAG00000005215 | IL10RA | 1.07986817 | 1.868314404 | 0.269789176 | 0.361699985 |
| ENSBTAG00000011150 | PFN2 | 1.078754786 | 6.303359605 | 9.77E−13 | 7.73E−12 |
| ENSBTAG00000011056 | IDS | 1.07815318 | 4.116301914 | 0.000192803 | 0.00060218 |
| ENSBTAG00000053672 | processed | 1.076653745 | 2.076809569 | 0.241859291 | 0.338713856 |
| | pseudogene | | | | |
| ENSBTAG00000013621 | DPY19L4 | 1.075993864 | 5.319687683 | 4.18E−08 | 2.15E−07 |
| ENSBTAG00000038281 | CSMD3 | 1.07577548 | 1.585064076 | 0.290910266 | 0.385192821 |
| ENSBTAG00000018248 | MGLL | 1.075125513 | 6.261592951 | 1.74E−12 | 1.35E−11 |
| ENSBTAG00000005762 | LYNX1 | 1.072584726 | 1.819345392 | 0.390409834 | 0.493420167 |
| ENSBTAG00000046041 | ABCC4 | 1.072078361 | 1.827612569 | 0.269789176 | 0.361699985 |
| ENSBTAG00000009218 | ANLN | 1.072060096 | 8.604056583 | 5.42E−25 | 8.49E−24 |
| ENSBTAG00000000290 | ATP11B | 1.071582166 | 6.900024766 | 7.95E−16 | 7.70E−15 |
| ENSBTAG00000021187 | ID2 | 1.071284627 | 5.931334425 | 8.50E−11 | 5.63E−10 |
| ENSBTAG00000044192 | MAF | 1.070823327 | 1.913536581 | 0.182431139 | 0.26547662 |
| ENSBTAG00000010309 | XPO6 | 1.067432018 | 7.808350627 | 1.27E−20 | 1.62E−19 |
| ENSBTAG00000033662 | NCKAP1 | 1.067354778 | 8.052272972 | 7.50E−22 | 1.02E−20 |
| ENSBTAG00000020436 | KIF27 | 1.067154607 | 4.96632688 | 9.58E−07 | 4.24E−06 |
| ENSBTAG00000000115 | VPS54 | 1.066998033 | 6.237762108 | 3.50E−12 | 2.63E−11 |
| ENSBTAG00000015036 | RAD51C | 1.066830011 | 4.816411401 | 3.19E−06 | 1.33E−05 |
| ENSBTAG00000045909 | FAM174A | 1.065276696 | 4.35390627 | 7.17E−05 | 0.000239542 |
| ENSBTAG00000011454 | FKBP10 | 1.065033939 | 2.961601892 | 0.021266995 | 0.042432732 |
| ENSBTAG00000000902 | OGT | 1.062837661 | 7.628318428 | 1.45E−19 | 1.76E−18 |
| ENSBTAG00000011155 | PUM2 | 1.061723035 | 6.907416235 | 1.27E−15 | 1.22E−14 |
| ENSBTAG00000017335 | HGFAC | 1.061552877 | 2.218326556 | 0.137805452 | 0.207856085 |
| ENSBTAG00000016943 | NEK7 | 1.060877327 | 7.855346461 | 1.15E−20 | 1.47E−19 |
| ENSBTAG00000012944 | USP35 | 1.060225118 | 3.253957348 | 0.007743401 | 0.017550991 |
| ENSBTAG00000016167 | ATL2 | 1.059624488 | 7.234929971 | 2.67E−17 | 2.83E−16 |
| ENSBTAG00000009087 | GNG10 | 1.059175835 | 6.32371768 | 2.11E−12 | 1.61E−11 |
| ENSBTAG00000021226 | SAR1B | 1.059047835 | 5.214896855 | 1.40E−07 | 6.82E−07 |
| ENSBTAG00000002938 | CPED1 | 1.058863864 | 1.386678548 | 0.376174287 | 0.478894911 |
| ENSBTAG00000038151 | ELOF1 | 1.058850098 | 5.008445486 | 7.60E−07 | 3.40E−06 |
| ENSBTAG00000007981 | GRK5 | 1.058628644 | 3.895488699 | 0.00061967 | 0.001769261 |
| ENSBTAG00000047077 | ENTPD7 | 1.058007421 | 3.786452281 | 0.000977418 | 0.002706399 |
| ENSBTAG00000011234 | FOXO3 | 1.056830152 | 4.269676109 | 0.000108698 | 0.000354282 |
| ENSBTAG00000012463 | ZMAT3 | 1.056544736 | 4.119430236 | 0.000291793 | 0.000881508 |
| ENSBTAG00000011628 | EGFR | 1.056260674 | 6.328233524 | 1.47E−12 | 1.14E−11 |
| ENSBTAG00000006143 | CREB3L1 | 1.055110769 | 4.842546982 | 2.83E−06 | 1.18E−05 |
| ENSBTAG00000046744 | PALM3 | 1.054828316 | 2.801262482 | 0.04213522 | 0.077114709 |
| ENSBTAG00000002668 | ZNF711 | 1.053577634 | 3.285611348 | 0.008905401 | 0.019591347 |
| ENSBTAG00000015147 | S100A10 | 1.053322115 | 9.106052577 | 4.88E−26 | 7.99E−25 |
| ENSBTAG00000033464 | SPA17 | 1.052862511 | 3.381247444 | 0.006360195 | 0.014616887 |
| ENSBTAG00000015220 | AGO4 | 1.051747027 | 4.336246547 | 8.23E−05 | 0.000273058 |
| ENSBTAG00000045689 | CAP1 | 1.051246228 | 6.34452887 | 1.73E−12 | 1.34E−11 |
| ENSBTAG00000055296 | LGALSL | 1.050255168 | 6.758991717 | 1.48E−14 | 1.34E−13 |
| ENSBTAG00000011268 | TMEM64 | 1.050136099 | 2.939446732 | 0.029143953 | 0.056571513 |
| ENSBTAG00000004895 | AFAP1L2 | 1.049968043 | 5.881441288 | 3.26E−10 | 2.06E−09 |
| ENSBTAG00000050848 | ZCCHC2 | 1.04949525 | 3.298732592 | 0.008905401 | 0.019591347 |
| ENSBTAG00000007237 | BUB1B | 1.048505186 | 6.916063601 | 3.02E−15 | 2.83E−14 |
| ENSBTAG00000043580 | MT tRNA | 1.048397174 | 2.661658217 | 0.044282377 | 0.080651884 |
| ENSBTAG00000012208 | BOLA | 1.047515783 | 2.034191428 | 0.21354755 | 0.304442255 |
| ENSBTAG00000048747 | KIF1B | 1.04708806 | 4.491382125 | 2.79E−05 | 0.000100457 |
| ENSBTAG00000009314 | FYB2 | 1.046804688 | 1.99749299 | 0.21354755 | 0.304442255 |
| ENSBTAG00000020938 | TWF1 | 1.045403722 | 6.938122358 | 2.40E−15 | 2.27E−14 |
| ENSBTAG00000008114 | CD99 | 1.044866919 | 8.218030494 | 7.45E−22 | 1.01E−20 |
| ENSBTAG00000008513 | RHEX | 1.044032647 | 1.830186253 | 0.269789176 | 0.361699985 |
| ENSBTAG00000017845 | BAG1 | 1.043003811 | 5.721177294 | 2.75E−09 | 1.60E−08 |
| ENSBTAG00000021766 | HBEGF | 1.04240426 | 5.99977585 | 1.28E−10 | 8.33E−10 |
| ENSBTAG00000053396 | env | 1.041899779 | 6.086238257 | 4.83E−11 | 3.26E−10 |
| ENSBTAG00000016711 | PPIF | 1.041849798 | 5.980464858 | 1.82E−10 | 1.18E−09 |
| ENSBTAG00000011096 | ERGIC2 | 1.039848173 | 6.060271603 | 8.27E−11 | 5.48E−10 |
| ENSBTAG00000011429 | CREB3 | 1.038111762 | 5.39335475 | 5.08E−08 | 2.60E−07 |
| ENSBTAG00000014694 | JTB | 1.037893582 | 4.879380883 | 3.23E−06 | 1.34E−05 |
| ENSBTAG00000019742 | FOXJ2 | 1.037865543 | 5.08306656 | 5.47E−07 | 2.49E−06 |
| ENSBTAG00000001349 | KIF1C | 1.037565569 | 7.478158108 | 5.48E−18 | 5.99E−17 |
| ENSBTAG00000015155 | HACD3 | 1.037306626 | 4.949977983 | 2.00E−06 | 8.55E−06 |
| ENSBTAG00000034885 | RGCC | 1.037130132 | 1.801178649 | 0.390409834 | 0.493420167 |
| ENSBTAG00000016643 | CYB561D1 | 1.036663937 | 3.269914977 | 0.012046963 | 0.025723222 |
| ENSBTAG00000048782 | B2M | 1.035193521 | 7.564209006 | 2.88E−18 | 3.20E−17 |
| ENSBTAG00000000539 | LRRC28 | 1.034819593 | 4.426886324 | 6.20E−05 | 0.000210482 |
| ENSBTAG00000021204 | TES | 1.034795846 | 7.570563231 | 2.72E−18 | 3.03E−17 |
| ENSBTAG00000034659 | UBTD2 | 1.034114263 | 3.914399232 | 0.000709784 | 0.002005084 |
| ENSBTAG00000006760 | OTX1 | 1.032697174 | 2.303670758 | 0.156188855 | 0.231653549 |
| ENSBTAG00000006396 | GPI | 1.031658553 | 8.537880382 | 5.39E−23 | 7.73E−22 |
| ENSBTAG00000016230 | BRWD3 | 1.031028407 | 5.531481678 | 1.69E−08 | 9.04E−08 |
| ENSBTAG00000019639 | SNX16 | 1.030647315 | 4.420559938 | 8.06E−05 | 0.00026779 |
| ENSBTAG00000051333 | ARID5B | 1.030446008 | 5.04027469 | 1.11E−06 | 4.88E−06 |
| ENSBTAG00000019692 | BCL2L2 | 1.029493062 | 4.947562506 | 2.55E−06 | 1.07E−05 |
| ENSBTAG00000006817 | CBL | 1.0291314 | 6.051478036 | 1.08E−10 | 7.10E−10 |
| ENSBTAG00000001902 | CEP295 | 1.028705769 | 6.247939264 | 1.87E−11 | 1.30E−10 |
| ENSBTAG00000002849 | IRF6 | 1.028690069 | 6.756782128 | 4.66E−14 | 4.10E−13 |
| ENSBTAG00000004568 | GALNS | 1.028505661 | 3.978153423 | 0.000695468 | 0.001966918 |
| ENSBTAG00000005824 | SPOP | 1.027597323 | 4.477198506 | 4.73E−05 | 0.000163388 |
| ENSBTAG00000014335 | CYP17A1 | 1.027375431 | 2.758040923 | 0.036727173 | 0.068221561 |
| ENSBTAG00000051082 | DST | 1.027022265 | 5.109648346 | 7.92E−07 | 3.54E−06 |
| ENSBTAG00000055257 | RAP2B | 1.026813405 | 5.598633657 | 9.44E−09 | 5.23E−08 |
| ENSBTAG00000004011 | GALK2 | 1.026569375 | 4.600609474 | 2.75E−05 | 9.92E−05 |
| ENSBTAG00000033441 | SHCBP1 | 1.026349097 | 6.465795683 | 1.29E−12 | 1.01E−11 |
| ENSBTAG00000021778 | SELENON | 1.026119227 | 4.54258295 | 3.17E−05 | 0.000112998 |
| ENSBTAG00000054686 | KIAA1211L | 1.023851936 | 3.646838073 | 0.003192798 | 0.007875676 |
| ENSBTAG00000023867 | RC3H2 | 1.023612865 | 4.986204608 | 2.01E−06 | 8.59E−06 |
| ENSBTAG00000008794 | ATF6B | 1.023506725 | 5.79966492 | 1.65E−09 | 9.82E−09 |
| ENSBTAG00000035226 | TOR1AIP1 | 1.023180232 | 5.984046072 | 3.39E−10 | 2.14E−09 |
| ENSBTAG00000044070 | SNX30 | 1.02159946 | 5.371740892 | 1.10E−07 | 5.44E−07 |
| ENSBTAG00000024603 | NCOR2 | 1.021517486 | 5.880699043 | 9.24E−10 | 5.61E−09 |
| ENSBTAG00000016046 | GOPC | 1.021251055 | 5.166888111 | 4.46E−07 | 2.05E−06 |
| ENSBTAG00000013468 | AHNAK | 1.020873176 | 11.77598278 | 3.99E−29 | 7.41E−28 |
| ENSBTAG00000038619 | H2-T24 | 1.019089556 | 3.386604386 | 0.006360195 | 0.014616887 |
| ENSBTAG00000048945 | lncRNA | 1.018814616 | 2.524590176 | 0.103470288 | 0.166989294 |
| ENSBTAG00000007436 | GGT6 | 1.018338479 | 1.636901262 | 0.50965681 | 0.608774039 |
| ENSBTAG00000021045 | E2F3 | 1.018331769 | 3.647241038 | 0.003192798 | 0.007875676 |
| ENSBTAG00000003076 | NT5DC1 | 1.018106575 | 5.055488432 | 1.42E−06 | 6.15E−06 |
| ENSBTAG00000021972 | DNAH5 | 1.01712009 | 2.122007149 | 0.170851525 | 0.250688206 |
| ENSBTAG00000017682 | TET2 | 1.016270095 | 4.911502622 | 3.24E−06 | 1.34E−05 |
| ENSBTAG00000021000 | USF3 | 1.015932599 | 4.851014275 | 5.97E−06 | 2.38E−05 |
| ENSBTAG00000010841 | FMO5 | 1.015363717 | 3.882483959 | 0.00163914 | 0.004337231 |
| ENSBTAG00000010234 | DHRS1 | 1.01530076 | 5.25881442 | 3.26E−07 | 1.52E−06 |
| ENSBTAG00000054889 | lncRNA | 1.015248457 | 2.615382551 | 0.084811919 | 0.140004098 |
| ENSBTAG00000001439 | HESX1 | 1.014934906 | 2.018693549 | 0.21354755 | 0.304442255 |
| ENSBTAG00000016229 | KLF9 | 1.014741264 | 2.497889257 | 0.064952308 | 0.110487105 |
| ENSBTAG00000011733 | GIPC2 | 1.014659449 | 6.374412971 | 9.40E−12 | 6.74E−11 |
| ENSBTAG00000011280 | TBKBP1 | 1.013185267 | 2.139340194 | 0.170851525 | 0.250688206 |
| ENSBTAG00000011079 | C18H19orf48 | 1.012455427 | 3.663292212 | 0.003192798 | 0.007875676 |
| ENSBTAG00000013071 | GPR153 | 1.012438191 | 5.011159973 | 2.01E−06 | 8.60E−06 |
| ENSBTAG00000004802 | CEP170B | 1.012331817 | 7.130705787 | 1.60E−15 | 1.52E−14 |
| ENSBTAG00000009315 | CDON | 1.012080031 | 3.720737688 | 0.003073283 | 0.00760004 |
| ENSBTAG00000006015 | POLH | 1.011962255 | 4.782518669 | 1.11E−05 | 4.23E−05 |
| ENSBTAG00000021634 | PIAS3 | 1.011952828 | 4.859388955 | 6.72E−06 | 2.66E−05 |
| ENSBTAG00000049029 | A0A3Q1NAQ8 | 1.011617841 | 1.799089402 | 0.390409834 | 0.493420167 |
| ENSBTAG00000035323 | TMX4 | 1.01117017 | 6.533014761 | 1.32E−12 | 1.03E−11 |
| ENSBTAG00000015107 | SLC16A1 | 1.010717296 | 10.24025371 | 7.74E−27 | 1.30E−25 |
| ENSBTAG00000047956 | NAGLU | 1.010680907 | 4.319747375 | 0.000206414 | 0.000642503 |
| ENSBTAG00000014728 | TAPBPL | 1.010186577 | 3.229822941 | 0.010562196 | 0.02286579 |
| ENSBTAG00000054926 | A0A3Q1MUE4 | 1.010185578 | 3.390615388 | 0.006360195 | 0.014616887 |
| ENSBTAG00000030669 | FGGY | 1.010057092 | 3.259268185 | 0.012046963 | 0.025723222 |
| ENSBTAG00000045523 | FARSB | 1.009747221 | 3.944849979 | 0.000808745 | 0.002267609 |
| ENSBTAG00000002075 | MME | 1.009611224 | 5.501727116 | 4.44E−08 | 2.27E−07 |
| ENSBTAG00000033140 | LRRC3B | 1.008925952 | 2.390860012 | 0.079072739 | 0.131618602 |
| ENSBTAG00000012050 | ORAI2 | 1.008739231 | 2.386685356 | 0.111869996 | 0.179106781 |
| ENSBTAG00000037686 | SRPX2 | 1.007512319 | 2.259372844 | 0.137805452 | 0.207856085 |
| ENSBTAG00000014982 | C7H19orf71 | 1.005575724 | 2.623326113 | 0.084811919 | 0.140004098 |
| ENSBTAG00000012447 | PPP1CB | 1.004931157 | 9.187621298 | 4.02E−24 | 6.08E−23 |
| ENSBTAG00000014979 | MFSD12 | 1.004450702 | 5.778418445 | 5.58E−09 | 3.15E−08 |
| ENSBTAG00000012012 | CYB5A | 1.003701503 | 6.454067806 | 5.54E−12 | 4.08E−11 |
| ENSBTAG00000034785 | DNAJC15 | 1.002822677 | 3.720935703 | 0.003073283 | 0.00760004 |
| ENSBTAG00000003196 | PAPSS2 | 1.00246701 | 2.040595438 | 0.336186943 | 0.435818799 |
| ENSBTAG00000005246 | DIP2B | 1.0023415 | 7.04501618 | 7.56E−15 | 6.95E−14 |
| ENSBTAG00000020650 | ADCYAPI | 1.001655349 | 1.397818574 | 0.688379477 | 0.77593466 |
| ENSBTAG00000018868 | METTL9 | 1.000681451 | 5.479515679 | 7.55E−08 | 3.79E−07 |

TABLE 3
Gene expression representative of the three germ layer lineages comparing
values for bTSCs and biPSCs (for heatmap shown in FIG. 3F).

| Genes | Lineage Specificity | TSC1 | TSC2 | TSC4 | biPSC1 | biPSC2 | biPSC3 |
|---|---|---|---|---|---|---|---|
| EGR2 | Ectoderm | 1.413441 | 1.068876 | 1.324685 | 2.165776 | 2.14854 | 3.225076 |
| FGF5 | Ectoderm | 0 | 0 | 0 | 0 | 0 | 0 |
| FOXJ3 | Ectoderm | 84.86537 | 88.89484 | 99.60372 | 52.61562 | 47.58229 | 45.96936 |
| GBX2 | Ectoderm | 0 | 0 | 0.126161 | 9.469962 | 6.026391 | 2.11796 |
| LHX5 | Ectoderm | 0 | 0 | 0 | 0 | 0 | 0 |
| LMX1A | Ectoderm | 0 | 0 | 0 | 0 | 0 | 0 |
| MEIS1 | Ectoderm | 3.47471 | 3.384774 | 2.838611 | 14.22618 | 12.57682 | 11.55251 |
| MEIS2 | Ectoderm | 5.123725 | 3.800448 | 4.163297 | 99.11611 | 88.71896 | 49.0019 |
| PARD6B | Ectoderm | 79.80054 | 80.40322 | 83.07669 | 15.28783 | 14.30613 | 18.1952 |
| PAX2 | Ectoderm | 0 | 0 | 0 | 0 | 0 | 0 |
| PAX6 | Ectoderm | | | | 9.639827 | 6.707636 | 0.866438 |
| PENK | Ectoderm | 0 | 0 | 0 | 0 | 0 | 0 |
| RBM27 | Ectoderm | 53.88745 | 52.37492 | 51.22116 | 79.92138 | 73.20756 | 94.39363 |
| SOX1 | Ectoderm | 0 | 0 | 0 | 0 | 0 | 0 |
| TFCP2L1 | Ectoderm | 1.295655 | 1.662696 | 2.081648 | 0 | 0 | 0 |
| TRIM33 | Ectoderm | 64.37048 | 67.57671 | 63.14333 | 68.83771 | 65.76627 | 75.09132 |
| ZIC1 | Ectoderm | 0 | 0 | 0 | 6.752125 | 8.436948 | 10.78234 |
| AFP | Endoderm | 0 | 0 | 0 | 0 | 0 | 0 |
| CALCR | Endoderm | 0 | 0 | 0 | 0 | 0 | 0 |
| CCKBR | Endoderm | 0 | 0 | 0 | 0 | 0 | 0 |
| CER1 | Endoderm | 0 | 0 | 0 | 0 | 0 | 0 |
| CXCR4 | Endoderm | 6.007126 | 10.39185 | 8.137353 | 3.184965 | 4.821113 | 1.395928 |
| CYP26A1 | Endoderm | 1.884588 | 1.247022 | 1.261605 | | | |
| DKK4 | Endoderm | 0 | 0 | 0 | 0 | 0 | 0 |
| DLX3 | Endoderm | 27.67989 | 23.39651 | 25.35826 | | | |
| DLX5 | Endoderm | 50.41274 | 67.39856 | 67.81127 | 36.01134 | 28.82187 | 2.839992 |
| ELF4 | Endoderm | 73.0278 | 60.92592 | 58.47539 | 28.66468 | 30.55118 | 51.07172 |
| EYA2 | Endoderm | 0 | 0 | 0.06308 | | | |
| FGF17 | Endoderm | 0 | 0 | 0 | 0 | 0 | 0 |
| FOXA2 | Endoderm | 0 | 0 | 0 | 0 | 0 | 0 |
| FOXA3 | Endoderm | 0.942294 | 0.89073 | 1.135445 | 3.439762 | 2.724977 | 4.043379 |
| FOXC1 | Endoderm | 0 | 0 | 0 | 0 | 0 | 0 |
| FOXG1 | Endoderm | 3.65139 | 2.07837 | 1.703167 | 0 | 0 | 0 |
| FOXH1 | Endoderm | 0.058893 | 0.059382 | 0 | 7.686382 | 7.7033 | 11.11929 |
| FOXQ1 | Endoderm | 0.35336 | 0.29691 | 0.189241 | 0 | 0 | 0 |
| GATA6 | Endoderm | 5.064832 | 5.878817 | 9.335877 | 6.0302 | 9.327806 | 34.85007 |
| GPC1 | Endoderm | 11.602 | 10.68876 | 12.55297 | 12.82479 | 10.7427 | 11.55251 |
| HHEX | Endoderm | 1.472335 | 1.425168 | 1.387766 | 1.868513 | 1.781716 | 0.722032 |
| ID4 | Endoderm | 0.235574 | 0.59382 | 0.252321 | 0 | 0 | 0 |
| NTS | Endoderm | 0 | 0 | 0 | 0 | 0 | 0 |
| ONECUT1 | Endoderm | 0 | 0 | 0 | 0 | 0 | 0 |
| PAX9 | Endoderm | 0 | 0 | 0 | 0 | 0 | 0 |
| PLXNA2 | Endoderm | 6.94942 | 5.581907 | 8.326593 | 0 | 0 | 0 |
| PRDM1 | Endoderm | 0.35336 | 0.178146 | 0.504642 | 3.439762 | 3.144204 | 5.535578 |
| PYY | Endoderm | 0.294467 | 1.068876 | 1.135445 | 0 | 0 | 0 |
| SHISA2 | Endoderm | 12.54429 | 21.97134 | 18.54559 | 0 | 0 | 0 |
| SOX17 | Endoderm | 1.060081 | 1.18764 | 2.018568 | 0 | 0 | 0 |
| SOX7 | Endoderm | 0 | 0 | 0 | 0 | 0 | 0 |
| SP6 | Endoderm | 16.13679 | 20.48679 | 26.80911 | 3.567161 | 3.196608 | 5.776255 |
| TLE2 | Endoderm | 2.120162 | 1.425168 | 1.640087 | 0 | 0 | 0 |
| TSPAN7 | Endoderm | 13.42769 | 17.8146 | 10.34516 | 8.068578 | 5.292744 | 2.310502 |
| CXCL12 | Mesoderm | 0.530041 | 0.59382 | 1.324685 | 39.66343 | 42.86598 | 60.60254 |
| EDNRB | Mesoderm | 5.653765 | 6.056963 | 6.686507 | 3.0151 | 2.986994 | 1.636606 |
| FOXF1 | Mesoderm | 0.294467 | 1.48455 | 1.640087 | 0 | 0 | 0 |
| LHX1 | Mesoderm | 4.122537 | 4.572413 | 4.604858 | 0 | 0 | 0 |
| LMO2 | Mesoderm | 0.530041 | 0.29691 | 0.693883 | 0 | 0 | 0 |
| MEOX1 | Mesoderm | 0 | 0 | 0 | 0 | 0 | 0 |
| MEOX2 | Mesoderm | 0 | 0 | 0 | 0 | 0 | 0 |
| MSX1 | Mesoderm | 4.711471 | 3.087864 | 6.560346 | 0 | 0 | 0 |
| MSX2 | Mesoderm | 65.9606 | 83.78799 | 82.50897 | 2.12331 | 1.834119 | 7.460996 |
| MYL4 | Mesoderm | 0.647827 | 0.653202 | 0.820043 | 0 | 0 | 0 |
| MYOCD | Mesoderm | 0 | 0 | 0 | 0 | 0 | 0 |
| PBX1 | Mesoderm | 7.420567 | 7.600895 | 4.794099 | 17.11388 | 12.89124 | 15.35521 |
| ROR2 | Mesoderm | 1.354548 | 1.959606 | 3.027852 | 21.27557 | 16.08784 | 2.743721 |
| SOX6 | Mesoderm | 3.180243 | 1.068876 | 1.703167 | 0 | 0 | 0 |
| TBX6 | Mesoderm | 0.588934 | 0.475056 | 0.567722 | 0 | 0 | 0 |
| TCF15 | Mesoderm | 0 | 0 | 0 | 0 | 0 | 0 |
| WNT5B | Mesoderm | 12.07314 | 15.32055 | 14.6977 | 100.1353 | 91.49634 | 11.93759 |
| WNT8A | Mesoderm | 0 | 0 | 0 | 1.953445 | 1.676909 | 3.128805 |
| ANXA4 | Mixed-lineage | 175.7968 | 184.3811 | 152.8435 | 14.14124 | 12.10519 | 5.679984 |
| BMP2 | Mixed-lineage | 63.54597 | 55.64093 | 64.15262 | 4.119221 | 2.829784 | 0.481355 |
| BMP4 | Mixed-lineage | 16.9024 | 10.80752 | 11.35445 | 5.690471 | 7.38888 | 22.28672 |
| DKK1 | Mixed-lineage | 0 | 0 | 0 | 0 | 0 | 0 |
| EOMES | Mixed-lineage | 0 | 0 | 0 | 0 | 0 | 0 |
| FGF8 | Mixed-lineage | 0 | 0 | 0 | 27.0085 | 15.03978 | 0.144406 |
| FOXA1 | Mixed-lineage | 33.98149 | 35.51043 | 31.72937 | 0 | 0 | 0 |
| GATA4 | Mixed-lineage | 0 | 0 | 0 | 0 | 0 | 0 |
| GSC | Mixed-lineage | 0 | 0 | 0.06308 | 0 | 0 | 0 |
| HAND2 | Mixed-lineage | 0 | 0 | 0 | 0 | 0 | 0 |
| HNF4A | Mixed-lineage | 0.058893 | 0 | 0.126161 | 5.011011 | 6.812443 | 37.30498 |
| ISL1 | Mixed-lineage | 1.295655 | 1.959606 | 1.640087 | 1.443851 | 2.096136 | 4.043379 |
| KDR | Mixed-lineage | 0.17668 | 0.118764 | 0.126161 | 0 | 0 | 0 |
| MESP1 | Mixed-lineage | 0 | 0 | 0 | 0 | 0 | 0 |
| MESP2 | Mixed-lineage | 0 | 0 | 0 | 0 | 0 | 0 |
| MIXL1 | Mixed-lineage | 0 | 0 | 0 | 0 | 0 | 0 |
| OTX | Mixed-lineage | 2.767989 | 6.532019 | 5.1095 | 3.991823 | 5.397551 | 5.343036 |
| OTX2 | Mixed-lineage | 0 | 0.118764 | 0.126161 | 15.37276 | 18.76042 | 46.6914 |
| PDGFRA | Mixed-lineage | 4.181431 | 4.334885 | 4.163297 | 0 | 0 | 0 |
| PDGFRB | Mixed-lineage | 8.303968 | 6.116345 | 8.263513 | 0 | 0 | 0 |
| RIPK4 | Mixed-lineage | 45.52459 | 43.58638 | 46.11166 | 0 | 0 | 0 |
| WNT3A | Mixed-lineage | 0 | 0.059382 | 0.441562 | 0 | 0 | 0 |
| CDX2 | Trophectoderm | 15.79933 | 16.15357 | 21.15703 | 72.9656 | 68.0545 | 6.37875 |
| ELF3 | Trophectoderm | 163.1297 | 174.4325 | 194.9617 | 1.68001 | 2.18448 | 1.22702 |
| GATA2 | Trophectoderm | 190.6322 | 237.8743 | 312.8071 | 0.049519 | 0.1005 | 0.382994 |
| GATA3 | Trophectoderm | 153.7021 | 156.9111 | 171.0481 | 0.231594 | 0.564035 | 1.50691 |
| HAND1 | Trophectoderm | 150.5162 | 162.7081 | 185.3825 | 0.119077 | 0.217503 | 0 |
| TFAP2A | Trophectoderm | 198.2393 | 214.4256 | 196.0644 | 6.39849 | 5.04681 | 0.140591 |

TABLE 4
Functional annotation of genes identified as part of ChEA analysis (FIG.
6); genes in both 16-CE and biPSCs are underlined and in bold.

| Symbol | Name | NCBI Entrez Gene |
|---|---|---|
| ZZZ3 | ZZ-TYPE ZINC FINGER- | |
| | CONTAINING PROTEIN 3 | |
| ZNF592 | Zinc finger protein 592 | This gene is thought to play a role in a complex developmental pathway and the regulation |
| | | of genes involved in cerebellar development. Mutations in this gene have been associated |
| | | with autosomal recessive spinocerebellar ataxia. [provided by RefSeq, January 2011] |
| ZNF318 | Zinc finger protein 318 | |
| ZNF165 | Zinc finger protein 165 | This gene encodes a member of the Kruppel family of zinc finger proteins. Members of |
| | | this DNA-binding protein family act as transcriptional regulators. This gene is located |
| | | within a cluster of zinc finger family members. The encoded protein may play a role in |
| | | spermatogenesis. [provided by RefSeq, July 2008] |
| THAP11 | THAP DOMAIN- | The protein encoded by this gene contains a THAP domain, which is a conserved DNA- |
| | CONTAINING PROTEIN | binding domain that has striking similarity to the site-specific DNA-binding domain |
| | 11 | (DBD) of Drosophila P element transposases. [provided by RefSeq, July 2008] |
| TGIF2 | Homeobox protein TGIF2 | The protein encoded by this gene is a DNA-binding homeobox protein and a |
| | | transcriptional repressor, which appears to repress transcription by recruiting histone |
| | | deacetylases to TGF beta-responsive genes. This gene is amplified and over-expressed in |
| | | some ovarian cancers. Alternative splicing results in multiple transcript variants. A related |
| | | pseudogene has been identified on chromosome 1. Read-through transcription also exists |
| | | between this gene and the neighboring downstream C20orf24 (chromosome 20 open |
| | | reading frame 24) gene. [provided by RefSeq, December 2010] |
| TEAD2 | TRANSCRIPTIONAL | |
| | ENHANCER FACTOR | |
| | TEF-4 | |
| TCF7 | TRANSCRIPTION | This gene encodes a member of the T-cell factor/lymphoid enhancer-binding factor |
| | FACTOR 7 | family of high mobility group (HMG) box transcriptional activators. This gene is |
| | | expressed predominantly in T-cells and plays a critical role in natural killer cell and |
| | | innate lymphoid cell development. The encoded protein forms a complex with beta- |
| | | catenin and activates transcription through a Wnt/beta-catenin signaling pathway. |
| | | Mice with a knockout of this gene are viable and fertile, but display a block in T- |
| | | lymphocyte differentiation. Alternative splicing results in multiple transcript |
| | | variants. Naturally-occurring isoforms lacking the N-terminal beta-catenin |
| | | interaction domain may act as dominant negative regulators of Wnt signaling. |
| | | [provided by RefSeq, October 2016] |
| SP2 | Transcription factor Sp2 | This gene encodes a member of the Sp subfamily of Sp/XKLF transcription factors. Sp |
| | | family proteins are sequence-specific DNA-binding proteins characterized by an amino- |
| | | terminal trans-activation domain and three carboxy-terminal zinc finger motifs. This |
| | | protein contains the least conserved DNA-binding domain within the Sp subfamily of |
| | | proteins, and its DNA sequence specificity differs from the other Sp proteins. It localizes |
| | | primarily within subnuclear foci associated with the nuclear matrix, and can activate or in |
| | | some cases repress expression from different promoters. [provided by RefSeq, July 2008] |
| SOX2 | Transcription factor SOX- | This intronless gene encodes a member of the SRY-related HMG-box (SOX) family |
| | 2 | of transcription factors involved in the regulation of embryonic development and in |
| | | the determination of cell fate. The product of this gene is required for stem-cell |
| | | maintenance in the central nervous system, and also regulates gene expression in the |
| | | stomach. Mutations in this gene have been associated with optic nerve hypoplasia |
| | | and with syndromic microphthalmia, a severe form of structural eye malformation. |
| | | This gene lies within an intron of another gene called SOX2 overlapping transcript |
| | | (SOX2OT). [provided by RefSeq, July 2008] |
| PRDM1 | PR DOMAIN ZINC | This gene encodes a protein that acts as a repressor of beta-interferon gene expression. The |
| | FINGER PROTEIN 1 | protein binds specifically to the PRDI (positive regulatory domain I element) of the beta- |
| | | IFN gene promoter. Transcription of this gene increases upon virus induction. Two |
| | | alternatively spliced transcript variants that encode different isoforms have been reported. |
| | | [provided by RefSeq, July 2008] |
| POU2F1 | POU DOMAIN, CLASS 2, | The OCT1 transcription factor was among the first identified members of the POU |
| | TRANSCRIPTION | transcription factor family (summarized by Sturm et al., 1993 [PubMed 8314572]). |
| | FACTOR 1 | Members of this family contain the POU domain, a 160-amino acid region necessary |
| | | for DNA binding to the octameric sequence ATGCAAAT. [supplied by OMIM, July |
| | | 2010] |
| PCGF2 | POLYCOMB GROUP | The protein encoded by this gene contains a RING finger motif and is similar to the |
| | RING FINGER PROTEIN 2 | polycomb group (PcG) gene products. PcG gene products form complexes via protein- |
| | | protein interaction and maintain the transcription repression of genes involved in |
| | | embryogenesis, cell cycles, and tumorigenesis. This protein was shown to act as a negative |
| | | regulator of transcription and has tumor suppressor activity. The expression of this gene |
| | | was detected in various tumor cells, but is limited in neural organs in normal tissues. |
| | | Knockout studies in mice suggested that this protein may negatively regulate the |
| | | expression of different cytokines, chemokines, and chemokine receptors, and thus plays an |
| | | important role in lymphocyte differentiation and migration, as well as in immune |
| | | responses. [provided by RefSeq, July 2008] |
| OTX2 | HOMEOBOX PROTEIN | This gene encodes a member of the bicoid subfamily of homeodomain-containing |
| | OTX2 | transcription factors. The encoded protein acts as a transcription factor and plays a |
| | | role in brain, craniofacial, and sensory organ development. The encoded protein also |
| | | influences the proliferation and differentiation of dopaminergic neuronal progenitor |
| | | cells during mitosis. Mutations in this gene cause syndromic microphthalmia 5 |
| | | (MCOPS5) and combined pituitary hormone deficiency 6 (CPHD6). This gene is also |
| | | suspected of having an oncogenic role in medulloblastoma. Alternative splicing |
| | | results in multiple transcript variants encoding distinct isoforms. Pseudogenes of this |
| | | gene are known to exist on chromosomes two and nine. [provided by RefSeq, July |
| | | 2012 |
| NR2C2 | NUCLEAR RECEPTOR | This gene encodes a protein that belongs to the nuclear hormone receptor family. |
| | SUBFAMILY 2 GROUP C | Members of this family act as ligand-activated transcription factors and function in many |
| | MEMBER 2 | biological processes such as development, cellular differentiation and homeostasis. The |
| | | activated receptor/ligand complex is translocated to the nucleus where it binds to hormone |
| | | response elements of target genes. The protein encoded by this gene plays a role in |
| | | protecting cells from oxidative stress and damage induced by ionizing radiation. The lack |
| | | of a similar gene in mouse results in growth retardation, severe spinal curvature, |
| | | subfertility, premature aging, and prostatic intraepithelial neoplasia (PIN) development. |
| | | Alternative splicing results in multiple transcript variants encoding different isoforms. |
| | | [provided by RefSeq, April 2014] |
| NFATC1 | Nuclear factor of activated | The product of this gene is a component of the nuclear factor of activated T cells DNA- |
| | T-cells | binding transcription complex. This complex consists of at least two components: a |
| | | preexisting cytosolic component that translocates to the nucleus upon T cell receptor |
| | | (TCR) stimulation, and an inducible nuclear component. Proteins belonging to this family |
| | | of transcription factors play a central role in inducible gene transcription during immune |
| | | response. The product of this gene is an inducible nuclear component. It functions as a |
| | | major molecular target for the immunosuppressive drugs such as cyclosporin A. Multiple |
| | | alternatively spliced transcript variants encoding distinct isoforms have been identified for |
| | | this gene. Different isoforms of this protein may regulate inducible expression of different |
| | | cytokine genes. [provided by RefSeq, July 2013] |
| NCOA3 | NUCLEAR RECEPTOR | The protein encoded by this gene is a nuclear receptor coactivator that interacts with |
| | COACTIVATOR 3 | nuclear hormone receptors to enhance their transcriptional activator functions. The |
| | | encoded protein has histone acetyltransferase activity and recruits p300/CBP-associated |
| | | factor and CREB binding protein as part of a multisubunit coactivation complex. This |
| | | protein is initially found in the cytoplasm but is translocated into the nucleus upon |
| | | phosphorylation. Several transcript variants encoding different isoforms have been found |
| | | for this gene. In addition, a polymorphic repeat region is found in the C-terminus of the |
| | | encoded protein. [provided by RefSeq, March 2010] |
| MYBL2 | MYB-RELATED | The protein encoded by this gene, a member of the MYB family of transcription factor |
| | PROTEIN B | genes, is a nuclear protein involved in cell cycle progression. The encoded protein is |
| | | phosphorylated by cyclin A/cyclin-dependent kinase 2 during the S-phase of the cell cycle |
| | | and possesses both activator and repressor activities. It has been shown to activate the cell |
| | | division cycle 2, cyclin D1, and insulin-like growth factor-binding protein 5 genes. Two |
| | | transcript variants encoding different isoforms have been found for this gene. [provided by |
| | | RefSeq, July 2013] |
| MEIS2 | HOMEOBOX PROTEIN | This gene encodes a homeobox protein belonging to the TALE (‘three amino acid loop |
| | MEIS2 | extension’) family of homeodomain-containing proteins. TALE homeobox proteins are |
| | | highly conserved transcription regulators, and several members have been shown to be |
| | | essential contributors to developmental programs. Multiple transcript variants encoding |
| | | distinct isoforms have been described for this gene. [provided by RefSeq, July 2008] |
| MEIS1 | HOMEOBOX PROTEIN | Homeobox genes, of which the most well-characterized category is represented by the |
| | MEIS | HOX genes, play a crucial role in normal development. In addition, several homeoproteins |
| | | are involved in neoplasia. This gene encodes a homeobox protein belonging to the TALE |
| | | (‘three amino acid loop extension’) family of homeodomain-containing proteins. [provided |
| | | by RefSeq, July 2008] |
| MBD4 | METHYL-CPG-BINDING | The protein encoded by this gene is a member of a family of nuclear proteins related by |
| | DOMAIN PROTEIN 4 | the presence of a methyl-CpG binding domain (MBD). These proteins are capable of |
| | | binding specifically to methylated DNA, and some members can also repress transcription |
| | | from methylated gene promoters. This protein contains an MBD domain at the N-terminus |
| | | that functions both in binding to methylated DNA and in protein interactions and a C- |
| | | terminal mismatch-specific glycosylase domain that is involved in DNA repair. |
| | | Alternatively spliced transcript variants encoding multiple isoforms have been observed |
| | | for this gene. [provided by RefSeq, January 2013] |
| MAFK | Transcription factor MafK | The developmentally regulated expression of the globin genes depends on upstream |
| | | regulatory elements termed locus control regions (LCRs). LCRs are associated with |
| | | powerful enhancer activity that is mediated by the transcription factor NFE2 (nuclear |
| | | factor erythroid-2). NFE2 recognition sites are also present in the gene promoters of 2 |
| | | heme biosynthetic enzymes, porphobilinogen deaminase (PBGD; MIM 609806) and |
| | | ferrochelatase (FECH; MIM 612386). NFE2 DNA-binding activity consists of a |
| | | heterodimer containing an 18-kD Maf protein (MafF, MafG (MIM 602020), or MafK) and |
| | | p45 (MIM 601490). Both subunits are members of the activator protein-1 superfamily of |
| | | basic leucine zipper (bZIP) proteins (see MIM 165160). Maf homodimers suppress |
| | | transcription at NFE2 sites. [supplied by OMIM, November 2008] |
| MAFG | Transcription factor MafG | Globin gene expression is regulated through nuclear factor erythroid-2 (NFE2) elements |
| | | located in enhancer-like locus control regions positioned many kb upstream of alpha- and |
| | | beta-gene clusters (summarized by Blank et al., 1997 [PubMed 9166829]). NFE2 DNA- |
| | | binding activity consists of a heterodimer containing a ubiquitous small Maf protein |
| | | (MafF, MIM 604877; MafG; or MafK, MIM 600197) and the tissue-restricted protein p45 |
| | | NFE2 (MIM 601490). Both subunits are members of the activator protein-1-like |
| | | superfamily of basic leucine zipper (bZIP) proteins (see MIM 165160). [supplied by |
| | | OMIM, March 2010] |
| KMT2B | Histone-lysine N- | This gene encodes a protein which contains multiple domains including a CXXC zinc |
| | methyltransferase 2B | finger, three PHD zinc fingers, two FY-rich domains, and a SET (suppressor of |
| | | variegation, enhancer of zeste, and trithorax) domain. The SET domain is a conserved C- |
| | | terminal domain that characterizes proteins of the MLL (mixed-lineage leukemia) family. |
| | | This gene is ubiquitously expressed in adult tissues. It is also amplified in solid tumor cell |
| | | lines, and may be involved in human cancer. Two alternatively spliced transcript variants |
| | | encoding distinct isoforms have been reported for this gene, however, the full length |
| | | nature of the shorter transcript is not known. [provided by RefSeq, July 2008] |
| KMT2A | Histone-lysine N- | This gene encodes a transcriptional coactivator that plays an essential role in regulating |
| | methyltransferase 2A | gene expression during early development and hematopoiesis. The encoded protein |
| | | contains multiple conserved functional domains. One of these domains, the SET domain, |
| | | is responsible for its histone H3 lysine 4 (H3K4) methyltransferase activity which |
| | | mediates chromatin modifications associated with epigenetic transcriptional activation. |
| | | This protein is processed by the enzyme Taspase 1 into two fragments, MLL-C and MLL- |
| | | N. These fragments reassociate and further assemble into different multiprotein complexes |
| | | that regulate the transcription of specific target genes, including many of the HOX genes. |
| | | Multiple chromosomal translocations involving this gene are the cause of certain acute |
| | | lymphoid leukemias and acute myeloid leukemias. Alternate splicing results in multiple |
| | | transcript variants. [provided by RefSeq, October 2010] |
| KLF3 | KRUEPPEL-LIKE | |
| | FACTOR 3 | |
| IRF3 | Interferon regulatory factor | This gene encodes a member of the interferon regulatory transcription factor (IRF) family. |
| | 3 | The encoded protein is found in an inactive cytoplasmic form that upon serine/threonine |
| | | phosphorylation forms a complex with CREBBP. This complex translocates to the nucleus |
| | | and activates the transcription of interferons alpha and beta, as well as other interferon- |
| | | induced genes. The protein plays an important role in the innate immune response against |
| | | DNA and RNA viruses. Mutations in this gene are associated with Encephalopathy, acute, |
| | | infection-induced, herpes-specific, 7. [provided by RefSeq, September 2020] |
| HPF1 | HISTONE PARYLATION | |
| | FACTOR 1 | |
| HOXC11 | HOMEOBOX PROTEIN | This gene belongs to the homeobox family of genes. The homeobox genes encode a highly |
| | HOX-C11 | conserved family of transcription factors that play an important role in morphogenesis in |
| | | all multicellular organisms. Mammals possess four similar homeobox gene clusters, |
| | | HOXA, HOXB, HOXC and HOXD, which are located on different chromosomes and |
| | | consist of 9 to 11 genes arranged in tandem. This gene is one of several homeobox HOXC |
| | | genes located in a cluster on chromosome 12. The product of this gene binds to a promoter |
| | | element of the lactase-phlorizin hydrolase. It also may play a role in early intestinal |
| | | development. An alternatively spliced variant encoding a shorter isoform has been |
| | | described but its full-length nature has not been determined. [provided by RefSeq, July |
| | | 2008] |
| HOXB7 | HOMEOBOX PROTEIN | This gene is a member of the Antp homeobox family and encodes a protein with a |
| | HOX-B7 | homeobox DNA-binding domain. It is included in a cluster of homeobox B genes located |
| | | on chromosome 17. The encoded nuclear protein functions as a sequence-specific |
| | | transcription factor that is involved in cell proliferation and differentiation. Increased |
| | | expression of this gene is associated with some cases of melanoma and ovarian carcinoma. |
| | | [provided by RefSeq, July 2008] |
| HOXA9 | HOMEOBOX PROTEIN | In vertebrates, the genes encoding the class of transcription factors called homeobox genes |
| | HOX-A9 | are found in clusters named A, B, C, and D on four separate chromosomes. Expression of |
| | | these proteins is spatially and temporally regulated during embryonic development. This |
| | | gene is part of the A cluster on chromosome 7 and encodes a DNA-binding transcription |
| | | factor which may regulate gene expression, morphogenesis, and differentiation. This gene |
| | | is highly similar to the abdominal-B (Abd-B) gene of Drosophila. A specific translocation |
| | | event which causes a fusion between this gene and the NUP98 gene has been associated |
| | | with myeloid leukemogenesis. Read-through transcription exists between this gene and the |
| | | upstream homeobox A10 (HOXA10) gene. [provided by RefSeq, March 2011] |
| HNF1B | HEPATOCYTE | This gene encodes a member of the homeodomain-containing superfamily of |
| | NUCLEAR FACTOR 1- | transcription factors. The protein binds to DNA as either a homodimer, or a |
| | BETA | heterodimer with the related protein hepatocyte nuclear factor 1-alpha. The gene has |
| | | been shown to function in nephron development, and regulates development of the |
| | | embryonic pancreas. Mutations in this gene result in renal cysts and diabetes |
| | | syndrome and noninsulin-dependent diabetes mellitus, and expression of this gene is |
| | | altered in some types of cancer. Multiple transcript variants encoding different |
| | | isoforms have been found for this gene. [provided by RefSeq, September 2009] |
| HMBOX1 | HOMEOBOX- | |
| | CONTAINING PROTEIN 1 | |
| HINFP | HISTONE H4 | This gene encodes a transcription factor that interacts with methyl-CpG-binding |
| | TRANSCRIPTION | protein-2 (MBD2), a component of the MeCP1 histone deacetylase (HDAC) complex, |
| | FACTOR | and plays a role in DNA methylation and transcription repression. Alternatively |
| | | spliced transcript variants have been found for this gene. [provided by RefSeq, August |
| | | 2011] |
| HHEX | HEMATOPOIETICALLY- | This gene encodes a member of the homeobox family of transcription factors, many |
| | EXPRESSED | of which are involved in developmental processes. Expression in specific |
| | HOMEOBOX PROTEIN | hematopoietic lineages suggests that this protein may play a role in hematopoietic |
| | HHEX | differentiation. [provided by RefSeq, July 2008] |
| GLYR1 | OXIDOREDUCTASE | |
| | GLYR1-RELATED | |
| GATA2 | ENDOTHELIAL | This gene encodes a member of the GATA family of zinc-finger transcription factors that |
| | TRANSCRIPTION | are named for the consensus nucleotide sequence they bind in the promoter regions of |
| | FACTOR GATA-2 | target genes. The encoded protein plays an essential role in regulating transcription of |
| | | genes involved in the development and proliferation of hematopoietic and endocrine cell |
| | | lineages. Alternative splicing results in multiple transcript variants. [provided by RefSeq, |
| | | March 2009] |
| FOXP1 | FORKHEAD BOX | This gene belongs to subfamily P of the forkhead box (FOX) transcription factor family. |
| | PROTEIN P1 | Forkhead box transcription factors play important roles in the regulation of tissue- and cell |
| | | type-specific gene transcription during both development and adulthood. Forkhead box P1 |
| | | protein contains both DNA-binding- and protein-protein binding-domains. This gene may |
| | | act as a tumor suppressor as it is lost in several tumor types and maps to a chromosomal |
| | | region (3p14.1) reported to contain a tumor suppressor gene(s). Alternative splicing results |
| | | in multiple transcript variants encoding different isoforms. [provided by RefSeq, July 2008] |
| FOXM1 | FORKHEAD BOX | The protein encoded by this gene is a transcriptional activator involved in cell |
| | PROTEIN M1 | proliferation. The encoded protein is phosphorylated in M phase and regulates the |
| | | expression of several cell cycle genes, such as cyclin B1 and cyclin D1. Several |
| | | transcript variants encoding different isoforms have been found for this gene. |
| | | [provided by RefSeq, July 2011] |
| FOXK1 | FORKHEAD BOX | |
| | PROTEIN K1 | |
| FOXJ2 | FORKHEAD BOX | |
| | PROTEIN J2 | |
| ELK1 | ETS DOMAIN- | This gene is a member of the Ets family of transcription factors and of the ternary complex |
| | CONTAINING PROTEIN | factor (TCF) subfamily. Proteins of the TCF subfamily form a ternary complex by binding |
| | ELK-1 | to the the serum response factor and the serum response element in the promoter of the c- |
| | | fos proto-oncogene. The protein encoded by this gene is a nuclear target for the ras-raf- |
| | | MAPK signaling cascade. This gene produces multiple isoforms by using alternative |
| | | translational start codons and by alternative splicing. Related pseudogenes have been |
| | | identified on chromosomes 7 and 14. [provided by RefSeq, March 2012] |
| E2F7 | TRANSCRIPTION | E2F transcription factors, such as E2F7, play an essential role in the regulation of |
| | FACTOR E2F7 | cell cycle progression (Di Stefano et al., 2003 [PubMed 14633988]). [supplied by |
| | | OMIM, May 2008] |
| E2F5 | TRANSCRIPTION | The protein encoded by this gene is a member of the E2F family of transcription factors. |
| | FACTOR E2F5 | The E2F family plays a crucial role in the control of cell cycle and action of tumor |
| | | suppressor proteins and is also a target of the transforming proteins of small DNA tumor |
| | | viruses. The E2F proteins contain several evolutionarily conserved domains that are |
| | | present in most members of the family. These domains include a DNA binding domain, a |
| | | dimerization domain which determines interaction with the differentiation regulated |
| | | transcription factor proteins (DP), a transactivation domain enriched in acidic amino acids, |
| | | and a tumor suppressor protein association domain which is embedded within the |
| | | transactivation domain. This protein is differentially phosphorylated and is expressed in a |
| | | wide variety of human tissues. It has higher identity to E2F4 than to other family |
| | | members. Both this protein and E2F4 interact with tumor suppressor proteins p130 and |
| | | p107, but not with pRB. Alternative splicing results in multiple variants encoding different |
| | | isoforms. [provided by RefSeq, July 2008] |
| DEAF1 | DEFORMED EPIDERMAL | This gene encodes a zinc finger domain-containing protein that functions as a regulator of |
| | AUTOREGULATORY | transcription. The encoded proteins binds to its own promoter as well as to that of several |
| | FACTOR 1 HOMOLOG | target genes. Activity of this protein is important in the regulation of embryonic |
| | | development. Mutations in this gene have been found in individuals with autosomal |
| | | dominant cognitive disability. Alternative splicing results in multiple transcript variants. |
| | | [provided by RefSeq, June 2014] |
| CEBPZ | CCAAT/ENHANCER- | This gene belongs to the CBF/Mak21 family. The encoded protein plays a role in cellular |
| | BINDING PROTEIN ZETA | response to environmental stimuli through a transcriptional process that involves heat |
| | | shock factors, conserved DNA elements (heat shock elements or HSEs) and CCAAT |
| | | boxes. The protein acts as a DNA-binding transcriptional activator and regulates the heat- |
| | | shock protein 70 (HSP70) promoter in a CCAAT-dependent manner. The protein is also |
| | | involved in cell growth and differentiation, particularly, hematopoietic differentiation. |
| | | [provided by RefSeq, November 2020] |
| CEBPG | CCAAT/ENHANCER- | The C/EBP family of transcription factors regulates viral and cellular CCAAT/enhancer |
| | BINDING PROTEIN | element-mediated transcription. C/EBP proteins contain the bZIP region, which is |
| | GAMMA | characterized by two motifs in the C-terminal half of the protein: a basic region involved |
| | | in DNA binding and a leucine zipper motif involved in dimerization. The C/EBP family |
| | | consist of several related proteins, C/EBP alpha, C/EBP beta, C/EBP gamma, and C/EBP |
| | | delta, that form homodimers and that form heterodimers with each other. |
| | | CCAAT/enhancer binding protein gamma may cooperate with Fos to bind PRE-I enhancer |
| | | elements. Two transcript variants encoding the same protein have been found for this |
| | | gene. [provided by RefSeq, November 2011] |
| CEBPB | CCAAT/ENHANCER- | This intronless gene encodes a transcription factor that contains a basic leucine zipper |
| | BINDING PROTEIN BETA | (bZIP) domain. The encoded protein functions as a homodimer but can also form |
| | | heterodimers with CCAAT/enhancer-binding proteins alpha, delta, and gamma. Activity |
| | | of this protein is important in the regulation of genes involved in immune and |
| | | inflammatory responses, among other processes. The use of alternative in-frame AUG |
| | | start codons results in multiple protein isoforms, each with distinct biological functions. |
| | | [provided by RefSeq, October 2013] |
| AHR | ARYL HYDROCARBON | The protein encoded by this gene is a ligand-activated helix-loop-helix transcription factor |
| | RECEPTOR | involved in the regulation of biological responses to planar aromatic hydrocarbons. This |
| | | receptor has been shown to regulate xenobiotic-metabolizing enzymes such as cytochrome |
| | | P450. Before ligand binding, the encoded protein is sequestered in the cytoplasm; upon |
| | | ligand binding, this protein moves to the nucleus and stimulates transcription of target |
| | | genes. [provided by RefSeq, September 2015] |

TABLE 5
Genes upregulated in biPSCs and 16-CE compared to bovine fibroblasts (FDR < 0.05). (FIG. 7A, common genes).

| | | Fibroblasts vs 16-CE | | | | Fibroblasts vs biPSC | | | |
|---|---|---|---|---|---|---|---|---|---|
| | Gene | logFC | logCPM | PValue | FDR | logFC | logCPM | PValue | FDR |
| ENSBTAG00000002065 | TFAP2C | 9.470355 | 5.532433 | 6.77E−60 | 1.89E−58 | 12.0255 | 8.034965 | 6.27E−44 | 2.00E−41 |
| ENSBTAG00000021158 | SATB1 | 6.234696 | 2.730832 | 1.53E−08 | 4.39E−08 | 11.54375 | 7.557032 | 4.02E−40 | 7.70E−38 |
| ENSBTAG00000037756 | TET1 | 3.066376 | 4.512679 | 2.05E−17 | 1.07E−16 | 7.404996 | 8.582076 | 2.75E−38 | 4.13E−36 |
| ENSBTAG00000044139 | ZFP42 | 9.475288 | 5.538191 | 4.56E−60 | 1.28E−58 | 10.95715 | 6.978451 | 1.46E−35 | 1.70E−33 |
| ENSBTAG00000001005 | ZNF280B | 2.406672 | 4.502052 | 7.27E−13 | 2.86E−12 | 6.531072 | 8.276189 | 2.39E−33 | 2.10E−31 |
| ENSBTAG00000016190 | ZNF394 | 2.683775 | 4.962588 | 5.59E−19 | 3.16E−18 | 6.384116 | 8.391248 | 7.26E−33 | 6.12E−31 |
| ENSBTAG00000003339 | ZBTB10 | 3.132062 | 5.389932 | 9.89E−29 | 9.17E−28 | 6.315466 | 8.378156 | 1.93E−32 | 1.57E−30 |
| ENSBTAG00000012074 | MYB | 6.36298 | 2.822893 | 2.24E−09 | 6.87E−09 | 10.30439 | 6.338278 | 1.42E−30 | 9.70E−29 |
| ENSBTAG00000010392 | ESRRG | 5.176466 | 2.068575 | 0.000284 | 0.000542 | 9.650887 | 5.704437 | 8.78E−26 | 3.29E−24 |
| ENSBTAG00000003638 | ZNF215 | 7.183192 | 3.46171 | 1.48E−15 | 6.96E−15 | 9.634155 | 5.687617 | 1.21E−25 | 4.46E−24 |
| ENSBTAG00000006511 | MTF1 | 0.812511 | 3.987008 | 0.024536 | 0.035751 | 5.326547 | 7.718107 | 1.64E−25 | 6.00E−24 |
| ENSBTAG00000013412 | NFAT5 | 0.750807 | 4.163744 | 0.023284 | 0.034041 | 5.240734 | 7.868976 | 2.01E−25 | 7.26E−24 |
| ENSBTAG00000012124 | GPBP1 | 1.57171 | 6.748042 | 5.83E−18 | 3.13E−17 | 4.866776 | 9.652906 | 6.37E−25 | 2.22E−23 |
| ENSBTAG00000040169 | Zfy1 | 5.03337 | 5.612287 | 3.47E−50 | 6.97E−49 | 5.938147 | 6.472636 | 2.05E−24 | 6.64E−23 |
| ENSBTAG00000016137 | ZNF608 | 3.384532 | 4.10963 | 5.30E−15 | 2.40E−14 | 5.918173 | 6.403707 | 6.89E−24 | 2.09E−22 |
| ENSBTAG00000000060 | PRDM14 | 6.51234 | 2.933427 | 3.34E−10 | 1.10E−09 | 9.328429 | 5.39413 | 1.80E−23 | 5.17E−22 |
| ENSBTAG00000013702 | ZNF296 | 8.877931 | 4.97078 | 2.20E−43 | 3.55E−42 | 9.306817 | 5.374463 | 2.41E−23 | 6.76E−22 |
| ENSBTAG00000011642 | RBM27 | 2.285676 | 5.690046 | 8.49E−22 | 5.58E−21 | 4.830201 | 7.973789 | 3.70E−23 | 1.02E−21 |
| ENSBTAG00000019885 | ZNF280C | 3.000473 | 3.641352 | 1.12E−09 | 3.56E−09 | 5.890856 | 6.190519 | 6.86E−23 | 1.84E−21 |
| ENSBTAG00000006760 | OTX1 | 5.33262 | 2.154728 | 7.49E−05 | 0.000153 | 9.21075 | 5.283286 | 1.09E−22 | 2.83E−21 |
| ENSBTAG00000003101 | SALL4 | 10.11141 | 6.149877 | 7.25E−83 | 3.84E−81 | 9.079407 | 5.157175 | 9.30E−22 | 2.22E−20 |
| ENSBTAG00000022450 | THAP9 | 2.178175 | 4.849033 | 2.09E−13 | 8.51E−13 | 4.781472 | 7.139215 | 2.48E−21 | 5.64E−20 |
| ENSBTAG00000014602 | CAMTA1 | 4.779811 | 1.868623 | 0.002134 | 0.003643 | 8.99964 | 5.081298 | 3.27E−21 | 7.32E−20 |
| ENSBTAG00000044074 | MLLT10 | 1.732231 | 5.845247 | 3.69E−15 | 1.70E−14 | 4.461774 | 8.221876 | 4.43E−21 | 9.85E−20 |
| ENSBTAG00000012285 | ESRRB | 10.3788 | 6.413486 | 6.18E−94 | 4.14E−92 | 8.936434 | 5.023903 | 8.48E−21 | 1.80E−19 |
| ENSBTAG00000012064 | ZNF473 | 2.51507 | 4.88432 | 7.15E−17 | 3.63E−16 | 4.757596 | 6.87298 | 9.64E−21 | 2.04E−19 |
| ENSBTAG00000007718 | TGIF1 | 2.754611 | 5.334981 | 1.64E−23 | 1.18E−22 | 4.70487 | 7.094028 | 1.00E−20 | 2.12E−19 |
| ENSBTAG00000000363 | BDP1 | 2.058658 | 6.118831 | 2.91E−22 | 1.97E−21 | 4.375795 | 8.162316 | 1.67E−20 | 3.43E−19 |
| ENSBTAG00000015138 | MGA | 2.651976 | 6.996176 | 2.61E−44 | 4.32E−43 | 4.326792 | 8.513994 | 1.67E−20 | 3.44E−19 |
| ENSBTAG00000015386 | REL | 4.922268 | 1.937288 | 0.001088 | 0.00193 | 8.858098 | 4.950685 | 2.93E−20 | 5.82E−19 |
| ENSBTAG00000009415 | NUFIP1 | 2.802819 | 5.374592 | 6.85E−25 | 5.26E−24 | 4.557989 | 6.950913 | 6.70E−20 | 1.28E−18 |
| ENSBTAG00000009426 | EZH2 | 4.029283 | 6.739648 | 1.14E−68 | 4.17E−67 | 4.48021 | 7.166184 | 8.29E−20 | 1.58E−18 |
| ENSBTAG00000011052 | F1N7R8 | 1.063426 | 3.311729 | 0.024397 | 0.035561 | 4.764106 | 6.236903 | 3.65E−19 | 6.44E−18 |
| ENSBTAG00000038888 | ZBTB9 | 3.327424 | 5.430036 | 1.85E−31 | 1.94E−30 | 4.551523 | 6.540999 | 6.82E−19 | 1.18E−17 |
| ENSBTAG00000004136 | NFE2L3 | 7.695026 | 3.895279 | 2.16E−21 | 1.40E−20 | 8.636013 | 4.742973 | 7.43E−19 | 1.27E−17 |
| ENSBTAG00000006844 | LEF1 | 8.684916 | 4.786451 | 7.11E−39 | 9.98E−38 | 8.492773 | 4.608122 | 5.96E−18 | 9.05E−17 |
| ENSBTAG00000020552 | PRDM2 | 1.42921 | 6.17514 | 9.97E−13 | 3.89E−12 | 3.908964 | 8.264643 | 1.29E−17 | 1.89E−16 |
| ENSBTAG00000013949 | AHCTF1 | 2.451523 | 7.660567 | 6.11E−47 | 1.09E−45 | 3.767473 | 8.830246 | 5.51E−17 | 7.45E−16 |
| ENSBTAG00000002613 | MIS18BP1 | 3.543171 | 5.864807 | 4.62E−41 | 6.93E−40 | 4.146304 | 6.417362 | 1.71E−16 | 2.19E−15 |
| ENSBTAG00000011828 | ARID3B | 4.542154 | 5.369643 | 1.59E−41 | 2.42E−40 | 4.620978 | 5.441785 | 3.31E−16 | 4.07E−15 |
| ENSBTAG00000005734 | GATA6 | 7.07256 | 3.371469 | 1.66E−14 | 7.31E−14 | 8.198967 | 4.342946 | 3.86E−16 | 4.70E−15 |
| ENSBTAG00000019120 | WDHD1 | 4.072773 | 6.282752 | 6.24E−58 | 1.62E−56 | 4.067857 | 6.283285 | 4.53E−16 | 5.49E−15 |
| ENSBTAG00000018131 | ATF1 | 0.535682 | 5.612678 | 0.014839 | 0.022553 | 3.664897 | 8.049929 | 6.25E−16 | 7.45E−15 |
| ENSBTAG00000014088 | MBNL3 | 5.332491 | 2.154635 | 7.49E−05 | 0.000153 | 8.150246 | 4.298615 | 8.03E−16 | 9.39E−15 |
| ENSBTAG00000005564 | PIAS1 | 1.202223 | 5.186688 | 1.34E−06 | 3.29E−06 | 3.715091 | 7.228099 | 1.79E−15 | 2.01E−14 |
| ENSBTAG00000024534 | POU2F1 | 3.735428 | 3.595766 | 5.75E−12 | 2.14E−11 | 5.219693 | 4.86341 | 1.91E−15 | 2.14E−14 |
| ENSBTAG00000021706 | TBX3 | 2.437708 | 3.618096 | 9.20E−08 | 2.50E−07 | 4.478143 | 5.309502 | 2.61E−15 | 2.87E−14 |
| ENSBTAG00000009126 | YBX2 | 4.475896 | 1.733756 | 0.008256 | 0.012889 | 8.042937 | 4.20128 | 3.70E−15 | 3.98E−14 |
| ENSBTAG00000007329 | SETDB2 | 1.17093 | 4.262826 | 0.000421 | 0.000792 | 3.868199 | 6.409466 | 3.79E−15 | 4.08E−14 |
| ENSBTAG00000010291 | RBAK | 1.800432 | 4.282083 | 1.06E−07 | 2.85E−07 | 3.94405 | 6.047938 | 5.61E−15 | 5.91E−14 |
| ENSBTAG00000015015 | HINFP | 2.685105 | 5.333128 | 9.39E−23 | 6.53E−22 | 3.839088 | 6.342984 | 6.54E−15 | 6.82E−14 |
| ENSBTAG00000017840 | BAZ2A | 1.723616 | 7.493452 | 2.16E−25 | 1.71E−24 | 3.423616 | 8.932126 | 8.67E−15 | 8.95E−14 |
| ENSBTAG00000021111 | POU5F1 | 9.870166 | 5.917937 | 1.35E−73 | 5.68E−72 | 7.916544 | 4.089571 | 1.86E−14 | 1.84E−13 |
| ENSBTAG00000007746 | AHR | 1.420007 | 3.581786 | 0.000795 | 0.001439 | 4.015294 | 5.621145 | 2.52E−14 | 2.44E−13 |
| ENSBTAG00000013095 | FOXN1 | 4.332297 | 1.6755 | 0.016287 | 0.02429 | 7.889283 | 4.062459 | 2.84E−14 | 2.71E−13 |
| ENSBTAG00000046612 | ZNF292 | 1.863451 | 5.574234 | 3.95E−15 | 1.81E−14 | 3.535234 | 6.97955 | 3.10E−14 | 2.96E−13 |
| ENSBTAG00000019695 | RLF | 1.81328 | 5.717858 | 1.52E−15 | 7.12E−15 | 3.494947 | 7.128677 | 3.43E−14 | 3.27E−13 |
| ENSBTAG00000003021 | SP1 | 1.068727 | 6.057994 | 1.11E−07 | 3.00E−07 | 3.358578 | 7.893336 | 5.60E−14 | 5.14E−13 |
| ENSBTAG00000011981 | MYBL1 | 2.636238 | 3.830519 | 1.11E−09 | 3.53E−09 | 4.263263 | 5.188297 | 5.84E−14 | 5.36E−13 |
| ENSBTAG00000021000 | USF3 | 2.200009 | 4.807531 | 3.67E−13 | 1.48E−12 | 3.711091 | 6.080464 | 8.42E−14 | 7.54E−13 |
| ENSBTAG00000015504 | ZNF131 | 1.751944 | 6.054986 | 5.87E−17 | 2.99E−16 | 3.356149 | 7.394297 | 1.18E−13 | 1.04E−12 |
| ENSBTAG00000038635 | Zfy1 | 1.84962 | 5.105362 | 5.03E−12 | 1.88E−11 | 3.508994 | 6.484727 | 1.54E−13 | 1.35E−12 |
| ENSBTAG00000046561 | LCORL | 1.595751 | 4.112877 | 1.00E−05 | 2.27E−05 | 3.708707 | 5.804211 | 2.79E−13 | 2.36E−12 |
| ENSBTAG00000008132 | SOX13 | 5.426722 | 4.923574 | 1.05E−35 | 1.32E−34 | 4.99112 | 4.531376 | 3.23E−13 | 2.72E−12 |
| ENSBTAG00000024199 | KMT2C | 1.88983 | 6.635403 | 2.53E−23 | 1.81E−22 | 3.243566 | 7.776456 | 3.28E−13 | 2.75E−12 |
| ENSBTAG00000014554 | SNAI1 | 1.100817 | 3.53465 | 0.011247 | 0.017333 | 3.758462 | 5.547626 | 4.17E−13 | 3.44E−12 |
| ENSBTAG00000017776 | CHD2 | 1.680821 | 4.936031 | 1.55E−09 | 4.85E−09 | 3.438362 | 6.372615 | 5.81E−13 | 4.71E−12 |
| ENSBTAG00000038240 | ZNF134 | 2.688653 | 4.147783 | 3.41E−12 | 1.28E−11 | 3.9109 | 5.173354 | 7.05E−13 | 5.62E−12 |
| ENSBTAG00000007053 | ZFHX2 | 1.797622 | 3.346935 | 0.000274 | 0.000529 | 4.037613 | 5.091067 | 7.12E−13 | 5.67E−12 |
| ENSBTAG00000040093 | FOXI3 | 4.072962 | 1.578832 | 0.032192 | 0.045433 | 7.604212 | 3.814138 | 8.33E−13 | 6.60E−12 |
| ENSBTAG00000007074 | ZKSCAN8 | 2.935322 | 3.863682 | 2.03E−11 | 7.31E−11 | 4.099253 | 4.830389 | 1.51E−12 | 1.15E−11 |
| ENSBTAG00000010217 | ZNF318 | 2.023671 | 6.213987 | 1.68E−22 | 1.15E−21 | 3.181786 | 7.187155 | 1.72E−12 | 1.31E−11 |
| ENSBTAG00000000252 | POLE3 | 2.509745 | 6.451489 | 1.02E−33 | 1.18E−32 | 3.213053 | 7.060226 | 1.76E−12 | 1.33E−11 |
| ENSBTAG00000018093 | KMT2A | 1.728363 | 6.413692 | 1.04E−18 | 5.79E−18 | 3.114853 | 7.560088 | 2.66E−12 | 1.96E−11 |
| ENSBTAG00000004079 | ZNF106 | 0.829211 | 7.544256 | 1.55E−07 | 4.12E−07 | 2.999624 | 9.23177 | 3.26E−12 | 2.38E−11 |
| ENSBTAG00000013460 | ZBTB11 | 1.762413 | 5.427025 | 5.65E−13 | 2.25E−12 | 3.210327 | 6.616869 | 4.13E−12 | 2.97E−11 |
| ENSBTAG00000017994 | MAX | 1.043173 | 4.979757 | 8.73E−05 | 0.000177 | 3.199036 | 6.6537 | 4.54E−12 | 3.25E−11 |
| ENSBTAG00000014451 | YEATS2 | 1.487647 | 6.497641 | 3.65E−15 | 1.68E−14 | 3.044138 | 7.76154 | 4.94E−12 | 3.51E−11 |
| ENSBTAG00000006404 | CENPT | 4.3767 | 6.504594 | 5.04E−68 | 1.82E−66 | 3.452415 | 5.667149 | 5.01E−12 | 3.55E−11 |
| ENSBTAG00000007833 | PIAS2 | 0.858806 | 5.295648 | 0.000323 | 0.000613 | 3.063992 | 6.980921 | 1.07E−11 | 7.29E−11 |
| ENSBTAG00000021433 | F1N5B8 | 1.226781 | 3.101685 | 0.021078 | 0.030997 | 3.789086 | 4.983027 | 1.22E−11 | 8.24E−11 |
| ENSBTAG00000015101 | HMGB2 | 1.676587 | 4.82416 | 5.25E−09 | 1.57E−08 | 3.246282 | 6.091304 | 1.31E−11 | 8.88E−11 |
| ENSBTAG00000002295 | ATF2 | 0.900652 | 5.43984 | 0.000109 | 0.000219 | 3.045705 | 7.088924 | 1.34E−11 | 9.04E−11 |
| ENSBTAG00000012938 | JARID2 | 3.788079 | 5.400027 | 1.99E−35 | 2.47E−34 | 3.576554 | 5.21528 | 1.45E−11 | 9.74E−11 |
| ENSBTAG00000013314 | PBX3 | 1.427034 | 4.205409 | 3.79E−05 | 8.05E−05 | 3.313822 | 5.676356 | 2.81E−11 | 1.82E−10 |
| ENSBTAG00000002849 | IRF6 | 5.035525 | 1.994395 | 0.000556 | 0.001022 | 7.304968 | 3.562474 | 3.02E−11 | 1.94E−10 |
| ENSBTAG00000018948 | HIF3A | 4.275909 | 1.653544 | 0.016287 | 0.02429 | 7.30993 | 3.565975 | 3.02E−11 | 1.94E−10 |
| ENSBTAG00000009265 | NR5A2 | 4.091959 | 1.585652 | 0.032192 | 0.045433 | 7.260721 | 3.523208 | 5.26E−11 | 3.27E−10 |
| ENSBTAG00000020860 | CHD1 | 1.393644 | 6.771202 | 8.39E−15 | 3.77E−14 | 2.82644 | 7.915683 | 7.78E−11 | 4.73E−10 |
| ENSBTAG00000001002 | TCF7 | 3.398907 | 5.484047 | 5.12E−33 | 5.75E−32 | 3.281045 | 5.379812 | 1.11E−10 | 6.62E−10 |
| ENSBTAG00000008571 | CUX2 | 5.911884 | 2.510027 | 7.43E−07 | 1.86E−06 | 7.190993 | 3.465575 | 1.24E−10 | 7.31E−10 |
| ENSBTAG00000008248 | DMD | 1.850403 | 4.337283 | 2.49E−08 | 7.09E−08 | 3.228683 | 5.417709 | 1.29E−10 | 7.61E−10 |
| ENSBTAG00000011313 | CASP8AP2 | 2.413698 | 5.549699 | 3.97E−22 | 2.65E−21 | 3.014661 | 6.055258 | 1.43E−10 | 8.41E−10 |
| ENSBTAG00000018488 | AFF1 | 1.630688 | 5.851927 | 5.81E−14 | 2.46E−13 | 2.860282 | 6.837342 | 1.74E−10 | 1.01E−09 |
| ENSBTAG00000003541 | ZNF614 | 3.399769 | 5.247923 | 1.95E−29 | 1.87E−28 | 3.271247 | 5.136958 | 2.46E−10 | 1.40E−09 |
| ENSBTAG00000037375 | A0A3Q1LX57 | 2.707654 | 4.08696 | 2.04E−11 | 7.37E−11 | 3.554017 | 4.780908 | 2.55E−10 | 1.45E−09 |
| ENSBTAG00000006933 | PPP1R10 | 1.436303 | 6.255127 | 3.35E−13 | 1.35E−12 | 2.776201 | 7.319692 | 2.63E−10 | 1.49E−09 |
| ENSBTAG00000017488 | KLF3 | 0.906891 | 4.899264 | 0.000724 | 0.001317 | 2.894895 | 6.396438 | 2.69E−10 | 1.53E−09 |
| ENSBTAG00000025146 | F1N7M4 | 1.635895 | 3.859507 | 2.72E−05 | 5.86E−05 | 3.287081 | 5.129997 | 2.72E−10 | 1.54E−09 |
| ENSBTAG00000038755 | Zfp11 | 4.840114 | 4.302994 | 1.83E−23 | 1.31E−22 | 4.422114 | 3.944864 | 2.93E−10 | 1.65E−09 |
| ENSBTAG00000016077 | ZNF317 | 1.42721 | 4.250023 | 2.55E−05 | 5.50E−05 | 3.123342 | 5.556714 | 3.05E−10 | 1.71E−09 |
| ENSBTAG00000008414 | ZFR | 0.598 | 7.078286 | 0.000347 | 0.000658 | 2.65428 | 8.603367 | 4.93E−10 | 2.68E−09 |
| ENSBTAG00000001241 | ISL1 | 4.407652 | 1.705626 | 0.008256 | 0.012889 | 7.022822 | 3.330777 | 7.44E−10 | 3.91E−09 |
| ENSBTAG00000020355 | KLF4 | 1.059684 | 7.520794 | 3.71E−11 | 1.31E−10 | 2.601429 | 8.713962 | 9.68E−10 | 5.04E−09 |
| ENSBTAG00000007802 | BCLAF1 | 1.734626 | 7.833661 | 9.52E−28 | 8.46E−27 | 2.597214 | 8.533896 | 1.05E−09 | 5.42E−09 |
| ENSBTAG00000016328 | SFPQ | 2.672324 | 8.404264 | 1.41E−62 | 4.29E−61 | 2.588038 | 8.333881 | 1.33E−09 | 6.77E−09 |
| ENSBTAG00000011789 | REST | 1.333278 | 5.850123 | 5.36E−10 | 1.75E−09 | 2.669276 | 6.891636 | 1.80E−09 | 8.98E−09 |
| ENSBTAG00000016931 | E2F7 | 8.060895 | 4.217626 | 6.36E−27 | 5.41E−26 | 6.873677 | 3.204624 | 2.64E−09 | 1.28E−08 |
| ENSBTAG00000017824 | IRF8 | 5.059025 | 2.00652 | 0.000556 | 0.001022 | 6.900555 | 3.228696 | 2.64E−09 | 1.28E−08 |
| ENSBTAG00000020685 | Hmgb2 | 2.975791 | 2.898361 | 4.74E−06 | 1.11E−05 | 4.161735 | 3.783787 | 2.80E−09 | 1.36E−08 |
| ENSBTAG00000007020 | ZNF502 | 1.606217 | 2.523361 | 0.019315 | 0.028566 | 3.813756 | 4.029195 | 2.95E−09 | 1.43E−08 |
| ENSBTAG00000011368 | NOC3L | 0.847382 | 5.645385 | 0.000124 | 0.000249 | 2.598814 | 6.948181 | 3.70E−09 | 1.76E−08 |
| ENSBTAG00000013531 | CENPS | 1.863871 | 4.84476 | 7.79E−11 | 2.69E−10 | 2.851627 | 5.631911 | 3.75E−09 | 1.78E−08 |
| ENSBTAG00000045744 | NKRF | 2.502484 | 5.147033 | 5.60E−19 | 3.17E−18 | 2.883458 | 5.462049 | 3.79E−09 | 1.80E−08 |
| ENSBTAG00000010819 | HMBOX1 | 1.315535 | 4.049334 | 0.000294 | 0.00056 | 2.944227 | 5.268912 | 4.16E−09 | 1.97E−08 |
| ENSBTAG00000006428 | ZNF354A | 1.024923 | 3.732716 | 0.008244 | 0.012889 | 2.973874 | 5.139799 | 4.25E−09 | 2.01E−08 |
| ENSBTAG00000040442 | Zfy1 | 1.421464 | 3.002661 | 0.014385 | 0.021882 | 3.404243 | 4.408382 | 6.25E−09 | 2.90E−08 |
| ENSBTAG00000011598 | sox2 | 11.32308 | 7.339674 | 2.95E−140 | 3.91E−138 | 6.789306 | 3.144532 | 7.11E−09 | 3.25E−08 |
| ENSBTAG00000008138 | HOXA2 | 5.381928 | 2.182779 | 7.49E−05 | 0.000153 | 6.79561 | 3.151967 | 7.11E−09 | 3.25E−08 |
| ENSBTAG00000008410 | OVOL2 | 5.155462 | 2.057315 | 0.000284 | 0.000542 | 6.788269 | 3.142432 | 7.11E−09 | 3.25E−08 |
| ENSBTAG00000017651 | F1MQU6 | 0.980874 | 3.78782 | 0.013807 | 0.021053 | 2.916434 | 5.180474 | 7.65E−09 | 3.48E−08 |
| ENSBTAG00000038702 | ZNF721 | 4.13816 | 4.019562 | 1.69E−17 | 8.86E−17 | 3.910444 | 3.833375 | 8.00E−09 | 3.63E−08 |
| ENSBTAG00000010546 | LIN54 | 1.267 | 5.226757 | 3.29E−07 | 8.50E−07 | 2.616107 | 6.254951 | 8.00E−09 | 3.63E−08 |
| ENSBTAG00000018199 | ZBTB8A | 1.895408 | 2.59922 | 0.007924 | 0.012568 | 3.817783 | 3.935744 | 8.30E−09 | 3.76E−08 |
| ENSBTAG00000014389 | SP4 | 1.97834 | 3.401853 | 3.49E−05 | 7.45E−05 | 3.242166 | 4.342345 | 1.63E−08 | 7.07E−08 |
| ENSBTAG00000021645 | MBD4 | 1.899567 | 4.91115 | 1.61E−11 | 5.83E−11 | 2.691301 | 5.53583 | 1.64E−08 | 7.09E−08 |
| ENSBTAG00000001024 | ARID1A | 2.543262 | 7.949548 | 3.47E−53 | 7.83E−52 | 2.407294 | 7.836379 | 1.65E−08 | 7.16E−08 |
| ENSBTAG00000014677 | TADA2A | 1.762235 | 5.710414 | 1.01E−14 | 4.52E−14 | 2.537071 | 6.326464 | 1.92E−08 | 8.28E−08 |
| ENSBTAG00000016723 | GFI1 | 6.471276 | 2.902714 | 6.29E−10 | 2.03E−09 | 6.676527 | 3.055331 | 1.99E−08 | 8.48E−08 |
| ENSBTAG00000021289 | POGZ | 2.400469 | 6.379915 | 1.04E−30 | 1.06E−29 | 2.508201 | 6.470757 | 2.20E−08 | 9.34E−08 |
| ENSBTAG00000021357 | ZNF518A | 2.666152 | 5.689128 | 5.66E−27 | 4.83E−26 | 2.629673 | 5.659475 | 2.42E−08 | 1.02E−07 |
| ENSBTAG00000016074 | ZNF143 | 1.291581 | 4.704886 | 7.92E−06 | 1.81E−05 | 2.623112 | 5.706226 | 2.77E−08 | 1.17E−07 |
| ENSBTAG00000008544 | SALL1 | 9.23774 | 5.308525 | 5.68E−53 | 1.27E−51 | 6.605674 | 3.004438 | 4.04E−08 | 1.65E−07 |
| ENSBTAG00000020754 | ZNF526 | 1.825313 | 4.288427 | 1.06E−07 | 2.85E−07 | 2.775677 | 5.018785 | 4.12E−08 | 1.67E−07 |
| ENSBTAG00000000943 | ZNF286A | 4.653586 | 6.511817 | 5.13E−72 | 2.05E−70 | 2.817127 | 4.87934 | 4.68E−08 | 1.89E−07 |
| ENSBTAG00000020594 | ELF2 | 0.882028 | 5.842434 | 3.04E−05 | 6.52E−05 | 2.37583 | 6.940732 | 5.15E−08 | 2.08E−07 |
| ENSBTAG00000005195 | ZNF200 | 1.04898 | 4.255104 | 0.001408 | 0.002474 | 2.616069 | 5.389209 | 5.36E−08 | 2.15E−07 |
| ENSBTAG00000003653 | LYAR | 3.39915 | 6.260191 | 2.97E−47 | 5.36E−46 | 2.571555 | 5.549112 | 5.39E−08 | 2.16E−07 |
| ENSBTAG00000037981 | E1BKR7 | 2.187218 | 3.105343 | 7.82E−05 | 0.000159 | 3.414757 | 4.000725 | 5.60E−08 | 2.24E−07 |
| ENSBTAG00000019001 | PRDM10 | 2.560448 | 4.629865 | 1.15E−14 | 5.15E−14 | 2.838526 | 4.854852 | 5.89E−08 | 2.33E−07 |
| ENSBTAG00000039770 | CEBPZ | 1.919305 | 7.686692 | 8.51E−32 | 9.06E−31 | 2.291887 | 7.9872 | 6.28E−08 | 2.48E−07 |
| ENSBTAG00000003034 | GATAD2A | 1.889221 | 7.572528 | 3.01E−30 | 3.00E−29 | 2.285782 | 7.892428 | 6.95E−08 | 2.74E−07 |
| ENSBTAG00000039316 | ZNF268 | 2.463732 | 3.812048 | 1.35E−08 | 3.92E−08 | 3.101563 | 4.306519 | 7.36E−08 | 2.89E−07 |
| ENSBTAG00000001160 | GMEB1 | 1.254584 | 4.20843 | 0.00019 | 0.000372 | 2.629267 | 5.216832 | 8.18E−08 | 3.20E−07 |
| ENSBTAG00000000819 | RFX3 | 4.715732 | 1.838881 | 0.004193 | 0.006841 | 6.505844 | 2.929343 | 8.37E−08 | 3.25E−07 |
| ENSBTAG00000008048 | GCFC2 | 1.557155 | 3.607921 | 0.000208 | 0.000405 | 2.827973 | 4.528304 | 1.44E−07 | 5.38E−07 |
| ENSBTAG00000012159 | CNBP | 1.453633 | 8.560573 | 6.49E−23 | 4.55E−22 | 2.190166 | 9.132729 | 1.44E−07 | 5.39E−07 |
| ENSBTAG00000017729 | NR2C1 | 1.092942 | 4.671562 | 0.000166 | 0.000325 | 2.45524 | 5.665269 | 1.51E−07 | 5.63E−07 |
| ENSBTAG00000038715 | 9130023H24Rik | 5.640297 | 2.336667 | 1.03E−05 | 2.30E−05 | 6.451321 | 2.888292 | 1.77E−07 | 6.47E−07 |
| ENSBTAG00000033642 | ZNF721 | 3.422949 | 3.768247 | 4.53E−12 | 1.70E−11 | 3.465249 | 3.802716 | 1.90E−07 | 6.93E−07 |
| ENSBTAG00000014273 | NRF1 | 2.249417 | 4.096365 | 4.98E−09 | 1.49E−08 | 2.816162 | 4.53451 | 2.40E−07 | 8.67E−07 |
| ENSBTAG00000016757 | ZKSCAN1 | 1.91759 | 4.662532 | 3.21E−10 | 1.06E−09 | 2.535621 | 5.139173 | 2.50E−07 | 9.02E−07 |
| ENSBTAG00000018710 | TTF1 | 1.821135 | 5.261482 | 1.17E−12 | 4.55E−12 | 2.388543 | 5.70233 | 2.62E−07 | 9.37E−07 |
| ENSBTAG00000017682 | TET2 | 1.131633 | 4.682275 | 6.84E−05 | 0.000141 | 2.397861 | 5.602828 | 2.71E−07 | 9.69E−07 |
| ENSBTAG00000003064 | PAXBP1 | 2.066626 | 6.673253 | 3.71E−27 | 3.20E−26 | 2.228257 | 6.805622 | 3.16E−07 | 1.12E−06 |
| ENSBTAG00000013757 | CTCF | 1.927522 | 7.018364 | 5.30E−27 | 4.53E−26 | 2.192865 | 7.23224 | 3.30E−07 | 1.17E−06 |
| ENSBTAG00000009242 | ZNF638 | 1.297727 | 8.027409 | 1.94E−17 | 1.02E−16 | 2.129006 | 8.660334 | 3.31E−07 | 1.17E−06 |
| ENSBTAG00000014680 | ZBTB49 | 1.564087 | 4.156708 | 9.43E−06 | 2.14E−05 | 2.581078 | 4.907802 | 3.36E−07 | 1.19E−06 |
| ENSBTAG00000008213 | ATMIN | 1.587251 | 5.67307 | 2.03E−12 | 7.76E−12 | 2.259203 | 6.190672 | 4.00E−07 | 1.39E−06 |
| ENSBTAG00000017573 | RBM26 | 1.552076 | 6.275779 | 4.81E−15 | 2.20E−14 | 2.198179 | 6.773657 | 4.29E−07 | 1.48E−06 |
| ENSBTAG00000011087 | ARID2 | 1.757772 | 5.817368 | 1.91E−15 | 8.94E−15 | 2.239382 | 6.193039 | 4.88E−07 | 1.68E−06 |
| ENSBTAG00000015808 | ZNF609 | 1.978214 | 5.691021 | 1.52E−17 | 8.01E−17 | 2.279251 | 5.930105 | 4.92E−07 | 1.69E−06 |
| ENSBTAG00000006954 | ZNF432 | 2.108345 | 3.839447 | 3.89E−07 | 9.98E−07 | 2.812223 | 4.37076 | 4.98E−07 | 1.71E−06 |
| ENSBTAG00000003399 | SMARCA5 | 1.414534 | 9.00643 | 5.71E−23 | 4.01E−22 | 2.068393 | 9.508854 | 5.68E−07 | 1.92E−06 |
| ENSBTAG00000020861 | CHAMP1 | 1.695062 | 6.193374 | 8.78E−17 | 4.43E−16 | 2.191997 | 6.581444 | 5.67E−07 | 1.92E−06 |
| ENSBTAG00000023989 | MSANTD4 | 1.257573 | 5.133732 | 6.84E−07 | 1.72E−06 | 2.265862 | 5.878922 | 6.04E−07 | 2.04E−06 |
| ENSBTAG00000014636 | ZFHX3 | 2.899791 | 4.933325 | 1.14E−20 | 7.10E−20 | 2.555133 | 4.661171 | 8.40E−07 | 2.78E−06 |
| ENSBTAG00000004710 | HNF1B | 4.327664 | 1.673685 | 0.016287 | 0.02429 | 6.2599 | 2.747209 | 8.44E−07 | 2.78E−06 |
| ENSBTAG00000003971 | E2F1 | 3.874278 | 5.372873 | 1.15E−35 | 1.43E−34 | 2.695218 | 4.375593 | 9.60E−07 | 3.15E−06 |
| ENSBTAG00000013716 | NR2C2 | 1.196721 | 4.607226 | 5.53E−05 | 0.000115 | 2.301237 | 5.409224 | 1.22E−06 | 3.94E−06 |
| ENSBTAG00000019187 | ZNF462 | 1.903604 | 6.285506 | 6.45E−21 | 4.07E−20 | 2.12902 | 6.462361 | 1.22E−06 | 3.96E−06 |
| ENSBTAG00000015462 | BNC1 | 7.995913 | 4.161829 | 9.59E−26 | 7.69E−25 | 6.191733 | 2.698864 | 1.27E−06 | 4.06E−06 |
| ENSBTAG00000006650 | ZNF668 | 0.915419 | 3.643376 | 0.031544 | 0.045401 | 2.506208 | 4.728747 | 1.26E−06 | 4.06E−06 |
| ENSBTAG00000003784 | DNAJC2 | 1.452488 | 6.444577 | 3.30E−14 | 1.42E−13 | 2.083277 | 6.924269 | 1.35E−06 | 4.32E−06 |
| ENSBTAG00000018625 | ZNF263 | 2.315582 | 4.536371 | 2.38E−12 | 9.06E−12 | 2.47605 | 4.660698 | 1.59E−06 | 5.06E−06 |
| ENSBTAG00000014751 | ZIC1 | 6.134631 | 2.660904 | 5.54E−08 | 1.52E−07 | 6.154466 | 2.674471 | 1.91E−06 | 5.96E−06 |
| ENSBTAG00000027017 | SIX3 | 5.351811 | 2.165609 | 7.49E−05 | 0.000153 | 6.122692 | 2.649111 | 1.91E−06 | 5.96E−06 |
| ENSBTAG00000005980 | RREB1 | 0.511495 | 5.716919 | 0.016199 | 0.02429 | 2.040305 | 6.766724 | 2.25E−06 | 6.97E−06 |
| ENSBTAG00000010113 | ZBTB24 | 1.003847 | 4.557734 | 0.000684 | 0.001248 | 2.209372 | 5.409283 | 2.58E−06 | 7.95E−06 |
| ENSBTAG00000034090 | ZNF721 | 4.164396 | 3.60287 | 4.70E−13 | 1.88E−12 | 3.667856 | 3.221686 | 2.77E−06 | 8.49E−06 |
| ENSBTAG00000040585 | NR6A1 | 8.384288 | 4.51103 | 1.27E−32 | 1.40E−31 | 6.071055 | 2.615848 | 2.90E−06 | 8.79E−06 |
| ENSBTAG00000015875 | FOXM1 | 4.023611 | 4.931225 | 5.39E−29 | 5.07E−28 | 2.815001 | 3.929859 | 3.37E−06 | 1.02E−05 |
| ENSBTAG00000014123 | ZNF507 | 1.210297 | 6.498405 | 8.16E−11 | 2.82E−10 | 1.961616 | 7.051296 | 4.25E−06 | 1.27E−05 |
| ENSBTAG00000045581 | ZNF845 | 5.67443 | 2.357698 | 5.31E−06 | 1.23E−05 | 6.027656 | 2.587926 | 4.44E−06 | 1.31E−05 |
| ENSBTAG00000004925 | F1MJ44 | 2.425658 | 2.842495 | 0.000288 | 0.000549 | 3.270539 | 3.42542 | 4.67E−06 | 1.38E−05 |
| ENSBTAG00000009059 | PITX2 | 1.544242 | 2.618791 | 0.017125 | 0.02548 | 2.999528 | 3.548397 | 4.89E−06 | 1.44E−05 |
| ENSBTAG00000023938 | CENPA | 3.068443 | 4.867507 | 2.70E−21 | 1.74E−20 | 2.442528 | 4.368571 | 4.94E−06 | 1.45E−05 |
| ENSBTAG00000000868 | ZFP1 | 3.048811 | 2.942043 | 1.57E−06 | 3.81E−06 | 3.377205 | 3.170383 | 5.08E−06 | 1.49E−05 |
| ENSBTAG00000003546 | TFAM | 2.620066 | 4.955075 | 2.43E−18 | 1.33E−17 | 2.312104 | 4.711289 | 5.13E−06 | 1.50E−05 |
| ENSBTAG00000017263 | MXI1 | 2.793505 | 4.343966 | 8.04E−14 | 3.38E−13 | 2.577685 | 4.175411 | 5.42E−06 | 1.59E−05 |
| ENSBTAG00000006034 | RNF113A | 1.972024 | 4.647003 | 2.14E−10 | 7.16E−10 | 2.263483 | 4.869221 | 5.62E−06 | 1.64E−05 |
| ENSBTAG00000008883 | ZNF207 | 1.604335 | 7.615735 | 3.62E−23 | 2.57E−22 | 1.890616 | 7.835025 | 5.98E−06 | 1.74E−05 |
| ENSBTAG00000000801 | ZNF583 | 0.99559 | 4.298407 | 0.001814 | 0.003154 | 2.169981 | 5.114223 | 5.99E−06 | 1.74E−05 |
| ENSBTAG00000015146 | TOX4 | 0.478692 | 6.170172 | 0.012797 | 0.0196 | 1.925826 | 7.155566 | 5.97E−06 | 1.74E−05 |
| ENSBTAG00000032982 | TERF1 | 1.87018 | 6.09674 | 4.92E−19 | 2.79E−18 | 1.986197 | 6.185539 | 6.48E−06 | 1.88E−05 |
| ENSBTAG00000006175 | KDM5B | 1.674878 | 7.201185 | 1.59E−22 | 1.09E−21 | 1.894657 | 7.369378 | 7.27E−06 | 2.08E−05 |
| ENSBTAG00000020819 | YY1 | 1.493232 | 6.683325 | 3.59E−16 | 1.75E−15 | 1.913587 | 7.00118 | 7.50E−06 | 2.14E−05 |
| ENSBTAG00000038926 | ZNF805 | 2.194473 | 3.398941 | 8.99E−06 | 2.04E−05 | 2.741235 | 3.792261 | 7.62E−06 | 2.17E−05 |
| ENSBTAG00000013961 | MYSM1 | 1.296879 | 5.6998 | 5.41E−09 | 1.62E−08 | 1.968304 | 6.194494 | 8.50E−06 | 2.41E−05 |
| ENSBTAG00000000784 | MTERF4 | 1.449108 | 5.136314 | 1.55E−08 | 4.46E−08 | 2.038126 | 5.56996 | 8.54E−06 | 2.42E−05 |
| ENSBTAG00000003878 | ZUP1 | 1.273591 | 4.774525 | 5.16E−06 | 1.20E−05 | 2.093224 | 5.366677 | 8.99E−06 | 2.54E−05 |
| ENSBTAG00000043973 | LIN28B | 7.772209 | 3.962252 | 1.28E−22 | 8.80E−22 | 5.908690 | 2.508047 | 1.06E−05 | 2.95E−05 |
| ENSBTAG00000000098 | SETDB1 | 1.694723 | 5.67888 | 1.28E−13 | 5.29E−13 | 1.975203 | 5.891816 | 1.12E−05 | 3.10E−05 |
| ENSBTAG00000038498 | ZBTB39 | 3.339063 | 4.343222 | 6.00E−17 | 3.05E−16 | 2.677283 | 3.823426 | 1.35E−05 | 3.71E−05 |
| ENSBTAG00000004512 | MKRN2 | 1.394339 | 5.447924 | 4.27E−09 | 1.28E−08 | 1.959413 | 5.861435 | 1.35E−05 | 3.72E−05 |
| ENSBTAG00000008482 | SON | 1.373746 | 8.720078 | 3.46E−21 | 2.21E−20 | 1.786438 | 9.028346 | 1.36E−05 | 3.74E−05 |
| ENSBTAG00000004746 | HELZ | 0.582295 | 5.775156 | 0.006231 | 0.009981 | 1.869902 | 6.649436 | 1.45E−05 | 3.97E−05 |
| ENSBTAG00000044032 | KDM2A | 1.413669 | 7.488437 | 3.71E−18 | 2.01E−17 | 1.798323 | 7.776493 | 1.61E−05 | 4.41E−05 |
| ENSBTAG00000027431 | ZNF227 | 0.993874 | 3.994381 | 0.005956 | 0.00956 | 2.135304 | 4.770104 | 1.86E−05 | 4.99E−05 |
| ENSBTAG00000037988 | ZSCAN31 | 1.321379 | 3.094304 | 0.012396 | 0.019027 | 2.502305 | 3.863047 | 1.86E−05 | 5.00E−05 |
| ENSBTAG00000030453 | ZC3H14 | 1.516135 | 7.159679 | 6.19E−19 | 3.49E−18 | 1.798576 | 7.37203 | 1.87E−05 | 5.01E−05 |
| ENSBTAG00000031686 | ZNF566 | 1.86779 | 3.77485 | 6.69E−06 | 1.54E−05 | 2.375055 | 4.137287 | 1.92E−05 | 5.13E−05 |
| ENSBTAG00000008409 | MYC | 3.514892 | 7.60936 | 8.74E−78 | 4.12E−76 | 1.847154 | 6.196599 | 2.57E−05 | 6.79E−05 |
| ENSBTAG00000039782 | ZNF12 | 2.299782 | 3.948418 | 7.80E−09 | 2.30E−08 | 2.350101 | 3.985305 | 2.73E−05 | 7.10E−05 |
| ENSBTAG00000020654 | BAZ2B | 3.294341 | 4.982863 | 1.01E−24 | 7.67E−24 | 2.288175 | 4.170389 | 2.85E−05 | 7.41E−05 |
| ENSBTAG00000013142 | MYNN | 0.728067 | 5.430922 | 0.001671 | 0.00292 | 1.832043 | 6.185845 | 2.92E−05 | 7.59E−05 |
| ENSBTAG00000021253 | PRDM15 | 2.162647 | 4.085914 | 7.50E−09 | 2.22E−08 | 2.265195 | 4.159079 | 3.35E−05 | 8.64E−05 |
| ENSBTAG00000020312 | NCOA2 | 0.701454 | 5.093976 | 0.004681 | 0.007598 | 1.823773 | 5.8514 | 4.03E−05 | 0.000103 |
| ENSBTAG00000012014 | CDX1 | 10.16569 | 6.201856 | 4.59E−85 | 2.58E−83 | 5.711029 | 2.378986 | 4.14E−05 | 0.000105 |
| ENSBTAG00000014741 | OTX2 | 7.75547 | 3.948901 | 3.95E−22 | 2.64E−21 | 5.70275 | 2.376541 | 4.14E−05 | 0.000105 |
| ENSBTAG00000008756 | ELF3 | 4.528606 | 1.756049 | 0.008256 | 0.012889 | 5.714901 | 2.381274 | 4.14E−05 | 0.000105 |
| ENSBTAG00000012063 | E2F4 | 2.492897 | 6.930487 | 2.68E−39 | 3.79E−38 | 1.780677 | 6.363371 | 4.24E−05 | 0.000107 |
| ENSBTAG00000014211 | FOXP4 | 2.123817 | 6.269321 | 1.19E−24 | 9.02E−24 | 1.80758 | 6.021989 | 4.42E−05 | 0.000111 |
| ENSBTAG00000023867 | RC3H2 | 0.922097 | 5.123395 | 0.000256 | 0.000494 | 1.825273 | 5.746274 | 4.81E−05 | 0.00012 |
| ENSBTAG00000024657 | ZFP91 | 1.046513 | 5.8174 | 7.65E−07 | 1.91E−06 | 1.742593 | 6.308006 | 5.68E−05 | 0.000141 |
| ENSBTAG00000013253 | THAP4 | 1.161642 | 5.693101 | 1.32E−07 | 3.54E−07 | 1.7516 | 6.113846 | 6.37E−05 | 0.000157 |
| ENSBTAG00000037721 | pseudogene | 2.685358 | 2.607766 | 0.000218 | 0.000424 | 3.198944 | 2.938861 | 6.49E−05 | 0.00016 |
| ENSBTAG00000008306 | MKRN1 | 3.022463 | 6.838064 | 2.57E−50 | 5.18E−49 | 1.780353 | 5.822296 | 6.93E−05 | 0.000168 |
| ENSBTAG00000000565 | RFX7 | 1.574131 | 6.241912 | 3.49E−15 | 1.61E−14 | 1.715838 | 6.350493 | 7.25E−05 | 0.000175 |
| ENSBTAG00000021018 | KHSRP | 3.18667 | 8.619261 | 7.43E−85 | 4.15E−83 | 1.654118 | 7.345382 | 7.56E−05 | 0.000183 |
| ENSBTAG00000008771 | MYEF2 | 3.276024 | 5.191001 | 2.60E−27 | 2.27E−26 | 2.073343 | 4.233025 | 9.42E−05 | 0.000226 |
| ENSBTAG00000030744 | NFYB | 1.272022 | 3.061865 | 0.017594 | 0.026147 | 2.297805 | 3.711083 | 0.000112 | 0.000263 |
| ENSBTAG00000020356 | GON4L | 2.087649 | 6.05206 | 3.41E−22 | 2.29E−21 | 1.718607 | 5.772158 | 0.000113 | 0.000264 |
| ENSBTAG00000020012 | CDC5L | 1.429585 | 6.713435 | 3.39E−15 | 1.56E−14 | 1.612258 | 6.848377 | 0.000138 | 0.000319 |
| ENSBTAG00000015899 | F1MVJ7 | 2.239033 | 4.589207 | 4.82E−12 | 1.80E−11 | 1.965711 | 4.385599 | 0.000147 | 0.00034 |
| ENSBTAG00000000753 | PIAS4 | 1.355583 | 5.534862 | 5.50E−09 | 1.64E−08 | 1.694612 | 5.778444 | 0.000147 | 0.00034 |
| ENSBTAG00000031567 | SMARCC1 | 3.108591 | 8.186968 | 2.80E−75 | 1.23E−73 | 1.584567 | 6.935744 | 0.000179 | 0.000404 |
| ENSBTAG00000002055 | ZFP28 | 1.530504 | 4.850102 | 4.02E−08 | 1.12E−07 | 1.744394 | 5.003581 | 0.000229 | 0.000511 |
| ENSBTAG00000047739 | TSC22D1 | 0.476142 | 5.812492 | 0.022037 | 0.032316 | 1.562684 | 6.521036 | 0.000252 | 0.00056 |
| ENSBTAG00000025246 | ZIC2 | 8.738197 | 4.837316 | 4.01E−40 | 5.83E−39 | 5.322272 | 2.148872 | 0.000286 | 0.000621 |
| ENSBTAG00000005938 | BARHL2 | 6.966305 | 3.284408 | 1.03E−13 | 4.29E−13 | 5.401115 | 2.194031 | 0.000286 | 0.000621 |
| ENSBTAG00000004498 | ESR2 | 6.150726 | 2.672047 | 5.54E−08 | 1.52E−07 | 5.34497 | 2.160877 | 0.000286 | 0.000621 |
| ENSBTAG00000015686 | TERF2IP | 1.358487 | 5.968711 | 7.63E−11 | 2.64E−10 | 1.575085 | 6.122363 | 0.000302 | 0.000655 |
| ENSBTAG00000019313 | ZMIZ1 | 0.979838 | 6.775826 | 3.02E−08 | 8.51E−08 | 1.500689 | 7.139826 | 0.000338 | 0.000726 |
| ENSBTAG00000007488 | ZFP90 | 0.893873 | 4.188164 | 0.008782 | 0.013662 | 1.75106 | 4.749088 | 0.00035 | 0.000751 |
| ENSBTAG00000016287 | UNK | 1.904755 | 5.329854 | 4.71E−14 | 2.01E−13 | 1.645721 | 5.1418 | 0.000392 | 0.000836 |
| ENSBTAG00000034529 | HMGA1 | 6.315301 | 9.700059 | 8.67E−227 | 3.17E−224 | 1.589672 | 5.428106 | 0.000453 | 0.000961 |
| ENSBTAG00000021458 | DLX6 | 6.27945 | 2.762436 | 8.04E−09 | 2.36E−08 | 5.287995 | 2.12859 | 0.000474 | 0.000987 |
| ENSBTAG00000008280 | HNF4G | 5.001737 | 1.977123 | 0.000556 | 0.001022 | 5.289024 | 2.12958 | 0.000474 | 0.000987 |
| ENSBTAG00000023885 | ZNF79 | 2.467581 | 5.021449 | 2.31E−17 | 1.20E−16 | 1.765785 | 4.4952 | 0.000527 | 0.001092 |
| ENSBTAG00000039341 | E1B8F8 | 1.96613 | 2.478496 | 0.00841 | 0.013099 | 2.634905 | 2.869866 | 0.000538 | 0.001113 |
| ENSBTAG00000021438 | ZNF821 | 1.139661 | 3.120028 | 0.021078 | 0.030997 | 1.994594 | 3.638924 | 0.000571 | 0.001177 |
| ENSBTAG00000017836 | ZFP57 | 0.743762 | 4.720808 | 0.00673 | 0.010751 | 1.559411 | 5.244691 | 0.000665 | 0.001363 |
| ENSBTAG00000017446 | E2F8 | 2.461006 | 3.269239 | 4.61E−06 | 1.08E−05 | 2.358638 | 3.203919 | 0.000706 | 0.001443 |
| ENSBTAG00000021045 | E2F3 | 1.404395 | 4.792736 | 6.71E−07 | 1.69E−06 | 1.601666 | 4.932373 | 0.000711 | 0.001452 |
| ENSBTAG00000007730 | ZFX | 0.44836 | 6.077277 | 0.023205 | 0.033947 | 1.426889 | 6.704939 | 0.000716 | 0.001462 |
| ENSBTAG00000009950 | PAX3 | 2.51479 | 3.2318 | 2.38E−06 | 5.70E−06 | 2.311127 | 3.093977 | 0.000729 | 0.001486 |
| ENSBTAG00000007827 | RCOR2 | 5.63102 | 7.197993 | 5.72E−108 | 5.16E−106 | 1.884812 | 3.939094 | 0.000764 | 0.001553 |
| ENSBTAG00000037803 | ZNF197 | 0.968514 | 3.630213 | 0.021874 | 0.032097 | 1.788811 | 4.149731 | 0.000832 | 0.001666 |
| ENSBTAG00000038316 | GPATCH8 | 2.049575 | 6.541457 | 8.07E−26 | 6.52E−25 | 1.44703 | 6.089482 | 0.000842 | 0.001684 |
| ENSBTAG00000012946 | HSF2 | 0.989343 | 5.332855 | 3.44E−05 | 7.34E−05 | 1.486578 | 5.666226 | 0.000858 | 0.001715 |
| ENSBTAG00000010533 | HMGXB4 | 0.712198 | 4.393988 | 0.021877 | 0.032099 | 1.551185 | 4.924663 | 0.001066 | 0.002105 |
| ENSBTAG00000002890 | RBM10 | 2.510086 | 7.865266 | 4.13E−51 | 8.51E−50 | 1.365171 | 6.972036 | 0.001102 | 0.002172 |
| ENSBTAG00000023372 | MYCN | 8.360541 | 4.488779 | 3.53E−32 | 3.81E−31 | 5.062321 | 2.008001 | 0.001344 | 0.002583 |
| ENSBTAG00000012656 | OVOL1 | 6.447944 | 2.88526 | 1.19E−09 | 3.74E−09 | 5.069571 | 2.011826 | 0.001344 | 0.002583 |
| ENSBTAG00000004159 | SIX2 | 5.965343 | 2.545481 | 3.87E−07 | 9.94E−07 | 5.032871 | 1.992907 | 0.001344 | 0.002583 |
| ENSBTAG00000030444 | Zfp11 | 4.472773 | 1.732442 | 0.008256 | 0.012889 | 5.086332 | 2.020612 | 0.001344 | 0.002583 |
| ENSBTAG00000019697 | NPAS2 | 0.683204 | 9.732589 | 5.21E−07 | 1.33E−06 | 1.288826 | 10.13412 | 0.001366 | 0.002623 |
| ENSBTAG00000014248 | MIER3 | 1.529879 | 4.212755 | 6.36E−06 | 1.46E−05 | 1.621765 | 4.274966 | 0.001488 | 0.002842 |
| ENSBTAG00000002201 | NFXL1 | 1.428682 | 4.547767 | 3.31E−06 | 7.81E−06 | 1.523491 | 4.613666 | 0.002172 | 0.004075 |
| ENSBTAG00000000418 | TOX3 | 7.492685 | 3.719895 | 6.80E−19 | 3.82E−18 | 4.882484 | 1.917498 | 0.002299 | 0.004237 |
| ENSBTAG00000011889 | NOC4L | 2.486453 | 6.126597 | 1.51E−29 | 1.46E−28 | 1.372565 | 5.278125 | 0.002396 | 0.004402 |
| ENSBTAG00000003687 | FOXK2 | 1.31275 | 6.438022 | 5.14E−12 | 1.92E−11 | 1.280448 | 6.417268 | 0.002622 | 0.004796 |
| ENSBTAG00000009905 | NFYA | 0.917652 | 4.543725 | 0.001916 | 0.003326 | 1.423968 | 4.869445 | 0.002666 | 0.004871 |
| ENSBTAG00000007617 | TERF2 | 2.320091 | 6.586887 | 1.11E−31 | 1.17E−30 | 1.312571 | 5.824756 | 0.002747 | 0.005007 |
| ENSBTAG00000031874 | ZNF165 | 1.373294 | 2.781332 | 0.028265 | 0.040947 | 2.012874 | 3.153387 | 0.003093 | 0.0056 |
| ENSBTAG00000007629 | THAP1 | 1.686758 | 4.018914 | 5.82E−06 | 1.34E−05 | 1.59214 | 3.957537 | 0.003497 | 0.006293 |
| ENSBTAG00000016448 | ZBTB40 | 1.582411 | 5.100745 | 1.13E−09 | 3.59E−09 | 1.382916 | 4.965345 | 0.003568 | 0.006415 |
| ENSBTAG00000006914 | PRR3 | 3.521632 | 5.9253 | 2.27E−42 | 3.58E−41 | 1.491469 | 4.313081 | 0.003692 | 0.006626 |
| ENSBTAG00000018699 | KAT7 | 1.715900 | 6.789044 | 5.75E−21 | 3.64E−20 | 1.224035 | 6.434986 | 0.003988 | 0.007007 |
| ENSBTAG00000046218 | KLF11 | 1.774153 | 4.463826 | 3.76E−08 | 1.05E−07 | 1.471449 | 4.25759 | 0.004042 | 0.007094 |
| ENSBTAG00000000340 | ADNP | 1.427864 | 7.333503 | 7.65E−18 | 4.08E−17 | 1.173977 | 7.156918 | 0.004595 | 0.008023 |
| ENSBTAG00000017872 | ZNF304 | 1.86198 | 4.376993 | 1.79E−08 | 5.11E−08 | 1.449951 | 4.095703 | 0.005459 | 0.00945 |
| ENSBTAG00000013592 | ZNF248 | 1.590284 | 3.846606 | 3.90E−05 | 8.23E−05 | 1.500741 | 3.78988 | 0.006082 | 0.010476 |
| ENSBTAG00000004262 | ZNF454 | 1.922023 | 2.273841 | 0.014216 | 0.02164 | 2.289691 | 2.463868 | 0.006401 | 0.010995 |
| ENSBTAG00000030366 | RBM22 | 1.932639 | 6.591887 | 1.02E−23 | 7.41E−23 | 1.16924 | 6.038185 | 0.006923 | 0.01169 |
| ENSBTAG00000003438 | ZBTB43 | 1.63602 | 3.64993 | 9.87E−05 | 0.000199 | 1.532875 | 3.586357 | 0.007769 | 0.012974 |
| ENSBTAG00000012385 | NFX1 | 1.843656 | 6.255025 | 1.16E−19 | 6.78E−19 | 1.147392 | 5.757225 | 0.008532 | 0.014181 |
| ENSBTAG00000010541 | ZNF346 | 1.280839 | 3.912493 | 0.000682 | 0.001245 | 1.420572 | 4.000764 | 0.009324 | 0.015422 |
| ENSBTAG00000010046 | ZNF350 | 1.847978 | 3.318071 | 0.000125 | 0.000251 | 1.646893 | 3.195111 | 0.009671 | 0.015957 |
| ENSBTAG00000002736 | DNMT1 | 2.869815 | 7.411774 | 8.77E−56 | 2.12E−54 | 1.116727 | 6.048658 | 0.009837 | 0.016208 |
| ENSBTAG00000017580 | RFX5 | 1.875225 | 4.591011 | 1.22E−09 | 3.84E−09 | 1.327149 | 4.216973 | 0.009909 | 0.016313 |
| ENSBTAG00000003456 | ZNF606 | 2.176343 | 4.306556 | 4.25E−10 | 1.39E−09 | 1.411191 | 3.783216 | 0.01018 | 0.016727 |
| ENSBTAG00000011074 | BRD2 | 1.005461 | 8.576886 | 4.21E−12 | 1.58E−11 | 1.033831 | 8.596041 | 0.010506 | 0.017241 |
| ENSBTAG00000000222 | ARID4B | 0.741068 | 5.7296 | 0.000585 | 0.001074 | 1.108383 | 5.964661 | 0.01051 | 0.017244 |
| ENSBTAG00000031895 | TFB1M | 0.759312 | 4.861019 | 0.004854 | 0.007861 | 1.154703 | 5.107009 | 0.011344 | 0.018538 |
| ENSBTAG00000011997 | ZMIZ2 | 2.153001 | 6.326046 | 9.12E−26 | 7.35E−25 | 1.117932 | 5.570355 | 0.011664 | 0.019026 |
| ENSBTAG00000007776 | BRPF1 | 2.300376 | 5.977383 | 1.12E−24 | 8.50E−24 | 1.143506 | 5.130144 | 0.013007 | 0.020714 |
| ENSBTAG00000038088 | F1MBW2 | 1.839577 | 3.74154 | 9.89E−06 | 2.24E−05 | 1.477696 | 3.511068 | 0.013547 | 0.021532 |
| ENSBTAG00000017542 | PPARD | 0.890755 | 5.688913 | 4.37E−05 | 9.17E−05 | 1.050374 | 5.790987 | 0.015881 | 0.025042 |
| ENSBTAG00000008943 | ZSCAN12 | 1.430255 | 3.368529 | 0.00158 | 0.002768 | 1.441758 | 3.378045 | 0.016073 | 0.025324 |
| ENSBTAG00000008139 | HOXA3 | 2.07222 | 2.99283 | 0.000487 | 0.000908 | 1.822014 | 2.840471 | 0.018311 | 0.028615 |
| ENSBTAG00000015965 | ZC3H8 | 2.013933 | 4.364916 | 2.60E−09 | 7.96E−09 | 1.268271 | 3.862881 | 0.021977 | 0.033567 |
| ENSBTAG00000000678 | CSDE1 | 0.700415 | 9.087899 | 5.28E−07 | 1.34E−06 | 0.91951 | 9.227478 | 0.021867 | 0.033567 |
| ENSBTAG00000023611 | ZBTB32 | 4.107957 | 1.591271 | 0.032192 | 0.045433 | 4.267329 | 1.650329 | 0.022122 | 0.033567 |
| ENSBTAG00000003077 | DPF2 | 2.314782 | 7.66617 | 5.41E−43 | 8.65E−42 | 0.936319 | 6.644186 | 0.025179 | 0.037823 |
| ENSBTAG00000030556 | ZNF217 | 1.968138 | 4.583309 | 5.77E−10 | 1.87E−09 | 1.174262 | 4.048067 | 0.026948 | 0.040368 |
| ENSBTAG00000039512 | ZSCAN25 | 2.85579 | 4.284847 | 3.83E−14 | 1.64E−13 | 1.343891 | 3.23989 | 0.029838 | 0.044352 |
| ENSBTAG00000021789 | ZNF574 | 1.072302 | 5.130886 | 2.60E−05 | 5.61E−05 | 1.004821 | 5.087511 | 0.030156 | 0.0448 |
| ENSBTAG00000007067 | ZC3H13 | 1.70231 | 5.295191 | 1.01E−11 | 3.71E−11 | 1.014374 | 4.830228 | 0.030306 | 0.044999 |
| ENSBTAG00000014790 | ZBTB2 | 2.49059 | 5.019462 | 8.60E−18 | 4.57E−17 | 1.124409 | 4.050075 | 0.03055 | 0.045295 |
| ENSBTAG00000020878 | DMTF1 | 1.569613 | 4.849204 | 1.71E−08 | 4.90E−08 | 1.036315 | 4.497625 | 0.033334 | 0.049052 |

TABLE 6
Functional annotation of genes identified as part of quadrant II and the transcriptional
network (in FIG. 7A-D) underlying the bovine pluripotent state.

| Symbol | Name | NCBI Entrez Gene |
|---|---|---|
| TFAP2C | Transcription | The protein encoded by this gene is a sequence-specific DNA-binding transcription factor involved in |
| | Factor AP-2 | the activation of several developmental genes. The encoded protein can act as either a homodimer or |
| | Gamma | heterodimer with other family members and is induced during retinoic acid-mediated differentiation. |
| | | It plays a role in the development of the eyes, face, body wall, limbs, and neural tube. [provided by |
| | | RefSeq, July 2008] |
| SATB1 | SATB | This gene encodes a matrix protein which binds nuclear matrix and scaffold-associating DNAs |
| | Homeobox 1 | through a unique nuclear architecture. The protein recruits chromatin-remodeling factors in order to |
| | | regulate chromatin structure and gene expression. [provided by RefSeq, April 2016] |
| ZFP42 | ZFP42 Zinc | |
| | Finger Protein | |
| MYB | MYB Proto- | This gene encodes a protein with three HTH DNA-binding domains that functions as a transcription |
| | Oncogene, | regulator. This protein plays an essential role in the regulation of hematopoiesis. This gene may be |
| | Transcription | aberrently expressed or rearranged or undergo translocation in leukemias and lymphomas, and is |
| | Factor | considered to be an oncogene. Alternative splicing results in multiple transcript variants. [provided by |
| | | RefSeq, January 2016] |
| ESRRG | Estrogen | This gene encodes a member of the estrogen receptor-related receptor (ESRR) family, which belongs |
| | Related | to the nuclear hormone receptor superfamily. All members of the ESRR family share an almost |
| | Receptor | identical DNA binding domain, which is composed of two C4-type zinc finger motifs. The ESRR |
| | Gamma | members are orphan nuclear receptors; they bind to the estrogen response element and steroidogenic |
| | | factor 1 response element, and activate genes controlled by both response elements in the absence of |
| | | any ligands. The ESRR family is closely related to the estrogen receptor (ER) family. They share |
| | | target genes, co-regulators and promoters, and by targeting the same set of genes, the ESRRs seem to |
| | | interfere with the ER-mediated estrogen response in various ways. It has been reported that the family |
| | | member encoded by this gene functions as a transcriptional activator of DNA cytosine-5- |
| | | methyltransferases 1 (Dnmt1) expression by direct binding to its response elements in the DNMT1 |
| | | promoters, modulates cell proliferation and estrogen signaling in breast cancer, and negatively |
| | | regulates bone morphogenetic protein 2-induced osteoblast differentiation and bone formation. |
| | | Multiple alternatively spliced transcript variants have been identified, which mainly differ at the 5′ |
| | | end and some of which encode protein isoforms differing in the N-terminal region. [provided by |
| | | RefSeq, August 2011] |
| ZNF215 | Zinc Finger | This gene is imprinted in a tissue-specific manner with preferential expression in the testis, and |
| | Protein 215 | encodes a zinc finger protein that belongs to a family of zinc finger transcription factors. The encoded |
| | | protein contains an N-terminal SRE-ZBP, Ctfin51, AW-1, and Number 18 (SCAN) domain, a |
| | | kruppel-associated box A (KRABA) domain, and four C-terminal zinc finger domains. This gene is |
| | | located within one of three regions on chromosome 11p15 associated with Beckwith-Wiedemann |
| | | syndrome, called Beckwith-Wiedemann syndrome chromosome region-2 (BWSCR2), and is thought |
| | | to play a role in the etiology of this disease. [provided by RefSeq, August 2017] |
| PRDM14 | PR/SET Domain | This gene encodes a member of the PRDI-BF1 and RIZ homology domain containing (PRDM) |
| | 14 | family of transcriptional regulators. The encoded protein may possess histone methyltransferase |
| | | activity and plays a critical role in cell pluripotency by suppressing the expression of differentiation |
| | | marker genes. Expression of this gene may play a role in breast cancer. [provided by RefSeq, December |
| | | 2011] |
| ZNF296 | Zinc Finger | |
| | Protein 296 | |
| OTX1 | Orthodenticle | This gene encodes a member of the bicoid sub-family of homeodomain-containing transcription |
| | Homeobox 1 | factors. The encoded protein acts as a transcription factor and may play a role in brain and sensory |
| | | organ development. A similar protein in mouse is required for proper brain and sensory organ |
| | | development and can cause epilepsy. Alternative splicing results in multiple transcript variants. |
| | | [provided by RefSeq, January 2015] |
| SALL4 | Spalt Like | This gene encodes a zinc finger transcription factor thought to play a role in the development of |
| | Transcription | abducens motor neurons. Defects in this gene are a cause of Duane-radial ray syndrome (DRRS). |
| | Factor 4 | Alternative splicing results in multiple transcript variants encoding different isoforms. [provided by |
| | | RefSeq, December 2015] |
| CAMTA1 | Calmodulin | The protein encoded by this gene contains a CGI DNA-binding domain, a transcription factor |
| | Binding | immunoglobulin domain, ankyrin repeats, and calmodulin-binding IQ motifs. The encoded protein is |
| | Transcription | thought to be a transcription factor and may be a tumor suppressor. However, a translocation event is |
| | Activator 1 | sometimes observed between this gene and the WWTRI gene, with the resulting WWTR1-CAMTA1 |
| | | oncoprotein leading to epithelioid hemangioendothelioma, a malignant vascular cancer. [provided by |
| | | RefSeq, March 2017] |
| ESRRB | Estrogen | This gene encodes a protein with similarity to the estrogen receptor. Its function is unknown; |
| | Related | however, a similar protein in mouse plays an essential role in placental development. [provided by |
| | Receptor Beta | RefSeq, July 2008] |
| REL | REL Proto- | This gene encodes a protein that belongs to the Rel homology domain/immunoglobulin-like fold, |
| | Oncogene, NF- | plexin, transcription factor (RHD/IPT) family. Members of this family regulate genes involved in |
| | KB Subunit | apoptosis, inflammation, the immune response, and oncogenic processes. This proto-oncogene plays a |
| | | role in the survival and proliferation of B lymphocytes. Mutation or amplification of this gene is |
| | | associated with B-cell lymphomas, including Hodgkin's lymphoma. Single nucleotide polymorphisms |
| | | in this gene are associated with susceptibility to ulcerative colitis and rheumatoid arthritis. Alternative |
| | | splicing results in multiple transcript variants encoding different isoforms. [provided by RefSeq, April |
| | | 2014] |
| NFE2L3 | Nuclear Factor, | This gene encodes a member of the cap 'n' collar basic-region leucine zipper family of transcription |
| | Erythroid 2 Like | factors. The encoded protein heterodimerizes with small musculoaponeurotic fibrosarcoma factors to |
| | 3 | bind antioxidant response elements in target genes. This protein is a membrane bound glycoprotein |
| | | that is targeted to the endoplasmic reticulum and the nuclear envelope. Pseudogenes of this gene are |
| | | found on chromosomes 16, 17, and 18. [provided by RefSeq, March 2009] |
| LEF1 | Lymphoid | This gene encodes a transcription factor belonging to a family of proteins that share homology with |
| | Enhancer | the high mobility group protein-1. The protein encoded by this gene can bind to a functionally |
| | Binding Factor 1 | important site in the T-cell receptor-alpha enhancer, thereby conferring maximal enhancer activity. |
| | | This transcription factor is involved in the Wnt signaling pathway, and it may function in hair cell |
| | | differentiation and follicle morphogenesis. Mutations in this gene have been found in somatic |
| | | sebaceous tumors. This gene has also been linked to other cancers, including androgen-independent |
| | | prostate cancer. Alternative splicing results in multiple transcript variants. [provided by RefSeq, October |
| | | 2009] |
| GATA6 | GATA Binding | This gene is a member of a small family of zinc finger transcription factors that play an important role |
| | Protein 6 | in the regulation of cellular differentiation and organogenesis during vertebrate development. This |
| | | gene is expressed during early embryogenesis and localizes to endo- and mesodermally derived cells |
| | | during later embryogenesis and thereby plays an important role in gut, lung, and heart development. |
| | | Mutations in this gene are associated with several congenital defects. [provided by RefSeq, March |
| | | 2012] |
| MBNL3 | Muscleblind | This gene encodes a member of the muscleblind-like family of proteins. The encoded protein may |
| | Like Splicing | function in regulation of alternative splicing and may play a role in the pathophysiology of myotonic |
| | Regulator 3 | dystrophy. Alternatively spliced transcript variants have been described. [provided by RefSeq, December |
| | | 2009 |
| YBX2 | Y-Box Binding | This gene encodes a nucleic acid binding protein which is highly expressed in germ cells. The |
| | Protein 2 | encoded protein binds to a Y-box element in the promoters of certain genes but also binds to mRNA |
| | | transcribed from these genes. Pseudogenes for this gene are located on chromosome 10 and 15. |
| | | [provided by RefSeq, February 2012] |
| POU5F1 | POU Class 5 | This gene encodes a transcription factor containing a POU homeodomain that plays a key role in |
| | Homeobox 1 | embryonic development and stem cell pluripotency. Aberrant expression of this gene in adult tissues |
| | | is associated with tumorigenesis. This gene can participate in a translocation with the Ewing's |
| | | sarcoma gene on chromosome 21, which also leads to tumor formation. Alternative splicing, as well |
| | | as usage of alternative AUG and non-AUG translation initiation codons, results in multiple isoforms. |
| | | One of the AUG start codons is polymorphic in human populations. Related pseudogenes have been |
| | | identified on chromosomes 1, 3, 8, 10, and 12. [provided by RefSeq, October 2013] |
| FOXN1 | Forkhead Box | Mutations in the winged-helix transcription factor gene at the nude locus in mice and rats produce the |
| | N1 | pleiotropic phenotype of hairlessness and athymia, resulting in a severely compromised immune |
| | | system. This gene is orthologous to the mouse and rat genes and encodes a similar DNA-binding |
| | | transcription factor that is thought to regulate keratin gene expression. A mutation in this gene has |
| | | been correlated with T-cell immunodeficiency, the skin disorder congenital alopecia, and nail |
| | | dystrophy. Alternative splicing in the 5′ UTR of this gene has been observed. [provided by RefSeq, |
| | | July 2008] |
| FOXI3 | Forkhead Box I3 | |
| TET1 | Tet | DNA methylation is an epigenetic mechanism that is important for controlling gene expression. The |
| | Methylcytosine | protein encoded by this gene is a demethylase that belongs to the TET (ten-eleven translocation) |
| | Dioxygenase 1 | family. Members of the TET protein family play a role in the DNA methylation process and gene |
| | | activation. [provided by RefSeq, September 2015] |
| HIF3A | Hypoxia | The protein encoded by this gene is the alpha-3 subunit of one of several alpha/beta-subunit |
| | Inducible Factor | heterodimeric transcription factors that regulate many adaptive responses to low oxygen tension |
| | 3 Subunit Alpha | (hypoxia). The alpha-3 subunit lacks the transactivation domain found in factors containing either the |
| | | alpha-1 or alpha-2 subunits. It is thought that factors containing the alpha-3 subunit are negative |
| | | regulators of hypoxia-inducible gene expression. Multiple alternatively spliced transcript variants |
| | | have been found for this gene. [provided by RefSeq, March 2011] |
| IRF6 | Interferon | This gene encodes a member of the interferon regulatory transcription factor (IRF) family. Family |
| | Regulatory | members share a highly-conserved N-terminal helix-turn-helix DNA-binding domain and a less |
| | Factor 6 | conserved C-terminal protein-binding domain. The encoded protein may be a transcriptional |
| | | activator. Mutations in this gene can cause van der Woude syndrome and popliteal pterygium |
| | | syndrome. Mutations in this gene are also associated with non-syndromic orofacial cleft type 6. |
| | | Alternate splicing results in multiple transcript variants. [provided by RefSeq, May 2011] |
| NR5A2 | Nuclear | The protein encoded by this gene is a DNA-binding zinc finger transcription factor and is a member |
| | Receptor | of the fushi tarazu factor-1 subfamily of orphan nuclear receptors. The encoded protein is involved in |
| | Subfamily 5 | the expression of genes for hepatitis B virus and cholesterol biosynthesis, and may be an important |
| | Group A | regulator of embryonic development. [provided by RefSeq, June 2016] |
| | Member 2 | |
| CUX2 | Cut Like | This gene encodes a protein which contains three CUT domains and a homeodomain; both domains |
| | Homeobox 2 | are DNA-binding motifs. A similar gene, whose gene product possesses different DNA-binding |
| | | activities, is located on chromosome on chromosome 7. Two pseudogenes of this gene have been |
| | | identified on chromosomes 10 and 4. [provided by RefSeq, January 2013] |
| ISL1 | ISL LIM | This gene encodes a member of the LIM/homeodomain family of transcription factors. The encoded |
| | Homeobox 1 | protein binds to the enhancer region of the insulin gene, among others, and may play an important |
| | | role in regulating insulin gene expression. The encoded protein is central to the development of |
| | | pancreatic cell lineages and may also be required for motor neuron generation. Mutations in this gene |
| | | have been associated with maturity-onset diabetes of the young. [provided by RefSeq, July 2008] |
| IRF8 | Interferon | Interferon consensus sequence-binding protein (ICSBP) is a transcription factor of the interferon |
| | Regulatory | (IFN) regulatory factor (IRF) family. Proteins of this family are composed of a conserved DNA- |
| | Factor 8 | binding domain in the N-terminal region and a divergent C-terminal region that serves as the |
| | | regulatory domain. The IRF family proteins bind to the IFN-stimulated response element (ISRE) and |
| | | regulate expression of genes stimulated by type I IFNs, namely IFN-alpha and IFN-beta. IRF family |
| | | proteins also control expression of IFN-alpha and IFN-beta-regulated genes that are induced by viral |
| | | infection. [provided by RefSeq, July 2008] |
| E2F7 | E2F | E2F transcription factors, such as E2F7, play an essential role in the regulation of cell cycle |
| | Transcription | progression (Di Stefano et al., 2003 [PubMed 14633988]). [supplied by OMIM, May 2008] |
| | Factor 7 | |
| HOXA2 | Homeobox A2 | In vertebrates, the genes encoding the class of transcription factors called homeobox genes are found |
| | | in clusters named A, B, C, and D on four separate chromosomes. Expression of these proteins is |
| | | spatially and temporally regulated during embryonic development. This gene is part of the A cluster |
| | | on chromosome 7 and encodes a DNA-binding transcription factor which may regulate gene |
| | | expression, morphogenesis, and differentiation. The encoded protein may be involved in the |
| | | placement of hindbrain segments in the proper location along the anterior-posterior axis during |
| | | development. [provided by RefSeq, July 2008] |
| SOX2 | SRY-Box | This intronless gene encodes a member of the SRY-related HMG-box (SOX) family of transcription |
| | Transcription | factors involved in the regulation of embryonic development and in the determination of cell fate. The |
| | Factor 2 | product of this gene is required for stem-cell maintenance in the central nervous system, and also |
| | | regulates gene expression in the stomach. Mutations in this gene have been associated with optic |
| | | nerve hypoplasia and with syndromic microphthalmia, a severe form of structural eye malformation. |
| | | This gene lies within an intron of another gene called SOX2 overlapping transcript (SOX2OT). |
| | | [provided by RefSeq, July 2008] |
| OVOL2 | Ovo Like Zinc | This gene encodes a member of the evolutionarily conserved ovo-like protein family. Mammalian |
| | Finger 2 | members of this family contain a single zinc finger domain composed of a tetrad of C2H2 zinc fingers |
| | | with variable N- and C-terminal extensions that contain intrinsically disordered domains. Members of |
| | | this family are involved in epithelial development and differentiation. Knockout of this gene in mouse |
| | | results in early embryonic lethality with phenotypes that include neurectoderm expansion, impaired |
| | | vascularization, and heart anomalies. In humans, allelic variants of this gene have been associated |
| | | with posterior polymorphous corneal dystrophy. [provided by RefSeq, April 2016] |
| GFI1 | Growth Factor | This gene encodes a nuclear zinc finger protein that functions as a transcriptional repressor. This |
| | Independent 1 | protein plays a role in diverse developmental contexts, including hematopoiesis and oncogenesis. It |
| | Transcriptional | functions as part of a complex along with other cofactors to control histone modifications that lead to |
| | Repressor | silencing of the target gene promoters. Mutations in this gene cause autosomal dominant severe |
| | | congenital neutropenia, and also dominant nonimmune chronic idiopathic neutropenia of adults, |
| | | which are heterogeneous hematopoietic disorders that cause predispositions to leukemias and |
| | | infections. Multiple alternatively spliced variants, encoding the same protein, have been identified for |
| | | this gene. [provided by RefSeq, July 2008] |
| SALL1 | Spalt Like | The protein encoded by this gene is a zinc finger transcriptional repressor and may be part of the |
| | Transcription | NuRD histone deacetylase complex (HDAC). Defects in this gene are a cause of Townes-Brocks |
| | Factor 1 | syndrome (TBS) as well as bronchio-oto-renal syndrome (BOR). Two transcript variants encoding |
| | | different isoforms have been found for this gene. [provided by RefSeq, July 2008] |
| RFX3 | Regulatory | This gene is a member of the regulatory factor X gene family, which encodes transcription factors |
| | Factor X3 | that contain a highly-conserved winged helix DNA binding domain. The protein encoded by this gene |
| | | is structurally related to regulatory factors X1, X2, X4, and X5. It is a transcriptional activator that |
| | | can bind DNA as a monomer or as a heterodimer with other RFX family members. Multiple transcript |
| | | variants encoding different isoforms have been described for this gene. [provided by RefSeq, August |
| | | 2013] |
| ZBTB10 | Zinc Finger And | |
| | BTB Domain | |
| | Containing 10 | |
| HNF1B | HNF1 | This gene encodes a member of the homeodomain-containing superfamily of transcription factors. |
| | Homeobox B | The protein binds to DNA as either a homodimer, or a heterodimer with the related protein hepatocyte |
| | | nuclear factor 1-alpha. The gene has been shown to function in nephron development, and regulates |
| | | development of the embryonic pancreas. Mutations in this gene result in renal cysts and diabetes |
| | | syndrome and noninsulin-dependent diabetes mellitus, and expression of this gene is altered in some |
| | | types of cancer. Multiple transcript variants encoding different isoforms have been found for this |
| | | gene. [provided by RefSeq, September 2009] |
| BNC1 | Basonuclin 1 | The protein encoded by this gene is a zinc finger protein present in the basal cell layer of the |
| | | epidermis and in hair follicles. It is also found in abundance in the germ cells of testis and ovary. This |
| | | protein is thought to play a regulatory role in keratinocyte proliferation and it may also be a regulator |
| | | for rRNA transcription. Alternative splicing of this gene results in multiple transcript variants, and |
| | | multiple polyadenylation sites are indicated. [provided by RefSeq, July 2014] |
| ZIC1 | Zic Family | This gene encodes a member of the ZIC family of C2H2-type zinc finger proteins. Members of this |
| | Member 1 | family are important during development. Aberrant expression of this gene is seen in |
| | | medulloblastoma, a childhood brain tumor. This gene is closely linked to the gene encoding zinc |
| | | finger protein of the cerebellum 4, a related family member on chromosome 3. This gene encodes a |
| | | transcription factor that can bind and transactivate the apolipoprotein E gene. [provided by RefSeq, |
| | | July 2008] |
| SIX3 | SIX Homeobox | This gene encodes a member of the sine oculis homeobox transcription factor family. The encoded |
| | 3 | protein plays a role in eye development. Mutations in this gene have been associated with |
| | | holoprosencephaly type 2. [provided by RefSeq, October 2009] |
| NR6A1 | Nuclear | This gene encodes an orphan nuclear receptor which is a member of the nuclear hormone receptor |
| | Receptor | family. Its expression pattern suggests that it may be involved in neurogenesis and germ cell |
| | Subfamily 6 | development. The protein can homodimerize and bind DNA, but in vivo targets have not been |
| | Group A | identified. Alternate splicing results in multiple transcript variants. [provided by RefSeq, June 2013] |
| | Member 1 | |
| ZNF845 | Zinc Finger | |
| | Protein 845 | |
| ZNF608 | Zinc Finger | |
| | Protein 608 | |
| LIN28B | Lin-28 Homolog | The protein encoded by this gene belongs to the lin-28 family, which is characterized by the presence |
| | B | of a cold-shock domain and a pair of CCHC zinc finger domains. This gene is highly expressed in |
| | | testis, fetal liver, placenta, and in primary human tumors and cancer cell lines. It is negatively |
| | | regulated by microRNAs that target sites in the 3′ UTR, and overexpression of this gene in primary |
| | | tumors is linked to the repression of let-7 family of microRNAs and derepression of let-7 targets, |
| | | which facilitates cellular transformation. [provided by RefSeq, June 2012] |
| ZNF280C | Zinc Finger | This gene encodes a member of the zinc finger domain-containing protein family. This family |
| | Protein 280C | member contains multiple Cys2-His2(C2H2)-type zinc finger domains, the most common type of zinc |
| | | finger domain that self-folds to form a beta-beta-alpha structure that binds a zinc ion. [provided by |
| | | RefSeq, August 2011] |
| ELF3 | E74 Like ETS | |
| | Transcription | |
| | Factor 3 | |
| CDX1 | Caudal Type | This gene is a member of the caudal-related homeobox transcription factor gene family. The encoded |
| | Homeobox 1 | DNA-binding protein regulates intestine-specific gene expression and enterocyte differentiation. It |
| | | has been shown to induce expression of the intestinal alkaline phosphatase gene, and inhibit beta- |
| | | catenin/T-cell factor transcriptional activity. [provided by RefSeq, July 2008] |
| OTX2 | Orthodenticle | This gene encodes a member of the bicoid subfamily of homeodomain-containing transcription |
| | Homeobox 2 | factors. The encoded protein acts as a transcription factor and plays a role in brain, craniofacial, and |
| | | sensory organ development. The encoded protein also influences the proliferation and differentiation |
| | | of dopaminergic neuronal progenitor cells during mitosis. Mutations in this gene cause syndromic |
| | | microphthalmia 5 (MCOPS5) and combined pituitary hormone deficiency 6 (CPHD6). This gene is |
| | | also suspected of having an oncogenic role in medulloblastoma. Alternative splicing results in |
| | | multiple transcript variants encoding distinct isoforms. Pseudogenes of this gene are known to exist |
| | | on chromosomes two and nine. [provided by RefSeq, July 2012] |
| BARHL2 | BarH Like | |
| | Homeobox 2 | |
| ESR2 | Estrogen | This gene encodes a member of the family of estrogen receptors and superfamily of nuclear receptor |
| | Receptor 2 | transcription factors. The gene product contains an N-terminal DNA binding domain and C-terminal |
| | | ligand binding domain and is localized to the nucleus, cytoplasm, and mitochondria. Upon binding to |
| | | 17beta-estradiol or related ligands, the encoded protein forms homo- or hetero-dimers that interact |
| | | with specific DNA sequences to activate transcription. Some isoforms dominantly inhibit the activity |
| | | of other estrogen receptor family members. Several alternatively spliced transcript variants of this |
| | | gene have been described, but the full-length nature of some of these variants has not been fully |
| | | characterized. [provided by RefSeq, July 2008] |
| ZIC2 | Zic Family | This gene encodes a member of the ZIC family of C2H2-type zinc finger proteins. This protein |
| | Member 2 | functions as a transcriptional repressor and may regulate tissue specific expression of dopamine |
| | | receptor D1. Expansion of an alanine repeat in the C-terminus of the encoded protein and other |
| | | mutations in this gene cause holoprosencephaly type 5. Holoprosencephaly is the most common |
| | | structural anomaly of the human brain. A polyhistidine tract polymorphism in this gene may be |
| | | associated with increased risk of neural tube defects. This gene is closely linked to a gene encoding |
| | | zinc finger protein of the cerebellum 5, a related family member on chromosome 13. [provided by |
| | | RefSeq, July 2016] |
| HNF4G | Hepatocyte | |
| | Nuclear Factor 4 | |
| | Gamma | |
| DLX6 | Distal-Less | This gene encodes a member of a homeobox transcription factor gene family similiar to the |
| | Homeobox 6 | Drosophila distal-less gene. This family is comprised of at least 6 different members that encode |
| | | proteins with roles in forebrain and craniofacial development. This gene is in a tail-to-tail |
| | | configuration with another member of the family on the long arm of chromosome 7. [provided by |
| | | RefSeq, July 2008] |
| POU2F1 | POU Class 2 | The OCT1 transcription factor was among the first identified members of the POU transcription |
| | Homeobox 1 | factor family (summarized by Sturm et al., 1993 [PubMed 8314572]). Members of this family contain |
| | | the POU domain, a 160-amino acid region necessary for DNA binding to the octameric sequence |
| | | ATGCAAAT. [supplied by OMIM, July 2010] |
| OVOL1 | Ovo Like | This gene encodes a putative zinc finger containing transcription factor that is highly similar to |
| | Transcriptional | homologous protein in Drosophila and mouse. Based on known functions in these species, this |
| | Repressor 1 | protein is likely involved in hair formation and spermatogenesis in human as well. [provided by |
| | | RefSeq, August 2011] |
| MYCN | MYCN Proto- | This gene is a member of the MYC family and encodes a protein with a basic helix-loop-helix |
| | Oncogene, | (bHLH) domain. This protein is located in the nucleus and must dimerize with another bHLH protein |
| | BHLH | in order to bind DNA. Amplification of this gene is associated with a variety of tumors, most notably |
| | Transcription | neuroblastomas. Multiple alternatively spliced transcript variants encoding different isoforms have |
| | Factor | been found for this gene. [provided by RefSeq, June 2014] |
| SIX2 | SIX Homeobox | This gene is a member of the vertebrate gene family which encode proteins homologous to the |
| | 2 | Drosophila ‘sine oculis’ homeobox protein. The encoded protein is a transcription factor which, like |
| | | other members of this gene family, may be involved in limb or eye development. [provided by |
| | | RefSeq, December 2008] |
| SOX13 | SRY-Box | This gene encodes a member of the SOX (SRY-related HMG-box) family of transcription factors |
| | Transcription | involved in the regulation of embryonic development and in the determination of cell fate. The |
| | Factor 13 | encoded protein may act as a transcriptional regulator after forming a protein complex with other |
| | | proteins. It has also been determined to be a type-1 diabetes autoantigen, also known as islet cell |
| | | antibody 12. [provided by RefSeq, July 2008] |
| TOX3 | TOX High | The protein encoded by this gene contains an HMG-box, indicating that it may be involved in |
| | Mobility Group | bending and unwinding of DNA and alteration of chromatin structure. The C-terminus of the encoded |
| | Box Family | protein is glutamine-rich due to CAG repeats in the coding sequence. A minor allele of this gene has |
| | Member 3 | been implicated in an elevated risk of breast cancer. Two transcript variants encoding different |
| | | isoforms have been found for this gene. [provided by RefSeq, April 2009] |
| ARID3B | AT-Rich | This gene encodes a member of the ARID (AT-rich interaction domain) family of DNA-binding |
| | Interaction | proteins. The encoded protein is homologous with two proteins that bind to the retinoblastoma gene |
| | Domain 3B | product, and also with the mouse Bright and Drosophila dead ringer proteins. A pseudogene on |
| | | chromosome 1p31 exists for this gene. Members of the ARID family have roles in embryonic |
| | | patterning, cell lineage gene regulation, cell cycle control, transcriptional regulation and possibly in |
| | | chromatin structure modification. [provided by RefSeq, July 2008] |
| ZBTB9 | Zinc Finger And | |
| | BTB Domain | |
| | Containing 9 | |
| EZH2 | Enhancer Of | This gene encodes a member of the Polycomb-group (PcG) family. PcG family members form |
| | Zeste 2 | multimeric protein complexes, which are involved in maintaining the transcriptional repressive state |
| | Polycomb | of genes over successive cell generations. This protein associates with the embryonic ectoderm |
| | Repressive | development protein, the VAVI oncoprotein, and the X-linked nuclear protein. This protein may play |
| | Complex 2 | a role in the hematopoietic and central nervous systems. Multiple alternatively splcied transcript |
| | Subunit | variants encoding distinct isoforms have been identified for this gene. [provided by RefSeq, February |
| | | 2011] |
| ZBTB32 | Zinc Finger And | |
| | BTB Domain | |
| | Containing 32 | |
| MIS18BP1 | MIS18 Binding | |
| | Protein 1 | |
| WDHD1 | WD Repeat And | The protein encoded by this gene contains multiple N-terminal WD40 domains and a C-terminal high |
| | HMG-Box DNA | mobility group (HMG) box. WD40 domains are found in a variety of eukaryotic proteins and may |
| | Binding Protein | function as adaptor/regulatory modules in signal transduction, pre-mRNA processing and |
| | 1 | cytoskeleton assembly. HMG boxes are found in many eukaryotic proteins involved in chromatin |
| | | assembly, transcription and replication. Alternative splicing results in two transcript variants encoding |
| | | different isoforms. [provided by RefSeq, July 2008] |
| JARID2 | Jumonji And | This gene encodes a Jumonji- and AT-rich interaction domain (ARID)-domain-containing protein. |
| | AT-Rich | The encoded protein is a DNA-binding protein that functions as a transcriptional repressor. This |
| | Interaction | protein interacts with the Polycomb repressive complex 2 (PRC2) which plays an essential role in |
| | Domain | regulating gene expression during embryonic development. This protein facilitates the recruitment of |
| | Containing 2 | the PRC2 complex to target genes. Alternate splicing results in multiple transcript variants. Mutations |
| | | in this gene are associated with chronic myeloid malignancies. [provided by RefSeq, May 2012] |
| CENPT | Centromere | The centromere is a specialized chromatin domain, present throughout the cell cycle, that acts as a |
| | Protein T | platform on which the transient assembly of the kinetochore occurs during mitosis. All active |
| | | centromeres are characterized by the presence of long arrays of nucleosomes in which CENPA (MIM |
| | | 117139) replaces histone H3 (see MIM 601128). CENPT is an additional factor required for |
| | | centromere assembly (Foltz et al., 2006 [PubMed 16622419]). [supplied by OMIM, March 2008] |
| ZFP1 | ZFP1 Zinc | This gene belongs to the zinc finger protein family. Some members of this family bind to DNA by |
| | Finger Protein | zinc-mediated secondary structures called zinc fingers, and are involved in transcriptional regulation. |
| | | Alternative splicing results in multiple transcript variants encoding different isoforms. [provided by |
| | | RefSeq, January 2016] |
| TCF7 | Transcription | This gene encodes a member of the T-cell factor/lymphoid enhancer-binding factor family of high |
| | Factor 7 | mobility group (HMG) box transcriptional activators. This gene is expressed predominantly in T-cells |
| | | and plays a critical role in natural killer cell and innate lymphoid cell development. The encoded |
| | | protein forms a complex with beta-catenin and activates transcription through a Wnt/beta-catenin |
| | | signaling pathway. Mice with a knockout of this gene are viable and fertile, but display a block in T- |
| | | lymphocyte differentiation. Alternative splicing results in multiple transcript variants. Naturally - |
| | | occurring isoforms lacking the N-terminal beta-catenin interaction domain may act as dominant |
| | | negative regulators of Wnt signaling. [provided by RefSeq, October 2016] |
| ZNF614 | Zinc Finger | |
| | Protein 614 | |

### EXAMPLE 1 REFERENCES

Acampora, D., Di Giovannantonio, L. G. and Simeone, A. (2013). Otx2 is an intrinsic determinant of the embryonic stem cell state and is required for transition to a stable epiblast stem cell condition. Dev. 140, 43-55.
Ahuja, D., Sáenz-Robles, M. T. and Pipas, J. M. (2005). SV40 large T antigen targets multiple cellular pathways to elicit cellular transformation. Oncogene 24, 7729-7745.
Ait-Si-Ali, S., Guasconi, V., Fritsch, L., Yahi, H., Sekhri, R., Naguibneva, I., Robin, P., Cabon, F., Polesskaya, A. and Harel-Bellan, A. (2004). A Suv39h-dependent mechanism for silencing S-phase genes in differentiating but not in cycling cells. EMBO J. 23, 605-615.
Ali, S. H. and DeCaprio, J. A. (2001). Cellular transformation by SV40 large T antigen: Interaction with host proteins. Semin. Cancer Biol. 11, 15-22.
Ambrosetti, D. C., Basilico, C. and Dailey, L. (1997). Synergistic activation of the fibroblast growth factor 4 enhancer by Sox2 and Oct-3 depends on protein-protein interactions facilitated by a specific spatial arrangement of factor binding sites. Mol. Cell. Biol. 17, 6321-6329.
Avilion, A. A., Nicolis, S. K., Pevny, L. H., Perez, L., Vivian, N. and Lovell-Badge, R. (2003). Multipotent cell lineages in early mouse development depend on SOX2 function. Genes Dev. 17, 126-140.
Bai, C., Li, X., Gao, Y., Yuan, Z., Hu, P., Wang, H., Liu, C., Guan, W. and Ma, Y. (2016). Melatonin improves reprogramming efficiency and proliferation of bovine-induced pluripotent stem cells. J Pineal Res 61, 154-167.
Bao, L., He, L., Chen, J., Wu, Z., Liao, J., Rao, L., Ren, J., Li, H., Zhu, H., Qian, L., et al. (2011). Reprogramming of ovine adult fibroblasts to pluripotency via drug-inducible expression of defined factors. Cell Res. 21, 600-608.
Bashamboo, A., Taylor, A. H., Samuel, K., Panthier, J. J., Whetton, A. D. and Forrester, L. M. (2006). The survival of differentiating embryonic stem cells is dependent on the SCF-KIT pathway. J. Cell Sci. 119, 3039-3046.
Bogliotti, Y. S., Wu, J., Vilarino, M., Okamura, D., Soto, D. A., Zhong, C., Sakurai, M., Sampaio, R. V, Suzuki, K., Izpisua Belmonte, J. C., et al. (2018). Efficient derivation of stable primed pluripotent embryonic stem cells from bovine blastocysts. Proc Natl Acad Sci USA 115, 2090-2095.
Britt, J. H., Cox, N. M. and Stevenson, J. S. (1981). Advances in Reproduction in Dairy Cattle. J. Dairy Sci. 64, 1378-1402.
Cadigan, K. M. and Waterman, M. L. (2012). TCF/LEFs and Wnt signaling in the nucleus. Cold Spring Harb. Perspect. Biol. 4, a007906.
Canizo, J. R., Vazquez Echegaray, C., Klisch, D., Aller, J. F., Paz, D. A., Alberio, R. H., Alberio, R. and Guberman, A. S. (2018). Exogenous human OKSM factors maintain pluripotency gene expression of bovine and porcine iPS-like cells obtained with STEMCCA delivery system. BMC Res Notes 11, 509.
Cao, S., Wang, F., Chen, Z., Liu, Z., Mei, C., Wu, H., Huang, J., Li, C., Zhou, L. and Liu, L. (2009). Isolation and culture of primary bovine embryonic stem cell colonies by a novel method. J Exp Zool A Ecol Genet Physiol 311, 368-376.
Cao, H., Yang, P., Pu, Y., Sun, X., Yin, H., Zhang, Y., Zhang, Y., Li, Y., Liu, Y., Fang, F., et al. (2012). Characterization of bovine induced pluripotent stem cells by lentiviral transduction of reprogramming factor fusion proteins. Int J Biol Sci 8, 498-511.
Carvajal, L. A., Hamard, P. J., Tonnessen, C. and Manfredi, J. J. (2012). E2F7, a novel target, is up-regulated by p53 and mediates DNA damage-dependent transcriptional repression. Genes Dev. 26, 1533-1545.
Cherny, R. A., Stokes, T. M., Merei, J., Lom, L., Brandon, M. R. and Williams, R. L. (1994). Strategies for the isolation and characterization of bovine embryonic stem cells. Reprod Fertil Dev 6, 569-575.
Christ, A., Christa, A., Kur, E., Lioubinski, O., Bachmann, S., Willnow, T. E. and Hammes, A. (2012). LRP2 Is an Auxiliary SHH Receptor Required to Condition the Forebrain Ventral Midline for Inductive Signals. Dev. Cell 22, 268-278.
Cibelli, J. B., Stice, S. L., Golueke, P. J., Kane, J. J., Jerry, J., Blackwell, C., Ponce De León, F. A. and Robl, J. M. (1998). Transgenic bovine chimeric offspring produced from somatic cell-derived stem-like cells. Nat. Biotechnol. 16, 642-646.
Cong, S., Cao, G. and Liu, D. (2014). Effects of different feeder layers on culture of bovine embryonic stem cell-like cells in vitro. Cytotechnology 66, 995-1005.
Cravero, D., Martignani, E., Miretti, S., Accornero, P., Pauciullo, A., Sharma, R., Donadeu, F. X. and Baratta, M. (2015). Generation of Induced Pluripotent Stem Cells from Bovine Epithelial Cells and Partial Redirection Toward a Mammary Phenotype In Vitro. Cell Reprogr. 17, 211-220.
Cselenyi, C. S., Jernigan, K. K., Tahinci, E., Thorne, C. A., Lee, L. A. and Lee, E. (2008). LRP6 transduces a canonical Wnt signal independently of Axin degradation by inhibiting GSK3's phosphorylation of β-catenin. Proc. Natl. Acad. Sci. U.S.A 105, 8032-8037.
Dautry Varsat, A., Ciechanover, A. and Lodish, H. F. (1983). pH and the recycling of transferrin during receptor-mediated endocytosis. Proc. Natl. Acad. Sci. U.S.A 80, 2258-2262.
de Souza, M. M., Zerlotini, A., Geistlinger, L., Tizioto, P. C., Taylor, J. F., Rocha, M. I. P., Diniz, W. J. S., Coutinho, L. L. and Regitano, L. C. A. (2018). A comprehensive manually-curated compendium of bovine transcription factors. Sci. Rep. 8, 13747.
Di Stefano, L., Jensen, M. R. and Helin, K. (2003). E2F7, a novel E2F featuring DP-independent repression of a subset of E2F-regulated genes. EMBO J. 22, 6289-6298.
Dobin, A. and Gingeras, T. R. (2015). Mapping RNA-seq Reads with STAR. Curr. Protoc. Bioinforma. 51, 11.14.1-11.14.19.
Evans, M. J. and Kaufman, M. H. (1981). Establishment in culture of pluripotential cells from mouse embryos. Nature 292, 154-156.
Ezashi, T., Yuan, Y. and Roberts, R. M. (2016). Pluripotent Stem Cells from Domesticated Mammals. Annu Rev Anim Biosci 4, 223-253.
First, N. L., Sims, M. M., Park, S. P. and Kent-First, M. J. (1994). Systems for production of calves from cultured bovine embryonic cells. Reprod. Fertil. Dev. 6, 553-562.
Fletcher, C., Heintz, N. and Roeder, R. G. (1987). Purification and characterization of OTF-1, a transcription factor regulating cell cycle expression of a human histone H2b gene. Cell 51, 773-781.
Fraser, L., Taylor, A. H. and Forrester, L. M. (2013). SCF/KIT inhibition has a cumulative but reversible effect on the self-renewal of embryonic stem cells and on the survival of differentiating cells. Cell. Reprogram. 15, 259-268.
Ge, S. X., Son, E. W. and Yao, R. (2018). iDEP: An integrated web application for differential expression and pathway analysis of RNA-Seq data. BMC Bioinformatics 19, 1-24.
Graf, A., Krebs, S., Zakhartchenko, V., Schwalb, B., Blum, H. and Wolf, E. (2014). Fine mapping of genome activation in bovine embryos by RNA sequencing. Proc. Natl. Acad. Sci. U.S.A 111, 4139-4144.
Green, B. B., McKay, S. D. and Kerr, D. E. (2015). Age dependent changes in the LPS induced transcriptome of bovine dermal fibroblasts occurs without major changes in the methylome. BMC Genomics 16, 30.
Guo, G., Von Meyenn, F., Santos, F., Chen, Y., Reik, W., Bertone, P., Smith, A. and Nichols, J. (2016). Naive Pluripotent Stem Cells Derived Directly from Isolated Cells of the Human Inner Cell Mass. Stem Cell Reports 6, 437-446.
Han, X., Han, J., Ding, F., Cao, S., Lim, S. S., Dai, Y., Zhang, R., Zhang, Y., Lim, B. and Li, N. (2011). Generation of induced pluripotent stem cells from bovine embryonic fibroblast cells. Cell Res 21, 1509-1512.
Heo, J. S., Lee, M. Y. and Han, H. J. (2007). Sonic Hedgehog Stimulates Mouse Embryonic Stem Cell Proliferation by Cooperation of Ca²⁺/Protein Kinase C and Epidermal Growth Factor Receptor As Well as Gli1 Activation. Stem Cells 25, 3069-3080.
Heo, Y. T., Quan, X., Xu, Y. N., Baek, S., Choi, H., Kim, N. H. and Kim, J. (2015). CRISPR/Cas9 nuclease-mediated gene knock-in in bovine-induced pluripotent cells. Stem Cells Dev 24, 393-402.
Heuberger, D. M. and Schuepbach, R. A. (2019). Protease-activated receptors (PARs): Mechanisms of action and potential therapeutic modulators in PAR-driven inflammatory diseases. Thromb. J. 17, 4.
Huang, B., Li, T., Alonso-Gonzalez, L., Gorre, R., Keatley, S., Green, A., Turner, P., Kallingappa, P. K., Verma, V. and Oback, B. (2011). A virus-free poly-promoter vector induces pluripotency in quiescent bovine cells under chemically defined conditions of dual kinase inhibition. PLoS One 6, e24501.
Huang, X., Han, X., Uyunbilig, B., Zhang, M., Duo, S., Zuo, Y., Zhao, Y., Yun, T., Tai, D., Wang, C., et al. (2014). Establishment of Bovine Trophoblast Stem-Like Cells from In Vitro—Produced Blastocyst-Stage Embryos Using Two Inhibitors. Stem Cells Dev. 23, 1501-1514.
Iwasaki, S., Campbell, K. H. S., Galli, C., Akiyama, K. and Iwasaki, S. (2000). Production of Live Calves Derived from Embryonic Stem-Like Cells Aggregated with Tetraploid Embryos1. Biol. Reprod. 62, 470-475.
Izzi, L., Lévesque, M., Morin, S., Laniel, D., Wilkes, B. C., Mille, F., Krauss, R. S., McMahon, A. P., Allen, B. L. and Charron, F. (2011). Boc and gas1 each form distinct shh receptor complexes with ptch1 and are required for shh-mediated cell proliferation. Dev. Cell 20, 788-801.
James, D., Levine, A. J., Besser, D. and Hemmati-Brivanlou, A. (2005a). TGFβ/activin/nodal signaling is necessary for the maintenance of pluripotency in human embryonic stem cells. Development 132, 1273-1282.
James, D., Levine, A. J., Besser, D. and Hemmati-Brivanlou, A. (2005b). TGFβ/activin/nodal signaling is necessary for the maintenance of pluripotency in human embryonic stem cells. Development 132, 1273-1282.
Jandl, J. H., Inman, J. K., Simmons, R. L. and Allen, D. W. (1959). Transfer of iron from serum iron-binding protein to human reticulocytes. J. Clin. Invest. 38, 161-185.
Kabir, M. H., Patrick, R., Ho, J. W. K. and O'Connor, M. D. (2018). Identification of active signaling pathways by integrating gene expression and protein interaction data. BMC Syst. Biol. 12, 120.
Kareta, M. S., Gorges, L. L., Hafeez, S., Benayoun, B. A., Marro, S., Zmoos, A. F., Cecchini, M. J., Spacek, D., Batista, L. F. Z., O'Brien, M., et al. (2015). Inhibition of pluripotency networks by the Rb tumor suppressor restricts reprogramming and tumorigenesis. Cell Stem Cell 16, 39-50.
Kawaguchi, T., Tsukiyama, T., Kimura, K., Matsuyama, S., Minami, N., Yamada, M. and Imai, H. (2015). Generation of Naive Bovine Induced Pluripotent Stem Cells Using PiggyBac Transposition of Doxycycline-Inducible Transcription Factors. PLoS One 10, e0135403.
Kawaguchi, T., Cho, D., Hayashi, M., Tsukiyama, T., Kimura, K., Matsuyama, S., Minami, N., Yamada, M. and Imai, H. (2016). Derivation of induced trophoblast cell lines in cattle by doxycycline-inducible piggyBac vectors. PLoS One 11.
Kawamura, T., Suzuki, J., Wang, Y. V, Menendez, S., Morera, L. B., Raya, A., Wahl, G. M. and Izpisua Belmonte, J. C. (2009). Linking the p53 tumour suppressor pathway to somatic cell reprogramming. Nature 460, 1140-1144.
Keefer, C. L., Pant, D., Blomberg, L. and Talbot, N. C. (2007). Challenges and prospects for the establishment of embryonic stem cell lines of domesticated ungulates. Anim. Reprod. Sci. 98, 147-168.
Keenan, A. B., Torre, D., Lachmann, A., Leong, A. K., Wojciechowicz, M. L., Utti, V., Jagodnik, K. M., Kropiwnicki, E., Wang, Z. and Ma'ayan, A. (2019). ChEA3: transcription factor enrichment analysis by orthogonal omics integration. Nucleic Acids Res. 47, W212-W224.
Kim, E. Y., Noh, E. J., Park, H. Y., Park, M. J., Noh, E. H., Lee, J. B., Jeong, C. J., Lee, D. S., Riu, K. Z. and Park, S. P. (2012). Establishment of Bovine Embryonic Stem Cell Lines Using a Minimized Feeder Cell Drop. Cell. Reprogram. 14, 520-529.
Larsen, J. V. and Petersen, C. M. (2017). SorLA in Interleukin-6 Signaling and Turnover. Mol. Cell. Biol. 37.
Lau, S. T., Li, Z., Pui-Ling Lai, F., Nga-Chu Lui, K., Li, P., Munera, J. O., Pan, G., Mahe, M. M., Hui, C. C., Wells, J. M., et al. (2019). Activation of Hedgehog Signaling Promotes Development of Mouse and Human Enteric Neural Crest Cells, Based on Single-Cell Transcriptome Analyses. Gastroenterology 157, 1556-1571.e5.
Li, Q., Lex, R. K., Chung, H., Giovanetti, S. M., Ji, Z., Ji, H., Person, M. D., Kim, J. and Vokes, S. A. (2016). The pluripotency factor NANOG binds to GLI proteins and represses hedgehog-mediated transcription. J Biol. Chem. 291, 7171-7182.
Lim, M. L., Vassiliev, I., Richings, N. M., Firsova, A. B., Zhang, C. and Verma, P. J. (2011). A novel, efficient method to derive bovine and mouse embryonic stem cells with in vivo differentiation potential by treatment with 5-azacytidine. Theriogenology 76, 133-142.
Lin, Y. C., Kuo, K. K., Wuputra, K., Lin, S. H., Ku, C. C., Yang, Y. H., Wang, S. W., Wang, S. W., Wu, D. C., Wu, C. C., et al. (2014). Bovine induced pluripotent stem cells are more resistant to apoptosis than testicular cells in response to mono-(2-ethylhexyl) phthalate. Int J Mol Sci 15, 5011-5031.
Liu, Y., Charles, L. F., Zarembinski, T. I., Johnson, K. I., Atzet, S. K., Wesselschmidt, R. L., Wight, M. E. and Kuhn, L. T. (2012). Modified Hyaluronan Hydrogels Support the Maintenance of Mouse Embryonic Stem Cells and Human Induced Pluripotent Stem Cells. Macromol. Biosci. 12, 1034-1042.
Liu, B., Shats, I., Angus, S. P., Gatza, M. L. and Nevins, J. R. (2013). Interaction of E2F7 transcription factor with E2F1 and c-terminal-binding protein (CtBP) provides a mechanism for E2F7-dependent transcription repression. J. Biol. Chem. 288, 24581-24589.
Liu, X., Wang, Y., Gao, Y., Su, J., Zhang, J., Xing, X., Zhou, C., Yao, K., An, Q. and Zhang, Y. (2018). H3K9 demethylase KDM4E is an epigenetic regulator for bovine embryonic development and a defective factor for nuclear reprogramming. Dev. 145.
Maguire, C. T., Demarest, B. L., Hill, J. T., Palmer, J. D., Brothman, A. R., Yost, H. J. and Condic, M. L. (2013). Genome-Wide Analysis Reveals the Unique Stem Cell Identity of Human Amniocytes. PLoS One 8, e53372.
Maherali, N. and Hochedlinger, K. (2009). Tgfβ Signal Inhibition Cooperates in the Induction of iPSCs and Replaces Sox2 and cMyc. Curr. Biol. 19, 1718-1723.
Malaver-Ortega, L. F., Sumer, H., Liu, J. and Verma, P. J. (2012). The state of the art for pluripotent stem cells derivation in domestic ungulates. Theriogenology 78, 1749-1762.
Mali, P., Ye, Z., Hommond, H. H., Yu, X., Lin, J., Chen, G., Zou, J. and Cheng, L. (2008a). Improved Efficiency and Pace of Generating Induced Pluripotent Stem Cells from Human Adult and Fetal Fibroblasts. Stem Cells 26, 1998-2005.
Mali, P., Ye, Z., Hommond, H. H., Yu, X., Lin, J., Chen, G., Zou, J. and Cheng, L. (2008b). Improved Efficiency and Pace of Generating Induced Pluripotent Stem Cells from Human Adult and Fetal Fibroblasts. Stem Cells 26, 1998-2005.
Mandala, A., Armstrong, A., Girresch, B., Zhu, J., Chilakala, A., Chavalmane, S., Chaudhary, K., Biswas, P., Ogilvie, J. and Gnana-Prakasam, J. P. (2020). Fenofibrate prevents iron induced activation of canonical Wnt/β-catenin and oxidative stress signaling in the retina. npj Aging Mech. Dis. 6, 1-11.
Mao, C. D. and Byers, S. W. (2011). Cell-context dependent TCF/LEF expression and function: Alternative tales of repression, de-repression and activation potentials. Crit. Rev. Eukaryot. Gene Expr. 21, 207-236.
Martin, G. R. (1981). Isolation of a pluripotent cell line from early mouse embryos cultured in medium conditioned by teratocarcinoma stem cells. Proc Natl Acad Sci USA 78, 7634-7638.
Mikkelsen, T. S., Hanna, J., Zhang, X., Ku, M., Wernig, M., Schorderet, P., Bernstein, B. E., Jaenisch, R., Lander, E. S. and Meissner, A. (2008). Dissecting direct reprogramming through integrative genomic analysis. Nature 454, 49-55.
Mitalipova, M., Beyhan, Z. and First, N. L. (2001). Pluripotency of bovine embryonic cell line derived from precompacting embryos. Cloning 3, 59-67.
Miura, T., Yuasa, N., Ota, H., Habu, M., Kawano, M., Nakayama, F. and Nishihara, S. (2019). Highly sulfated hyaluronic acid maintains human induced pluripotent stem cells under feeder-free and bFGF-free conditions. Biochem. Biophys. Res. Commun. 518, 506-512.
Morohaku, K., Phuong, N. S. and Selvaraj, V. (2013). Developmental expression of translocator protein/peripheral benzodiazepine receptor in reproductive tissues. PLoS One 8, e74509.
Morohaku, K., Pelton, S. H., Daugherty, D. J., Butler, W. R., Deng, W. and Selvaraj, V. (2014). Translocator Protein/Peripheral Benzodiazepine Receptor Is Not Required for Steroid Hormone Biosynthesis. Endocrinology 155, 89-97.
Nichols, J., Chambers, I. and Smith, A. (1994). Short Note: Derivation of germline competent embryonic stem cells with a combination of interleukin-6 and soluble interleukin-6 receptor. Exp. Cell Res. 215, 237-239.
Nielsen, S. J., Schneider, R., Bauer, U. M., Bannister, A. J., Morrison, A., O'Carroll, D., Firestein, R., Cleary, M., Jenuwein, T., Herrera, R. E., et al. (2001). Rb targets histone H3 methylation and HP1 to promoters. Nature 412, 561-565.
Niwa, H., Burdon, T., Chambers, I. and Smith, A. (1998). Self-renewal of pluripotent embryonic stem cells is mediated via activation of STAT3. Genes Dev. 12, 2048-2060.
Palmqvist, L., Glover, C. H., Hsu, L., Lu, M., Bossen, B., Piret, J. M., Humphries, R. K. and Helgason, C. D. (2005). Correlation of Murine Embryonic Stem Cell Gene Expression Profiles with Functional Measures of Pluripotency. Stem Cells 23, 663-680.
Pant, D. and Keefer, C. L. (2009). Expression of pluripotency-related genes during bovine inner cell mass explant culture. Cloning Stem Cells 11, 355-365.
Park, I. H., Zhao, R., West, J. A., Yabuuchi, A., Huo, H., Ince, T. A., Lerou, P. H., Lensch, M. W. and Daley, G. Q. (2008a). Reprogramming of human somatic cells to pluripotency with defined factors. Nature 451, 141-146.
Park, I. H., Arora, N., Huo, H., Maherali, N., Ahfeldt, T., Shimamura, A., Lensch, M. W., Cowan, C., Hochedlinger, K. and Daley, G. Q. (2008b). Disease-Specific Induced Pluripotent Stem Cells. Cell 134, 877-886.
Pashaiasl, M., Khodadadi, K., Holland, M. K. and Verma, P. J. (2010). The efficient generation of cell lines from bovine parthenotes. Cell. Reprogram. 12, 571-579.
Pashaiasl, M., Khodadadi, K., Richings, N. M., Holland, M. K. and Verma, P. J. (2013). Cryopreservation and long-term maintenance of bovine embryo-derived cell lines. Reprod. Fertil. Dev. 25, 707-718.
Pieri, N. C. G., de Souza, A. F., Botigelli, R. C., Machado, L. S., Ambrosio, C. E., dos Santos Martins, D., de Andrade, A. F. C., Meirelles, F. V., Hyttel, P. and Bressan, F. F. (2019). Stem cells on regenerative and reproductive science in domestic animals. Vet. Res. Commun. 43, 7-16.
Pillai, V. V, Siqueira, L. G., Das, M., Kei, T. G., Tu, L. N., Herren, A. W., Phinney, B. S., Cheong, S. H., Hansen, P. J. and Selvaraj, V. (2019a). Physiological profile of undifferentiated bovine blastocyst-derived trophoblasts. Biol Open 8.
Pillai, V. V., Kei, T. G., Reddy, S. E., Das, M., Abratte, C., Cheong, S. H. and Selvaraj, V. (2019b). Induced pluripotent stem cell generation from bovine somatic cells indicates unmet needs for pluripotency sustenance. Anim. Sci. J. 90, 1149-1160.
Pinto, J. P., Kalathur, R. K., Oliveira, D. V, Barata, T., Machado, R. S. R., Machado, S., Pacheco-Leyva, I., Duarte, I. and Futschik, M. E. (2015). StemChecker: a web-based tool to discover and explore stemness signatures in gene sets. Nucleic Acids Res. 43, W72-7.
Ren, J., Pak, Y., He, L., Qian, L., Gu, Y., Li, H., Rao, L., Liao, J., Cui, C., Xu, X., et al. (2011). Generation of hircine-induced pluripotent stem cells by somatic cell reprogramming. Cell Res. 21, 849-853.
Ritchie, M. E., Phipson, B., Wu, D., Hu, Y., Law, C. W., Shi, W. and Smyth, G. K. (2015). limma powers differential expression analyses for RNA-sequencing and microarray studies. Nucleic Acids Res. 43, e47.
Robinson, M. D., McCarthy, D. J. and Smyth, G. K. (2010). edgeR: a Bioconductor package for differential expression analysis of digital gene expression data. Bioinformatics 26, 139-140.
Rodda, D. J., Chew, J. L., Lim, L. H., Loh, Y. H., Wang, B., Ng, H. H. and Robson, P. (2005). Transcriptional regulation of Nanog by OCT4 and SOX2. J. Biol. Chem. 280, 24731-24737.
Saito, S., Strelchenko, N. and Niemann, H. (1992). Bovine embryonic stem cell-like cell lines cultured over several passages. Roux Arch Dev Biol 201, 134-141.
Saito, S., Sawai, K., Ugai, H., Moriyasu, S., Minamihashi, A., Yamamoto, Y., Hirayama, H., Kageyama, S., Pan, J., Murata, T., et al. (2003). Generation of cloned calves and transgenic chimeric embryos from bovine embryonic stem-like cells. Biochem. Biophys. Res. Commun. 309, 104-113.
Sato, N., Meijer, L., Skaltsounis, L., Greengard, P. and Brivanlou, A. H. (2004). Maintenance of pluripotency in human and mouse embryonic stem cells through activation of Wnt signaling by a pharmacological GSK-3-specific inhibitor. Nat. Med 10, 55-63.
Schneider, C. A., Rasband, W. S. and Eliceiri, K. W. (2012). NIH Image to ImageJ: 25 years of image analysis. Nat. Methods 9, 671-675.
Shakya, A., Kang, J., Chumley, J., Williams, M. A. and Tantin, D. (2011). Oct1 is a switchable, bipotential stabilizer of repressed and inducible transcriptional states. J. Biol. Chem. 286, 450-459.
Shultz, L. D., Lyons, B. L., Burzenski, L. M., Gott, B., Chen, X., Chaleff, S., Kotb, M., Gillies, S. D., King, M., Mangada, J., et al. (2005). Human Lymphoid and Myeloid Cell Development in NOD/LtSz-scid IL2R γ null Mice Engrafted with Mobilized Human Hemopoietic Stem Cells. J Immunol. 174, 6477-6489.
Sims, M. and First, N. L. (1994a). Production of calves by transfer of nuclei from cultured inner cell mass cells. Proc. Natl. Acad Sci. U.S.A 91, 6143-6147.
Sims, M. and First, N. L. (1994b). Production of calves by transfer of nuclei from cultured inner cell mass cells. Proc. Natl. Acad Sci. U.S.A 91, 6143-6147.
Smith, A. G., Heath, J. K., Donaldson, D. D., Wong, G. G., Moreau, J., Stahl, M. and Rogers, D. (1988). Inhibition of pluripotential embryonic stem cell differentiation by purified polypeptides. Nature 336, 688-690.
Sommer, C. A., Stadtfeld, M., Murphy, G. J., Hochedlinger, K., Kotton, D. N. and Mostoslavsky, G. (2009). Induced pluripotent stem cell generation using a single lentiviral stem cell cassette. Stem Cells 27, 543-549.
Song, S., Christova, T., Perusini, S., Alizadeh, S., Bao, R. Y., Miller, B. W., Hurren, R., Jitkova, Y., Gronda, M., Isaac, M., et al. (2011). Wnt inhibitor screen reveals iron dependence of β-catenin signaling in cancers. Cancer Res. 71, 7628-7639.
Stice, S. L. (1996). Pluripotent bovine embryonic cell lines direct embryonic development following nuclear transfer. Biol. Reprod 54, 100-110.
Stice, S. L., Strelchenko, N. S., Keefer, C. L. and Matthews, L. (1996). Pluripotent bovine embryonic cell lines direct embryonic development following nuclear transfer. Biol Reprod 54, 100-110.
Su, Y., Zhu, J., Salman, S. and Tang, Y. (2020). Induced pluripotent stem cells from farm animals. J. Anim. Sci. 98, 1-15.
Sumer, H., Liu, J., Malaver-Ortega, L. F., Lim, M. L., Khodadadi, K. and Verma, P. J. (2011). NANOG is a key factor for induction of pluripotency in bovine adult fibroblasts. J Anim Sci 89, 2708-2716.
Szklarczyk, D., Morris, J. H., Cook, H., Kuhn, M., Wyder, S., Simonovic, M., Santos, A., Doncheva, N. T., Roth, A., Bork, P., et al. (2017). The STRING database in 2017: Quality-controlled protein-protein association networks, made broadly accessible. Nucleic Acids Res. 45, D362-D368.
Takahashi, K. and Yamanaka, S. (2006). Induction of pluripotent stem cells from mouse embryonic and adult fibroblast cultures by defined factors. Cell 126, 663-676.
Takahashi, K., Tanabe, K., Ohnuki, M., Narita, M., Ichisaka, T., Tomoda, K. and Yamanaka, S. (2007). Induction of pluripotent stem cells from adult human fibroblasts by defined factors. Cell 131, 861-872.
Talbot, N. C. and Blomberg, L. A. (2008). The pursuit of ES cell lines of domesticated ungulates. Stem Cell Rev. 4, 235-254.
Talbot, N. C., Powell, A. M. and Rexroad, C. E. (1995). In vitro pluripotency of epiblasts derived from bovine blastocysts. Mol. Reprod. Dev. 42, 35-52.
Talbot, N. C., Sparks, W. O., Phillips, C. E., Ealy, A. D., Powell, A. M., Caperna, T. J., Garrett, W. M., Donovan, D. M. and Blomberg, L. A. (2017). Bovine trophectoderm cells induced from bovine fibroblasts with induced pluripotent stem cell reprogramming factors. Mol. Reprod. Dev. 84, 468-485.
Talluri, T. R., Kumar, D., Glage, S., Garrels, W., Ivics, Z., Debowski, K., Behr, R., Niemann, H. and Kues, W. A. (2015). Derivation and characterization of bovine induced pluripotent stem cells by transposon-mediated reprogramming. Cell Reprogr. 17, 131-140.
Tan, F., Qian, C., Tang, K., Abd-Allah, S. M. and Jing, N. (2015). Inhibition of transforming growth factor β (TGF-β) signaling can substitute for Oct4 protein in reprogramming and maintain pluripotency. J. Biol. Chem. 290, 4500-4511.
Tan, L., Ke, Z., Tombline, G., Macoretta, N., Hayes, K., Tian, X., Lv, R., Ablaeva, J., Gilbert, M., Bhanu, N. V., et al. (2017). Naked Mole Rat Cells Have a Stable Epigenome that Resists iPSC Reprogramming. Stem Cell Reports 9, 1721-1734.
Thomson, J. A., Itskovitz-Eldor, J., Shapiro, S. S., Waknitz, M. A., Swiergiel, J. J., Marshall, V. S. and Jones, J. M. (1998). Embryonic stem cell lines derived from human blastocysts. Science (80-.). 282, 1145-1147.
Tojo, M., Hamashima, Y., Hanyu, A., Kajimoto, T., Saitoh, M., Miyazono, K., Node, M. and Imamura, T. (2005). The ALK-5 inhibitor A-83-01 inhibits Smad signaling and epithelial-to-mesenchymal transition by transforming growth factor-beta. Cancer Sci. 96, 791-800.
Tomilin, A., Reményi, A., Lins, K., Bak, H., Leidel, S., Vriend, G., Wilmanns, M. and Schöler, H. R. (2000). Synergism with the coactivator OBF-1 (OCA-B, BOB-1) is mediated by a specific POU dimer configuration. Cell 103, 853-864.
Tu, L. N., Morohaku, K., Manna, P. R., Pelton, S. H., Butler, W. R., Stocco, D. M. and Selvaraj, V. (2014). Peripheral benzodiazepine receptor/translocator protein global knock-out mice are viable with no effects on steroid hormone biosynthesis. J. Biol. Chem. 289, 27444-27454.
Vallier, L., Alexander, M. and Pedersen, R. A. (2005). Activin/Nodal and FGF pathways cooperate to maintain pluripotency of human embryonic stem cells. J. Cell Sci. 118, 4495-4509.
Van Stekelenburg-Hamers, A. E. P., Van Achterberg, T. A. E., Rebel, H. G., Flechon, J. E., Campbell, K. H. S., Weima, S. M. and Mummery, C. L. (1995). Isolation and characterization of permanent cell lines from inner cell mass cells of bovine blastocysts. Mol. Reprod. Dev. 40, 444-454.
Wang, L., Duan, E., Sung, L. Y., Jeong, B. S., Yang, X. and Tian, X. C. (2005). Generation and characterization of pluripotent stem cells from cloned bovine embryos. Biol Reprod 73, 149-155.
Wang, S. W., Wang, S. S., Wu, D. C., Lin, Y. C., Ku, C. C., Wu, C. C., Chai, C. Y., Lee, J. N., Tsai, E. M., Lin, C. L., et al. (2013). Androgen receptor-mediated apoptosis in bovine testicular induced pluripotent stem cells in response to phthalate esters. Cell Death Dis 4, e907.
Weirauch, M. T., Yang, A., Albu, M., Cote, A. G., Montenegro-Montero, A., Drewe, P., Najafabadi, H. S., Lambert, S. A., Mann, I., Cook, K., et al. (2014). Determination and inference of eukaryotic transcription factor sequence specificity. Cell 158, 1431-1443.
Willett, E. L. (1956). Developments in the Physiology of Reproduction of Dairy Cattle and in Artificial Insemination. J. Dairy Sci. 39, 695-711.
Williams, R. L., Hilton, D. J., Pease, S., Willson, T. A., Stewart, C. L., Gearing, D. P., Wagner, E. F., Metcalf, D., Nicola, N. A. and Gough, N. M. (1988). Myeloid leukaemia inhibitory factor maintains the developmental potential of embryonic stem cells. Nature 336, 684-687.
Wooldridge, L. K. and Ealy, A. D. (2019). Interleukin-6 increases inner cell mass numbers in bovine embryos. BMC Dev. Biol. 19, 2.
Wu, M. Y. and Hill, C. S. (2009). TGF-β Superfamily Signaling in Embryonic Development and Homeostasis. Dev. Cell 16, 329-343.
Wu, S. M., Choo, A. B. H., Yap, M. G. S. and Chan, K. K. K. (2010). Role of Sonic hedgehog signaling and the expression of its components in human embryonic stem cells. Stem Cell Res. 4, 38-49.
Xu, H., Baroukh, C., Dannenfelser, R., Chen, E. Y., Tan, C. M., Kou, Y., Kim, Y. E., Lemischka, I. R. and Ma'ayan, A. (2013). ESCAPE. database for integrating high-content published data collected from human and mouse embryonic stem cells. Database (Oxford). 2013, bat045.
Ying, Q. L., Wray, J., Nichols, J., Batlle-Morera, L., Doble, B., Woodgett, J., Cohen, P. and Smith, A. (2008). The ground state of embryonic stem cell self-renewal. Nature 453, 519-523.
Yuan, Y., Park, J., Tian, Y., Choi, J., Pasquariello, R., Alexenko, A. P., Dai, A., Behura, S. K., Roberts, R. M. and Ezashi, T. (2019). A six-inhibitor culture medium for improving naïve-type pluripotency of porcine pluripotent stem cells. Cell Death Discov. 5, 104.
Zhang, H. M., Chen, H., Liu, W., Liu, H., Gong, J., Wang, H. and Guo, A. Y. (2012). AnimalTFDB: A comprehensive animal transcription factor database. Nucleic Acids Res. 40.
Zhao, Y., Yin, X., Qin, H., Zhu, F., Liu, H., Yang, W., Zhang, Q., Xiang, C., Hou, P., Song, Z., et al. (2008). Two supporting factors greatly improve the efficiency of human iPSC generation. In Cell Stem Cell, pp. 475-479. United States.
Zhao, L., Wang, Z., Zhang, J., Yang, J., Gao, X., Wu, B., Zhao, G., Bao, S., Hu, S., Liu, P., et al. (2017). Characterization of the single-cell derived bovine induced pluripotent stem cells. Tissue Cell 49, 521-527.

### Example 2

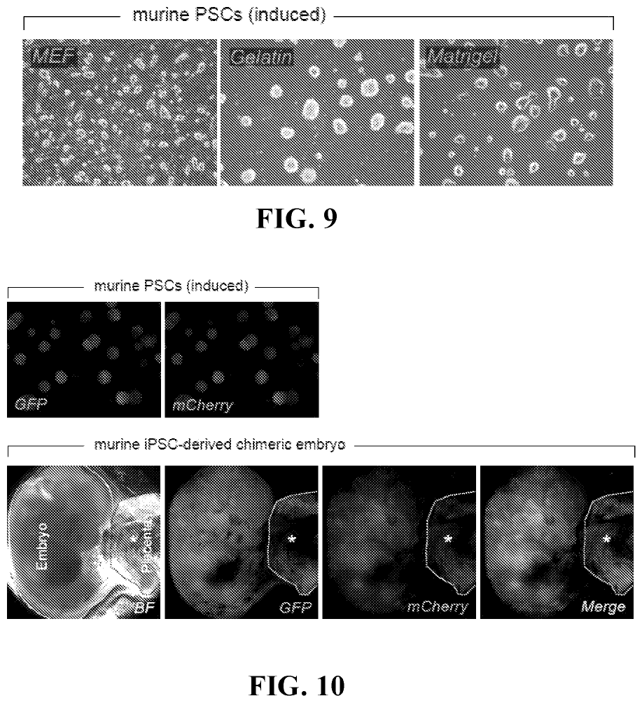

Murine induced pluripotent stem cells can be generated and cultured with the GMTi medium (FIG. 9) These cells also exhibit robust morphological characteristics of pluripotency. They sustain these characteristics even under feeder-free conditions such as surface coating with gelatin and Matrigel. Murine iPSCs generated and sustained using GMTi medium could contribute robustly to both embryonic and extraembryonic chimeras (FIG. 10). Stable expression of nuclear GFP and membrane-mCherry transgenes in murine iPSCs generated and cultured using GMTi medium. Images show miPSC colonies with GFP and mCherry fluorescence—dual transgene labeling. Injection of labeled miPSCs into murine blastocyst embryos resulted in incorporation of cells into both embryonic and extraembryonic (placental) tissues. GFP and mCherry transgene expression can be seen in both the embryo and placenta (asterisk; a dotted line has been used to demarcate bulk of the extraembryonic tissue).

### Example 3

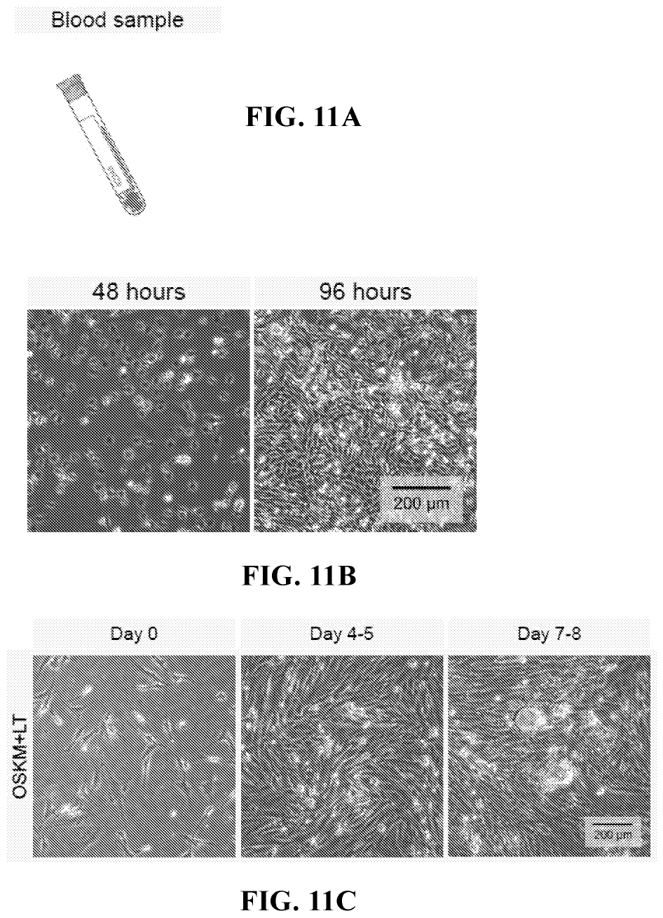

Adult blood-derived fibrocytes were derived and reprogrammed to induced pluripotent stem cells (iPSCs) (FIG. 11A-11C). Blood sample collected aseptically can be used to grow fibrocytes (FIG. 11A). FIG. 11B shows representative images showing attachment and growth of fibrocytes from a sheep blood sample. FIG. 11C shows representative images showing morphological changes during the reprogramming timeline using the OSKM+LT and culture conditions. Similar to fibroblasts, fibrocytes form compact iPSC colonies that contain rounded edges with individual cells not discernible.

### Example 4

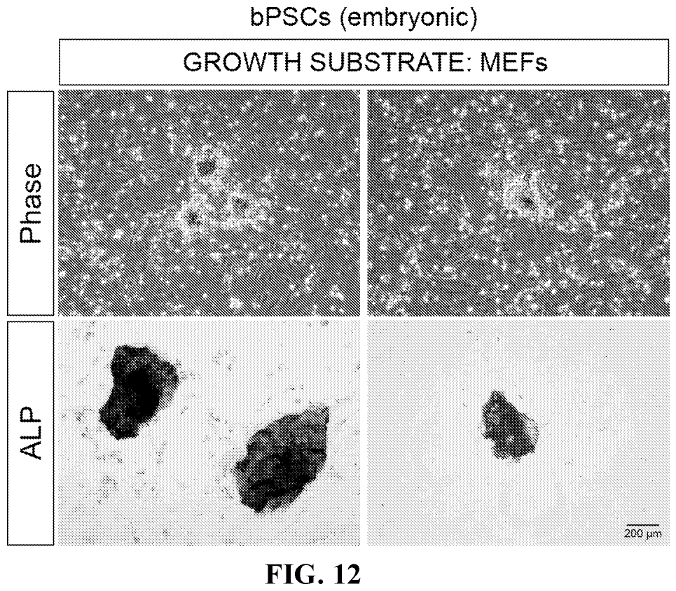

Bovine pluripotent stem cells were cultured on different growth substrates (FIG. 12).

### Example 5

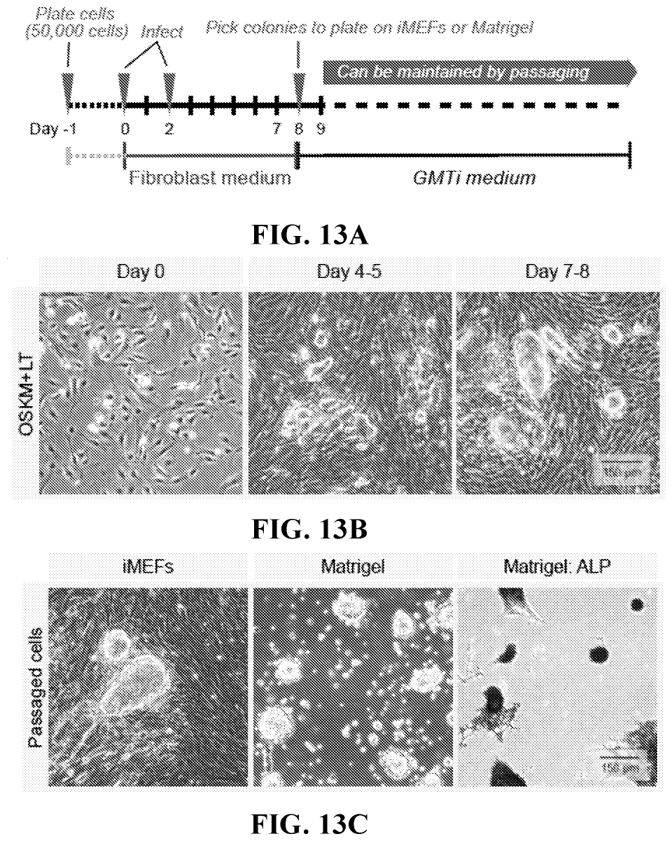

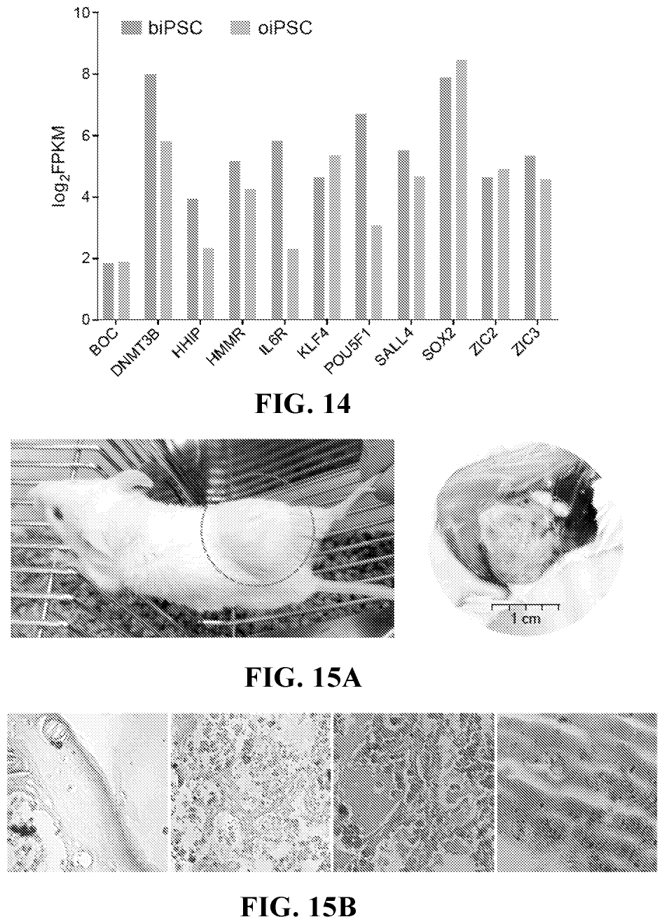

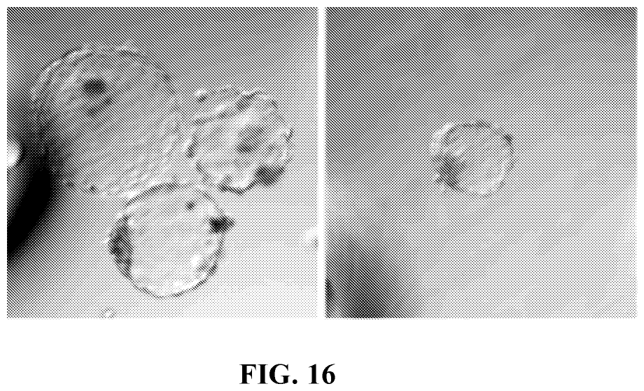

Sheep fibroblasts were reprogrammed to induced pluripotent stem cells (oiPSCs) using OSKM+LT. FIG. 13A shows the reprogramming method timeline showing procedures and culture conditions. FIG. 13B shows representative images showing morphological changes during the reprogramming timeline. Fibroblasts form compact colonies that contain rounded edges with individual cells not discernible. FIG. 13C shows representative images showing passaged oiPSCs on MEFs and Matrigel®. Compact colonies are positive for alkaline phosphatase even with repeated passages. Pluripotency gene expression in ovine iPSCs was evaluated. FIG. 14 shows selected expression of core pluripotency genes compiled in ovine iPSCs are similar to that observed in bovine iPSCs. Results presented are from mRNA-sequencing in both bovine and ovine iPSCs. Pluripotent ovine iPSCs readily differentiated to cells of the three germ layers (FIG. 15A-15B). FIG. 15A shows sub-cutaneous introduction of sheep oiPSCs in immunodeficient NSG mice resulted in teratoma formation, with significant growth observed by 6 weeks. Teratomas collected measured more than 1 cm in rough diameter. FIG. 15B shows representative images of hematoxylin and eosin-stained histological sections of teratomas showing differentiation of oiPSCs into the three different germ layers: [1] Epidermis (ectoderm), [2] Bone marrow (mesoderm), [3] Hyaline cartilage (mesoderm), [4] Lung alveoli (endoderm), and [5] Cardiac muscle (mesoderm).

### Example 6

This Example at least demonstrates the successful use of iPSCs for somatic cell nuclear transfer (SCNT/Cloning).

Somatic cell nuclear transfer/SCNT (also known as cloning) is a procedure that uses an enucleated egg that can accept a donor nucleus to produce an embryo that is genetically identical to the donor. Use of iPSCs for SCNT may produce a higher blastocyst success rate, and if transferred to recipient cows for carrying a pregnancy, higher live calving rate.

### Methods

The brief procedure for SCNT used is as below; the starting material is bovine oocytes that are collected from slaughterhouse embryos.

1. In vitro maturation of oocytes: Medium M199 with Earle's salts and glutamine is buffered with 2.2% (g/L) NaHCO₃, osmolarity is set at 280-290 mOsmol, and the medium is sterilized through a 0.22 um filter. The buffered medium is supplemented prior use with 10 IU/ml penicillin, 10 IU/ml streptomycin, 0.05 IU/ml recombinant hFSH, 0.1 μM cysteamine and 10 ng/ml EGF.

2. Removal of cumulus cells: Done approximately 17-18 hours after the onset of maturation; cumulus removal can be achieved by pipetting vigorously using a 200 μl micropipette.

3. Zona removal: Oocytes with a visible polar body are selected and exposed to 0.5% Pronase-E in M199H+0.01% PVA, with visual monitoring of the digestion. After zona removal oocytes are washed in M199H+10% fetal bovine serum/FBS and allowed to recover for 20 minutes.

4. Oocyte splitting and elimination of karyoplasts: Oocyte splitting is carried out using an ultrasharp splitting blade (Bioniche). Split oocytes into karyoplasts and cytoplasts. Collect the demi-oocytes after re-shaping—that do not contain the metaphase chromosomes (cytoplasts), by checking under a fluorescence microscope after exposure to Hoechst 33342 (nuclear stain).

5. Embryo reconstruction: Use two cytoplasts and roll them together for adherence and also transfer a bovine iPSC to the periphery. Fuse using a BTX453 fusion chamber.

6. Chemical activation: Remove the fused structures and rinse in M199H+10% FBS for 1-2 hours. Expose the reconstructed embryos to M199H-FCS+5 μM ionomycin for 4-5 min at room temperature. Then wash and incubate embryos in 5 μL individual drops in mSOFaa+2% FCS+0.3% BSA+2 mM 6-DMAP, under oil, at 39° C. for 3-6 h (ideal time: 4 h).

7. In vitro culture: Place embryos in mSOFaa medium for culture from this zygote to blastocyst stage.

This procedure is adapted mainly from: Ribeiro et al., Cloning Stem Cells 2009; 11:377-386; Gerger et al., Genetics and Molecular Research 2010; 9:295-302. For further information, also see Vajta et al., Cloning 2001; 3:89-95; Theriogenology 2002; 57:453; Biol Reprod 2003; 68:571-578; Reprod Fertil Dev 2004; 16:159; Handmade Cloning (HMC) Manual, by Vajta & Vajta, 2003.

### Results

When bovine skin fibroblasts and bovine iPSCs were used as nuclear donors for SCNT experiments, the cleavage rate and blastocyst rate observed are provided in Table 6. Higher rates were observed for bovine iPSCs for both cleavage and blastocyst rates. Skin fibroblasts resulted in an 89.3% cleavage rate compared to 91.2% for bovine iPSCs. Skin fibroblasts resulted in a 16.1% blastocyst rate compared to 35.3% for bovine iPSCs.

TABLE 6
biPSC-derived SCNT embryo development rate

| | | Cultured | | | |
|---|---|---|---|---|---|
| | | embryos | Cleavage (%) | Blastocysts (%) | |
| SCNT | Skin fibroblast | 56 | 50 (89.3) | 9 | (16.1) |
| | iPSC | 34 | 31 (91.2) | 12 | (35.3) |

FIG. 16 shows blastocyst embryos resulting from use of iPSCs as nuclear donors in SCNT procedure as described herein.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of maintaining pluripotency of and/or inhibit or prevent differentiation of a cell or cell population, the method comprising:
culturing a cell or cell population in the stem cell culture medium, the stem cell culture medium comprising:
an amount of a pluripotency composition comprising:
a glycogen synthase kinase 3 beta (GSK3beta) inhibitor;
a mitogen-activated protein kinase kinase (MEK 1/2) inhibitor; and
a transforming growth factor beta (TGF beta)/activin/nodal pathway inhibitor,
wherein the pluripotency composition is effective to maintain pluripotency and/or inhibit and/or prevent differentiation of a cell.

2. The method of claim 1, wherein the cell comprises a reprogrammed and/or a pluripotent stem cell signature and/or program.

3. The method of claim 2, wherein the reprogrammed and/or a pluripotent stem cell program comprises a TGFbeta signaling program.

4. The method of claim 3, wherein the reprogrammed and/or a pluripotent stem signature comprises: TGFbeta receptor 1, TGFbeta receptor 2, or both.

5. The method of claim 1, wherein the cell comprises a 16-cell embryo signature and/or program.

6. The method of claim 1, wherein the cell does not comprise
a. a trophectoderm cell signature and/or program;
b. a trophoblast stem cell signature and/or program;
c. a trophoblast signature and/or program;
d. an endoderm cell signature and/or program;
e. a mesoderm cell signature and/or program;
f. an ectoderm cell signature and/or program;
g. a differentiated cell signature or program; or
h. any combination thereof.

7. The method of claim 1, wherein the cell is a pluripotent stem cell, optionally an induced pluripotent stem cell.

8. The method of claim 1, wherein the cell is a reprogrammed cell, optionally an induced pluripotent stem cell, that was reprogrammed by expression of Oct4, Sox2, Klf4, Myc, SV40 Large T antigen, or any combination thereof, optionally Oct4, Sox2, Klf4, Myc, and SV40 Large T antigen.

9. The method of claim 1, wherein the cell is a non-human mammalian cell.

10. The method of claim 1, wherein the cell is a ruminant cell.

11. The method of claim 1, wherein the cell is a bovine cell, an ovine cell, a caprine cell, a cervine cell, a giraffe cell, or a camel cell.

12. The method of claim 1, wherein the cell is a murine cell, an equine cell, a feline cell, or a canine cell.

13. The method of claim 1, wherein the cell expresses or has expressed
a. SV40 Large T antigen;
b. Oct4;
c. Sox2;
d. Klf4;
e. Myc; or
f. any combination thereof

14. The method of claim 1, wherein the cell expresses or has expressed Oct4, Sox2, Klf4, Myc, SV40 Large T antigen, or any combination thereof, optionally Oct4, Sox2, Klf4, Myc, and SV40 Large T antigen.

15. The method of claim 1, further comprising an amount of leukemia inhibitory factor (LIF), an amount of interleukin (IL-6), or both.

16. The method of claim 1, wherein the stem cell culture medium comprises DMEM, MEM, Essential 6 medium, Neurobasal™, mTesR™, Leibovitz L-15, McCoy's 5A, or any combination thereof.

17. The method of claim 1, wherein the cell culture medium comprises Ham's F12 nutrient composition, Ham's F10 nutrient composition, serum, Knockout serum replacement, N2 supplement, B-27 supplement, one or more supplement nutrients, one or more anti-infectives), a reducing agent, a pH indicator, a buffer, or any combination thereof.

18. The method of claim 1, wherein the GSK3beta inhibitor is selected from: a small molecule inhibitor, an antibody or fragment thereof, an aptamer, an RNAi molecule, a polypeptide ligand or antagonist, or any combination thereof.

19. The method of claim 1, wherein the GSK3beta inhibitor is selected from: CHIR99021, SB-216763, GSK-3 inhibitor IX, kenpaullone or an analog thereof, lithium chloride, GSK-3beta inhibitor XII, GSK-3beta inhibitor VII, GSK-3inhibitor XVI, 10Z-hymenialdsine, indirubin, CHIR-98014, GSK-3beta inhibitor VI, indirubin-3′-monoxime, GSK-3 inhibitor X, SB-415268, TWS 119 ditrifluoroacetate, 5-iodo-indirubin-3′-monoxime, GSK-3beta inhibitor I, indirubin-5-sulfonic acid sodium salt, hymenidin, 3F8, Bisindlylmaleimide X HCl, indirubin-3′-monoxime-5-sulphonic acid, GSK-3 inhibitor II, GSK-3beta inhibitor VIII, GSK-3beta inhibitor XI, TCS 2002, alsterpaullone 2-cyanoethyl, A 1070722, MeBIO, AR-AO 14418-d3, 6-N-[2-[[4-(2,4-dichlorophenyl)-5-(1H-imidazol-2-yl)pyrimidin-2-yl]amino]ethyl]-3-nitropyridine-2,6-diamine, or any combination thereof.

20. The method of claim 1, wherein the MEK 1/2 inhibitor is selected from: a small molecule inhibitor, an antibody or fragment thereof, an aptamer, an RNAi molecule, a polypeptide ligand or antagonist, or any combination thereof.

21. The method of claim 1, wherein the MEK 1/2 inhibitor is selected from: PD0325901, AS703988/MSC2015103B, PD184352, Selumetinib, MEK162, AZD8330, TAK-733, GDC-0623, Refametinib, RO4987655, WX-554, HL-085, Cobimetinib, Trametinib, binimetinib, Pimasertib, Mirdametinib, E6201, CH5126766, SHR7390, TQ-B3234, CS-3006, FCN-159, RO5126766, or any combination thereof.

22. The method of claim 1, wherein the TGF beta/activin/nodal pathway inhibitor is selected from: a small molecule inhibitor, an antibody or fragment thereof, an aptamer, an RNAi molecule, a polypeptide ligand or antagonist, or any combination thereof.

23. The method of claim 1, wherein the TGF beta/activin/nodal pathway inhibitor is selected from: A83-01, SB431542, SB505124, LY2157299 (Galunisertib), LY550410, fresolimumab, AP12009, AP1104/AP15012, Lucanix™, ISTH0036, GC1008, 2G7, 1D11, LY2382770, CAT-192, Ki 26894, SD208, LY2109761, IN-1130, LY2157299, TEW-7197, PF-03446962, Gemogenovatucel-T, Trx-xFoxH1b aptamer, an inhibitor of myosins (e.g., pentachloropseudilin (PClP) and pentabromopseudilin (PBrP)), sorafenib, 6.3G9 antibody, 264RAD, lerdelimumab, soluble TbetaRII/III, 5,6-dihydro-4H-pyrrolo[1,2-b]pyrazole and analogs thereof, PF-03446962, KRC203, KRC360, LDN193189, ACE-041/Dalantercept, SB-525334, LY-364947, GW-6604, SD-208, or any combination thereof.

24. The method of claim 1, wherein the stem cell culture medium does not comprise a growth factor.

25. The method of claim 1, wherein culturing comprises passaging the cells one or more times.

26. The method of claim 1, wherein culturing does not comprise passaging.

27. The method of claim 1, wherein culturing comprises culturing cells on feeder layer

28. The method of claim 1, wherein culturing wherein culturing does not include growing cells on feeder layer.

29. The method of claim 1, wherein culturing comprises culturing the cells on a 3D matrix.

30. The method of claim 1, wherein culturing comprises culturing the cells in suspension.

31. The method of claim 1, wherein culturing comprises culturing the cells adherently on a cell culture surface.

32. A method of generating induced pluripotent stem cells, the method comprising:
reprogramming a somatic cell; and
culturing the reprogrammed somatic cell using a method as in any one of claims 1-31.

33. The method of claim 32, wherein reprogramming comprises expressing SV40 large T antigen, Oct4, Sox2, Klf4, and Myc in the somatic cell.

34. The method of claim 32, wherein the somatic cell is a non-human somatic cell.

35. The method of claim 32, wherein the somatic cell is a ruminant somatic cell.

36. The method of claim 32, wherein the somatic cell is a bovine somatic cell, an ovine somatic cell, a caprine somatic cell, a cervine somatic cell, a giraffe somatic cell, or a camel somatic cell.

37. The method of claim 32, wherein the somatic cell is a murine somatic cell, an equine somatic cell, a feline somatic cell, or a canine somatic cell.

38. A method comprising:
differentiating a cell generated by a method of any one of claims 32-37 and/or cultured by a method of any one of claims 1-31; and/or modifying a cell generated by a method of any one of claims 32-37 and/or the cell cultured by a method of any one of claims 1-31.

39. The method of claim 38, wherein modifying comprises genomic modification, RNA modification, or both.

40. A cell or cell population produced by a method according to any one of claims 1-39.

41. The cell or population thereof of claim 40, wherein the cell or population thereof is a reprogrammed cell or population thereof, a pluripotent stem cell or population thereof, an induced pluripotent stem cell or population thereof, or a differentiated cell or population thereof, a modified cell or population thereof, or any combination thereof.

42. A differentiated cell and/or modified cell produced by a method as in any one of claims 38-39.

43. The differentiated cell and/or modified cell of claim 42, wherein the differentiated cell and/or modified cell comprises one or more genetic modifications, RNA modifications, or both.

44. An engineered cell comprising:
a bovine induced pluripotent stem cell signature and/or program, wherein the bovine induced pluripotent stem cell signature and/or program comprises one or more genes and/or programs as set forth in one or more of FIGS. 2A-2E, 3B-3C, 3F-3G, 6, 7A-7D, 8A-8C and Tables 2-5.

45. The engineered cell of claim 44, wherein the engineered cell is a genetically edited or otherwise modified cell.

46. The engineered cell of claim 44, wherein the engineered cell is a non-human mammal cell.

47. The engineered cell of claim 44, wherein the engineered cell is a ruminant cell.

48. The engineered cell of claim 44, wherein the engineered cell is a bovine cell, ovine cell, caprine cell, a cervine cell, a giraffe cell, or a camel cell.

49. The engineered cell of claim 44, wherein the engineered cell is a murine cell, an equine cell, a feline cell, or a canine cell.

50. The engineered cell of claim 44, wherein the engineered cell is a pluripotent stem cell, optionally an induced pluripotent stem cell.

51. The engineered cell of claim 44, wherein the engineered cell expresses or has expressed SV40 large T antigen, Oct4, Sox2, Klf4, Myc, or any combination thereof, optionally SV40 large T antigen, Oct4, Sox2, Klf4, and Myc.

52. The engineered cell of claim 44, wherein the engineered cell is or has been cultured and/or generated via a method as in any one of claims 1-39.

53. A stem cell culture medium comprising:
an amount of a pluripotency composition comprising:
a glycogen synthase kinase 3 beta (GSK3beta) inhibitor;
a mitogen-activated protein kinase kinase (MEK 1/2) inhibitor; and
a transforming growth factor beta (TGF beta)/activin/nodal pathway inhibitor,
wherein the pluripotency composition is effective to maintain pluripotency and/or inhibit and/or prevent differentiation of a cell.

54. The stem cell culture medium of claim 53, wherein the cell comprises a reprogrammed and/or a pluripotent stem cell signature and/or program.

55. The stem cell culture medium of claim 54, wherein the reprogrammed and/or a pluripotent stem cell program comprises a TGFbeta signaling program.

56. The stem cell culture medium of claim 55, wherein the reprogrammed and/or a pluripotent stem signature comprises: TGFbeta receptor 1, TGFbeta receptor 2, or both.

57. The stem cell culture medium of claim 53, wherein the cell comprises a 16-cell embryo signature and/or program.

58. The stem cell culture medium of claim 53 wherein the cell does not comprise
a. a trophectoderm cell signature and/or program;
b. a trophoblast stem cell signature and/or program;
c. a trophoblast signature and/or program;
d. an endoderm cell signature and/or program;
e. a mesoderm cell signature and/or program;
f. an ectoderm cell signature and/or program;
g. a differentiated cell signature or program; or
h. any combination thereof.

59. The stem cell culture medium of claim 53, wherein the cell is a pluripotent stem cell, optionally an induced pluripotent stem cell.

60. The stem cell culture medium of claim 53, wherein the cell is a reprogrammed cell, optionally an induced pluripotent stem cell, that was reprogrammed by expression of Oct4, Sox2, Klf4, Myc, SV40 Large T antigen or any combination thereof, optionally Oct4, Sox2, Klf4, Myc, and SV40 Large T antigen.

61. The stem cell culture medium of claim 53, wherein the cell is a non-human mammalian cell.

62. The stem cell culture medium of claim 53, wherein the cell is a ruminant cell.

63. The stem cell culture medium of claim 53, wherein the cell is a bovine cell, an ovine cell, a caprine cell, a cervine cell, a giraffe cell, or a camel cell.

64. The stem cell culture medium of claim 53, wherein the cell is a murine cell, an equine cell, a feline cell, or a canine cell.

65. The stem cell culture medium of claim 53, wherein the cell expresses or has expressed
a. SV40 Large T antigen;
b. Oct4;
c. Sox2;
d. Klf4;
e. Myc; or
f. any combination thereof

66. The stem cell culture medium of claim 53, wherein the cell expresses or has expressed Oct4, Sox2, Klf4, Myc, SV40 Large T antigen or any combination thereof, optionally, SV40 Large T antigen, Oct4, Sox2, Klf4, and Myc.

67. The stem cell culture medium of claim 53, further comprising an amount of leukemia inhibitory factor (LIF), an amount of interleukin (IL-6), or both.

68. The stem cell culture medium of claim 53, wherein the stem cell culture medium comprises DMEM, MEM, Essential 6 medium, Neurobasal™, mTesR™, Leibovitz L-15, McCoy's 5A, or any combination thereof.

69. The stem cell culture medium of claim 53, wherein the cell culture medium comprises Ham's F12 nutrient composition, Ham's F10 nutrient composition, serum, Knockout serum replacement, N2 supplement, B-27 supplement, one or more supplement nutrients, one or more anti-infectives), a reducing agent, a pH indicator, a buffer, or any combination thereof.

70. The stem cell culture medium of claim 53, wherein the GSK3beta inhibitor is selected from: a small molecule inhibitor, an antibody or fragment thereof, an aptamer, an RNAi molecule, a polypeptide ligand or antagonist, or any combination thereof.

71. The stem cell culture medium of claim 53, wherein the GSK3beta inhibitor is selected from: CHIR99021, SB-216763, GSK-3 inhibitor IX, kenpaullone or an analog thereof, lithium chloride, GSK-3beta inhibitor XII, GSK-3beta inhibitor VII, GSK-3inhibitor XVI, 10Z-hymenialdsine, indirubin, CHIR-98014, GSK-3beta inhibitor VI, indirubin-3′-monoxime, GSK-3 inhibitor X, SB-415268, TWS 119 ditrifluoroacetate, 5-iodo-indirubin-3′-monoxime, GSK-3beta inhibitor I, indirubin-5-sulfonic acid sodium salt, hymenidin, 3F8, Bisindlylmaleimide X HCl, indirubin-3′-monoxime-5-sulphonic acid, GSK-3 inhibitor II, GSK-3beta inhibitor VIII, GSK-3beta inhibitor XI, TCS 2002, alsterpaullone 2-cyanoethyl, A 1070722, MeBIO, AR-AO 14418-d3, 6-N-[2-[[4-(2,4-dichlorophenyl)-5-(1H-imidazol-2-yl)pyrimidin-2-yl]amino]ethyl]-3-nitropyridine-2,6-diamine, or any combination thereof.

72. The stem cell culture medium of claim 53, wherein the MEK 1/2 inhibitor is selected from: a small molecule inhibitor, an antibody or fragment thereof, an aptamer, an RNAi molecule, a polypeptide ligand or antagonist, or any combination thereof.

73. The stem cell culture medium of claim 53, wherein the MEK 1/2 inhibitor is selected from: PD0325901, AS703988/MSC2015103B, PD184352, Selumetinib, MEK162, AZD8330, TAK-733, GDC-0623, Refametinib, RO4987655, WX-554, HL-085, Cobimetinib, Trametinib, binimetinib, Pimasertib, Mirdametinib, E6201, CH5126766, SHR7390, TQ-B3234, CS-3006, FCN-159, RO5126766, or any combination thereof.

74. The stem cell culture medium of claim 53, wherein the TGF beta/activin/nodal pathway inhibitor is selected from: a small molecule inhibitor, an antibody or fragment thereof, an aptamer, an RNAi molecule, a polypeptide ligand or antagonist, or any combination thereof.

75. The stem cell culture medium of claim 53, wherein the TGF beta/activin/nodal pathway inhibitor is selected from: A83-01, SB431542, SB505124, LY2157299 (Galunisertib), LY550410, fresolimumab, AP12009, AP1104/AP15012, Lucanix™, ISTH0036, GC1008, 2G7, 1D11, LY2382770, CAT-192, Ki 26894, SD208, LY2109761, IN-1130, LY2157299, TEW-7197, PF-03446962, Gemogenovatucel-T, Trx-xFoxH1b aptamer, an inhibitor of myosins (e.g., pentachloropseudilin (PClP) and pentabromopseudilin (PBrP)), sorafenib, 6.3G9 antibody, 264RAD, lerdelimumab, soluble TbetaRII/III, 5,6-dihydro-4H-pyrrolo[1,2-b]pyrazole and analogs thereof, PF-03446962, KRC203, KRC360, LDN193189, ACE-041/Dalantercept, SB-525334, LY-364947, GW-6604, SD-208, or any combination thereof.

76. The stem cell culture medium of claim 53, wherein the stem cell culture medium does not comprise a growth factor.


SEQUENCE LISTING (4 sequences)

SEQ ID NO: 1 - DNA, 21 bp, synthetic construct
  source 1..21: mol_type = other DNA
  misc_feature 1..21: note = Primer
ttcacatgtcccagcactacc

SEQ ID NO: 2 - DNA, 20 bp, synthetic construct
  source 1..20: mol_type = other DNA
  misc_feature 1..20: note = Primer
gaagccgctccacatacagt

SEQ ID NO: 3 - DNA, 20 bp, synthetic construct
  source 1..20: mol_type = other DNA
  misc_feature 1..20: note = Primer
ggctacactgtttgttgccc

SEQ ID NO: 4 - DNA, 20 bp, synthetic construct
  source 1..20: mol_type = other DNA
  misc_feature 1..20: note = Primer
gcctgcagtgttttaggcac